SEQUENCE LISTING (113 sequences)

SEQ ID NO: 1 - DNA, 471 bp, Artificial Sequence
  SLAMF6 Affymetrix annotation
tagcattacccttctgacactctctatgtagcctccctgatcttctttcagctcctctattaaaggaaaagttctttatgttaattatttacatcttcctgcaggcccttcctctgcctgctggggtcctcctattctttaggtttaattttaaatatgtcacctcctaagagaaaccttcccagaccactctttctaaaatgaatcttctaggctgggcatggtggctcacacctgtaatcccagtactttgggaggccaaggggggagatcacttgaggtcaggagttcaagaccagcctggccaacttggtgaaaccccgtctttactaaaaatacaaaaaaattagccaggcgtggtggtgcacccctaaaatcccagctacttgagagactgaggcaggagaatcgcttgaacccaggaggtggaggttccagtgagccaaaatcatgccaatgtattccagtctg

SEQ ID NO: 2 - DNA, 55 bp, Artificial Sequence
  CDC42SE2 Affymetrix annotation
tgttctgctctgaagaagatactgtcagacgaatcctgcatttccttcagctggc

SEQ ID NO: 3 - DNA, 507 bp, Artificial Sequence
  misc_feature 33, 95, 161, 165, 166, 167, 183, 184, 199, 210, 211,213, 239, 240, 267, 278, 282, 286, 289, 290, 294, 333, 395, 419,432, 464: n = A,T,C or G
  misc_feature 33, 95, 161, 165, 166, 167, 183, 184, 199, 210, 211,213, 239, 240, 267, 278, 282, 286, 289, 290, 294, 333, 395, 419,432, 464: n = A,T,C or G
  CDC42SE2 Affymetrix annotation
  misc_feature 33, 95, 161, 165, 166, 167, 183, 184, 199, 210, 211,213, 239, 240, 267, 278, 282, 286, 289, 290, 294, 333, 395, 419,432, 464: n = A,T,C or G
gcatgcctttggactcatggacagagttctttnggattgtcactgaattttcaatgtttaatcagtatggatctgatcttcgcatgatctttttngtgaatgctaacaccattttgcagttttttttttctattttaaacatttttcttttcactgccgancccnnngccttacgattttatnnggaaagcaaggaccntgctattattnntntaatttgccatcatttatgtatattnnggaaggtatgagacccacaagcacaantgatcattttnattngttngtnngttngaaacttcagcagaatagatatctgcatgctttatgaangttgttgcttcggtaagagcccatgggatgccagaaattaacatttctttgctgccatgggntgatgatgctgctattagataaangtttagctgtggnaccaagtcacatcattttcatagaaaaagatnacttgtagcttattttagaagtatgaccttttggtctgtttga

SEQ ID NO: 4 - DNA, 486 bp, Artificial Sequence
  misc_feature 373: n = A,T,C or G
  misc_feature 373: n = A,T,C or G
  TC2N Affymetrix annotation
  misc_feature 373: n = A,T,C or G
caggtggcacaaattaaatccatcttgaagacttcacacattaatttggtgaagaacttgacattcttttagaagacttatgatttcaatttgctaccaatgagaagaggcaaatcaacaaatttgtcaatttatgggggctataattatggtatataatgtatctgatagaaaatttgataagaaaatgtaatgaattttatcagatatccaaagtaaaggaaatgttttaaaactgcaacaagagacacagacagtaaaatcaaagtattattaggatgactaaataaattataaagtctgtgagaatatcaaccatagatagttctttctatattatgtttttgcttttgtattttaagctttacttagnatattcaaaacctggtatatcaagtctctgttagtactattggcatttagaagactttaccattatttcagtgctaggcattattgattaggtcttggctccactgtttacct

SEQ ID NO: 5 - DNA, 239 bp, Artificial Sequence
  ITGAL Affymetrix annotation
acacttggttgggtcctcacatctttcacacttccaccagcctgcactactccctcaaagcacacgtcatgtttcttcatccggcagcctggatgttttttccctgtttaatgattgacgtacttagcagctatctctcagtgaactgtgagggtaaaggctatacttgtcttgttcaccttgggatgatgcctcatgatatgtcagggcgtgggacatctagtaggtgcttgacataa

SEQ ID NO: 6 - DNA, 128 bp, Artificial Sequence
  CCL5 Affymetrix annotation
cccgtgcccacatcaaggagtatttctacaccagtggcaagtgctccaacccagcagtcgtctttgtcacccgaaagaaccgccaagtgtgtgccaacccagagaagaaatgggttcgggagtacatc

SEQ ID NO: 7 - DNA, 354 bp, Artificial Sequence
  misc_feature 152, 157, 169: n = A,T,C or G
  PSMB9 R2.9 annotation
  misc_feature 152, 157, 169: n = A,T,C or G
ccattctgagtacttctccgcaaaccctttgtttcattaaggactgttttacatgaagggtgcaaaagtaggataaaaatgagaaccctagggtgaaacacgtgacagaagaataaagactattgaatagtcctcttctctacccatggacnttggnatttttatattngattttaaggaaatataacttagtagtaaagagatgagcattcaagtcaggcagacctgaatttgggtcaaggctgcgccactcaaaagctatatgacctctatatgagcagcttattcaacctcttttaacctccattttgtcatctgtagaatgatgataaatgcctagctcagaaggattcc

SEQ ID NO: 8 - DNA, 206 bp, Artificial Sequence
  JAK2 Affymetrix annotation
atgttcactgtatgtgccaagcctaatatgagagctatgtattatagagtttatgctacagccctaccttcaggaaacttatctactggacaaacaaaaattttcaaatatacaaaaaattctaaatcgaacattgtaattatctagcataggcaaatatagacagtaacagacaggtttacaattattaagaaagggcagccagg

SEQ ID NO: 9 - DNA, 391 bp, Artificial Sequence
  LOC284757 Affymetrix  annotation
atcgaggaagatatactgccaagtcaggaagaaaaaatccacctgttcagtgatttcaggaactgctgaagaaaatcaccagtgagtatcagtttctgcaagagaatctaatgcaggctttgcttctcatcggaatcccccagctggtgtcttggttgactgagagtctgggggagagggcagagaatggatttattctctgctaggtttttaacagtcaagaagggctgtggtcctaaggggcactggtcaaaccttagtgtgcatcagaattatctggataaggctaggcacagtggctcacgcctgtaatcacagcactttgggaggctgaggcgcgtggatcacctgaggtcagaagttcaagaccagcctggctcttttagtagag

SEQ ID NO: 10 - DNA, 500 bp, Artificial Sequence
  PPP1R16B Annotation from R2.6 that became NA in R2.9
gaaaattcctggcagtttcaactgtgatagacattgctaacctgttctccaaagaggctgaaccaatttctgtttcctcaacagtgtatgactgtttcccccatctattctccagcactgaggattaagtaactttcatttttgtcagtctgacagatataaagcagaacatttctgcataaggttctacagtaatttttagattttatgaccctttggattatgcctacataatgatgatcaaatattcagaaactacattgtacctggccttaggcttggaattggatacaaaattaaatgaaaccagcttttgccctcaggttgatcccatctcctggagttggcagacaaatgaacaaataaaatgagagcaaaactgtatggttcacattgtgctagagaaatgcataagcttagctaacttttgtttgataaactctatattcattaatatcacaaatgaattcataaaataccgtatgcattatgtcccaggg

SEQ ID NO: 11 - DNA, 462 bp, Artificial Sequence
  AP2B1 Affymetrix annotation
gggcaggacatgctgtaccaatccctgaagctcactaatggcatttggattttggccgaactacgtatccagccaggaaaccccaattacacgctgtcactgaagtgtagagctcctgaagtctctcaatacatctatcaggtctacgacagcattttgaaaaactaacaagactggtccagtacccttcaaccatgctgtgatcggtgcaagtcaagaactcttaactggaagaaattgtattgctgcgtagaatctgaacacactgaggccacctagcaaggtagtaactagtctaacctgtgctaacattagggcacaacctgttggatagttttagcttcctgtgaacatttgtaaccactgcttcagtcacctcccacctcttgccacctgctgctgctatctgtccttacttgtgggcttctccatgctgtgccaatggctggctttttctacacc

SEQ ID NO: 12 - DNA, 432 bp, Artificial Sequence
  ITGA3 Affymetrix annotation
gccacagactgaactcgcagggagtgcagcaggaaggaacaaagacaggcaaacggcaacgtagcctgggctcactgtgctggggcatggcgggatcctccacagagaggaggggaccaattctggacagacagatgttgggaggatacagaggagatgccacttctcactcaccactaccagccagcctccagaaggccccagagagaccctgcaagaccacggagggagccgacacttgaatgtagtaataggcagggggccctgccaccccatccagccagaccccagctgaaccatgcgtcaggggcctagaggtggagttcttagctatccttggctttctgtgccagcctggctctgcccctcccccatgggctgtgtcctaaggcccatttgagaagctgaggctagttccaaaaacctctcctg

SEQ ID NO: 13 - DNA, 502 bp, Artificial Sequence
  IRF1 Affymetri x annotation
acaggagtcagtgtctggctttttcctctgagcccagctgcctggagagggtctcgctgtcactggctggctcctaggggaacagaccagtgaccccagaaaagcataacaccaatcccagggctggctctgcactaagcgaaaattgcactaaatgaatctcgttccaaagaactaccccttttcagctgagccctggggactgttccaaagccagtgaatgtgaaggaaactcccctccttcggggcaatgctccctcagcctcagaggagctctaccctgctccctgctttggctgaggggcttgggaaaaaaacttggcactttttcgtgtggatcttgccacatttctgatcagaggtgtacactaacatttcccccgagctcttggcctttgcatttatttatacagtgccttgctcggggcccaccaccccctcaagccccagcagccctcaacaggcccagggagggaagtgtgagcgccttggtatgacttaa

SEQ ID NO: 14 - DNA, 521 bp, Artificial Sequence
  TNFAIP3 Affymetrix annotation
tctttgggttattactgtctttacttctaaagaagttagcttgaactgaggagtaaaagtgtgtacatatataatatacccttacattatgtatgagggatttttttaaattatattgaaatgctgccctagaagtacaataggaaggctaaataataataacctgttttctggttgttgttggggcatgagcttgtgtatacactgcttgcataaactcaaccagctgcctttttaaagggagctctagtcctttttgtgtaattcactttatttattttattacaaacttcaagattatttaagtgaagatatttcttcagctctggggaaaatgccacagtgttctcctgagagaacatccttgctttgagtcaggctgtgggcaagttcctgaccacagggagtaaattggcctctttgatacacttttgcttgcctccccaggaaagaaggaattgcatccaaggtatacatacatattcatcgatgtttcgtgcttctccttatgaaactccagc

SEQ ID NO: 15 - DNA, 502 bp, Artificial Sequence
  TNFAIP3 Affymetrix annotation
catcccatggtaccctggtattgggacagcaaaagccagtaaccatgagtatgaggaaatctctttctgttgctggcttacagtttctctgtgtgctttgtggttgctgtcatatttgctctagaagaaaaaaaaaaaaggaggggaaatgcattttccccagagataaaggctgccattttgggggtctgtacttatggcctgaaaatatttgtgatccataactctacacagcctttactcatactattaggcacactttccccttagagccccctaagtttttcccagacgaatctttataatttcctttccaaagataccaaataaacttcagtgttttcatctaattctcttaaagttgatatcttaatattttgtgttgatcattatttccattcttaatgtgaaaaaaagtaattatttatacttattataaaaagtatttgaaatttgcacatttaattgtccctaatagaaagccacctattctttgttggat

SEQ ID NO: 16 - DNA, 511 bp, Artificial Sequence
  PSMB10 Affymetrix annotation
tacacgcgttatctacgggccgcgagccccgcgtggccacggtcactcgcatcctgcgccagacgctcttcaggtaccagggccacgtgggtgcatcgctgatcgtgggcggcgtagacctgactggaccgcagctctacggcgtgcatccccatggctcctacagccgtctgcccttcacagccctgggctctggtcaggacgcggccctggcggtgctagaagaccggttccagccgaacatgacgctggaggctgctcaggggctgctggtggaagccgtcaccgccgggatcttgggtgacctgggctccgggggcaatgtggacgcatgtgtgatcacaaagactggcgccaagctgctgcggacactgagctcacccacagagcccgtgaagaggtctggccgctaccactttgtgcctggaaccacagctgtcctgacccagacagtgaagccactaaccctggagctagtggaggaaactgtgcaggctatggaggtggagta

SEQ ID NO: 17 - DNA, 367 bp, Artificial Sequence
  CXCL9 CXCL9 Affymetrix annotation
gattatcaattaccacaccatctcccatgaagaaagggaacggtgaagtactaagcgctagaggaagcagccaagtcggttagtggaagcatgattggtgcccagttagcctctgcaggatgtggaaacctccttccaggggaggttcagtgaattgtgtaggagaggttgtctgtggccagaatttaaacctatactcactttcccaaattgaatcactgctcacactgctgatgatttagagtgctgtccggtggagatcccacccgaacgtcttatctaatcatgaaactccctagttccttcatgtaacttccctgaaaaatctaagtgtttcataaatttgagagtctgtgacccacttacc

SEQ ID NO: 18 - DNA, 358 bp, Artificial Sequence
  RARRES3 Affymetrix annotation
gaaacgggggcgcctggaagatgtggtgggaggctgttgctatcgggtcaacaacagcttggaccatgagtaccaaccacggcccgtggaggtgatcatcagttctgcgaaggagatggttggtcagaagatgaagtacagtattgtgagcaggaactgtgagcactttgtcgcccagctgagatatggcaagtcccgctgtaaacaggtggaaaaggccaaggttgaagtcggtgtggccacggcgcttggaatcctggttgttgctggatgctcttttgcgattaggagataccaaaaaaaagcaacagcctgaagcagccacaaaatcctgtgttagaagcagctgtgggggtcc

SEQ ID NO: 19 - DNA, 411 bp, Artificial Sequence
  IL2RG Affymetrix annotation
ttctggctggaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctgaaccccaatcctctgacagaagaaccccagggtcctgtagccctaagtggtactaactttccttcattcaacccacctgcgtctcatactcacctcaccccactgtggctgatttggaattttgtgcccccatgtaagcacc

SEQ ID NO: 20 - DNA, 464 bp, Artificial Sequence
  GCH1 Affymetrix annotation
gtgatggttggcttgagtacctttttaaatctagcccagtataaacattagcctgcttaatatttagacatttataggtagaattctgagcactcaactcatgtttggcattttaaagtaaaaacaagtgtgacttcgaggaccaaagaaattgtcagctatacatttatctttatgaactcatttatattcctttttaatgactcgttgttctaacatttcctagaagtgttcttataaaggtctaatgtatccacaggctgttgtcttattagtaaatgcaaagtaatgactttgtctgttttactctagtctttagtacttcaaaattaccttttcatatccatgatcttgagtccatttgggggatttttaagaatttgatgtatttcaatacactgttcaaaattaaattgtttaattttatgtatgagtatgtatgttcctgaagttggtcctattta

SEQ ID NO: 21 - DNA, 551 bp, Artificial Sequence
  TOX Affymetrix annotation
atggcttgatgtagcagtcatagcaagtttgtaaatagcatctatgttacactctcctagagtataaaatgtgaatgtttttgtagctaaattgtaattgaaactggctcattccagtttattgatttcacaataggggttaaattggcaaacattcatatttttacttcatttttaaaacaactgactgatagttctatattttcaaaatatttgaaaataaaaagtattcccaagtgattttaatttaaaaacaaattggctttgtctcattgatcagacaaaaagaaactagtattaagggaagcgcaaacacatttattttgtactgcagaaaaattgcttttttgtatcactttttgtgtaatggttagtaaatgtcatttaagtccttttatgtataaaactgccaaatgcttacctggtattttattagatgcagaaacagattggaaacagctaaattacaacttttacatatggctctgtcttattgtttcttcatactgtgtctgtatttaatctttttttatggaacctgttgcgcctat

SEQ ID NO: 22 - DNA, 544 bp, Artificial Sequence
  CXCL10 Affymetrix annotation
taactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtataagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaataaaaatgaggtactctcctggaaatatt

SEQ ID NO: 23 - DNA, 548 bp, Artificial Sequence
  DZIP1 Affymetrix annotation
ggaactaatgtccctgagatgtttatcaaaaaagaagaattacaagaactaaagtgtgcggatgtggaggatgaagactgggacatatcatccctagaggaagagatatctttgggaaaaaaatctgggaaagaacagaaggaacctccacctgcgaaaaatgaaccacattttgctcatgtgctaaatgcctggggcgcatttaatcctaaggggccaaagggagaaggacttcaagaaaatgaatcaagcacattaaaaagcagcttagtaactgtgactgattggagcgacacttcagatgtctaattccacatgtcagaagattattccagaagccagcagtatttcagtatcacagtgtttcagtaatttgcctccatgattctagtgcttctgccttaccgtgtttcccacagcaacacagagactgattcaaagaacaatggtctctttaatggcacccaatacagtattgaaaatcagatcatcaacagtatttcgaagcatgtaaaggtgtttaagacttccgctgctgcttaaaaata

SEQ ID NO: 24 - DNA, 503 bp, Artificial Sequence
  HLA-F Affymetrix annotation
cagatcctccaaaggcacacgttgcccaccaccccatctctgaccatgaggccaccctgaggtgctgggccctgggcttctaccctgcggagatcacgctgacctggcagcgggatggggaggaacagacccaggacacagagcttgtggagaccaggcctgcaggggatggaaccttccagaagtgggccgctgtggtggtgccttctggagaggaacagagatacacatgccatgtgcagcacgaggggctgccccagcccctcatcctgagatgggagcagtctccccagcccaccatccccatcgtgggcatcgttgctggccttgttgtccttggagctgtggtcactggagctgtggtcgctgctgtgatgtggaggaagaagagctcagatagaaacagagggagctactctcaggctgcagtcactgacagtgcccagggctctggggtgtctctcacagctaataaagtgtgagacagcttccttgtgtgggac

SEQ ID NO: 25 - DNA, 339 bp, Artificial Sequence
  PTGER4 Affymetrix annotation
agcagcttattgtttctctgaaagtgtgtgtagttttactttcctaaggaattaccaagaatatcctttaaaatttaaaaggatggcaagttgcatcagaaagctttattttgagatgtaaaaagattcccaaacgtggttacattagccattcatgtatgtcagaagtgcagaattggggcacttaatggtcaccttgtaacagttttgtgtaactcccagtgatgctgtacacatatttgaagggtctttctcaaagaaatattaagcatgttttgttgctcagtgtttttgtgaattgcttggttgtaattaaattctgagcctgatattgatatg

SEQ ID NO: 26 - DNA, 402 bp, Artificial Sequence
  misc_feature 353: n = A,T,C or G
  misc_feature 353: n = A,T,C or G
  SLC26A2 Affymetrix annotation
  misc_feature 353: n = A,T,C or G
tactcatgcctttttgtttaggataaataggtaagcacaaagagctcttcaaaatcagaaaaaacaataggagtccttccttgtcttttctgtgatctctgtccttgtttctgagactttctctaccattaagctctattttagctttcagttattctagtttgtttcccatggaatctgtcctaaactggtgtttttgtcagtgacagtcttgccagtcagcaatttctaacagcattttaaatgagtttgatgtacagtaaatattgatgacaatgacagcttttaactcttcaagtcacctaaagctattatgcaggaggatttagaagtcacattcataaaacccaagngctatgggtgtattattcatgatagctggcccacaggtcatgaattgag

SEQ ID NO: 27 - DNA, 503 bp, Artificial Sequence
  SRPX2 Affymetrix annotation
gcggcatgtgaccatcattgaactggtgggacagccacctcaggaggtggggcgcatccgggagcaacagctgtcagccaacatcatcgaggagctcaggcaatttcagcgcctcactcgctcctacttcaacatggtgttgattgacaagcagggtattgaccgagaccgctacatggaacctgtcacccccgaggaaatcttcacattcattgatgactacctactgagcaatcaggagttgacccagcgtcgggagcaaagggacatatgcgagtgaacttgagccagggcatggttaaagtcaagggaaaagctcctctagttagctgaaactgggacctaataaaaggaggaaatgttttcccacagttctagggacaggactctgaggtgggtgagtttgacaaatcctgcagtgtttccaggcatccttttaggactgtgtaatagtttccctagaagctaggtagggactgaggacaggccttgggcagtgggtt

SEQ ID NO: 28 - DNA, 446 bp, Artificial Sequence
  CD86 Affymetrix annotation
gaaggaggcttaggactttccactcctggctgagagaggaagagctgcaacggaattaggaagaccaagacacagatcacccggggcttacttagcctacagatgtcctacgggaacgtgggctggcccagcatagggctagcaaatttgagttggatgattgtttttgctcaaggcaaccagaggaaacttgcatacagagacagatatactgggagaaatgactttgaaaacctggctctaaggtgggatcactaagggatggggcagtctctgcccaaacataaagagaactctggggagcctgagccacaaaaatgttcctttattttatgtaaaccctcaagggttatagactgccatgctagacaagcttgtccatgtaatattcccatgtttttaccctgcccctgccttgattagactcctagcacctggctagtttc

SEQ ID NO: 29 - DNA, 436 bp, Artificial Sequence
  misc_feature 199: n = A,T,C or G
  misc_feature 199: n = A,T,C or G
  CD8A Affymetrix annotation
  misc_feature 199: n = A,T,C or G
cagcccttgcattgcagaggggcccatgaaagaggacaggctacccctttacaaatagaatttgagcatcagtgaggttaaactaaggccctcttgaatctctgaatttgagatacaaacatgttcctgggatcactgatgactttttatactttgtaaagacaattgttggagagcccctcacacagccctggcctcngctcaactagcagatacagggatgaggcagacctgactctcttaaggaggctgagagcccaaactgctgtcccaaacatgcacttccttgcttaaggtatggtacaagcaatgcctgcccattggagagaaaaaacttaagtagataaggaaataagaaccactcataattcttcaccttaggaataatctcctgttaatatggtgtacattcttcctgattattttctacacatac

SEQ ID NO: 30 - DNA, 508 bp, Artificial Sequence
  GABBR1 /// UBD Affymetrix annotation
gatcttaaagccacggagaagcctctcatcttatggcattgacaaagagaagaccatccaccttaccctgaaagtggtgaagcccagtgatgaggagctgcccttgtttcttgtggagtcaggtgatgaggcaaagaggcacctcctccaggtgcgaaggtccagctcagtggcacaagtgaaagcaatgatcgagactaagacgggtataatccctgagacccagattgtgacttgcaatggaaagagactggaagatgggaagatgatggcagattacggcatcagaaagggcaacttactcttcctggcatcttattgtattggagggtgaccaccctggggatggggtgttggcaggggtcaaaaagcttatttcttttaatctcttactcaacgaacacatcttctgatgatttcccaaaattaatgagaatgagatgagtagagtaagatttgggtgggatgggtaggatgaagtatattgcccaactctatgtttctttga

SEQ ID NO: 31 - DNA, 473 bp, Artificial Sequence
  HCP5 Affymetrix Annotation
tgaaggatggtgactgcgccatggcctggatctgctgcagtgtcctttcctgtggaggctccactcaaagctggcatcctcctatgtcacctagagtgtgggtcaaagcaatacacctacatgtagaatgtgatgtcagaactcaaacaggctcaccaggcagtgtgcttcttccttgcatgaggatgcaagatgcaacagtttgtcttcacattggaaggacacccctggatgcccctaaccactagacctgtaaaacttcactgcagtggccacttctgaatctctgtaaggtttatttatcttcacccctctggagagaagatgttttaccaaagcctctagtgtaccgtcctcctcttactcatccatcccagtcaacatgatgttgtcaatgaaataaaggaatttaatattctatagtatatccaggttctccagatctcttaagactgtactatagaggcctgggg

SEQ ID NO: 32 - DNA, 489 bp, Artificial Sequence
  GZMK Affymetrix annotation
aaacctctcttagatctggaaccaaatgcaaggttactggctggggagccaccgatccagattcattaagaccttctgacaccctgcgagaagtcactgttactgtcctaagtcgaaaactttgcaacagccaaagttactacaacggcgacccttttatcaccaaagacatggtctgtgcaggagatgccaaaggccagaaggattcctgtaagggtgactcagggggccccttgatctgtaaaggtgtcttccacgctatagtctctggaggtcatgaatgtggtgttgccacaaagcctggaatctacaccctgttaaccaagaaataccagacttggatcaaaagcaaccttgtcccgcctcatacaaattaagttacaaataattttattggatgcacttgcttcttttttcctaatatgctcgcaggttagagttgggtgtaagtaaagcagagcacatatggggtccatttttgcacttgta

SEQ ID NO: 33 - DNA, 556 bp, Artificial Sequence
  TNFRSF9 Affymetrix annotation
agaccagtacaaactactcaagaggaagatggctgtagctgccgatttccagaagaagaagaaggaggatgtgaactgtgaaatggaagtcaatagggctgttgggactttcttgaaaagaagcaaggaaatatgagtcatccgctatcacagctttcaaaagcaagaacaccatcctacataatacccaggattcccccaacacacgttcttttctaaatgccaatgagttggcctttaaaaatgcaccactttttttttttttttggacagggtctcactctgtcacccaggctggagtgcagtggcaccaccatggctctctgcagccttgacctctgggagctcaagtgatcctcctgcctcagtctcctgagtagctggaactacaaggaagggccaccacacctgactaacttttttgttttttgttggtaaagatggcatttcgccatgttgtacaggctggtctcaaactcctaggttcactttggcctcccaaagtgctgggattacagacatgaactgccaggcccggccaaaataatgcaccact

SEQ ID NO: 34 - DNA, 405 bp, Artificial Sequence
  GPR171 Affymetrix annotation
ttgccttgtaattcgacagctctacagaaacaaagataatgaaaattacccaaatgtgaaaaaggctctcatcaacatacttttagtgaccacgggctacatcatatgctttgttccttaccacattgtccgaatcccgtataccctcagccagacagaagtcataactgattgctcaaccaggatttcactcttcaaagccaaagaggctacactgctcctggctgtgtcgaacctgtgctttgatcctatcctgtactatcacctctcaaaagcattccgctcaaaggtcactgagacttttgcctcacctaaagagaccaaggctcagaaagaaaaattaagatgtgaaaataatgcataaaagacaggattttttgtgctaccaattctggccttactgga

SEQ ID NO: 35 - DNA, 372 bp, Artificial Sequence
  KLRD1 Affymetrix annotation
ttctctacttcgctcttggaacataatttctcatggcagcttttactaaactgagtattgagccagcatttactccaggacccaacatagaactccagaaagactctgactgctgttcttgccaagaaaaatgggttgggtaccggtgcaactgttacttcatttccagtgaacagaaaacttggaacgaaagtcggcatctctgtgcttctcagaaatccagcctgcttcagcttcaaaacacagatgaactggattttatgagctccagtcaacaattttactggattggactctcttacagtgaggagcacaccgcctggttgtgggagaatggctctgcactctcccagtatctatttccatcatttg

SEQ ID NO: 36 - DNA, 517 bp, Artificial Sequence
  HLA-B Affymetrix annotation
gtggcggagcagctgagagcctacctggagggcgagtgcgtggagtggctccgcagatacctggagaacgggaaggagacgctgcagcgcgcggaccccccaaagacacacgtgacccaccaccccatctctgaccatgaggccaccctgaggtgctgggccctgggcttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagccgtcttcccagtccaccgtccccatcgtgggcattgttgctggcctggctgtcctagcagttgtggtcatcggagctgtggtcgctgctgtgatgtgtaggaggaagagctcaggtggaaaaggagggagctactctcaggctg

SEQ ID NO: 37 - DNA, 514 bp, Artificial Sequence
  LCP1 Affymetrix annotation
gaagtaagcctcatcatcagagcctttcctcaaaactggagtcccaaatgtcatcaggttttgttttttttcagccactaagaacccctctgcttttaactctagaatttgggcttggaccagatctaacatcttgaatactctgccctctagagccttcagccttaatggaaggttggatccaaggaggtgtaatggaatcggaatcaagccactcggcaggcatggagctataactaagcatccttagggttctgcctctccaggcattagccctcacattagatctagttactgtggtatggctaatacctgtcaacatttggaggcaatcctaccttgcttttgcttctagagcttagcatatctgattgttgtcaggccatattatcaatgtttacttttttggtactataaaagctttctgccacccctaaactccaggggggacaatatgtgccaatcaatagcacccctactcacatacacacacacctagccagctgtcaagggc

SEQ ID NO: 38 - DNA, 101 bp, Artificial Sequence
  HLA-DRA Affymetrix annotation
cgatcaccaatgtacctccagaggtaactgtgctcacgaacagccctgtggaactgagagagcccaacgtcctcatctgtttcatagacaagttcacccca

SEQ ID NO: 39 - DNA, 540 bp, Artificial Sequence
  CYTIP Affymetrix annotation
gaattgcaaaactgacatcccatttcacagcaatagtgacctttatttaaattgttgtgttatagtttatgcttcttaaatcatttttcaacctaaacagccaatttctaagcagacaggaaaactaaataataagttaattaatataacaaagatgcaggttcctgctcattccagtaatgtctttgaaagcaaaactaatatttattttctagattatccctgtgaataattgagaactttttggagtcaagtatgaataaaggtgtggcagaatataataatctggactattttctataggataattgctgggttataaaatcttaggtttgcttatgcccagtagctcctgcggaggcttaataataggcaattttgaatttgttcaaacctgtaatggcttgtaaacaaagatgaccatcagctgtttctcacatctatagtgacaataaagcgggaagtataagatttaataggaggggttaaggttcatgagaaccatggaaagatgtggtctgagatgggtgctgcaaagat

SEQ ID NO: 40 - DNA, 527 bp, Artificial Sequence
  TRA@ /// TRAC Affymetrix annotation
tctcgaaccgaacagcagtgcttccaagataatctttggatcagggaccagactcagcatccggccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgagatctgcaagattgtaagacagcctgtgctccct

SEQ ID NO: 41 - DNA, 521 bp, Artificial Sequence
  BTN3A1 Affymetrix annotation
ggaaatttggatgaagggagctagaagaaatacagggatttttttttttttttaagatggagtcttactctgttgctaggctggagtgcagtggtgcgatctcagctccctgcaacctccacctcctgggttcaaacaattctcctgcctcagcctcccgagtactgggaatataggtgcacgccaccacacccaacaaatttttgtacttttagtacagatgagggttcactatgttggccaggatggtctcgatctcttgacctcatgatccacccacctcggtctcccaaagtgctgggattacaggcttgagccaccgggtgaccggcttacagggatatttttaatcccgttatggactctgtctccaggagaggggtctatccacccctgctcattggtggatgttaaaccaatattcctttcaactgctgcctgctagggaaaaactactcctcattatcatcattattattgctctccactgtatcccctctacctggcatgtgcttgtcaag

SEQ ID NO: 42 - DNA, 571 bp, Artificial Sequence
  CXCL2 Affymetrix annotation
agagagacacagctgcagaggccacctggattgcgcctaatgtgtttgagcatcacttaggagaagtcttctatttatttatttatttatttatttatttgtttgttttagaagattctatgttaatattttatgtgtaaaataaggttatgattgaatctacttgcacactctcccattatatttattgtttattttaggtcaaacccaagttagttcaatcctgattcatatttaatttgaagatagaaggtttgcagatattctctagtcatttgttaatatttcttcgtgatgacatatcacatgtcagccactgtgatagaggctgaggaatccaagaaaatggccagtaagatcaatgtgacggcagggaaatgtatgtgtgtctattttgtaactgtaaagatgaatgtcagttgttatttattgaaatgatttcacagtgtgtggtcaacatttctcatgttgaagctttaagaactaaaatgttctaaatatcccttggacattttatgtctttcttgtaagatactgccttgtttaatgttaattatgcagtgtttccctc

SEQ ID NO: 43 - DNA, 532 bp, Artificial Sequence
  TARP Affymetrix annotation
aaatgatacactactgctgcagctcacaaacacctctgcatattacatgtacctcctcctgctcctcaagagtgtggtctattttgccatcatcacctgctgtctgcttagaagaacggctttctgctgcaatggagagaaatcataacagacggtggcacaaggaggccatcttttcctcatcggttattgtccctagaagcgtcttctgaggatctagttgggctttctttctgggtttgggccatttcagttctcatgtgtgtactattctatcattattgtataacggttttcaaaccagtgggcacacagagaacctcactctgtaataacaatgaggaatagccacggcgatctccagcaccaatctctccatgttttccacagctcctccagccaacccaaatagcgcctgctatagtgtagacatcctgcggcttctagccttgtccctctcttagtgttctttaatcagataactgcctggaagcctttcattttacacgccctgaagcagtcttctttgcta

SEQ ID NO: 44 - DNA, 459 bp, Artificial Sequence
  ICOS Affymetrix annotation
gcttctgaagcagccaatgtcgatgcaacaacatttgtaactttaggtaaactgggattatgttgtagtttaacattttgtaactgtgtgcttatagtttacaagtgagacccgatatgtcattatgcatacttatattatcttaagcatgtgtaatgctggatgtgtacagtacagtacttaacttgtaatttgaatctagtatggtgttctgttttcagctgacttggacaacctgactggctttgcacaggtgttccctgagttgtttgcaggtttctgtgtgtggggtggggtatggggaggagaaccttcatggtggcccacctggcctggttgtccaagctgtgcctcgacacatcctcatcccaagcatgggacacctcaagatgaataataattcacaaaatttctgtgaaatcaaatccagttttaagaggagccacttatcaaagagat

SEQ ID NO: 45 - DNA, 484 bp, Artificial Sequence
  KLRD1 Affymetrix annotation
gaaagactctgactgctgttcttgccaagaaaaatgggttgggtaccggtgcaactgttacttcatttccagtgaacagaaaacttggaacgaaagtcggcatctctgtgcttctcagaaatccagcctgcttcagcttcaaaacacagatgaactggattttatgagctccagtcaacaattttactggattggactctcttacagtgaggagcacaccgcctggttgtgggagaatggctctgcactctcccagtatctatttccatcatttgaaacttttaatacaaagaactgcatagcgtataatccaaatggaaatgctttagatgaatcctgtgaagataaaaatcgttatatctgtaagcaacagctcatttaaatgtttcttggggcagagaaggtggagagtaaagacccaacattactaacaatgatacagttgcatgttatattattactaattgtctacttctggagtcta

SEQ ID NO: 46 - DNA, 532 bp, Artificial Sequence
  TRBC1 Affymetrix annotation
aaaggccacactggtgtgcctggccacaggtatcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccatgtatgctgtgctggtcagcgcccttgtgttgatggccatggtcaagagaaaggatttctgaaggcagccctggaagtggagttaggagcttctaacccgtcatggtttcaatacacattcttcttttgccagc

SEQ ID NO: 47 - DNA, 484 bp, Artificial Sequence
  TRA@ /// TRAC /// TRAJ17 /// TRAV20 Affymetrix annotation
ggaacaagacttcaggtcacgctcgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgagatctgcaagattgtaagacagcctgtgctccct

SEQ ID NO: 48 - DNA, 445 bp, Artificial Sequence
  HLA-DRA Affymetrix annotation
gaaggagacggtctggcggcttgaagaatttggacgatttgccagctttgaggctcaaggtgcattggccaacatagctgtggacaaagccaacttggaaatcatgacaaagcgctccaactatactccgatcaccaatgacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactacagagaacgtggtgtgtgccctgggcctgactgtgggtctggtgggcatcattattgggaccatc

SEQ ID NO: 49 - DNA, 512 bp, Artificial Sequence
  TARP /// TRGC2 Affymetrix annotation
aaatgatacactactgctgcagctcacaaacacctctgcatattacatgtacctcctcctgctcctcaagagtgtggtctattttgccatcatcacctgctgtctgcttggaagaacggctttctgctgcaatggagagaaatcataacagacggtggcacaaggaggccatcttttcctcatcggttattgtccctagaagcgtcttctgaggatctagttgggctttctttctgggtttgggccatttcagttctcatgtgtgtactattctatcattattgtataatggttttcaaaccagtgggcacacagagaacctcagtctgtaataacaatgaggaatagccatggcgatctccagcaccaatctctccatgttttccacagctcctccagccaacccaaatagcgcctgctatagtgtagacagcctgcggcttctagccttgtccctctcttagtgttctttaatcagataactgcctggaagcctttcattttacacgccc

SEQ ID NO: 50 - DNA, 408 bp, Artificial Sequence
  LOC100130224 /// UTY Affymetrix annotation
cagaaacctcgatatataattgtatagattttaaaagttttattttttacatctatggtagtttttgaggtgcctattataaagtattacggaagtttgctgtttttaaagtaaatgtcttttagtgtgatttattaagttgtagtcaccatagtgatagcccataaataattgctggaaaattgtattttataacagtagaaaacatatagtcagtgaagtaaatattttaaaggaaacattatatagatttgataaatgttgtttataattaagagtttcttatggaaaagagattcagaatgataacctcttttagagaacaaataagtgacttatttttttaaagctagatgactttgaaatgctatactgtcctgcttgtacaacatggtttggggtgaaggg

SEQ ID NO: 51 - DNA, 444 bp, Artificial Sequence
  ITK Affymetrix annotation
ggtgttgcaattggctctttctaaatcatgtgacgttttgactggcttgagattcagatgcataatttttaattataattattgtgaagtggagagcctcaagataaaactctgtcattcagaagatgattttactcagcttatccaaaattatctctgtttactttttagaattttgtacattatcttttgggatccttaattagagatgatttctggaacattcagtctagaaagaaaacattggaattgactgatctctgtggtttggtttagaaaattcccctgtgcatggtattacctttttcaagctcagattcatctaatcctcaactgtacatgtgtacattcttcacctcctggtgccctatcccgcaaaatgggcttcctgcctggtttttctcttctcacattttttaaatggtcccctgtgtttgtagagaa

SEQ ID NO: 52 - DNA, 483 bp, Artificial Sequence
  TRBC1 /// TRBC2 Affymetrix annotation
gccatcagaagcagagatctcccacacccaaaaggccacactggtgtgcctggccacaggtttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgctgtgctggtcagtgccctcgtgctgatggccatggtcaagagaaagga

SEQ ID NO: 53 - DNA, 592 bp, Artificial Sequence
  TRA@ Affymettrix annotation
gaatcgtttctctgtgaacttccagaaagcagccaaatccttcagtctcaagatctcagactcacagctgggggatgccgcgatgtatttctgtgcttataggagtgcatactctggggctgggagttaccaactcactttcgggaaggggaccaaactctcggtcataccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggttgtggtcc

SEQ ID NO: 54 - DNA, 505 bp, Artificial Sequence
  HLA-B Affymetrix annotation
ctgagagcctacctggagggcctgtgcgtggagtggctccgcagatacctggagaacgggaaggagacgctgcagcgcgcggaccccccaaagacacatgtgacccaccaccccatctctgaccatgaggccaccctgaggtgctgggccctgggcttctaccctgcggagatcacactgacctggcagcgggatggcgaggaccaaactcaggacaccgagcttgtggagaccagaccagcaggagatagaaccttccagaagtgggcagctgtggtggtgccttctggagaagagcagagatacacatgccatgtacagcatgaggggctgccgaagcccctcaccctgagatgggagccatcttcccagtccaccatccccatcgtgggcattgttgctggcctggctgtcctagcagttgtggtcatcggagctgtggtcgctactgtgatgtgtaggaggaagagctcaggtggaaaaggagggagctactctcaggctg

SEQ ID NO: 55 - DNA, 295 bp, Artificial Sequence
  misc_feature 114, 224: n = A,T,C or G
  misc_feature 114, 224: n = A,T,C or G
  HLA-DQA1 /// HLA-DQA2 Affymetrix annotation
  misc_feature 114, 224: n = A,T,C or G
accaatgaggttcctgaggtcacagtgttttccaagtctcccgtgacactgggtcagcccaacaccctcatctgtcttgtggacaacatctttcctcctgtggtcaacatcacntggctgagcaatgggcactcagtcacagaaggtgtttctgagaccagcttcctctccaagagtgatcattccttcttcaagatcagttacctcaccttcctcccttctgntgatgagatttatgactgcaaggtggagcactggggcctggatgagcctcttctgaaacactgggagcctg

SEQ ID NO: 56 - DNA, 519 bp, Artificial Sequence
  misc_feature 284: n = A,T,C or G
  misc_feature 284: n = A,T,C or G
  TRBC1 Affymetrix annotation
  misc_feature 284: n = A,T,C or G
tgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcncccttgtgttgatggccatggtcaagagaaaggatttctgaaggcagccctggaagtggagttaggagcttctaacccgtcatggtttcaatacacattcttcttttgccagcgcttctgaagagctgctctcacctctctgcatcccaatagatatccccctatgtgcatgcacacctgcacactcacggctgaaatctccctaacccagggggaccttagcatgcctaagtga

SEQ ID NO: 57 - DNA, 419 bp, Artificial Sequence
  CD3D Affymetrix annotation
gggaacactgctctcagacattacaagactggacctgggaaaacgcatcctggacccacgaggaatatataggtgtaatgggacagatatatacaaggacaaagaatctaccgtgcaagttcattatcgaatgtgccagagctgtgtggagctggatccagccaccgtggctggcatcattgtcactgatgtcattgccactctgctccttgctttgggagtcttctgctttgctggacatgagactggaaggctgtctggggctgccgacacacaagctctgttgaggaatgaccaggtctatcagcccctccgagatcgagatgatgctcagtacagccaccttggaggaaactgggctcggaacaagtgaacctgagactggtggcttctagaagcagccattaccaactgtacct

SEQ ID NO: 58 - DNA, 540 bp, Artificial Sequence
  misc_feature 56, 165, 168, 171, 172, 174, 178, 183, 455, 480, 486: n = A,T,C or G
  misc_feature 56, 165, 168, 171, 172, 174, 178, 183, 455, 480, 486: n = A,T,C or G
  HOMER1 Affymetrix annotation
  misc_feature 56, 165, 168, 171, 172, 174, 178, 183, 455, 480, 486: n = A,T,C or G
tgctggagtccactgccaatgtgaaacaatggaaacagcaacttgctgcctatcangaggaagcagaacgtctgcacaagcgggtgactgaacttgaatgtgttagtagccaagcaaatgcagtacatactcataagacagaattaaatcagacaatacaagaantgnaanngncacngaaantgaaggaagaggaaatagaaaggttaaaacaagaaattgataatgccagagaactacaagaacagagggattctttgactcagaaactacaggaagtagaaattcggaacaaagacctggagggacaactgtctgacttagagcaacgtctggagaaaagtcagaatgaacaagaagcttttcgcaataacctgaagacactcttagaaattctggatggaaagatatttgaactaacagaattacgagataacttggccaagctactagantgcagctaaggaaagtgaaatttcngtgccnattaattaaaagatacactgtctctcttcataggactgtttaggctctgcatca

SEQ ID NO: 59 - DNA, 485 bp, Artificial Sequence
  misc_feature 407: n = A,T,C or G
  misc_feature 407: n = A,T,C or G
  KLRB1 Affymetrix annotation
  misc_feature 407: n = A,T,C or G
ggttcaccttggcatcaatttgccctgaaacttagctgtgctgggattattctccttgtcttggttgttactgggttgagtgtttcagtgacatccttaatacagaaatcatcaatagaaaaatgcagtgtggacattcaacagagcaggaataaaacaacagagagaccgggtctcttaaactgcccaatatattggcagcaactccgagagaaatgcttgttattttctcacactgtcaacccttggaataacagtctagctgattgttccaccaaagaatccagcctgctgcttattcgagataaggatgaattgatacacacacagaacctgatacgtgacaaagcaattctgttttggattggattaaatttttcattatcagaaaagaactggaagtgganaaacggctcttttttaaattctaatgacttagaaattagaggtgatgctaaagaaaacagctgtatttccatctcaca

SEQ ID NO: 60 - DNA, 532 bp, Artificial Sequence
  misc_feature 108, 111, 121, 122, 123, 142, 171, 172, 174, 176, 187,188, 431, 433, 434: n = A,T,C or G
  misc_feature 108, 111, 121, 122, 123, 142, 171, 172, 174, 176, 187,188, 431, 433, 434: n = A,T,C or G
  TARP /// TRGC2 Affymetrix annotation
  misc_feature 108, 111, 121, 122, 123, 142, 171, 172, 174, 176, 187,188, 431, 433, 434: n = A,T,C or G
aaatgatacactactgctgcagctcacaaacacctctgcatattacatgtacctcctcctgctcctcaagagtgtggtctattttgccatcatcacctgctgtctgcntgnaagaacggcnnnctgctgcaatggagagaantcataacagacggtggcacaaggaggccnncntntcctcatcggnnattgtccctagaagcgtcttctgaggatctagttgggctttctttctgggtttgggccatttcagttctcatgtgtgtactattctatcattattgtataatggttttcaaaccagtgggcacacagagaacctcagtctgtaataacaatgaggaatagccatggcgatctccagcaccaatctctccatgttttccacagctcctccagccaacccaaatagcgcctgctatagtgtaganannctgcggcttctagccttgtccctctcttagtgttctttaatcagataactgcctggaagcctttcattttacacgccctgaagcagtcttctttgcta

SEQ ID NO: 61 - DNA, 553 bp, Artificial Sequence
  misc_feature 102, 199, 200, 202, 203, 205, 206, 207, 208, 209, 210,211, 212: n = A,T,C or G
  misc_feature 102, 199, 200, 202, 203, 205, 206, 207, 208, 209, 210,211, 212: n = A,T,C or G
  TARP /// TRGC2 Affymetrix annotation
  misc_feature 102, 199, 200, 202, 203, 205, 206, 207, 208, 209, 210,211, 212: n = A,T,C or G
cactactgctgcagctcacaaacacctctgcatattacatgtacctcctcctgctcctcaagagtgtggtctattttgccatcatcacctgctgtctgcttngaagaacggctttctgctgcaatggagagaaatcataacagacggtggcacaaggaggccatcttttcctcatcggttattgtccctagaagcgtcnncnnannnnnnnnttgggctttctttctgggtttgggccatttcagttctcatgtgtgtactattctatctattgtataatggttttcaaaccagtgggcacacagagaacctcactctgtaataacaatgaggaatagccatggcgatctccagcaccaatctctccatgttttccacagctcctccagccaacccaaatagcgcctgctatagtgtagacagcctgcggcttctagccttgtccctctcttagtgttctttaatcagataactgcctggaagcctttcattttacacgccctgaagcagtcttctttgctagttgaattatgtggtgtgtttttccgtaata

SEQ ID NO: 62 - DNA, 523 bp, Artificial Sequence
  misc_feature 82, 102, 107, 108, 134, 323, 426, 427, 428, 429, 430,431, 432, 433, 434, 435, 436, 437, 438, 439, 440: n = A,T,C or G
  misc_feature 82, 102, 107, 108, 134, 323, 426, 427, 428, 429, 430,431, 432, 433, 434, 435, 436, 437, 438, 439, 440: n = A,T,C or G
  HLA-A /// HLA-A29.1 /// HLA-B /// HLA-G /// HLA-H /// HLA-J Affymetrix annotation
  misc_feature 82, 102, 107, 108, 134, 323, 426, 427, 428, 429, 430,431, 432, 433, 434, 435, 436, 437, 438, 439, 440: n = A,T,C or G
tacctggagggcacctgcatggagtggctccgcagacacctggagaacgggaaggagacgctgcagcgcgcggacccccccnaagacacacgtgacccaccnccctnnctctgaacatgaggcataacgaggtnctgggttctgggcttctaccctgcggagatcacattgacctggcagcgggatggggaggaccagacccaggacatggagctcgtggagaccaggcccacaggggatggaaccttccagaagtgggcggttgtggtagtgccttctggagaggaacagagatacacatgccatgtgcagcacaaggggcntgcccaagcccctcatcctgagatgggagccctctccccagcccaccatccccattgtgggtatcattgctggcctggttctccttggagctgtggtcactgnnnnnnnnnnnnnnnctgtgatgtggaggaagaagagctcagatagaaaaggagggagctactctcaggctgcaagcagccaaagtgcccagggctct

SEQ ID NO: 63 - DNA, 424 bp, Artificial Sequence
  HLA-DMA Affymetrix annotation
ctgttttgtcagtaatctcttcccacccatgctgacagtgaactggcagcatcattccgtccctgtggaaggatttgggcctacttttgtctcagctgtcgatggactcagcttccaggccttttcttacttaaacttcacaccagaaccttctgacattttctcctgcattgtgactcacgaaattgaccgctacacagcaattgcctattgggtaccccggaacgcactgccctcagatctgctggagaatgtgctgtgtggcgtggcctttggcctgggtgtgctgggcatcatcgtgggcattgttctcatcatctacttccggaagccttgctcaggtgactgattcttccagaccagagtttgatgccagcagcttcggccatccaaacagaggatgctcagatttctcacatcctgc

SEQ ID NO: 64 - DNA, 429 bp, Artificial Sequence
  EAF2 Affymetrix annotation
gaacaggtgaccataactctgccaaatatagaaagttgaaggaagtagtaaaattcagtatcgtaaagaacaacagcaacaacaaatgtggaattcagccaggactcccaatcttgtaaaacattctccatctgaagataagatgtccccagcatctccaatagatgatatcgaaagagaactgaaggcagaagctagtctaatggaccagatgagtagttgtgatagttcatcagattccaaaagttcatcatcttcaagtagtgaggatagttctagtgactcagaagatgaagattgcaaatcctctacttctgatacagggaattgtgtctcaggacatcctaccatgacacagtacaggattcctgatatagatgccagtcataatagatttcgagacaacagtggccttctgatgaatacttt

SEQ ID NO: 65 - DNA, 514 bp, Artificial Sequence
  DENND2D Affymetrix annotation
ttctcacttttcatccaggaagccgagaagagcaagaatcctcctgcaggctatttccaacagaaaatacttgaatatgaggaacagaagaaacagaagaaaccaagggaaaaaactgtgaaataagagctgtggtgaataagaatgactagagctacacaccatttctggacttcagcccctgccagtgtggcaggatcagcaaaactgtcagctcccaaaatccatatcctcactctgagtcttggtatccaggtattgcttcaaactggtgtctgagatttggatccctggtattgatttctcaggactttggagggctctgacaccatgctcacagaactgggctcagagctccattttttgcagaggtgacacaggtaggaaacagtagtacatgtgttgtagacacttggttagaagctgctgcaactgccctctcccatcattataacatcttcaacacagaacacactttgtggtcgaaaggctcagcctctctacatgaagtctg

SEQ ID NO: 66 - DNA, 429 bp, Artificial Sequence
  misc_feature 129: n = A,T,C or G
  misc_feature 129: n = A,T,C or G
  HLA-F Affymetrix annotation
  misc_feature 129: n = A,T,C or G
tctaccctgcggagatcacgctgacctggcagcgggatggggaggaacagacccaggacacagagcttgtggagaccaggcctgcaggggatggaaccttccagaagtgggccgctgtggtggtgcctnctggagaggaacagagatacacatgccatgtgcagcacgaggggctgccccagcccctcatcctgagatgggagcagtctccccagcccaccatccccatcgtgggcatcgttgctggccttgttgtccttggagctgtggtcactggagctgtggtcgctgctgtgatgtggaggaagaagagctcagatagaaacagagggagctactctcaggctgcagtgtgagacagcttccttgtgtgggactgagaagcaagatatcaatgtagcagaattgcacttgtgcctcacgaacata

SEQ ID NO: 67 - DNA, 299 bp, Artificial Sequence
  SLAMF7 Affymetrix annotation
aacacctgtgctaggtcagtctggcacgtaagatgaacatccctaccaacacagagctcaccatctcttatacttaagtgaaaaacatggggaaggggaaaggggaatggctgcttttgatatgttccctgacacatatcttgaatggagacctccctaccaagtgatgaaagtgttgaaaaacttaataacaaatgcttgttgggcaagaatgggattgaggattatcttctctcagaaaggcattgtgaaggaattgagccagatctctctccctactgcaaaaccctattgtagta

SEQ ID NO: 68 - DNA, 307 bp, Artificial Sequence
  MCM10 Affymetrix annotation
aaactttcccatctagataatgatgatcacatagtcttgatgtacggacattaaaagccagatttcttcattcaattctgttatctctgttttactctttgaaattgatcaagccactgaatcactttgcatttcagtttatatatatagagagaaagaaggtgtctgctcttacattattgtggagccctgtgatagaaatatgtaaaatctcatattattttttttttaatttttttattttttatgacagggtctcactatgtcaccctggctggagtgcagtagtgcgatcgcggcacactgc

SEQ ID NO: 69 - DNA, 378 bp, Artificial Sequence
  KIAA1549 Affymetrix annotation
aaatgactgcattcgtctcttttttaaaggtagagattaaactgtatagacagcatagggatgaaaggaaccaagcgtttctgtgggattgagactggtacgtgtacgatgaacctgctgctttgttttctgagaagaggtttgaagacattttattaacagcttaatttttctcttttactccataggaacttattttaatagtaacattaacaacaagaatactaagactgtttgggaattttaaaaagctactagtgagaaaccaaatgataggttgtagagcctgatgactccaaacaaagccatcacccgcattcttcctccttcttctggtgctacagctccaagggcccttcaccttcatgtctgaaatgg

SEQ ID NO: 70 - DNA, 492 bp, Artificial Sequence
  AADAT Affymetrix annotation
ggcagctgcagacaagtggttaactggtttggcagaatggcatgttcctgctgctggaatgtttttatggattaaagttaaaggcattaatgatgtaaaagaactgattgaagaaaaggccgttaagatgggggtattaatgctccctggaaatgctttctacgtcgatagctcagctcctagcccttacttgagagcatccttctcttcagcttctccagaacagatggatgtggccttccaggtattagcacaacttataaaagaatctttatgaagaaattaaactaggttgggcatggtgcgtcacacctataatcccagcactttgggaggcagaggagggaggatcacttgaacccaggaattcaggctgcagtaagctacgatcacaccactgcactctggcctgcatgcactctggcctgcatggcagaacaagaccctgtctctaaaaaaagagaaagaaatcaaactaatcatgctgctcat

SEQ ID NO: 71 - DNA, 474 bp, Artificial Sequence
  LONRF2 Affymetrix annotation
acagttcaaccagtgaccgacttctctctcatgctgtttaccccacacacaatttcccactcaattctgaaaataagaacctgttaataggttggaaagctgtgtactctattcatatattgttctttcatgctagtggagagtggtgtcattagcatcttaattttagagttgtgaaatgattttaccaattaggaattgaatgtgtattttttttctgtttaataagaagagcaaatttgaataaataagctggtgtagataaacttaataatcatgctttttcttgtttggagataggtgatgtgttgtcatatcctgtgatacaggtcactcatctggccttctgtttctgaagtttaagtctggtttgaatatgtaataatactactcagcatttcttgttgcctaagtgagacgaaacttaaatgttatgatatttacttcatgtattcttgtactgttcatttcaat

SEQ ID NO: 72 - DNA, 563 bp, Artificial Sequence
  misc_feature 35, 55, 96: n = A,T,C or G
  misc_feature 35, 55, 96: n = A,T,C or G
  MAP1B Afffymetrix annotation
  misc_feature 35, 55, 96: n = A,T,C or G
aatggcttctatgatcagaactgggaaaacagtgnatcttatggtggaagaggtnctcagcaagtgtacagtatttaccttcctttgtcttacatnggctttttaaattttccattaatttcaacataattatgggaacaagtgtacagaagaattttttttttaagatatgtgagaacttttcatagatgaactttttaacaaatgttttcatttacaggaaattgcaaagaaaattctcaagtgatagtctttttttttaagtgtttcgtaagacaaaaattgaataatgttttttgaagttctggcaagattgaagtctgatattgcagtaatgatatttattaaaaacccataactaccaggaataatgatacctcccaccccttgattcccataacataaaagtgctacttgagagtgggggagaatggcatggtaggctacttttcagggccttgacaagtacatcacccagtggtatcctacatacttctttcaagatcttcaaccatgaggtaaaagagccaagttcaaagaaccctagcacaaatttgctttgg

SEQ ID NO: 73 - DNA, 480 bp, Artificial Sequence
  C2orf63 Affymetrix annotation
acagggtcagactcatagggtcatggagtacatacagcagttgaaggactttactaccgatgacctgttgcagctattaatgtcatgtccccaagttgaattaattcagtgtctcactaaagagttgaatgagaaacaaccatctttatcttttggtcttgctatacttcatctgttctctgcagacatgaaaaaagttggcattaagctacttcaagaaatcaataaaggtgggatagatgcagtagaaagtcttatgataaatgattccttttgctccatagaaaagtggcaagaagtggcaaatatatgttcacagaatggctttgacaaattatctaatgacatcacgtctattcttcgatctcaggctgcagttacagaaatttctgaagaggatgacgcagtcaacctaatggaacatgtgttttggtagttctatatcttaaccagctgagggagcttgtacaacaccttatg

SEQ ID NO: 74 - DNA, 289 bp, Artificial Sequence
  FAM26F Affymetrix annotation
gtactggcccttcggattgaaagtatacagtgatgaaatttgctgccactctttcatgcttggagtgttatattcttttggatgcgagccctcaaagaaacatttaatattctcttttgccaattcagttgcatgctctgtggctttacttttaaggatctgctgctcctgttccaaatagattttccagaatttcagctgcagaaaactaactggagataggcatcgggtgacagatgtaaaaatcagaagaatgatgataacaactgctatcaagatccagcccaac

SEQ ID NO: 75 - DNA, 410 bp, Artificial Sequence
  SHROOM3 Affymetrix annotation
aataacttcatttcctacaaggtataaaaagtggtcaagtgaatgtgaaggggcttttctacacaggaatatattatcgggaacaaagtatttcctgctgccttaactctttgggatgcataggataaaatgataaagaccattttaatatcagaaagggttgtcttattaatttttaaataaaacttcacatttcttaatggggagctcattcagaaactaaataatggtttctcaaagtgtggtcaggatacgatctgcatcagaatccttggaatgcttgttaaaaataccaattgctatgacaaaaccaagtctgctggaaactgcatttcagcaggtttcccatgttattctgatgtattttaacatttgagagccactaccaatcatctgtacagttcctactg

SEQ ID NO: 76 - DNA, 488 bp, Artificial Sequence
  LOC100130216 /// USP9Y Affymetrix annotation
aaccaatacacaaaattttcctatgtcagaatgtggtggagcataatagattgtatttggtgtgcttgcgattttttttttccatagaatttattaagtgaagtttctaaaactttgcttctcctgatcccggtgaagtgtacatcataagaatccatagtactttgaagtaccattgcaccaagatgtctgactgaattcatagtcacacttttatttgaaagaaagaattgttgtagttttttttcattattctaaaactcttgttgttagatacaagatttaattaagatctaagctcctgcttatttaatgtaattctaaggtaccattttagaaaaaacatttgttttaagattccaagaaacctgtgagttaatactatatttaaaagagaattggtaaattttgaatgtgtgtaatattttggaacctgtttaaaaaccaaatatacctgcaaatagatacagcctatcctatactattta

SEQ ID NO: 77 - DNA, 537 bp, Artificial Sequence
  misc_feature 160, 161, 162, 163, 164, 165, 166, 167, 168, 169, 170,171, 172, 173, 174, 175, 176, 177, 178, 179, 180, 181, 182, 439,440, 492: n = A,T,C or G
  misc_feature 160, 161, 162, 163, 164, 165, 166, 167, 168, 169, 170,171, 172, 173, 174, 175, 176, 177, 178, 179, 180, 181, 182, 439,440, 492: n = A,T,C or G
  C1orf162 Affymetrix annotation
  misc_feature 160, 161, 162, 163, 164, 165, 166, 167, 168, 169, 170,171, 172, 173, 174, 175, 176, 177, 178, 179, 180, 181, 182, 439,440, 492: n = A,T,C or G
tgctgctgatagcctttatcttcctcatcataaagagctacagaaaatatcactccaagccccaggccccagatcctcactcagatcctccagccaagctttcatccatcccaggggaatcacttacctatgccagcacaactttcaaactctcagaagnnnnnnnnnnnnnnnnnnnnnnnatgctcaaattaaagtaacaaactaactcagcttttccaatgaggcttgaatccatttcctctcatctcagccctatcttcacacatcactttcacttttttacaaattttggaccaccacctgtgtgaaactgcagtcggagttgtttagatgtgatctggcaatgctatccagcatctttggagaccaatggtcagtcttttcctggccagaggaaagattgatggccctcccacttgaactgacagcctgtganncccttgggggcatagactgccttccttggacccttccaaagtgtgtggtacngagctcagtgcacagagtattcacccagcatcatgaatcaacttg

SEQ ID NO: 78 - DNA, 413 bp, Artificial Sequence
  C4orf7 Affymetrix annotation
tgaagaaagttctcctcctgatcacagccatcttggcagtggctgttggtttcccagtctctcaagaccaggaacgagaaaaaagaagtatcagtgacagcgatgaattagcttcagggttttttgtgttcccttacccatatccatttcgcccacttccaccaattccatttccaagatttccatggtttagacgtaattttcctattccaatacctgaatctgcccctacaactccccttcctagcgaaaagtaaacaagaaggaaaagtcacgataaacctggtcacctgaaattgaaattgagccacttccttgaagaatcaaaattcctgttaataaaagaaaaacaaatgtaattgaaatagcacacagcattctctagtcaatatctttagtgatcttctttaata

SEQ ID NO: 79 - DNA, 494 bp, Artificial Sequence
  misc_feature 50: n = A,T,C or G
  misc_feature 50: n = A,T,C or G
  FAM26F Affymetrix annotation
  misc_feature 50: n = A,T,C or G
gctgatttagcttatggaagaggaaccagaaatttgtccttgaataatgnttcccgtgttgggctggatcttgatagcagttgttatcatcattcttctgatttttacatctgtcacccgatgcctatctccagttagttttctgcagctgaaattctggaaaatctatttggaacaggagcagcagatccttaaaagtaaagccacagagcatgcaactgaattggcaaaagagaatattaaatgtttctttgagggctcgcatccaaaagaatataacactccaagcatgaaagagtggcagcaaatttcatcactgtatactttcaatccgaagggccagtactacagcatgttgcacaaatatgtcaacagaaaagagaagactcacagtatcaggtctactgaaggagatacggtgattcctgttcttggctttgtagattcatctggtataaacagcactcctgagttatgaccttttgaatgagtag

SEQ ID NO: 80 - DNA, 299 bp, Artificial Sequence
  FAM26F Affymetrix annotation
gtgttgggctggatcttgatagcagttgttatcatcattcttctgatttttacatctgtcacccgatgcctatctccagttagttttctgcagctgaaattctggaaaatctatttggaacaggagcagcagatccttaaaagtaaagccacagagcatgcaactgaattggcaaaagagaatattaaatgtttctttgagggctcgcatccaaaagaatataacactccaagcatgaaagagtggcagcaaatttcatcactgtatactttcaatccgaagggccagtactacagcat

SEQ ID NO: 81 - DNA, 136 bp, Artificial Sequence
  FAM26F Affymetrix annotation
tctactcattcaaaaggtcataactcaggagtgctgtttataccagatgaatctacaaagccaagaacaggaatcaccgtatctccttcagtagacctgatactgtgagtcttctcttttctgttgacatatttgt

SEQ ID NO: 82 - DNA, 549 bp, Artificial Sequence
  misc_feature 128, 129, 144, 145, 146, 147, 148, 149, 150, 151, 167: n = A,T,C or G
  GBP5 Affymetrix annotation
  misc_feature 128, 129, 144, 145, 146, 147, 148, 149, 150, 151, 167: n = A,T,C or G
  misc_feature 128, 129, 144, 145, 146, 147, 148, 149, 150, 151, 167: n = A,T,C or G
ttagctcctcaagcatatctgactggcatgatcctgcattgtggttacctggaagggaaaaacaacccctgggaattttatccaggaagttggaacaatcacaaacaaaagtgggaggcagaaggaannggcacattaatcctnnnnnnnnttatctttttctcctnagaggcacaagtgaaagcagaagctgaaaaggctgaagcgcaaaggttggcggcgattcaaaggcagaacgagcaaatgatgcaggagagggagagactccatcaggaacaagtgagacaaatggagatagccaaacaaaattggctggcagagcaacagaaaatgcaggaacaacagatgcaggaacaggctgcacagctcagcacaacattccaagctcaaaatagaagccttctcagtgagctccagcacgcccagaggactgttaataacgatgatccatgtgttttactctaaagtgctaaatatgggagtttcctttttttactctttgtcactgatgacacaacagaaaagaaactgtagaccttgggacaatca

SEQ ID NO: 83 - DNA, 435 bp, Artificial Sequence
  misc_feature 36, 55, 64, 65, 96, 177, 410: n = A,T,C or G
  HILS1 Affymetrix annotation
  misc_feature 36, 55, 64, 65, 96, 177, 410: n = A,T,C or G
  misc_feature 36, 55, 64, 65, 96, 177, 410: n = A,T,C or G
gcacgtccaaggtgatcctgagggctgtggcggacnaaggggacctgcaagtatntgtccctgnncaccctgaagaaggctgtttccaccacgggntacgacatggcccgaaatgcctatcacttcaagcgtgtgctcaaggggctggtggacaagggctcagcaggtgaccggcangggggcctcaggctccttcaccctgggcaagaagcaggcctccaagtccaagctcaaggtcaagaggcaacgacagcagaggtggcgctctgggcagcgcccctttggacagcacaggtcactactgggctccaaacaggggcacaagcggcttatcaagggggttcgaagggtggccaagtgccactgcaattaatgaggcaggccaggcaagcagtcaggggtgccaagancgccattggctcagtgcagtgggaa

SEQ ID NO: 84 - DNA, 262 bp, Artificial Sequence
  GBP1 Affymetrix annotation
ggaacaggagcaactactaaaagagggatttcaaaaagaaagcagaataatgaaaaatgagatacaggatctccagacgaaaatgagacgacgaaaggcatgtaccataagctaaagaccagagccttcctgtcacccctaaccaaggcataattgaaacaattttagaatttggaacaagcgtcactacatttgataataattagatcttgcatcataacaccaaaagtttataaaggcatgtggtacaatgatcaaaatc

SEQ ID NO: 85 - DNA, 413 bp, Artificial Sequence
  misc_feature 369, 370, 371, 372, 373, 374, 375: n = A,T,C or G
  SLA2 Affymetrix annotation
  misc_feature 369, 370, 371, 372, 373, 374, 375: n = A,T,C or G
  misc_feature 369, 370, 371, 372, 373, 374, 375: n = A,T,C or G
aacacctcttaagtctagcacactgcagtgaggccaggcacctcagtgctgggcaggggcatcagaaggtgctaagccctctctccacaatgccaagacggagaccacagcctacaccaaatccagcccttgatttccctgctgcctccataaacagaaagaggtctgctggatccgctaagggatcagggagaggaagaaagagggatggggtgggaggcaccccctccagtgctcctactggttcccaagctacaggtggggtgggaaaggctttatcaggtatcatcaacaggttctcaattaaagatttgatttattcaagtatgtgaaaaaattctacaatggaaactcttattagatgctgcnnnnnnngtgctatggaccacgcacatacagccatgctgtttcag

SEQ ID NO: 86 - DNA, 348 bp, Artificial Sequence
  misc_feature 189: n = A,T,C or G
  B2M Affymetrix annotation
  misc_feature 189: n = A,T,C or G
  misc_feature 189: n = A,T,C or G
acataccttgggttgatccacttaggaacctcagataataacatctgccacgtatagagcaattgctatgtcccaggcactctactagacacttcatacagtttagaaaatcagatgggtgtagatcaaggcaggagcaggaaccaaaaagaaaggcataaacataagaaaaaaaatggaaggggtggnaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctcc

SEQ ID NO: 87 - DNA, 411 bp, Artificial Sequence
  misc_feature 26, 28, 32, 38, 46, 49, 60, 63, 64, 65, 68, 76, 79, 80,81, 90, 98, 214: n = A,T,C or G
  STAT1 Annotation from R2.6 that became NA in R2.9
  misc_feature 26, 28, 32, 38, 46, 49, 60, 63, 64, 65, 68, 76, 79, 80,81, 90, 98, 214: n = A,T,C or G
  misc_feature 26, 28, 32, 38, 46, 49, 60, 63, 64, 65, 68, 76, 79, 80,81, 90, 98, 214: n = A,T,C or G
gaatatttgaatctacctagtgagtntntagngcatgnttttgtcnggnatcctggaaangcnnnccncaaaaagntannntttgccccnttcaaaancatgcaccctgaagaagctgtttgtacaggattgggtttattctgttattaagacaaaggcatcatggcctttgggtgagaggcccgtgtgtgtttgggatttggcaatcagcatnccatctctgtcatcaccattattgagaaaatagatggattggttccctctctgcagtcctgtggagcagttggactgctctctctgctctcaggatgatactgtgagaacaatttaaatatgctaagcacatgtcaggaaacagttttgtggtctttggacactcgctgtagccattccgttccatttcaggtgatt

SEQ ID NO: 88 - DNA, 559 bp, Artificial Sequence
  SLITRK6 Affymetrix annotation
gaagtccatcctttggtccaaagcatctggaagaggaagaagagaggaatgagaaagaaggaagtgatgcaaaacatctccaaagaagtcttttggaacaggaaaatcattcaccactcacagggtcaaatatgaaatacaaaaccacgaaccaatcaacagaatttttatccttccaagatgccagctcattgtacagaaacattttagaaaaagaaagggaacttcagcaactgggaatcacagaatacctaaggaaaaacattgctcagctccagcctgatatggaggcacattatcctggagcccacgaagagctgaagttaatggaaacattaatgtactcacgtccaaggaaggtattagtggaacagacaaaaaatgagtattttgaacttaaagctaatttacatgctgaacctgactatttagaagtcctggagcagcaaacatagatggagagtttgagggctttcgcagaaatgctgtgattctgttttaagtccataccttgtaaataagtgccttacgtgagtgtgtcatcaatcagaacctaagc

SEQ ID NO: 89 - DNA, 107 bp, Artificial Sequence
  GOLGA7 Annotation from R2.6 that became NA in R2.9
agaagagattctgctgtctacatcaatacacctgaatagttggacagaaaattgaaatcttttaactaattctaactatgaagcacagtgaaatagaaagttaggct

SEQ ID NO: 90 - DNA, 517 bp, Artificial Sequence
  misc_feature 152, 197, 233, 234, 241, 249, 262, 279, 285, 297, 311,312, 313, 314: n = A,T,C or G
  GBP4 Affymetrix annotation
  misc_feature 152, 197, 233, 234, 241, 249, 262, 279, 285, 297, 311,312, 313, 314: n = A,T,C or G
  misc_feature 152, 197, 233, 234, 241, 249, 262, 279, 285, 297, 311,312, 313, 314: n = A,T,C or G
gacagtgagctggcacagagttagggaaattgactgtgtctcatattggctagtgagagtgatctgttggaattgtatatcaaaattttaatgtacatacattttgtctagcaattctactattgggtatttatatagtacatataaatatnaatgtatatgtttagtaaatatatacttatagttagtaaatatantttatatctatttagtaaatatactaaatgtcaggnntctgagnccaagctnaagccatcatatnccctgtgacctgcatgntacatncgtccagatggnctgaagcaagtgannnntcacaaaagaagtgaaaatggcctgttcctgccttaactgatgacattaccttgtgaaattccttctcctggctcatcctggctcaaaagctcccccactaagcaacttgtgacacccacctctgcccgcagagaacaaccccctttgactgtaattttcctttaccaacccaaatcctgtaaaatggtcccaacctatctcc

SEQ ID NO: 91 - DNA, 305 bp, Artificial Sequence
  misc_feature 41: n = A,T,C or G
  EPSTI1 Affymetrix annotation
  misc_feature 41: n = A,T,C or G
  misc_feature 41: n = A,T,C or G
accctgcactcccaaagattttgtgcagatgggtagttccnttttttaaaaattgtgcagatatggaaaattgtgacttacttcatgaccagaactatctagaatatgtgtgggggtataaacatcttgcttaaccaaatatctatgtaggcagaggtaaccaggagagaagcaagacttgctgcctaaaggagcccaccattttacttttcacatttaatctgccacgttgaatcaattggaataaaacctgactcgcaggtgactggacaggaaatcccaaagttccaccatttctatgctta

SEQ ID NO: 92 - DNA, 361 bp, Artificial Sequence
  ZNF285A Affymetrix annotation
gaaacccatgctcttactatgaaagaacgttagtacccaggttttccatgagattctctacacaggcaagaagctccatagaagtggcatttgaagggtgtggcagaggcagtgctgtgtttatcacactggttccatttccttgcaaataagaagtctatttcccagtaacccttgcagttaagagtgtgcccatgtgattgagttctagccaatggagtgtgagcaaaagtgatataagccactttcaggtctagcctttacaaacatcctcaggcttctctatccctgccaaggtgaccttggaggctgcttattccagactgggttgatagaaggtcactacttcatctgtgttgga

SEQ ID NO: 93 - DNA, 350 bp, Artificial Sequence
  misc_feature 26, 324: n = A,T,C or G
  TMEM56 Affymetrix annotation
  misc_feature 26, 324: n = A,T,C or G
  misc_feature 26, 324: n = A,T,C or G
atgaatcagtgttactaggacttatncagtacttaaaatagcaacttggcattctttattttgtttcctggttgttttatttggagggataataaatgtctaagttatttccattaaaattttgaaatgtttgtatactttatgtgtgccattttaaagtatatgcaagttctaagcaataatctgcatgttatacaaggttgacatattttgtcctgaaatttttagttaacatttcaagaatgataaaatgaacaccctgtaaattacccttctccccctcccctccatgaaaaccttgggattttcttgtgctagaacacntaccacaatgtggtgcaaagctttgt

SEQ ID NO: 94 - DNA, 536 bp, Artificial Sequence
  misc_feature 117, 137, 139, 140, 141, 142, 143, 144, 145, 146, 147,149, 152, 153, 154, 155, 221: n = A,T,C or G
  NA
  misc_feature 117, 137, 139, 140, 141, 142, 143, 144, 145, 146, 147,149, 152, 153, 154, 155, 221: n = A,T,C or G
  misc_feature 117, 137, 139, 140, 141, 142, 143, 144, 145, 146, 147,149, 152, 153, 154, 155, 221: n = A,T,C or G
aaatgtacccttgatttgatgctaatgctgtatttagggctgaaggaagcacacactaaatatctgagtgcttttcagattccatctatgctgaaaaagaatctaggagaataaacncatttcaattagcccttaanannnnnnnnnanaannnnagcccactaaagcccagtagggcataggagagaacactgcaccaggattcagatctggattctaanttttgttctgaaaaatagcaagtgacactggcatgccatttaacctctccgggcctcaatttccactatagatagtacctgatgtgtcagtaagacaactgatgtaactttgccaaacaagtagaattatccttcctcctttgtcctgctctgtcctagcttttaatacttggtctgccctaacattttcctgtatgtatttctttatcccagatattcgaacaattgctagcaaggaaaagtaatgacggattttcatttcccaatatagtctggcaaagaaatgaaaggtttacttctccttgctaattcaat

SEQ ID NO: 95 - DNA, 403 bp, Artificial Sequence
  GBP5 Affymetrix annotation
aacaatgtgcagctttcaactgggtggaggctgctattctgtggacagtgagatgtttccttggcactgtcaatagacaatctgcgtagagaaattccaagctgaaagccaataatgttataataaaatagagattcttcagaagatgaaaggaattaccagcatggaaattgtgtcataggcttaagggctaaagaagaagccttttcttttctgttcaccctcaccaagagcacaacttaaatagggcattttataacctgaacacaatttatattggacttaattattatgtgtaatatgtttataatcctttagatcttataaatatgtggtataaggaatgccatataatgtgccaaaaatctgagtgcatttaatttaatgcttgcttatagtgcta

SEQ ID NO: 96 - DNA, 346 bp, Artificial Sequence
  UBASH3B Afymetrix annotation
gcttctacaagtgtgccacatcaatccggtaatgccccagtgttattcacagacagaactttgtttcctgtgattttaaaataccgcgtctgttcctccatggaccagagtaattggcacattttaatgcataagctgggggtttcattttcccaggctctcttcaccatcactgcattggtagctaggagcttattgcttcaccccagtatggagttcagattacagtgttttccattacatttagattcatagaatctgaatggctgattaaatggccatctgatggctgaaagaggggcgtatttttcactctgtagtgaaaggcttggaggagtttctactt

SEQ ID NO: 97 - DNA, 435 bp, Artificial Sequence
  misc_feature 220, 221, 225, 229, 231, 245, 248, 332: n = A,T,C or G
  RNF144B Affymetrix annotation
  misc_feature 220, 221, 225, 229, 231, 245, 248, 332: n = A,T,C or G
  misc_feature 220, 221, 225, 229, 231, 245, 248, 332: n = A,T,C or G
taaaaataagtcgccagctctctcctttataaacagtctttagactggtttgtatcatgccccttgatgtaccagagatatgtttaaccaacctagttttgttgattctgacaatctcacacacatttaagaatttaccatttttcaggcacttttcaatgttaaaaaaaattaaatccaattattgaaaatcagtttgacaaacaacccccactccatnncccnggcnanaaaaaaaaaaaaanaanaacaaaagcagctaattcagtgatacaaactctgtaaggtggcaaattcccccaactcgccaaggaaatagcacatatttattntctcccatctttactccaaatttgggacctcttcctctgataacacagtcttttaggttacttgaaatcagcccccatttaaagactctttgcggcaccaagc

SEQ ID NO: 98 - DNA, 320 bp, Artificial Sequence
  misc_feature 94, 95: n = A,T,C or G
  ARHGAP15 Annotation from R2.6 that became NA in R2.9
  misc_feature 94, 95: n = A,T,C or G
  misc_feature 94, 95: n = A,T,C or G
gaaatggcacattttctggatgtgagagttggtcaaaagatcacaaaaaaagtcaaaaaataattctactctgtgaatgaaaaatggatatttnngtacttaccctcataagcattaaaagaaaataatgcatgaaattccatagaaatgtgcctatcatgttatactgactcaaaccagaagacctagagtatgatattgctaatataatacatgtggtgggtatgagtggaagtatgtgtgtgagatttatcattgccatagtgtaaaagagttgaattagcttccacttgactagatgagagctcttagttcttatt

SEQ ID NO: 99 - DNA, 259 bp, Artificial Sequence
  misc_feature 103, 130: n = A,T,C or G
  AKR1C2* Annotation from R2.6 that became NA in R2.9
  misc_feature 103, 130: n = A,T,C or G
  misc_feature 103, 130: n = A,T,C or G
cccagccgctataacttttaacaattcccatatgtcctttattccactaagatgagtgcagtatatatttccatctgtccaaggcttcctaaatgtagccaangccaagccaacaccagtcacatgatcnaaatcaaagggcatttggggaatccaggctgtgattcagggaagttccaagtgtctgatgaagtgtttgttttacatctttgtgtcccttgcaggtctagcactgtgctatgtaggtaacatgtgctcc

SEQ ID NO: 100 - DNA, 589 bp, Artificial Sequence
  STAT1 Affymetrix annotation
ctggatatatcaagactgagttgatttctgtgtctgaagttcacccttctagacttcagaccacagacaacctgctccccatgtctcctgaggagtttgacgaggtgtctcggatagtgggctctgtagaattcgacagtatgatgaacacagtatagagcatgaatttttttcatcttctctggcgacagttttccttctcatctgtgattccctcctgctactctgttccttcacatcctgtgtttctagggaaatgaaagaaaggccagcaaattcgctgcaacctgttgatagcaagtgaatttttctctaactcagaaacatcagttactctgaagggcatcatgcatcttactgaaggtaaaattgaaaggcattctctgaagagtgggtttcacaagtgaaaaacatccagatacacccaaagtatcaggacgagaatgagggtcctttgggaaaggagaagttaagcaacatctagcaaatgttatgcataaagtcagtgcccaactgttataggttgttggataaatcagtggttatttagggaactgcttgacgtaggaacggtaaatttctgtgggag

SEQ ID NO: 101 - protein, 450 aa, Artificial Sequence
  Proetin D MAGEA3 fuison protein
MDPKTLALSLLAAGVLAGCSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETMDLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEEGGHHHHHHH

SEQ ID NO: 102 - DNA, 20 bp, Artificial Sequence
  CpG 1826
tccatgacgttcctgacgtt

SEQ ID NO: 103 - DNA, 18 bp, Artificial Sequence
  CpG 1758
tctcccagcgtgcgccat

SEQ ID NO: 104 - DNA, 30 bp, Artificial Sequence
  CpG 1758
accgatgacgtcgccggtgacggcaccacg

SEQ ID NO: 105 - DNA, 24 bp, Artificial Sequence
  CpG 2006, CpG 7909
tcgtcgttttgtcgttttgtcgtt

SEQ ID NO: 106 - DNA, 20 bp, Artificial Sequence
  CpG 1668
tccatgacgttcctgatgct

SEQ ID NO: 107 - protein, 9 aa, Artificial Sequence
  MAGE A3 peptide
FLWGPRALV

SEQ ID NO: 108 - protein, 10 aa, Artificial Sequence
  MAGE A3 peptide
MEVDPIGHLY

SEQ ID NO: 109 - protein, 10 aa, Artificial Sequence
  MAGE A3 peptide
VHFLLLKYRA

SEQ ID NO: 110 - protein, 10 aa, Artificial Sequence
  MAGE A3 peptide
LVHFLLLKYR

SEQ ID NO: 111 - protein, 10 aa, Artificial Sequence
  MAGE A3 peptide
LKYRAREPVT

SEQ ID NO: 112 - protein, 16 aa, Artificial Sequence
  MAGE A3 peptide
ACYEFLWGPRALVETS

SEQ ID NO: 113 - protein, 12 aa, Artificial Sequence
  MAGE A3 peptide
TQHFVQENYLEY

(19) United States
(12) Patent Application Publication
(10) Pub. No.: US 2011/0070268 A1
(43) Pub. Date: Mar. 24, 2011

(54) Method

(75) Inventors: Vincent Brichard, Rixensart (BE); Benjamin Georges Elie Lea Ghislain Dizier, Rixensart (BE); Olivier Gruselle, Rixensart (BE); Jamila Louahed, Rixensart (BE); Fernando Ulloa-Montoya, Rixensart (BE)

(73) Assignee: GlaxoSmithKline Biologicals SA, Rixensart (BE)

(21) Appl. No.: 12/884,407

(22) Filed: Sep. 17, 2010

Related U.S. Application Data
(60) Provisional application No. 61/278,387, filed on Oct. 6, 2009.
(60) Provisional application No. 61/277,046, filed on Sep. 18, 2009.

(30) Foreign Application Priority Data
Oct. 6, 2009 (GB) GB0917457.4

(51) Int. Cl. A61K 39/00; C12Q 1/68; C40B 30/00; C40B 40/06; C07H 21/00; C07K 2/00; C07K 14/47; A61P 35/00; A61P 37/04

(52) U.S. Cl. 424/277.1; 435/6; 506/7; 506/16; 536/24.31; 530/300; 530/350

(57) ABSTRACT

Methods for characterisation of patients as responders or non-responders to therapy based on differential expression of one or more genes are provided. Gene expression profiles, microarrays comprising nucleic acid sequences representing gene expression profiles, and new diagnostic kits and methods of treatment are also provided. The kits and methods relate to the treatment of specific populations of, for example, cancer patients, as characterised by their gene expression profile, suffering from MAGE expressing tumours.

### CROSS-REFERENCE TO RELATED APPLICATIONS

This application is filed pursuant to 35 U.S.C. 111(a) as a United States application which claims the benefit of U.S. Provisional Application No. 61/277,046 filed on 18 Sep. 2009 and U.S. Provisional Application No. 61/278,387 filed on 6 Oct. 2009 and claims priority to British Application No. GB0917457.4 filed on 6 Oct. 2009, each of which are incorporated herein by reference in their entirety.

### INCORPORATION-BY-REFERENCE OF MATERIAL SUBMITTED ON A COMPACT DISC

Applicants hereby incorporate-by-reference the material of the compact disc containing the files named: “VR63933P_pe.txt” created on 6 Oct. 2009 (file size 23.330 MB); and “VR63933P_rq.txt” created on 6 Oct. 2009 (file size 15.767 MB) filed in U.S. Provisional Application 61/278,387 filed 6 Oct. 2009, the benefit of which is claimed herein. A total of two compact discs (including duplicates) are incorporated by reference in the present paragraph.

To utilize the pe data on these disks, import the VR63933P_pe.txt ASCII file into an R session by typing in the following commands in a R session:

pe<-read.table(“VR63933P_pe.txt”)

pe<-unstack(pe)

To utilize the rq data on these disks, import the VR63933P_rq.txt ASCII file into an R session by typing in the following commands in a R session:

rq<-scan (“VR63933P_rq.txt.”)

### FIELD OF THE INVENTION

The present invention relates to gene expression profiles; methods for classifying patients; microarrays; and treatment of populations of patients selected through use of methods and microarrays as described herein.

### BACKGROUND

Melanomas are tumors originating from melanocyte cells in the epidermis. Patients with malignant melanoma in distant metastasis (stage IV according to the American Joint Commission on Cancer (AJCC) classification) have a median survival time of one year, with a long-term survival rate of only 5%. Even the standard chemotherapy for stage IV melanoma has therapeutic response rates of only 8-25%, but with no effect on overall survival. Patients with regional metastases (stage III) have a median survival of two to three years with very low chance of long-term survival, even after an adequate surgical control of the primary and regional metastases (Balch et al., 1992). Most Patients with stage I to III melanoma have their tumour removed surgically, but these patients maintain a substantial risk of relapse. Thus there remains a need to prevent melanoma progression, and to have improved treatment regimes for metastatic melanoma and adjuvant treatments for patients having had a primary tumour removed.

There are two types of lung cancer: non-small cell lung cancer (NSCLC) and small cell lung cancer (SCLC). The names simply describe the type of cell found in the tumours. NSCLC includes squamous-cell carcinoma, adenocarcinoma, and large-cell carcinoma and accounts for around 80% of lung cancers. NSCLC is hard to cure and treatments available tend to have the aim of prolonging life, as far as possible, and relieving symptoms of disease. NSCLC is the most common type of lung cancer and is associated with poor outcomes (Gatzmeier et al., 1994). Of all NSCLC patients, only about 25% have loco-regional disease at the time of diagnosis and are still amenable to surgical excision (stages IB, IIA or IIB according to the AJCC classification). However, more than 50% of these patients will relapse within the two years following the complete surgical resection. There is therefore a need to provide better treatment for these patients.

Traditional chemotherapy is based on administering toxic substances to the patient and relying, in part, on the aggressive uptake of the toxic agent by the tumour/cancer cells. These toxic substances adversely affect the patient's immune system, leaving the individual physically weakened and susceptible to infection.

It is known that not all patients with cancer respond to current cancer treatments. It is thought that only 30% or less of persons suffering from a cancer will respond to any given treatment. The cancers that do not respond to treatment are described as resistant. In many instances there have not been reliable methods for establishing if the patients will respond to treatment. However, administering treatment to patients who are both responders and non-responders because they cannot be differentiated is an inefficient use of resources and, even worse, can be damaging to the patient because, as discussed already, many cancer treatments have significant side effects, such as severe immunosuppression, emesis and/or alopecia. It is thought that in a number of cases patients receive treatment, when it is not necessary or when it will not be effective.

A new generation of cancer treatments based on antigens, peptides, DNA and the like is currently under investigation by a number of groups. The strategy behind many of these therapies, often referred to as cancer immunotherapy, is to stimulate the patient's immune system into fighting the cancer. These therapies are likely to be advantageous because the side effects, of taking such treatments, are expected to be minimal in comparison to the side effects currently encountered by patients undergoing cancer treatment. An antigen used in a cancer immunotherapy may be referred to as an ASCI, that is antigen-specific cancer immunotherapeutic.

In the early 1980s, Van Pel and Boon published the discovery of cytolytic T cells directed against an antigen presented on tumour cells. This led to the characterization of the first tumour-specific, shared antigen: Melanoma AGE-1 (MAGE-1, subsequently renamed MAGE-A1). It was followed by the identification of a large number of genes sharing the same expression pattern: they are expressed in a wide range of tumour types such as, melanoma, lung, bladder, breast, head and neck cancers. They are not expressed in normal cells, except testis. However, this expression in the testis does not normally lead to antigen expression, as these germ line cells do not express MHC class I molecules. From their peculiar expression profile, the name of Cancer Testis (CT) genes was proposed for these genes.

MAGE antigens are antigens encoded by the family of Melanoma-associated antigen genes (MAGE). MAGE genes are predominately expressed on melanoma cells (including malignant melanoma) and some other cancers including NSCLC (non small cell lung cancer), head and neck squamous cell carcinoma, bladder transitional cell carcinoma and oesophagus carcinoma, but are not detectable on normal tissues except in the testis and the placenta (Gaugler et al Human gene MAGE-3 codes for an antigen recognized on a melanoma by autologous cytolytic T lymphocytes J Exp Med. 1994 Mar. 1; 179(3):921-930); Weynants et al Expression of mage genes by non-small-cell lung carcinomas Int. J Cancer. 1994 Mar. 15; 56(6):826-829, Patard et al Int J. Cancer 64: 60, 1995). MAGE-A3 is expressed in 69% of melanomas (Gaugler, 1994), and can also be detected in 44% of NSCLC (Yoshimatsu 1988), 48% of head and neck squamous cell carcinoma, 34% of bladder transitional cell carcinoma, 57% of oesophageal carcinoma, 32% of colon cancers and 24% of breast cancers (Van Pel, et al Genes coding for tumor antigens recognized by cytolytic T lymphocytes Immunological Reviews 145, 229-250, 1995, 1995.); Inoue 1995; Fujie 1997; Nishimura 1997). Cancers expressing MAGE proteins are known as Mage associated tumours.

A large amount of work has been done in recent times to assist in the diagnosis and prognosis of cancer patients, for example to identify those patients who do not require further treatment because they have no risk of metastasis, recurrence or progression of the disease.

WO 2006/124836 identifies certain gene expression signatures over several oncogenic pathways, thereby defining the prognosis of the patient and sensitivity to therapeutic agents that target these pathways. The specific oncogenes are; Myc, Ras, E2, S3, Src and beta-catenin.

US 2006/0265138 discloses a method of generating a genetic profile, generally for identifying the primary tumour so that appropriate treatment can be given.

US 2006/0240441 and US 2006/0252057 describe methods of diagnosing lung cancer based on the differential expression of certain genes.

US 2006/0234259 relates to the identification and use of certain gene expression profiles of relevance to prostate cancer.

WO 2006/103442 describes gene expression profiles expressed in a subset of estrogen receptor (ER) positive tumours, which act, as a predictive signature for response to certain hormone therapies such as tamoxifen and also certain chemotherapies.

WO 2006/093507 describes a gene profile useful for characterising a patient with colorectal cancer as having a good prognosis or a bad prognosis, wherein patients with a good prognosis are suitable for chemotherapy.

WO 2006/092610 describes a method for monitoring melanoma progression based on differential expression of certain genes and novel markers for the disease, in particular TSBY1, CYBA and MT2A.

WO 2005/049829 describes an isolated set of marker genes that may be employed to predict the sensitivity of certain cancers to a chemotherapeutic agent, which is an erbB receptor kinase inhibitor, such as gefitinib.

Microarray gene profiling has been shown to be a powerful technique to predict whether cancer patients will respond to a therapy or to assess the prognosis of the disease, regardless of any therapeutic interventions. A number of large scale clinical trials are currently in progress to validate the profiles believed to be associated with different prognoses in breast cancer and follicular lymphoma (Dave, 2004; Hu, 2006; Weigelt, 2005).

Cells, including tumour cells, express many hundreds even thousands of genes. Differential expression of genes between patients who respond to a therapy compared to patients who do not respond, may enable specific tailoring of treatment to patients likely to respond.

### SUMMARY OF THE INVENTION

In one aspect the invention provides a method of classifying a patient as a responder or non-responder to an appropriate immunotherapy comprising the steps of:

(a) determining the expression levels of one or more genes in a patient-derived sample, wherein the gene(s) are selected from Table 1;

(b) classifying the patient to either a responder or non-responder group based on the expression levels of (a) by using an algorithm whose parameters were defined by a training set.

In one aspect the invention provides a method of characterising a patient as a responder or non-responder to a therapy comprising the steps:

(a) analysing a patient derived sample for differential expression of the gene products of one or more genes of Table 1, and

(b) characterising the patient from which the sample was derived as a responder or non-responder, based on the results of step (a), wherein the characterisation step is performed by reference or comparison to a standard or a training set or using an algorithm whose parameters were obtained from a standard or training set.

In one embodiment is provided a method of treating a patient by obtaining an analysis of a patient derived sample for differential expression of the gene products of one or more genes of Table 1. The results characterise a patient as a responder or non-responder to an immunotherapeutic and the characterisation step is performed by reference or comparison to a standard or a training set or using an algorithm whose parameters were obtained from a standard or training set. The patient is then selected for at least one administration of an appropriate immunotherapeutic if the patient is characterized as a responder to the immunotherapeutic.

In one embodiment is provided a method of determining whether a patient is a responder or a non-responder to an immunotherapeutic by obtaining a patient derived sample and analysing the patient derived sample for differential expression of the gene products of one or more genes of Table 1. The results determine whether the patient is characterised as a responder or non-responder to an immunotherapeutic and the characterisation step is performed by reference or comparison to a standard or a training set or using an algorithm whose parameters were obtained from a standard or training set.

In one embodiment, step (b) is based on a mathematical discriminant function or a decision tree. The decision tree may involve at least one bivariate classification step.

In a further embodiment, the present invention provides a method for characterising a patient as a responder or non-responder to therapy comprising analysing, in a patient-derived sample, a gene product recognised by one or more of the probe sets listed in Table 1, the target sequences of which are shown in Table 3, wherein the characterisation step is performed by reference or comparison to a standard or a training set or using an algorithm whose parameters were obtained from a standard or training set.

In an exemplary embodiment, the one or more genes or probe sets of Table 1 are at least 63 genes listed in Table 1 or at least the 74 probe sets listed in Table 1.

In an exemplary embodiment, the methods of the invention involve determining the expression levels of the genes or measurement of gene products of the probe sets specified in Tables 2, 5, 7 or 9. Each gene and probe set in these tables as well as groups of genes or probe sets form a specific aspect of this invention. The genes and probe sets in Tables 2, 5, 7 and 9 represent specific subsets of the genes and probe sets in Table 1.

Also provided is a predictive gene profile which may be used to differentiate between a responder patient and a non-responder patient to MAGE-A3 ASCI or any immunotherapeutic approach, wherein the profile comprises one or more genes selected from the genes listed in Table 1.

In one embodiment there is provided a gene profile as described herein, wherein the genes are genes recognised by the probe sets listed in Table 1.

In a further aspect a profile comprises or consists of all the genes listed in Table 1 or comprises or consists of all the genes recognised or targeted by the probe sets listed in Table 1.

In one aspect the invention provides a microarray comprising polynucleotide probes complementary and hybridisable to a sequence of the gene product of at least one gene selected from the genes listed in Table 1, in which polynucleotide probes or probe sets complementary and hybridisable to the genes of Table 1 constitute at least 50% of the probes or probe sets on said microarray.

In one aspect the invention provides a microarray comprising polynucleotide probes complementary and hybridisable to a sequence of the gene product of at least one gene selected from the genes listed in Table 1.

In one aspect the invention provides a solid surface to which are linked to a plurality of detection agents of at least 63 of the genes listed in Table 1, which detection agents are capable of detecting the expression of the genes or polypeptides encoded by the genes.

In one aspect the invention provides a diagnostic kit comprising means for detecting the expression of the one or more of the genes listed in Table 1 or of the gene products of the genes listed in Table 1. The expression may be detected by means of probes hybridising with mRNA or cDNA gene products.

In one aspect the invention provides one or more probes for identifying gene products, for example mRNA or cDNA, of one or more genes of Table 1 or of the gene products of the genes listed in Table 1.

In one aspect the invention provides use of PCR (or other known techniques) for identification of differential expression (such as upregulation) of one or more of the gene products of Table 1, or of the gene products of the gene profiles as described herein.

In a further embodiment, the present invention provides a method of treating a patient characterised as a responder to therapy, comprising administering a therapy, vaccine or immunogenic composition as described herein to the patient.

In a further embodiment, the present invention provides a method of treating a patient characterised as a non-responder to a therapy according to methods described herein or use of a diagnostic kit as described herein, comprising administering an alternative therapy or a combination of therapies, for example chemotherapy and/or radiotherapy may be used instead of or in addition to a vaccine or immunogenic composition as described herein.

In a further embodiment, the present invention provides use of a composition comprising a tumour associated antigen in the preparation of a medicament for the treatment of patients characterised as responders according to methods described herein, use of a microarray as described herein, use of a gene profile as described herein or use of a diagnostic kit as described herein.

### BRIEF DESCRIPTION OF THE DRAWINGS

FIG. 1/21 shows the scheme for the Leave One Out Cross Validation (LOOCV).

FIG. 2/21 shows the results of the LOOCV selecting the best 100 PS for classification in each loop. Open circles=non-responder, AS02B arm. Closed circles=responder, AS02B arm. Open triangle=non-responder, AS15 arm. Closed triangle=responder, AS15 arm.

FIG. 3/21 shows the number of times that a probe set (PS) was within the 100 top s2n (signal to noise) in each LOOCV (PS number on the X axis).

FIG. 4/21 shows the Kaplan-Meier curves (KM) for Overall Survival by adjuvant with all patients in the Phase II melanoma trial. Solid line=AS15 arm. Dotted line=AS02B arm.

FIG. 5/21 shows the KM for Overall Survival by gene signature based on LOOCV classification. Solid line=gene signature positive (GS+); dotted line=gene signature negative (GS−).

FIG. 6/21 shows Overall Survival Kaplan-Meier curves by adjuvant and gene signature based on LOOCV classification. Heavy solid line=AS15 arm, GS+. Heavy dotted line=AS15 arm, GS−. Light solid line=AS02B arm, GS+. Light dotted line=AS02B arm, GS−.

FIG. 7/21 shows classification of samples using the 100 PS (not leave one out). Open circles=non-responder, AS02B arm. Closed circles=responder, AS02B arm. Open triangle=non-responder, AS15 arm. Closed triangle=responder, AS15 arm.

FIG. 8/21 shows leave one out classification of corresponding samples using the 22 genes measured by PCR specified in Table 5. Open circles=non-responder, AS02B arm. Closed circles=responder, AS02B arm. Open triangle=non-responder, AS15 arm. Closed triangle=responder, AS15 arm.

FIG. 9/21 shows classification of samples using the 22 genes specified in Table 5 (not leave one out). Open circles=non-responder, AS02B arm. Closed circles=responder, AS02B arm. Open triangle=non-responder, AS15 arm. Closed triangle=responder, AS15 arm.

FIG. 10/21 shows the NSCLC Phase II trial design.

FIG. 11/21 shows the KM curve for Disease-Free Interval for the NSCLC trial. Solid line with circles=MAGE-A3; dashed line with squares=placebo.

FIG. 12/21 shows the Cox-SPCA methodology used in the examples of this application.

FIG. 13/21 shows survival curves by gene profile based on the LOOCV classification with median as cut-off using the 23 genes listed in Table 6 measured by PCR. Heavy solid line=MAGE immunotherapy, GS+. Heavy dotted line=MAGE immunotherapy, GS−. Light solid line=placebo, GS+. Light dotted line=placebo, GS−.

FIG. 14/21 shows distribution of risk score among placebo (left-hand panel) and vaccine arm (right-hand panel) in 129 NSCLC samples using the 23 genes listed in Table 6 measured by PCR using LOOCV classification. Closed diamonds=relapse; open diamonds=non-relapse.

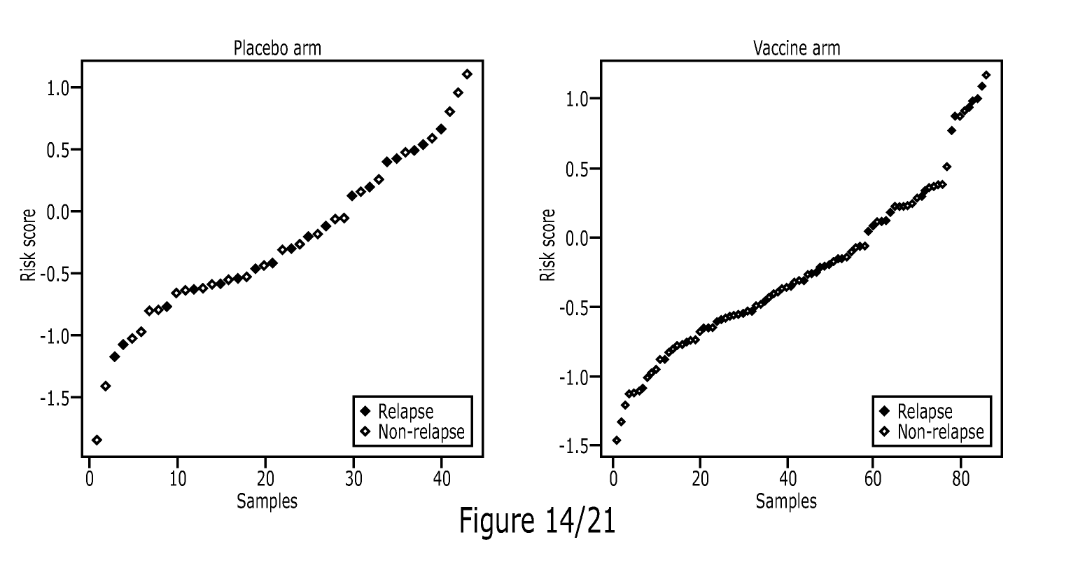

FIG. 15/21 shows the clinical outcome based on classification using the 23 genes by Q-PCR in the classifier as listed in Table 6 (not leave one out). Heavy solid line=MAGE immunotherapy, GS+. Heavy dotted line=MAGE immunotherapy, GS−. Light solid line=placebo, GS+. Light dotted line=placebo, GS−.

FIG. 16/21 shows the risk score among placebo (left-hand panel) and vaccine arm (right-hand panel) based on the classification using the 23 genes by Q-PCR in the classifier as listed in Table 6 (not leave one out). Closed diamonds=relapse; open diamonds=non-relapse.

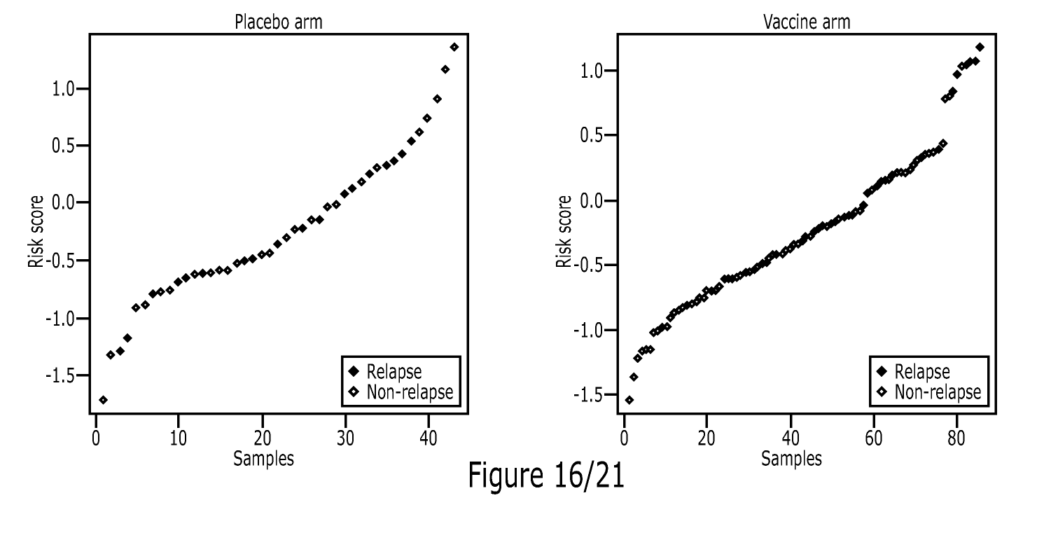

FIG. 17/21 shows survival curves by gene profile based on the LOOCV classification with median as cut-off in 129 NSCLC samples using the 22 genes listed in Table 5. Heavy solid line=MAGE immunotherapy, GS+. Heavy dotted line=MAGE immunotherapy, GS−. Light solid line=placebo, GS+. Light dotted line=placebo, GS−.

FIG. 18/21 shows distribution of risk score among placebo (left-hand panel) and vaccine arm (right-hand panel) in 129 NSCLC samples using the 22 genes listed in Table 5 using LOOCV classification. Closed diamonds=relapse; open diamonds=non-relapse.

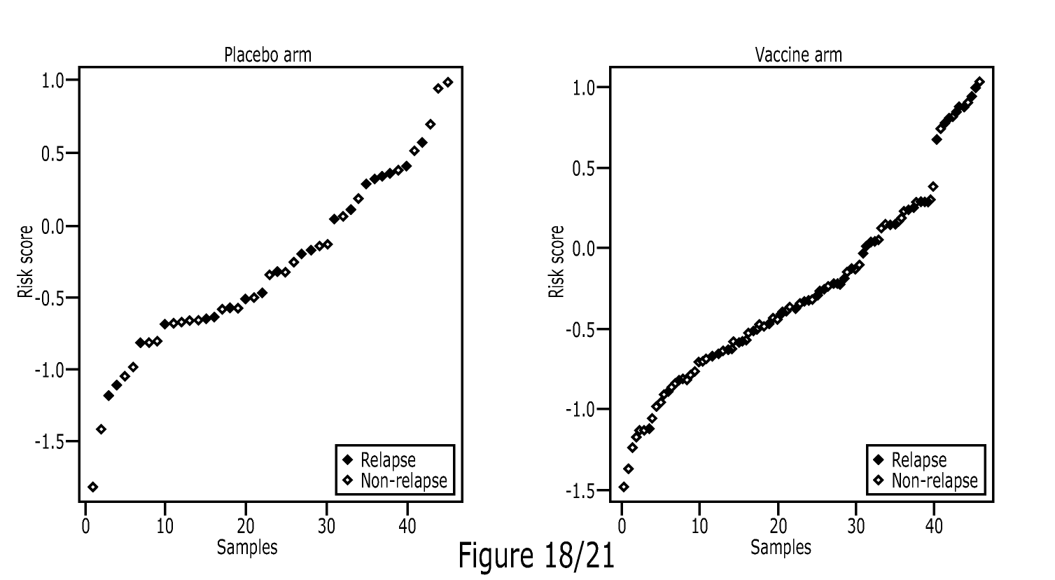

FIG. 19/21 shows the clinical outcome based on the classification using the 22 genes by Q-PCR in the classifier as listed in Table 5 (not leave one out). Heavy solid line=MAGE immunotherapy, GS+. Heavy dotted line=MAGE immunotherapy, GS−. Light solid line=placebo, GS+. Light dotted line=placebo, GS−.

FIG. 20/21 shows the risk score based on the classification using the 22 genes by Q-PCR in the classifier as listed in Table 5 (not leave one out). Closed diamonds=relapse; open diamonds=non-relapse.

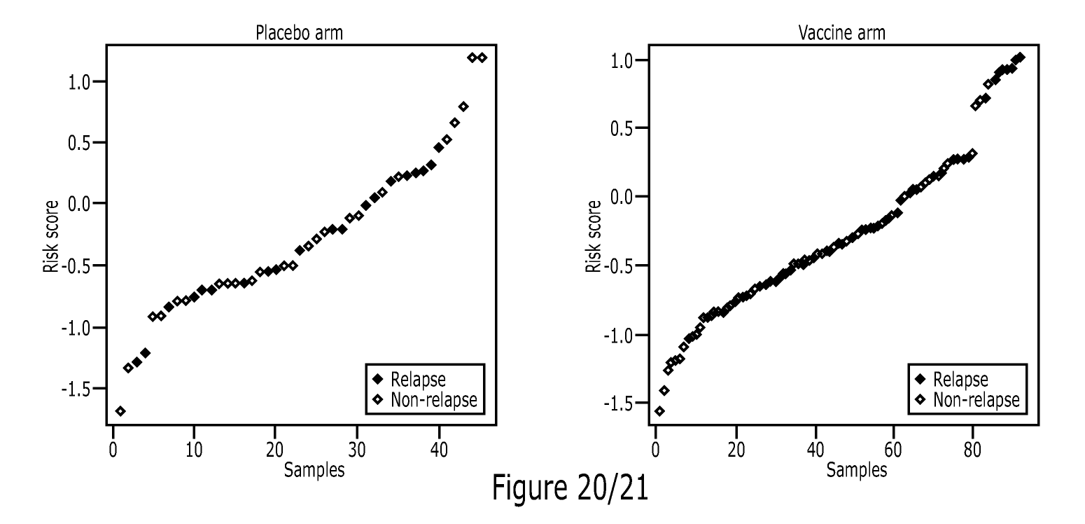

FIG. 21/21 shows the protein D 1/3-MAGE3-HIS protein.

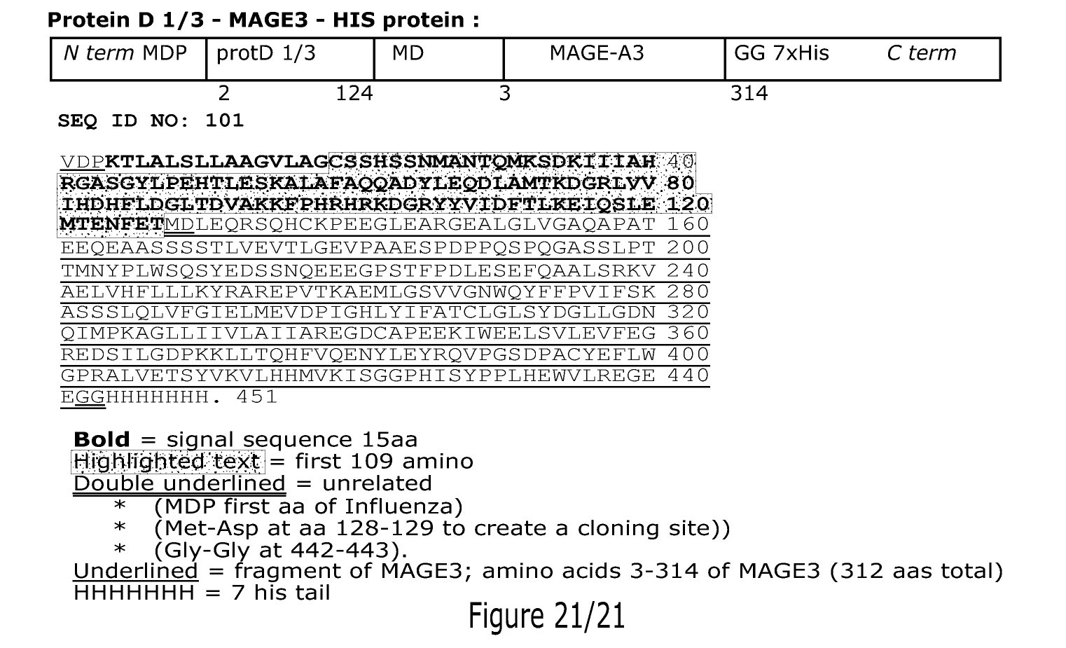

### SEQUENCE IDENTIFIERS AND TABLES

The following sequence identifiers are included in the sequence listing:

SEQ ID NO: 1-100—Probe set target sequences shown in Table 3

SEQ ID NO: 101—Protein D—MAGE-A3 fusion protein

SEQ ID NO: 102-106-CpG oligonucleotide sequences

SEQ ID NO:107-113—MAGE peptide sequences

Table 1: 100 PS and corresponding gene list.

Table 1A: 100 PS selected using all samples and the times selected in LOOCV

Table 2: Subset of 27 PS and 21 genes from Table 1.

Table 3: 100 PS target sequences.

Table 4: Mean, Standard Deviations (Sd) and PC₁Coefficients for the 100 PS classifier features.

Table 5: Suitable subset of 22 genes in melanoma.

Table 6: Mean, Standard deviations (Sd) and PC1 coefficients for 22 genes classifier features in melanoma.

Table 7: Suitable subset of 23 genes in NSCLC

Table 8: Mean, Standard deviations (Sd) and PC1 coefficients for 23 genes classifier features in NSCLC.

Table 9: Suitable subset of 22 genes in NSCLC

Table 10: Mean, Standard deviations (Sd) and PC1 coefficients for 22 genes classifier features in NSCLC.

Table 11: Classification performance of individual genes measured by Q-PCR in melanoma samples

Table 12: Classification performance of individual genes measured by Q-PCR in NSCLC samples

Table 13: Classification performance of individual genes measured by microarray in melanoma samples

### DETAILED DESCRIPTION OF THE INVENTION

### Predictive Gene Profile

Analysis performed on pre-treatment tumour tissue from patients having malignant melanoma, following surgical resection, identified that certain genes were differentially expressed in patients that were more likely to respond to therapy (responders), in comparison to those patients who were less likely to respond (non-responders).

The present inventors have discovered a gene profile that is predictive of the likelihood of a patient's response to therapy.

By “gene profile” is intended a gene or a set of genes the expression of which correlates with patient response to therapy because the gene or set of genes exhibit(s) differential expression between patients having a favourable response to therapy and patients having a poor response to therapy. In one embodiment of the invention the term “gene profile” refers to the genes listed in Table 1 or to any selection of the genes of Table 1 which is described herein.

As used herein, a ‘favorable response’ (or ‘favorable clinical response’) to, for example, an anticancer treatment refers to a biological or physical response that is recognized by those skilled in the art as indicating a decreased rate of tumor growth, compared to tumor growth that would occur with an alternate treatment or the absence of any treatment. A favorable clinical response to therapy may include a lessening of symptoms experienced by the subject, an increase in the expected or achieved survival time, a decreased rate of tumor growth, cessation of tumor growth (stable disease), regression in the number or mass of metastatic lesions, and/or regression of the overall tumor mass (each as compared to that which would occur in the absence of therapy, or in response to an alternate therapy). In the case of adjuvant cancer therapy, a favorable clinical response may include an absence or relapse or delay in relapse rate or increase in disease free survival time or interval time.

“Differential expression” in the context of the present invention means the gene is up-regulated or down-regulated in comparison to its normal expression. Statistical methods for calculating differential expression of genes are discussed elsewhere herein.

In some aspects, the invention provides a gene profile for characterising a patient as a responder or non-responder to therapy, in which the profile comprises differential expression of at least one gene of Table 1, or in which the profile comprises or consists of the genes listed in Table 1. A profile may be indicative of a responder or non-responder. In one embodiment, the gene profiles described herein are indicative of responders.

The gene sequences recognised or targeted by the probe sets of Table 1 are listed in Table 3.

By “genes of Table 1” is meant the genes listed under “Gene name” in Table 1, 2, 5, 7 or 9. By “gene product” is meant any product of transcription or translation of the genes, whether produced by natural or artificial means.

In one embodiment of the invention, the genes referred to herein are those listed in Table 1, 2, 5, 7 or 9 as defined in the column indicating “Gene name”. In another embodiment, the genes referred to herein are genes the product of which are capable of being recognised by the probe sets listed in Table 1.

Whilst not wishing to be bound by theory it is hypothesised that the gene signature identified in Table 1 is in fact indicative of an immune/inflammatory, such as a T cell infiltration/activation response in the patients who are designated as responders, for example, the signature may represent a T-cell activation marker. The signature may also represent Th1 markers including members of interferon pathway which tend to favour the induction of cell mediated immune responses. The presence of this response is thought to assist the patient's body to fight the disease, such as cancer, after administration of the immunotherapy thereby rendering a patient more responsive to said immunotherapy.

Thus the signatures of the present invention do not generally focus on markers/genes specifically associated with the diagnosis and/or prognosis of the relevant disease, for example cancer such as oncogenes, but rather is predictive of whether the patient will respond to an appropriate immunotherapy, such as cancer immunotherapy.

The gene profile identified herein is thought to be indicative of the microenvironment of the tumor. At least in this aspect the correct microenvironment of the tumor seems to be key to whether the patient responds to appropriate cancer immunotherapy.

The biology of the signature is relevant to the ASCI mode of action since it contains genes that suggest the presence of a specific tumor microenvironment (chemokines) that favor presence of immune effector cells in the tumor of responder patients which show upregulation of T-cell markers and Th1 markers including members of interferon pathway. A recent gene expression profiling study in metastatic melanoma revealed that tumors could be segregated based on presence or absence of T-cell associated transcripts (Harlin, 2009). The presence of lymphocytes in tumors correlated with the expression of a subset of six chemokines (CCL2, CCL3, CCL4, CCL5, CXCL9, CXCL10), three out of these six genes (CCL5, CXCL9, CXCL10) are present in the 100 PS of Table 1.

In one embodiment the invention employs one or more (such as substantially all) the genes listed in Table 1. Suitably the invention employs at least 63 of the genes or 74 of Probe Sets listed in Table 1.

Suitably, the one or more genes of Table 1 are at least 63, at least 64, at least 65, at least 66, at least 67, at least 68, at least 69, at least 70, at least 71, at least 72, at least 73, at least 74, at least 75, at least 76, at least 77, at least 78, at least 79, at least 80 or substantially all the genes listed in Table 1 and/or any combination thereof.

Suitably, the one or more probe sets of Table 1 are at least 74, at least 75, at least 76, at least 77, at least 78, at least 79, at least 80, at least 81, at least 82, at least 83, at least 84, at least 85, at least 86, at least 87, at least 88, at least 89, at least 90 or substantially all the probe sets listed in Table 1 and/or any combination thereof.

Substantially all in the context of the gene lists will be at least 90%, such a 95%, particularly 96, 97, 98 or 99% of the genes in the given list.

In one aspect the invention is employed in a metastatic setting.

If a gene is always upregulated or always down regulated in patients that are deemed to be responders (or alternatively non-responders) then this single gene can be used to establish if the patient is a responder or a non-responder once a threshold is established and provided the separation of the two groups is adequate.

In one aspect the invention provides a gene profile for identifying a responder comprising one or more of said genes wherein 50, 60, 70, 75, 80, 85, 90, 95, 99 or 100% of the genes are upregulated. In contrast in non-responders the gene/genes is/are not upregulated or is/are down regulated.

In the context of the present invention, the sample may be of any biological tissue or fluid derived from a patient potentially in need of treatment. The sample maybe derived from sputum, blood, urine, or from solid tissues such as biopsy from a primary tumour or metastasis, or from sections of previously removed tissues.

Samples could comprise or consist of, for example, needle biopsy cores, surgical resection samples or lymph node tissue. These methods include obtaining a biopsy, which is optionally fractionated by cryostat sectioning to enrich tumour cells to about 80% of the total cell population. In certain embodiments, nucleic acids extracted from these samples may be amplified using techniques well known in the art. The levels of selected markers can be detected and can be compared with statistically valid groups of, for example, Mage positive non responder patients.

For analysis in relation to cancer, the biological sample will be taken so as to maximise the opportunity for the sample to contain cancer or tumour cells and may, for example, be derived from the cancer or tumour such as a fresh sample (including frozen samples) or a sample that has been preserved in paraffin. Having said this, samples preserved in paraffin can suffer from degradation and the profile observed may be modified. A person working in the field is well able to compensate of these changes observed by recalibrating the parameters of the profile.

In one aspect the biological sample is a biopsy sample, for example from a tumor or cancerous tissue.

In one aspect the cancer immunotherapy is for the treatment of melanoma, lung cancer for example NSCLC, bladder cancer, neck cancer, colon cancer, breast cancer, esophageal carcinoma and/or prostate cancer, such as lung cancer and/or melanoma, in particular melanoma.

“Responder” in the context of the present invention includes persons where the cancer/tumor(s) is eradicated, reduced or improved (Complete Responder or Partial Responder; Mixed Responder) or simply stabilised such that the disease is not progressing (“Stable Disease”). “Complete clinical responder” in respect of cancer is wherein all of the target lesions Disappear.

“Partial clinical responder” or “Partial Responder” in respect of cancer is wherein all of the tumors/cancers respond to treatment to some extent, for example where said cancer is reduced by 30, 40, 50, 60% or more.

“Progressive disease” represents 20% increase in size of target lesions or the appearance of one or more new lesions or both of these.

Patients with progressive disease (PD) can further be classifier to PD with no-Mixed Response or progressive disease with “Mixed clinical responder” of type I or II or “Mixed Responder” in respect of cancer is defined as wherein some of the tumors/cancers respond to treatment and others remain unchanged or progress.

Non-Responders (NR) are defined as patients with progressive disease without mixed response and progressive disease with mixed response II that did not show disappearance of at least one target lesion.

In responders where the cancer is stabilised then the period of stabilisation is such that the quality of life and/or patients life expectancy is increased (for example stable disease for more than 6 months) in comparison to a patient that does not receive treatment.

In some embodiments, the term “responder” may not include a “Mixed Responder”

A predicted characterisation of a new patient as a responder (gene signature positive) or non-responder (gene signature negative) can be performed by reference to a “standard” or a training set or by using a mathematical model/algorithm (classifier) whose parameters were obtained from a training set. The standard may be the profile of a person/patient(s) who is known to be a responder or non-responder or alternatively may be a numerical value. Such pre-determined standards may be provided in any suitable form, such as a printed list or diagram, computer software program, or other media.

The standard is suitably a value for, or a function of, the expression of a gene product or products in a patient or patients who have a known responder or non responder status, such that comparison of the standard information with information concerning expression of the same genes in the patient derived sample allows a conclusion to be drawn about responder or non-responder status in the patient. The standard may be obtained using one or more genes of Table 1, and from analysis of one or more individuals who are known to be responders or non-responders.

Non-limiting examples of training data or parameters obtained from the training set are the reference data set, reference quantiles, probe effects or the R object format data used for sample normalisation as discussed in Example 1 below. Use of these specific examples in the classification of patients as responders or non-responders forms a specific aspect of this invention.

In one aspect the statistical analysis is performed by reference to a standard or training set. The gene list in Table 1 was generated by calculating the signal to noise of each probeset using the clinical outcome (Responder and Non-Responder) of the patients in the training set as the groups in the comparison. Classifier parameters derived from the training set are then used to predict the classification for new samples.

Training set in the context of the present specification is intended to refer to a group of samples for which the clinical results can be correlated with the gene profile and can be employed for training an appropriate statistical model/programme to identify responders and/or non-responder for new samples.

Whilst not wishing to be bound by theory it is thought that at least 68 out of the 100 genes in Table 1 are resistant to changes in the training set. These genes form a specific aspect of this invention. These genes can be identified from column 5 of Table 1A.

In one aspect a mathematical model/algorithm/statistical method is employed to characterise the patient as responder or non-responder.

The algorithm for characterisation uses gene expression information from any one gene and any one known responder or non-responder and is suitably based on supervised principal component analysis, although any suitable characterisation algorithm may be used, for example any algorithms of Examples 1-7.

Specifically the algorithm may generate a standard from an individual or a training set with a known clinical outcome using the Supervised Principal Component Analysis with Discriminant analysis algorithm as shown in example 1 or the Supervised Principal Component Analysis with the cox decisions rule as shown in example 3.

Therefore, in one aspect the invention also relates to the development of a classifier for characterisation of a new patient as a responder or non-responder, the parameters of the classifier being obtained from a training set with known clinical outcome (Responder and Non-Responder).

The gene lists may be generated using signal to noise, Baldi analysis a variation of the classical T test, and/or Pearsons Correlation Coefficient and/or Linear Discriminant analysis. See for example Golub T, Slonim D, Tamayo P et al. Molecular classification of cancer: class discovery and class prediction by gene expression monitoring. Science 1999; 286: 531-536. Van 't Veer L J, Dai H, van de Vijver M J, He Y D, Hart A A, Mao M, Peterse H L, van der Kooy K, Marton M J, Witteveen A T, et al. (2002) Gene expression profiling predicts clinical outcome of breast cancer. Nature, 415(6871), 530-556.

The classifier might use a supervised principal components, discriminant analysis, nearest centroid, kNN, support vector machines or other algorithms appropriate for classification; including algorithms that use time (e.g. survival time, disease free interval time) for classification. Alternatively, classification can be achieved using other mathematical methods that are well known in the art.

The classifier may comprise a SPCA with DA decision rule exemplified in example 1 and/or 2 or a SPCA-Cox decision rule exemplified in example 3 and/or 4. In some embodiments, the disclosed methods are greater than 50%, 60% or 70% accurate such as about 70% accurate at predicting responders and non-responders correctly.

In one embodiment the responder and non-responder are defined by reference to the Time to Treatment Failure (TTF)/Overall survival (OS), which is a continuous variable and may for example be measured in months. Where the time to treatment failure variable is large then the patient will be considered to be a responder. Where the time to treatment failure variable is small then patient will be considered to be a non-responder. Generally using this approach the mixed responders are also grouped with the responders.

Treatment failure is where the patient does not fall with the definition of responder, partial responder, mixed responder or stable disease as defined herein.

In one aspect non-responders may be defined as those with a TTF of 6 months or less.

In one aspect the responders may be defined as those with a TTF of more than 6 months, for example 7, 8, 9, 10, 11, 12, 13, 14, 15, 16, 17, 18, 19, 20, 21, 22, 23, 24 or more months.

In one aspect of the invention, the patient response to a treatment is the disease free interval (DFI) or disease free survival (DFS) which are continuous variables and may for example be measured in months. DFI and DFS are used for example in an adjuvant treatment; which is the case when the tumor has been removed and the treatment is provided to avoid or delay relapse or equivalently to extend the disease free interval or survival.

DFI and DFS can be correlated to patients clinical information or measured patients parameters such as biomarkers or a gene expression profile and can be used to build a mathematical model to predict the response of a new patient.

In one aspect, the methods of the invention involve determining the expression levels of the genes or measurement of gene products of the probe sets listed in Table 1.

In one aspect, the invention involves use of one or more (such as substantially all) the genes or probe sets listed in Table 1 for predicting or identifying a patient as a responder or non-responder to immunotherapy for both lung cancer and melanoma, suitably immunotherapy based on a cancer testis antigen such as Mage. Suitably the invention employs at least 63 of the genes or 74 of Probe Sets listed in Table 1.

TABLE 1

| | | Gene symbol | |
|---|---|---|---|
| | | according to | Gene symbol |
| | | R2.9 | according to |
| | Affy ID | annotation | Affymetrix annotation |
| 1.1 | AFFX- | STAT1 | STAT1 |
| | HUMISGF3A/M97935_MB_at | | |
| 1.2 | 1555852_at | PSMB9 | NA |
| 1.3 | 1562031_at | JAK2 | JAK2 |
| 1.4 | 201474_s_at | ITGA3 | ITGA3 |
| 1.5 | 202659_at | PSMB10 | PSMB10 |
| 1.6 | 203915_at | CXCL9 | CXCL9 |
| 1.7 | 204070_at | RARRES3 | RARRES3 |
| 1.8 | 204116_at | IL2RG | IL2RG |
| 1.9 | 204533_at | CXCL10 | CXCL10 |
| 1.10 | 205758_at | CD8A | CD8A |
| 1.11 | 205890_s_at | UBD | GABBR1 /// UBD |
| 1.12 | 207651_at | GPR171 | GPR171 |
| 1.13 | 207795_s_at | KLRD1 | KLRD1 |
| 1.14 | 208729_x_at | HLA-B | HLA-B |
| 1.15 | 208885_at | LCP1 | LCP1 |
| 1.16 | 208894_at | HLA-DRA | HLA-DRA |
| 1.17 | 209606_at | CYTIP | CYTIP |
| 1.18 | 210915_x_at | IL23A | TRBC1 |
| 1.19 | 210972_x_at | TRA@ | TRA@ /// TRAC /// TRAJ17 /// |
| | | | TRAV20 |
| 1.20 | 210982_s_at | HLA-DRA | HLA-DRA |
| 1.21 | 211144_x_at | TARP | TARP /// TRGC2 |
| 1.22 | 211339_s_at | ITK | ITK |
| 1.23 | 211796_s_at | IL23A | TRBC1 /// TRBC2 |
| 1.24 | 211911_x_at | HLA-B | HLA-B |
| 1.25 | 212671_s_at | HLA-DQA1 | HLA-DQA1 /// HLA-DQA2 |
| 1.26 | 213793_s_at | HOMER1 | HOMER1 |
| 1.27 | 215806_x_at | TRGC2 | TARP /// TRGC2 |
| 1.28 | 216920_s_at | TARP | TARP /// TRGC2 |
| 1.29 | 217436_x_at | HLA-A | HLA-A /// HLA-A29.1 /// HLA-B /// |
| | | | HLA-G /// HLA-H /// HLA-J |
| 1.30 | 217478_s_at | HLA-DMA | HLA-DMA |
| 1.31 | 221875_x_at | HLA-F | HLA-F |
| 1.32 | 222838_at | SLAMF7 | SLAMF7 |
| 1.33 | 223575_at | KIAA1549 | KIAA1549 |
| 1.34 | 225996_at | LONRF2 | LONRF2 |
| 1.35 | 228362_s_at | FAM26F | FAM26F |
| 1.36 | 228532_at | C1orf162 | C1orf162 |
| 1.37 | 229391_s_at | FAM26F | FAM26F |
| 1.38 | 229625_at | GBP5 | GBP5 |
| 1.39 | 232375_at | STAT1* | NA |
| 1.40 | 232481_s_at | SLITRK6 | SLITRK6 |
| 1.41 | 235175_at | GBP4 | GBP4 |
| 1.42 | 235276_at | EPSTI1 | EPSTI1 |
| 1.43 | 244393_x_at | AKR1C2* | NA |
| 1.44 | 1554240_a_at | ITGAL | ITGAL |
| 1.45 | 1552613_s_at | CDC42SE2 | CDC42SE2 |
| 1.46 | 204556_s_at | DZIP1 | DZIP1 |
| 1.47 | 204897_at | PTGER4 | PTGER4 |
| 1.48 | 206082_at | HCP5 | HCP5 |
| 1.49 | 211149_at | UTY | LOC100130224 /// UTY |
| 1.50 | 214470_at | KLRB1 | KLRB1 |
| 1.51 | 229543_at | FAM26F | FAM26F |
| 1.52 | 231229_at | HILS1 | HILS1 |
| 1.53 | 232234_at | C20orf24 | SLA2 |
| 1.54 | 232311_at | B2M | B2M |
| 1.55 | 236328_at | ZNF285A | ZNF285A |
| 1.56 | 237515_at | TMEM56 | TMEM56 |
| 1.57 | 202531_at | IRF1 | IRF1 |
| 1.58 | 209813_x_at | TRGV9 | TARP |
| 1.59 | 238524_at | NA | NA |
| 1.60 | 205097_at | SLC26A2 | SLC26A2 |
| 1.61 | 209774_x_at | CXCL2 | CXCL2 |
| 1.62 | 210439_at | ICOS | ICOS |
| 1.63 | 213193_x_at | IL23A | TRBC1 |
| 1.64 | 1555759_a_at | CCL5 | CCL5 |
| 1.65 | 1562051_at | LOC284757 | LOC284757 |
| 1.66 | 205685_at | CD86 | CD86 |
| 1.67 | 210606_x_at | KLRD1 | KLRD1 |
| 1.68 | 211902_x_at | TRA@ | TRA@ |
| 1.69 | 1552497_a_at | SLAMF6 | SLAMF6 |
| 1.70 | 204529_s_at | TOX | TOX |
| 1.71 | 206666_at | GZMK | GZMK |
| 1.72 | 1552612_at | CDC42SE2 | CDC42SE2 |
| 1.73 | 1563473_at | PPP1R16B* | NA |
| 1.74 | 219551_at | EAF2 | EAF2 |
| 1.75 | 228492_at | USP9Y | LOC100130216 /// USP9Y |
| 1.76 | 229390_at | FAM26F | FAM26F |
| 1.77 | 228316_at | FLJ31438* | C2orf63 |
| 1.78 | 228400_at | SHROOM3 | SHROOM3 |
| 1.79 | 202643_s_at | TNFAIP3 | TNFAIP3 |
| 1.80 | 204806_x_at | HLA-F | HLA-F |
| 1.81 | 213539_at | CD3D | CD3D |
| 1.82 | 226084_at | MAP1B | MAP1B |
| 1.83 | 205499_at | SRPX2 | SRPX2 |
| 1.84 | 223593_at | AADAT | AADAT |
| 1.85 | 244061_at | ARHGAP15* | NA |
| 1.86 | 222962_s_at | MCM10 | MCM10 |
| 1.87 | 1553132_a_at | TC2N | TC2N |
| 1.88 | 200615_s_at | AP2B1 | AP2B1 |
| 1.89 | 234907_x_at | GOLGA7* | NA |
| 1.90 | 207536_s_at | TNFRSF9 | TNFRSF9 |
| 1.91 | 239012_at | RNF144B | RNF144B |
| 1.92 | 209671_x_at | TRA@ | TRA@ /// TRAC |
| 1.93 | 238587_at | UBASH3B | UBASH3B |
| 1.94 | 209770_at | BTN3A1 | BTN3A1 |
| 1.95 | 204224_s_at | GCH1 | GCH1 |
| 1.96 | 221081_s_at | DENND2D | DENND2D |
| 1.97 | 229152_at | C4orf7 | C4orf7 |
| 1.98 | 202644_s_at | TNFAIP3 | TNFAIP3 |
| 1.99 | 238581_at | GBP5 | GBP5 |
| 1.100 | 231577_s_at | GBP1 | GBP1 |

*Annotation from R2.6 that became NA in R2.9

In one aspect, the methods of the invention involve determining the expression levels of the genes or measurement of gene products of the probe sets listed in Table 2.

TABLE 2

| | | Gene symbol |
|---|---|---|
| | | according to |
| | Gene Name | Affymetrix |
| Probe set | R2.9 annotation | annotation |
| AFFX- | STAT1 | STAT1 |
| HUMISGF3A/M97935_MB_at | | |
| 232375_at | STAT1* | NA |
| 209770_at | BTN3A1 | BTN3A1 |
| 204556_s_at | DZIP1 | DZIP1 |
| 228316_at | FLJ31438* | C2orf63 |
| 238581_at | GBP5 | GBP5 |
| 234907_x_at | GOLGA7* | NA |
| 213793_s_at | HOMER1 | HOMER1 |
| 210439_at | ICOS | ICOS |
| 223575_at | KIAA1549 | KIAA1549 |
| 207795_s_at | KLRD1 | KLRD1 |
| 210606_x_at | KLRD1 | KLRD1 |
| 1562051_at | LOC284757 | LOC284757 |
| 217436_x_at | HLA-A | HLA-A /// |
| | | HLA-A29.1 /// |
| | | HLA-B /// |
| | | HLA-G /// HLA-H |
| | | /// HLA-J |
| 225996_at | LONRF2 | LONRF2 |
| 226084_at | MAP1B | MAP1B |
| 222962_s_at | MCM10 | MCM10 |
| 238524_at | NA | NA |
| 239012_at | RNF144B | RNF144B |
| 228400_at | SHROOM3 | SHROOM3 |
| 205097_at | SLC26A2 | SLC26A2 |
| 232481_s_at | SLITRK6 | SLITRK6 |
| 238587_at | UBASH3B | UBASH3B |
| 237515_at | TMEM56 | TMEM56 |
| 207536_s_at | TNFRSF9 | TNFRSF9 |
| 204529_s_at | TOX | TOX |
| 236328_at | ZNF285A | ZNF285A |

*Annotation from R2.6 that became NA in R2.9

The target sequences for the probe sets listed in Table 1 are provided below.

TABLE 3

| | |
|---|---|
| Probe Set ID | Target Sequence |

| | |
|---|---|
| 1552497_a_at | [SEQ ID NO: 1] |
| | Tagcattacccttctgacactctctatgtagcctccctgatcttctttcagctcctctattaaa |
| | ggaaaagttctttatgttaattatttacatcttcctgcaggcccttcctctgcctgctggggtc |
| | ctcctattctttaggtttaattttaaatatgtcacctcctaagagaaaccttcccagaccact |
| | ctttctaaaatgaatcttctaggctgggcatggtggctcacacctgtaatcccagtactttg |
| | ggaggccaaggggggagatcacttgaggtcaggagttcaagaccagcctggccaa |
| | cttggtgaaaccccgtctttactaaaaatacaaaaaaattagccaggcgtggtggtgc |
| | acccctaaaatcccagctacttgagagactgaggcaggagaatcgcttgaacccag |
| | gaggtggaggttccagtgagccaaaatcatgccaatgtattccagtctg |
| 1552612_at | [SEQ ID NO: 2] |
| | tgttctgctctgaagaagatactgtcagacgaatcctgcatttccttcagctggc |
| 1552613_s_at | [SEQ ID NO: 3] |
| | gcatgcctttggactcatggacagagttctttnggattgtcactgaattttcaatgtttaatc |
| | agtatggatctgatcttcgcatgatctttttngtgaatgctaacaccattttgcagttttttttttc |
| | tattttaaacatttttcttttcactgccgancccnnngccttacgattttatnnggaaagcaa |
| | ggaccntgctattattnntntaatttgccatcatttatgtatattnnggaaggtatgagacc |
| | cacaagcacaantgatcattttnattngttngtnngttngaaacttcagcagaatagata |
| | tctgcatgctttatgaangttgttgcttcggtaagagcccatgggatgccagaaattaac |
| | atttctttgctgccatgggntgatgatgctgctattagataaangtttagctgtggnaccaa |
| | gtcacatcattttcatagaaaaagatnacttgtagcttattttagaagtatgaccttttggtct |
| | gtttga |
| 1553132_a_at | [SEQ ID NO: 4] |
| | Caggtggcacaaattaaatccatcttgaagacttcacacattaatttggtgaagaactt |
| | gacattcttttagaagacttatgatttcaatttgctaccaatgagaagaggcaaatcaac |
| | aaatttgtcaatttatgggggctataattatggtatataatgtatctgatagaaaatttgata |
| | agaaaatgtaatgaattttatcagatatccaaagtaaaggaaatgttttaaaactgcaa |
| | caagagacacagacagtaaaatcaaagtattattaggatgactaaataaattataaa |
| | gtctgtgagaatatcaaccatagatagttctttctatattatgtttttgcttttgtattttaagcttt |
| | acttagnatattcaaaacctggtatatcaagtctctgttagtactattggcatttagaagac |
| | tttaccattatttcagtgctaggcattattgattaggtcttggctccactgtttacct |
| 1554240_a_at | [SEQ ID NO: 5] |
| | Acacttggttgggtcctcacatctttcacacttccaccagcctgcactactccctcaaag |
| | cacacgtcatgtttcttcatccggcagcctggatgttttttccctgtttaatgattgacgtactt |
| | agcagctatctctcagtgaactgtgagggtaaaggctatacttgtcttgttcaccttggga |
| | tgatgcctcatgatatgtcagggcgtgggacatctagtaggtgcttgacataa |
| 1555759_a_at | [SEQ ID NO: 6] |
| | cccgtgcccacatcaaggagtatttctacaccagtggcaagtgctccaacccagcagt |
| | cgtctttgtcacccgaaagaaccgccaagtgtgtgccaacccagagaagaaatgggt |
| | tcgggagtacatc |
| 1555852_at | [SEQ ID NO: 7] |
| | ccattctgagtacttctccgcaaaccctttgtttcattaaggactgttttacatgaagggtgc |
| | aaaagtaggataaaaatgagaaccctagggtgaaacacgtgacagaagaataaa |
| | gactattgaatagtcctcttctctacccatggacnttggnatttttatattngattttaaggaa |
| | atataacttagtagtaaagagatgagcattcaagtcaggcagacctgaatttgggtcaa |
| | ggctgcgccactcaaaagctatatgacctctatatgagcagcttattcaacctcttttaac |
| | ctccattttgtcatctgtagaatgatgataaatgcctagctcagaaggattcc |
| 1562031_at | [SEQ ID NO: 8] |
| | atgttcactgtatgtgccaagcctaatatgagagctatgtattatagagtttatgctacagc |
| | cctaccttcaggaaacttatctactggacaaacaaaaattttcaaatatacaaaaaattc |
| | taaatcgaacattgtaattatctagcataggcaaatatagacagtaacagacaggttta |
| | caattattaagaaagggcagccagg |
| 1562051_at | [SEQ ID NO: 9] |
| | Atcgaggaagatatactgccaagtcaggaagaaaaaatccacctgttcagtgatttca |
| | ggaactgctgaagaaaatcaccagtgagtatcagtttctgcaagagaatctaatgcag |
| | gctttgcttctcatcggaatcccccagctggtgtcttggttgactgagagtctgggggaga |
| | gggcagagaatggatttattctctgctaggtttttaacagtcaagaagggctgtggtccta |
| | aggggcactggtcaaaccttagtgtgcatcagaattatctggataaggctaggcacag |
| | tggctcacgcctgtaatcacagcactttgggaggctgaggcgcgtggatcacctgagg |
| | tcagaagttcaagaccagcctggctcttttagtagag |
| 1563473_at | [SEQ ID NO: 10] |
| | gaaaattcctggcagtttcaactgtgatagacattgctaacctgttctccaaagaggctg |
| | aaccaatttctgtttcctcaacagtgtatgactgtttcccccatctattctccagcactgagg |
| | attaagtaactttcatttttgtcagtctgacagatataaagcagaacatttctgcataaggtt |
| | ctacagtaatttttagattttatgaccctttggattatgcctacataatgatgatcaaatattc |
| | agaaactacattgtacctggccttaggcttggaattggatacaaaattaaatgaaacca |
| | gcttttgccctcaggttgatcccatctcctggagttggcagacaaatgaacaaataaaat |
| | gagagcaaaactgtatggttcacattgtgctagagaaatgcataagcttagctaactttt |
| | gtttgataaactctatattcattaatatcacaaatgaattcataaaataccgtatgcattatg |
| | tcccaggg |
| 200615_s_at | [SEQ ID NO: 11] |
| | Gggcaggacatgctgtaccaatccctgaagctcactaatggcatttggattttggccga |
| | actacgtatccagccaggaaaccccaattacacgctgtcactgaagtgtagagctcct |
| | gaagtctctcaatacatctatcaggtctacgacagcattttgaaaaactaacaagactg |
| | gtccagtacccttcaaccatgctgtgatcggtgcaagtcaagaactcttaactggaaga |
| | aattgtattgctgcgtagaatctgaacacactgaggccacctagcaaggtagtaacta |
| | gtctaacctgtgctaacattagggcacaacctgttggatagttttagcttcctgtgaacattt |
| | gtaaccactgcttcagtcacctcccacctcttgccacctgctgctgctatctgtccttacttg |
| | tgggcttctccatgctgtgccaatggctggctttttctacacc |
| 201474_s_at | [SEQ ID NO: 12] |
| | Gccacagactgaactcgcagggagtgcagcaggaaggaacaaagacaggcaaa |
| | cggcaacgtagcctgggctcactgtgctggggcatggcgggatcctccacagagag |
| | gaggggaccaattctggacagacagatgttgggaggatacagaggagatgccactt |
| | ctcactcaccactaccagccagcctccagaaggccccagagagaccctgcaagac |
| | cacggagggagccgacacttgaatgtagtaataggcagggggccctgccaccccat |
| | ccagccagaccccagctgaaccatgcgtcaggggcctagaggtggagttcttagcta |
| | tccttggctttctgtgccagcctggctctgcccctcccccatgggctgtgtcctaaggccc |
| | atttgagaagctgaggctagttccaaaaacctctcctg |
| 202531_at | [SEQ ID NO: 13] |
| | Acaggagtcagtgtctggctttttcctctgagcccagctgcctggagagggtctcgctgt |
| | cactggctggctcctaggggaacagaccagtgaccccagaaaagcataacaccaa |
| | tcccagggctggctctgcactaagcgaaaattgcactaaatgaatctcgttccaaaga |
| | actaccccttttcagctgagccctggggactgttccaaagccagtgaatgtgaaggaa |
| | actcccctccttcggggcaatgctccctcagcctcagaggagctctaccctgctccctg |
| | ctttggctgaggggcttgggaaaaaaacttggcactttttcgtgtggatcttgccacatttc |
| | tgatcagaggtgtacactaacatttcccccgagctcttggcctttgcatttatttatacagtg |
| | ccttgctcggggcccaccaccccctcaagccccagcagccctcaacaggcccaggg |
| | agggaagtgtgagcgccttggtatgacttaa |
| 202643_s_at | [SEQ ID NO: 14] |
| | tctttgggttattactgtctttacttctaaagaagttagcttgaactgaggagtaaaagtgtg |
| | tacatatataatatacccttacattatgtatgagggatttttttaaattatattgaaatgctgcc |
| | ctagaagtacaataggaaggctaaataataataacctgttttctggttgttgttggggcat |
| | gagcttgtgtatacactgcttgcataaactcaaccagctgcctttttaaagggagctctag |
| | tcctttttgtgtaattcactttatttattttattacaaacttcaagattatttaagtgaagatatttct |
| | tcagctctggggaaaatgccacagtgttctcctgagagaacatccttgctttgagtcagg |
| | ctgtgggcaagttcctgaccacagggagtaaattggcctctttgatacacttttgcttgcct |
| | ccccaggaaagaaggaattgcatccaaggtatacatacatattcatcgatgtttcgtgct |
| | tctccttatgaaactccagc |
| 202644_s_at | [SEQ ID NO: 15] |
| | catcccatggtaccctggtattgggacagcaaaagccagtaaccatgagtatgagga |
| | aatctctttctgttgctggcttacagtttctctgtgtgctttgtggttgctgtcatatttgctctaga |
| | agaaaaaaaaaaaaggaggggaaatgcattttccccagagataaaggctgccatttt |
| | gggggtctgtacttatggcctgaaaatatttgtgatccataactctacacagcctttactca |
| | tactattaggcacactttccccttagagccccctaagtttttcccagacgaatctttataattt |
| | cctttccaaagataccaaataaacttcagtgttttcatctaattctcttaaagttgatatctta |
| | atattttgtgttgatcattatttccattcttaatgtgaaaaaaagtaattatttatacttattataa |
| | aaagtatttgaaatttgcacatttaattgtccctaatagaaagccacctattctttgttggat |
| 202659_at | [SEQ ID NO: 16] |
| | Tacacgcgttatctacgggccgcgagccccgcgtggccacggtcactcgcatcctgc |
| | gccagacgctcttcaggtaccagggccacgtgggtgcatcgctgatcgtgggcggcg |
| | tagacctgactggaccgcagctctacggcgtgcatccccatggctcctacagccgtct |
| | gcccttcacagccctgggctctggtcaggacgcggccctggcggtgctagaagaccg |
| | gttccagccgaacatgacgctggaggctgctcaggggctgctggtggaagccgtcac |
| | cgccgggatcttgggtgacctgggctccgggggcaatgtggacgcatgtgtgatcaca |
| | aagactggcgccaagctgctgcggacactgagctcacccacagagcccgtgaaga |
| | ggtctggccgctaccactttgtgcctggaaccacagctgtcctgacccagacagtgaa |
| | gccactaaccctggagctagtggaggaaactgtgcaggctatggaggtggagta |
| 203915_at | [SEQ ID NO: 17] |
| | Gattatcaattaccacaccatctcccatgaagaaagggaacggtgaagtactaagcg |
| | ctagaggaagcagccaagtcggttagtggaagcatgattggtgcccagttagcctctg |
| | caggatgtggaaacctccttccaggggaggttcagtgaattgtgtaggagaggttgtct |
| | gtggccagaatttaaacctatactcactttcccaaattgaatcactgctcacactgctgat |
| | gatttagagtgctgtccggtggagatcccacccgaacgtcttatctaatcatgaaactcc |
| | ctagttccttcatgtaacttccctgaaaaatctaagtgtttcataaatttgagagtctgtgac |
| | ccacttacc |
| 204070_at | [SEQ ID NO: 18] |
| | Gaaacgggggcgcctggaagatgtggtgggaggctgttgctatcgggtcaacaaca |
| | gcttggaccatgagtaccaaccacggcccgtggaggtgatcatcagttctgcgaagg |
| | agatggttggtcagaagatgaagtacagtattgtgagcaggaactgtgagcactttgtc |
| | gcccagctgagatatggcaagtcccgctgtaaacaggtggaaaaggccaaggttga |
| | agtcggtgtggccacggcgcttggaatcctggttgttgctggatgctcttttgcgattagg |
| | agataccaaaaaaaagcaacagcctgaagcagccacaaaatcctgtgttagaagc |
| | agctgtgggggtcc |
| 204116_at | [SEQ ID NO: 19] |
| | ttctggctggaacggacgatgccccgaattcccaccctgaagaacctagaggatcttg |
| | ttactgaataccacgggaacttttcggcctggagtggtgtgtctaagggactggctgag |
| | agtctgcagccagactacagtgaacgactctgcctcgtcagtgagattcccccaaaag |
| | gaggggcccttggggaggggcctggggcctccccatgcaaccagcatagcccctac |
| | tgggcccccccatgttacaccctaaagcctgaaacctgaaccccaatcctctgacaga |
| | agaaccccagggtcctgtagccctaagtggtactaactttccttcattcaacccacctgc |
| | gtctcatactcacctcaccccactgtggctgatttggaattttgtgcccccatgtaagcacc |
| 204224_s_at | [SEQ ID NO: 20] |
| | Gtgatggttggcttgagtacctttttaaatctagcccagtataaacattagcctgcttaata |
| | tttagacatttataggtagaattctgagcactcaactcatgtttggcattttaaagtaaaaa |
| | caagtgtgacttcgaggaccaaagaaattgtcagctatacatttatctttatgaactcattt |
| | atattcctttttaatgactcgttgttctaacatttcctagaagtgttcttataaaggtctaatgta |
| | tccacaggctgttgtcttattagtaaatgcaaagtaatgactttgtctgttttactctagtcttt |
| | agtacttcaaaattaccttttcatatccatgatcttgagtccatttgggggatttttaagaattt |
| | gatgtatttcaatacactgttcaaaattaaattgtttaattttatgtatgagtatgtatgttcctg |
| | aagttggtcctattta |
| 204529_s_at | [SEQ ID NO: 21] |
| | Atggcttgatgtagcagtcatagcaagtttgtaaatagcatctatgttacactctcctaga |
| | gtataaaatgtgaatgtttttgtagctaaattgtaattgaaactggctcattccagtttattga |
| | tttcacaataggggttaaattggcaaacattcatatttttacttcatttttaaaacaactgact |
| | gatagttctatattttcaaaatatttgaaaataaaaagtattcccaagtgattttaatttaaa |
| | aacaaattggctttgtctcattgatcagacaaaaagaaactagtattaagggaagcgc |
| | aaacacatttattttgtactgcagaaaaattgcttttttgtatcactttttgtgtaatggttagta |
| | aatgtcatttaagtccttttatgtataaaactgccaaatgcttacctggtattttattagatgc |
| | agaaacagattggaaacagctaaattacaacttttacatatggctctgtcttattgtttcttc |
| | atactgtgtctgtatttaatctttttttatggaacctgttgcgcctat |
| 204533_at | [SEQ ID NO: 22] |
| | Taactctaccctggcactataatgtaagctctactgaggtgctatgttcttagtggatgttc |
| | tgaccctgcttcaaatatttccctcacctttcccatcttccaagggtactaaggaatctttct |
| | gctttggggtttatcagaattctcagaatctcaaataactaaaaggtatgcaatcaaatct |
| | gctttttaaagaatgctctttacttcatggacttccactgccatcctcccaaggggcccaa |
| | attctttcagtggctacctacatacaattccaaacacatacaggaaggtagaaatatctg |
| | aaaatgtatgtgtaagtattcttatttaatgaaagactgtacaaagtataagtcttagatgt |
| | atatatttcctatattgttttcagtgtacatggaataacatgtaattaagtactatgtatcaat |
| | gagtaacaggaaaattttaaaaatacagatagatatatgctctgcatgttacataagat |
| | aaatgtgctgaatggttttcaaataaaaatgaggtactctcctggaaatatt |
| 204556_s_at | [SEQ ID NO: 23] |
| | ggaactaatgtccctgagatgtttatcaaaaaagaagaattacaagaactaaagtgtg |
| | cggatgtggaggatgaagactgggacatatcatccctagaggaagagatatctttggg |
| | aaaaaaatctgggaaagaacagaaggaacctccacctgcgaaaaatgaaccaca |
| | ttttgctcatgtgctaaatgcctggggcgcatttaatcctaaggggccaaagggagaag |
| | gacttcaagaaaatgaatcaagcacattaaaaagcagcttagtaactgtgactgattg |
| | gagcgacacttcagatgtctaattccacatgtcagaagattattccagaagccagcagt |
| | atttcagtatcacagtgtttcagtaatttgcctccatgattctagtgcttctgccttaccgtgttt |
| | cccacagcaacacagagactgattcaaagaacaatggtctctttaatggcacccaat |
| | acagtattgaaaatcagatcatcaacagtatttcgaagcatgtaaaggtgtttaagactt |
| | ccgctgctgcttaaaaata |
| 204806_x_at | [SEQ ID NO: 24] |
| | Cagatcctccaaaggcacacgttgcccaccaccccatctctgaccatgaggccacc |
| | ctgaggtgctgggccctgggcttctaccctgcggagatcacgctgacctggcagcggg |
| | atggggaggaacagacccaggacacagagcttgtggagaccaggcctgcagggg |
| | atggaaccttccagaagtgggccgctgtggtggtgccttctggagaggaacagagat |
| | acacatgccatgtgcagcacgaggggctgccccagcccctcatcctgagatgggag |
| | cagtctccccagcccaccatccccatcgtgggcatcgttgctggccttgttgtccttggag |
| | ctgtggtcactggagctgtggtcgctgctgtgatgtggaggaagaagagctcagatag |
| | aaacagagggagctactctcaggctgcagtcactgacagtgcccagggctctggggt |
| | gtctctcacagctaataaagtgtgagacagcttccttgtgtgggac |
| 204897_at | [SEQ ID NO: 25] |
| | Agcagcttattgtttctctgaaagtgtgtgtagttttactttcctaaggaattaccaagaata |
| | tcctttaaaatttaaaaggatggcaagttgcatcagaaagctttattttgagatgtaaaaa |
| | gattcccaaacgtggttacattagccattcatgtatgtcagaagtgcagaattggggca |
| | cttaatggtcaccttgtaacagttttgtgtaactcccagtgatgctgtacacatatttgaag |
| | ggtctttctcaaagaaatattaagcatgttttgttgctcagtgtttttgtgaattgcttggttgta |
| | attaaattctgagcctgatattgatatg |
| 205097_at | [SEQ ID NO: 26] |
| | Tactcatgcctttttgtttaggataaataggtaagcacaaagagctcttcaaaatcagaa |
| | aaaacaataggagtccttccttgtcttttctgtgatctctgtccttgtttctgagactttctctac |
| | cattaagctctattttagctttcagttattctagtttgtttcccatggaatctgtcctaaactggt |
| | gtttttgtcagtgacagtcttgccagtcagcaatttctaacagcattttaaatgagtttgatgt |
| | acagtaaatattgatgacaatgacagcttttaactcttcaagtcacctaaagctattatgc |
| | aggaggatttagaagtcacattcataaaacccaagngctatgggtgtattattcatgata |
| | gctggcccacaggtcatgaattgag |
| 205499_at | [SEQ ID NO: 27] |
| | Gcggcatgtgaccatcattgaactggtgggacagccacctcaggaggtggggcgca |
| | tccgggagcaacagctgtcagccaacatcatcgaggagctcaggcaatttcagcgcc |
| | tcactcgctcctacttcaacatggtgttgattgacaagcagggtattgaccgagaccgct |
| | acatggaacctgtcacccccgaggaaatcttcacattcattgatgactacctactgagc |
| | aatcaggagttgacccagcgtcgggagcaaagggacatatgcgagtgaacttgagc |
| | cagggcatggttaaagtcaagggaaaagctcctctagttagctgaaactgggaccta |
| | ataaaaggaggaaatgttttcccacagttctagggacaggactctgaggtgggtgagtt |
| | tgacaaatcctgcagtgtttccaggcatccttttaggactgtgtaatagtttccctagaagc |
| | taggtagggactgaggacaggccttgggcagtgggtt |
| 205685_at | [SEQ ID NO: 28] |
| | Gaaggaggcttaggactttccactcctggctgagagaggaagagctgcaacggaat |
| | taggaagaccaagacacagatcacccggggcttacttagcctacagatgtcctacgg |
| | gaacgtgggctggcccagcatagggctagcaaatttgagttggatgattgtttttgctca |
| | aggcaaccagaggaaacttgcatacagagacagatatactgggagaaatgactttg |
| | aaaacctggctctaaggtgggatcactaagggatggggcagtctctgcccaaacata |
| | aagagaactctggggagcctgagccacaaaaatgttcctttattttatgtaaaccctcaa |
| | gggttatagactgccatgctagacaagcttgtccatgtaatattcccatgtttttaccctgc |
| | ccctgccttgattagactcctagcacctggctagtttc |
| 205758_at | [SEQ ID NO: 29] |
| | Cagcccttgcattgcagaggggcccatgaaagaggacaggctacccctttacaaat |
| | agaatttgagcatcagtgaggttaaactaaggccctcttgaatctctgaatttgagatac |
| | aaacatgttcctgggatcactgatgactttttatactttgtaaagacaattgttggagagcc |
| | cctcacacagccctggcctcngctcaactagcagatacagggatgaggcagacctg |
| | actctcttaaggaggctgagagcccaaactgctgtcccaaacatgcacttccttgcttaa |
| | ggtatggtacaagcaatgcctgcccattggagagaaaaaacttaagtagataaggaa |
| | ataagaaccactcataattcttcaccttaggaataatctcctgttaatatggtgtacattctt |
| | cctgattattttctacacatac |
| 205890_s_at | [SEQ ID NO: 30] |
| | Gatcttaaagccacggagaagcctctcatcttatggcattgacaaagagaagaccat |
| | ccaccttaccctgaaagtggtgaagcccagtgatgaggagctgcccttgtttcttgtgga |
| | gtcaggtgatgaggcaaagaggcacctcctccaggtgcgaaggtccagctcagtgg |
| | cacaagtgaaagcaatgatcgagactaagacgggtataatccctgagacccagatt |
| | gtgacttgcaatggaaagagactggaagatgggaagatgatggcagattacggcat |
| | cagaaagggcaacttactcttcctggcatcttattgtattggagggtgaccaccctgggg |
| | atggggtgttggcaggggtcaaaaagcttatttcttttaatctcttactcaacgaacacat |
| | cttctgatgatttcccaaaattaatgagaatgagatgagtagagtaagatttgggtggga |
| | tgggtaggatgaagtatattgcccaactctatgtttctttga |
| 206082_at | [SEQ ID NO: 31] |
| | Tgaaggatggtgactgcgccatggcctggatctgctgcagtgtcctttcctgtggaggct |
| | ccactcaaagctggcatcctcctatgtcacctagagtgtgggtcaaagcaatacaccta |
| | catgtagaatgtgatgtcagaactcaaacaggctcaccaggcagtgtgcttcttccttgc |
| | atgaggatgcaagatgcaacagtttgtcttcacattggaaggacacccctggatgccc |
| | ctaaccactagacctgtaaaacttcactgcagtggccacttctgaatctctgtaaggttta |
| | tttatcttcacccctctggagagaagatgttttaccaaagcctctagtgtaccgtcctcctct |
| | tactcatccatcccagtcaacatgatgttgtcaatgaaataaaggaatttaatattctata |
| | gtatatccaggttctccagatctcttaagactgtactatagaggcctgggg |
| 206666_at | [SEQ ID NO: 32] |
| | aaacctctcttagatctggaaccaaatgcaaggttactggctggggagccaccgatcc |
| | agattcattaagaccttctgacaccctgcgagaagtcactgttactgtcctaagtcgaaa |
| | actttgcaacagccaaagttactacaacggcgacccttttatcaccaaagacatggtct |
| | gtgcaggagatgccaaaggccagaaggattcctgtaagggtgactcagggggcccc |
| | ttgatctgtaaaggtgtcttccacgctatagtctctggaggtcatgaatgtggtgttgccac |
| | aaagcctggaatctacaccctgttaaccaagaaataccagacttggatcaaaagcaa |
| | ccttgtcccgcctcatacaaattaagttacaaataattttattggatgcacttgcttcttttttc |
| | ctaatatgctcgcaggttagagttgggtgtaagtaaagcagagcacatatggggtccat |
| | ttttgcacttgta |
| 207536_s_at | [SEQ ID NO: 33] |
| | agaccagtacaaactactcaagaggaagatggctgtagctgccgatttccagaaga |
| | agaagaaggaggatgtgaactgtgaaatggaagtcaatagggctgttgggactttctt |
| | gaaaagaagcaaggaaatatgagtcatccgctatcacagctttcaaaagcaagaac |
| | accatcctacataatacccaggattcccccaacacacgttcttttctaaatgccaatgag |
| | ttggcctttaaaaatgcaccactttttttttttttttggacagggtctcactctgtcacccaggc |
| | tggagtgcagtggcaccaccatggctctctgcagccttgacctctgggagctcaagtg |
| | atcctcctgcctcagtctcctgagtagctggaactacaaggaagggccaccacacctg |
| | actaacttttttgttttttgttggtaaagatggcatttcgccatgttgtacaggctggtctcaaa |
| | ctcctaggttcactttggcctcccaaagtgctgggattacagacatgaactgccaggcc |
| | cggccaaaataatgcaccact |
| 207651_at | [SEQ ID NO: 34] |
| | ttgccttgtaattcgacagctctacagaaacaaagataatgaaaattacccaaatgtga |
| | aaaaggctctcatcaacatacttttagtgaccacgggctacatcatatgctttgttccttac |
| | cacattgtccgaatcccgtataccctcagccagacagaagtcataactgattgctcaac |
| | caggatttcactcttcaaagccaaagaggctacactgctcctggctgtgtcgaacctgt |
| | gctttgatcctatcctgtactatcacctctcaaaagcattccgctcaaaggtcactgagac |
| | ttttgcctcacctaaagagaccaaggctcagaaagaaaaattaagatgtgaaaataat |
| | gcataaaagacaggattttttgtgctaccaattctggccttactgga |
| 207795_s_at | [SEQ ID NO: 35] |
| | Ttctctacttcgctcttggaacataatttctcatggcagcttttactaaactgagtattgagc |
| | cagcatttactccaggacccaacatagaactccagaaagactctgactgctgttcttgc |
| | caagaaaaatgggttgggtaccggtgcaactgttacttcatttccagtgaacagaaaa |
| | cttggaacgaaagtcggcatctctgtgcttctcagaaatccagcctgcttcagcttcaaa |
| | acacagatgaactggattttatgagctccagtcaacaattttactggattggactctcttac |
| | agtgaggagcacaccgcctggttgtgggagaatggctctgcactctcccagtatctattt |
| | ccatcatttg |
| 208729_x_at | [SEQ ID NO: 36] |
| | Gtggcggagcagctgagagcctacctggagggcgagtgcgtggagtggctccgca |
| | gatacctggagaacgggaaggagacgctgcagcgcgcggaccccccaaagaca |
| | cacgtgacccaccaccccatctctgaccatgaggccaccctgaggtgctgggccctg |
| | ggcttctaccctgcggagatcacactgacctggcagcgggatggcgaggaccaaac |
| | tcaggacactgagcttgtggagaccagaccagcaggagatagaaccttccagaagt |
| | gggcagctgtggtggtgccttctggagaagagcagagatacacatgccatgtacagc |
| | atgaggggctgccgaagcccctcaccctgagatgggagccgtcttcccagtccaccg |
| | tccccatcgtgggcattgttgctggcctggctgtcctagcagttgtggtcatcggagctgt |
| | ggtcgctgctgtgatgtgtaggaggaagagctcaggtggaaaaggagggagctactc |
| | tcaggctg |
| 208885_at | [SEQ ID NO: 37] |
| | Gaagtaagcctcatcatcagagcctttcctcaaaactggagtcccaaatgtcatcagg |
| | ttttgttttttttcagccactaagaacccctctgcttttaactctagaatttgggcttggaccag |
| | atctaacatcttgaatactctgccctctagagccttcagccttaatggaaggttggatcca |
| | aggaggtgtaatggaatcggaatcaagccactcggcaggcatggagctataactaa |
| | gcatccttagggttctgcctctccaggcattagccctcacattagatctagttactgtggta |
| | tggctaatacctgtcaacatttggaggcaatcctaccttgcttttgcttctagagcttagcat |
| | atctgattgttgtcaggccatattatcaatgtttacttttttggtactataaaagctttctgcca |
| | cccctaaactccaggggggacaatatgtgccaatcaatagcacccctactcacatac |
| | acacacacctagccagctgtcaagggc |
| 208894_at | [SEQ ID NO: 38] |
| | Cgatcaccaatgtacctccagaggtaactgtgctcacgaacagccctgtggaactga |
| | gagagcccaacgtcctcatctgtttcatagacaagttcacccca |
| 209606_at | [SEQ ID NO: 39] |
| | Gaattgcaaaactgacatcccatttcacagcaatagtgacctttatttaaattgttgtgtta |
| | tagtttatgcttcttaaatcatttttcaacctaaacagccaatttctaagcagacaggaaa |
| | actaaataataagttaattaatataacaaagatgcaggttcctgctcattccagtaatgtc |
| | tttgaaagcaaaactaatatttattttctagattatccctgtgaataattgagaactttttgga |
| | gtcaagtatgaataaaggtgtggcagaatataataatctggactattttctataggataat |
| | tgctgggttataaaatcttaggtttgcttatgcccagtagctcctgcggaggcttaataata |
| | ggcaattttgaatttgttcaaacctgtaatggcttgtaaacaaagatgaccatcagctgttt |
| | ctcacatctatagtgacaataaagcgggaagtataagatttaataggaggggttaagg |
| | ttcatgagaaccatggaaagatgtggtctgagatgggtgctgcaaagat |
| 209671_x_at | [SEQ ID NO: 40] |
| | Tctcgaaccgaacagcagtgcttccaagataatctttggatcagggaccagactcag |
| | catccggccaaatatccagaaccctgaccctgccgtgtaccagctgagagactctaa |
| | atccagtgacaagtctgtctgcctattcaccgattttgattctcaaacaaatgtgtcacaa |
| | agtaaggattctgatgtgtatatcacagacaaaactgtgctagacatgaggtctatgga |
| | cttcaagagcaacagtgctgtggcctggagcaacaaatctgactttgcatgtgcaaac |
| | gccttcaacaacagcattattccagaagacaccttcttccccagcccagaaagttcctg |
| | tgatgtcaagctggtcgagaaaagctttgaaacagatacgaacctaaactttcaaaac |
| | ctgtcagtgattgggttccgaatcctcctcctgaaagtggccgggtttaatctgctcatga |
| | cgctgcggctgtggtccagctgagatctgcaagattgtaagacagcctgtgctccct |
| 209770_at | [SEQ ID NO: 41] |
| | Ggaaatttggatgaagggagctagaagaaatacagggatttttttttttttttaagatgga |
| | gtcttactctgttgctaggctggagtgcagtggtgcgatctcagctccctgcaacctccac |
| | ctcctgggttcaaacaattctcctgcctcagcctcccgagtactgggaatataggtgcac |
| | gccaccacacccaacaaatttttgtacttttagtacagatgagggttcactatgttggcca |
| | ggatggtctcgatctcttgacctcatgatccacccacctcggtctcccaaagtgctggga |
| | ttacaggcttgagccaccgggtgaccggcttacagggatatttttaatcccgttatggact |
| | ctgtctccaggagaggggtctatccacccctgctcattggtggatgttaaaccaatattc |
| | ctttcaactgctgcctgctagggaaaaactactcctcattatcatcattattattgctctcca |
| | ctgtatcccctctacctggcatgtgcttgtcaag |
| 209774_x_at | [SEQ ID NO: 42] |
| | Agagagacacagctgcagaggccacctggattgcgcctaatgtgtttgagcatcactt |
| | aggagaagtcttctatttatttatttatttatttatttatttgtttgttttagaagattctatgttaatat |
| | tttatgtgtaaaataaggttatgattgaatctacttgcacactctcccattatatttattgtttatt |
| | ttaggtcaaacccaagttagttcaatcctgattcatatttaatttgaagatagaaggtttgc |
| | agatattctctagtcatttgttaatatttcttcgtgatgacatatcacatgtcagccactgtga |
| | tagaggctgaggaatccaagaaaatggccagtaagatcaatgtgacggcagggaa |
| | atgtatgtgtgtctattttgtaactgtaaagatgaatgtcagttgttatttattgaaatgatttca |
| | cagtgtgtggtcaacatttctcatgttgaagctttaagaactaaaatgttctaaatatccctt |
| | ggacattttatgtctttcttgtaagatactgccttgtttaatgttaattatgcagtgtttccctc |
| 209813_x_at | [SEQ ID NO: 43] |
| | Aaatgatacactactgctgcagctcacaaacacctctgcatattacatgtacctcctcct |
| | gctcctcaagagtgtggtctattttgccatcatcacctgctgtctgcttagaagaacggctt |
| | tctgctgcaatggagagaaatcataacagacggtggcacaaggaggccatcttttcct |
| | catcggttattgtccctagaagcgtcttctgaggatctagttgggctttctttctgggtttggg |
| | ccatttcagttctcatgtgtgtactattctatcattattgtataacggttttcaaaccagtgggc |
| | acacagagaacctcactctgtaataacaatgaggaatagccacggcgatctccagc |
| | accaatctctccatgttttccacagctcctccagccaacccaaatagcgcctgctatagt |
| | gtagacatcctgcggcttctagccttgtccctctcttagtgttctttaatcagataactgcctg |
| | gaagcctttcattttacacgccctgaagcagtcttctttgcta |
| 210439_at | [SEQ ID NO: 44] |
| | Gcttctgaagcagccaatgtcgatgcaacaacatttgtaactttaggtaaactgggatt |
| | atgttgtagtttaacattttgtaactgtgtgcttatagtttacaagtgagacccgatatgtcatt |
| | atgcatacttatattatcttaagcatgtgtaatgctggatgtgtacagtacagtacttaactt |
| | gtaatttgaatctagtatggtgttctgttttcagctgacttggacaacctgactggctttgca |
| | caggtgttccctgagttgtttgcaggtttctgtgtgtggggtggggtatggggaggagaa |
| | ccttcatggtggcccacctggcctggttgtccaagctgtgcctcgacacatcctcatccc |
| | aagcatgggacacctcaagatgaataataattcacaaaatttctgtgaaatcaaatcc |
| | agttttaagaggagccacttatcaaagagat |
| 210606_x_at | [SEQ ID NO: 45] |
| | gaaagactctgactgctgttcttgccaagaaaaatgggttgggtaccggtgcaactgtt |
| | acttcatttccagtgaacagaaaacttggaacgaaagtcggcatctctgtgcttctcaga |
| | aatccagcctgcttcagcttcaaaacacagatgaactggattttatgagctccagtcaa |
| | caattttactggattggactctcttacagtgaggagcacaccgcctggttgtgggagaat |
| | ggctctgcactctcccagtatctatttccatcatttgaaacttttaatacaaagaactgcat |
| | agcgtataatccaaatggaaatgctttagatgaatcctgtgaagataaaaatcgttatat |
| | ctgtaagcaacagctcatttaaatgtttcttggggcagagaaggtggagagtaaagac |
| | ccaacattactaacaatgatacagttgcatgttatattattactaattgtctacttctggagt |
| | cta |
| 210915_x_at | [SEQ ID NO: 46] |
| | aaaggccacactggtgtgcctggccacaggtatcttccctgaccacgtggagctgagc |
| | tggtgggtgaatgggaaggaggtgcacagtggggtcagcacggacccgcagcccct |
| | caaggagcagcccgccctcaatgactccagatactgcctgagcagccgcctgaggg |
| | tctcggccaccttctggcagaacccccgcaaccacttccgctgtcaagtccagttctac |
| | gggctctcggagaatgacgagtggacccaggatagggccaaacccgtcacccaga |
| | tcgtcagcgccgaggcctggggtagagcagactgtggctttacctcggtgtcctacca |
| | gcaaggggtcctgtctgccaccatcctctatgagatcctgctagggaaggccaccatgt |
| | atgctgtgctggtcagcgcccttgtgttgatggccatggtcaagagaaaggatttctgaa |
| | ggcagccctggaagtggagttaggagcttctaacccgtcatggtttcaatacacattctt |
| | cttttgccagc |
| 210972_x_at | [SEQ ID NO: 47] |
| | ggaacaagacttcaggtcacgctcgatatccagaaccctgaccctgccgtgtaccag |
| | ctgagagactctaaatccagtgacaagtctgtctgcctattcaccgattttgattctcaaa |
| | caaatgtgtcacaaagtaaggattctgatgtgtatatcacagacaaaactgtgctagac |
| | atgaggtctatggacttcaagagcaacagtgctgtggcctggagcaacaaatctgact |
| | ttgcatgtgcaaacgccttcaacaacagcattattccagaagacaccttcttccccagc |
| | ccagaaagttcctgtgatgtcaagctggtcgagaaaagctttgaaacagatacgaac |
| | ctaaactttcaaaacctgtcagtgattgggttccgaatcctcctcctgaaagtggccggg |
| | tttaatctgctcatgacgctgcggctgtggtccagctgagatctgcaagattgtaagaca |
| | gcctgtgctccct |
| 210982_s_at | [SEQ ID NO: 48] |
| | Gaaggagacggtctggcggcttgaagaatttggacgatttgccagctttgaggctcaa |
| | ggtgcattggccaacatagctgtggacaaagccaacttggaaatcatgacaaagcgc |
| | tccaactatactccgatcaccaatgacaagttcaccccaccagtggtcaatgtcacgtg |
| | gcttcgaaatggaaaacctgtcaccacaggagtgtcagagacagtcttcctgcccag |
| | ggaagaccaccttttccgcaagttccactatctccccttcctgccctcaactgaggacgtt |
| | tacgactgcagggtggagcactggggcttggatgagcctcttctcaagcactgggagtt |
| | tgatgctccaagccctctcccagagactacagagaacgtggtgtgtgccctgggcctg |
| | actgtgggtctggtgggcatcattattgggaccatc |
| 211144_x_at | [SEQ ID NO: 49] |
| | aaatgatacactactgctgcagctcacaaacacctctgcatattacatgtacctcctcct |
| | gctcctcaagagtgtggtctattttgccatcatcacctgctgtctgcttggaagaacggctt |
| | tctgctgcaatggagagaaatcataacagacggtggcacaaggaggccatcttttcct |
| | catcggttattgtccctagaagcgtcttctgaggatctagttgggctttctttctgggtttggg |
| | ccatttcagttctcatgtgtgtactattctatcattattgtataatggttttcaaaccagtgggc |
| | acacagagaacctcagtctgtaataacaatgaggaatagccatggcgatctccagca |
| | ccaatctctccatgttttccacagctcctccagccaacccaaatagcgcctgctatagtgt |
| | agacagcctgcggcttctagccttgtccctctcttagtgttctttaatcagataactgcctgg |
| | aagcctttcattttacacgccc |
| 211149_at | [SEQ ID NO: 50] |
| | Cagaaacctcgatatataattgtatagattttaaaagttttattttttacatctatggtagttttt |
| | gaggtgcctattataaagtattacggaagtttgctgtttttaaagtaaatgtcttttagtgtga |
| | tttattaagttgtagtcaccatagtgatagcccataaataattgctggaaaattgtattttat |
| | aacagtagaaaacatatagtcagtgaagtaaatattttaaaggaaacattatatagattt |
| | gataaatgttgtttataattaagagtttcttatggaaaagagattcagaatgataacctcttt |
| | tagagaacaaataagtgacttatttttttaaagctagatgactttgaaatgctatactgtcct |
| | gcttgtacaacatggtttggggtgaaggg |
| 211339_s_at | [SEQ ID NO: 51] |
| | ggtgttgcaattggctctttctaaatcatgtgacgttttgactggcttgagattcagatgcat |
| | aatttttaattataattattgtgaagtggagagcctcaagataaaactctgtcattcagaa |
| | gatgattttactcagcttatccaaaattatctctgtttactttttagaattttgtacattatcttttg |
| | ggatccttaattagagatgatttctggaacattcagtctagaaagaaaacattggaattg |
| | actgatctctgtggtttggtttagaaaattcccctgtgcatggtattacctttttcaagctcag |
| | attcatctaatcctcaactgtacatgtgtacattcttcacctcctggtgccctatcccgcaa |
| | aatgggcttcctgcctggtttttctcttctcacattttttaaatggtcccctgtgtttgtagagaa |
| 211796_s_at | [SEQ ID NO: 52] |
| | Gccatcagaagcagagatctcccacacccaaaaggccacactggtgtgcctggcc |
| | acaggtttctaccccgaccacgtggagctgagctggtgggtgaatgggaaggaggtg |
| | cacagtggggtcagcacagacccgcagcccctcaaggagcagcccgccctcaatg |
| | actccagatactgcctgagcagccgcctgagggtctcggccaccttctggcagaacc |
| | cccgcaaccacttccgctgtcaagtccagttctacgggctctcggagaatgacgagtg |
| | gacccaggatagggccaaacctgtcacccagatcgtcagcgccgaggcctggggta |
| | gagcagactgtggcttcacctccgagtcttaccagcaaggggtcctgtctgccaccatc |
| | ctctatgagatcttgctagggaaggccaccttgtatgctgtgctggtcagtgccctcgtgc |
| | tgatggccatggtcaagagaaagga |
| 211902_x_at | [SEQ ID NO: 53] |
| | Gaatcgtttctctgtgaacttccagaaagcagccaaatccttcagtctcaagatctcag |
| | actcacagctgggggatgccgcgatgtatttctgtgcttataggagtgcatactctgggg |
| | ctgggagttaccaactcactttcgggaaggggaccaaactctcggtcataccaaatat |
| | ccagaaccctgaccctgccgtgtaccagctgagagactctaaatccagtgacaagtct |
| | gtctgcctattcaccgattttgattctcaaacaaatgtgtcacaaagtaaggattctgatgt |
| | gtatatcacagacaaaactgtgctagacatgaggtctatggacttcaagagcaacagt |
| | gctgtggcctggagcaacaaatctgactttgcatgtgcaaacgccttcaacaacagca |
| | ttattccagaagacaccttcttccccagcccagaaagttcctgtgatgtcaagctggtcg |
| | agaaaagctttgaaacagatacgaacctaaactttcaaaacctgtcagtgattgggttc |
| | cgaatcctcctcctgaaagtggccgggtttaatctgctcatgacgctgcggttgtggtcc |
| 211911_x_at | [SEQ ID NO: 54] |
| | Ctgagagcctacctggagggcctgtgcgtggagtggctccgcagatacctggagaa |
| | cgggaaggagacgctgcagcgcgcggaccccccaaagacacatgtgacccacca |
| | ccccatctctgaccatgaggccaccctgaggtgctgggccctgggcttctaccctgcgg |
| | agatcacactgacctggcagcgggatggcgaggaccaaactcaggacaccgagct |
| | tgtggagaccagaccagcaggagatagaaccttccagaagtgggcagctgtggtgg |
| | tgccttctggagaagagcagagatacacatgccatgtacagcatgaggggctgccga |
| | agcccctcaccctgagatgggagccatcttcccagtccaccatccccatcgtgggcatt |
| | gttgctggcctggctgtcctagcagttgtggtcatcggagctgtggtcgctactgtgatgtg |
| | taggaggaagagctcaggtggaaaaggagggagctactctcaggctg |
| 212671_s_at | [SEQ ID NO: 55] |
| | Accaatgaggttcctgaggtcacagtgttttccaagtctcccgtgacactgggtcagcc |
| | caacaccctcatctgtcttgtggacaacatctttcctcctgtggtcaacatcacntggctg |
| | agcaatgggcactcagtcacagaaggtgtttctgagaccagcttcctctccaagagtg |
| | atcattccttcttcaagatcagttacctcaccttcctcccttctgntgatgagatttatgactg |
| | caaggtggagcactggggcctggatgagcctcttctgaaacactgggagcctg |
| 213193_x_at | [SEQ ID NO: 56] |
| | Tgactccagatactgcctgagcagccgcctgagggtctcggccaccttctggcagaa |
| | cccccgcaaccacttccgctgtcaagtccagttctacgggctctcggagaatgacgag |
| | tggacccaggatagggccaaacccgtcacccagatcgtcagcgccgaggcctggg |
| | gtagagcagactgtggctttacctcggtgtcctaccagcaaggggtcctgtctgccacc |
| | atcctctatgagatcctgctagggaaggccaccctgtatgctgtgctggtcagcnccctt |
| | gtgttgatggccatggtcaagagaaaggatttctgaaggcagccctggaagtggagtt |
| | aggagcttctaacccgtcatggtttcaatacacattcttcttttgccagcgcttctgaagag |
| | ctgctctcacctctctgcatcccaatagatatccccctatgtgcatgcacacctgcacact |
| | cacggctgaaatctccctaacccagggggaccttagcatgcctaagtga |
| 213539_at | [SEQ ID NO: 57] |
| | gggaacactgctctcagacattacaagactggacctgggaaaacgcatcctggacc |
| | cacgaggaatatataggtgtaatgggacagatatatacaaggacaaagaatctaccg |
| | tgcaagttcattatcgaatgtgccagagctgtgtggagctggatccagccaccgtggct |
| | ggcatcattgtcactgatgtcattgccactctgctccttgctttgggagtcttctgctttgctg |
| | gacatgagactggaaggctgtctggggctgccgacacacaagctctgttgaggaatg |
| | accaggtctatcagcccctccgagatcgagatgatgctcagtacagccaccttggagg |
| | aaactgggctcggaacaagtgaacctgagactggtggcttctagaagcagccattac |
| | caactgtacct |
| 213793_s_at | [SEQ ID NO: 58] |
| | tgctggagtccactgccaatgtgaaacaatggaaacagcaacttgctgcctatcanga |
| | ggaagcagaacgtctgcacaagcgggtgactgaacttgaatgtgttagtagccaagc |
| | aaatgcagtacatactcataagacagaattaaatcagacaatacaagaantgnaan |
| | ngncacngaaantgaaggaagaggaaatagaaaggttaaaacaagaaattgata |
| | atgccagagaactacaagaacagagggattctttgactcagaaactacaggaagta |
| | gaaattcggaacaaagacctggagggacaactgtctgacttagagcaacgtctgga |
| | gaaaagtcagaatgaacaagaagcttttcgcaataacctgaagacactcttagaaatt |
| | ctggatggaaagatatttgaactaacagaattacgagataacttggccaagctactag |
| | antgcagctaaggaaagtgaaatttcngtgccnattaattaaaagatacactgtctctct |
| | tcataggactgtttaggctctgcatca |
| 214470_at | [SEQ ID NO: 59] |
| | ggttcaccttggcatcaatttgccctgaaacttagctgtgctgggattattctccttgtcttgg |
| | ttgttactgggttgagtgtttcagtgacatccttaatacagaaatcatcaatagaaaaatg |
| | cagtgtggacattcaacagagcaggaataaaacaacagagagaccgggtctcttaa |
| | actgcccaatatattggcagcaactccgagagaaatgcttgttattttctcacactgtcaa |
| | cccttggaataacagtctagctgattgttccaccaaagaatccagcctgctgcttattcg |
| | agataaggatgaattgatacacacacagaacctgatacgtgacaaagcaattctgtttt |
| | ggattggattaaatttttcattatcagaaaagaactggaagtgganaaacggctctttttt |
| | aaattctaatgacttagaaattagaggtgatgctaaagaaaacagctgtatttccatctc |
| | aca |
| 215806_x_at | [SEQ ID NO: 60] |
| | Aaatgatacactactgctgcagctcacaaacacctctgcatattacatgtacctcctcct |
| | gctcctcaagagtgtggtctattttgccatcatcacctgctgtctgcntgnaagaacggc |
| | nnnctgctgcaatggagagaantcataacagacggtggcacaaggaggccnncnt |
| | ntcctcatcggnnattgtccctagaagcgtcttctgaggatctagttgggctttctttctggg |
| | tttgggccatttcagttctcatgtgtgtactattctatcattattgtataatggttttcaaaccag |
| | tgggcacacagagaacctcagtctgtaataacaatgaggaatagccatggcgatctc |
| | cagcaccaatctctccatgttttccacagctcctccagccaacccaaatagcgcctgct |
| | atagtgtaganannctgcggcttctagccttgtccctctcttagtgttctttaatcagataac |
| | tgcctggaagcctttcattttacacgccctgaagcagtcttctttgcta |
| 216920_s_at | [SEQ ID NO: 61] |
| | Cactactgctgcagctcacaaacacctctgcatattacatgtacctcctcctgctcctca |
| | agagtgtggtctattttgccatcatcacctgctgtctgcttngaagaacggctttctgctgc |
| | aatggagagaaatcataacagacggtggcacaaggaggccatcttttcctcatcggtt |
| | attgtccctagaagcgtcnncnnannnnnnnnttgggctttctttctgggtttgggccatt |
| | tcagttctcatgtgtgtactattctatctattgtataatggttttcaaaccagtgggcacaca |
| | gagaacctcactctgtaataacaatgaggaatagccatggcgatctccagcaccaat |
| | ctctccatgttttccacagctcctccagccaacccaaatagcgcctgctatagtgtagac |
| | agcctgcggcttctagccttgtccctctcttagtgttctttaatcagataactgcctggaagc |
| | ctttcattttacacgccctgaagcagtcttctttgctagttgaattatgtggtgtgtttttccgta |
| | ata |
| 217436_x_at | [SEQ ID NO: 62] |
| | tacctggagggcacctgcatggagtggctccgcagacacctggagaacgggaagg |
| | agacgctgcagcgcgcggacccccccnaagacacacgtgacccaccnccctnnct |
| | ctgaacatgaggcataacgaggtnctgggttctgggcttctaccctgcggagatcacat |
| | tgacctggcagcgggatggggaggaccagacccaggacatggagctcgtggagac |
| | caggcccacaggggatggaaccttccagaagtgggcggttgtggtagtgccttctgga |
| | gaggaacagagatacacatgccatgtgcagcacaaggggcntgcccaagcccctc |
| | atcctgagatgggagccctctccccagcccaccatccccattgtgggtatcattgctgg |
| | cctggttctccttggagctgtggtcactgnnnnnnnnnnnnnnnctgtgatgtggagg |
| | aagaagagctcagatagaaaaggagggagctactctcaggctgcaagcagccaa |
| | agtgcccagggctct |
| 217478_s_at | [SEQ ID NO: 63] |
| | ctgttttgtcagtaatctcttcccacccatgctgacagtgaactggcagcatcattccgtcc |
| | ctgtggaaggatttgggcctacttttgtctcagctgtcgatggactcagcttccaggcctttt |
| | cttacttaaacttcacaccagaaccttctgacattttctcctgcattgtgactcacgaaattg |
| | accgctacacagcaattgcctattgggtaccccggaacgcactgccctcagatctgct |
| | ggagaatgtgctgtgtggcgtggcctttggcctgggtgtgctgggcatcatcgtgggcat |
| | tgttctcatcatctacttccggaagccttgctcaggtgactgattcttccagaccagagttt |
| | gatgccagcagcttcggccatccaaacagaggatgctcagatttctcacatcctgc |
| 219551_at | [SEQ ID NO: 64] |
| | Gaacaggtgaccataactctgccaaatatagaaagttgaaggaagtagtaaaattca |
| | gtatcgtaaagaacaacagcaacaacaaatgtggaattcagccaggactcccaatct |
| | tgtaaaacattctccatctgaagataagatgtccccagcatctccaatagatgatatcga |
| | aagagaactgaaggcagaagctagtctaatggaccagatgagtagttgtgatagttc |
| | atcagattccaaaagttcatcatcttcaagtagtgaggatagttctagtgactcagaaga |
| | tgaagattgcaaatcctctacttctgatacagggaattgtgtctcaggacatcctaccatg |
| | acacagtacaggattcctgatatagatgccagtcataatagatttcgagacaacagtg |
| | gccttctgatgaatacttt |
| 221081_s_at | [SEQ ID NO: 65] |
| | Ttctcacttttcatccaggaagccgagaagagcaagaatcctcctgcaggctatttcca |
| | acagaaaatacttgaatatgaggaacagaagaaacagaagaaaccaagggaaa |
| | aaactgtgaaataagagctgtggtgaataagaatgactagagctacacaccatttctg |
| | gacttcagcccctgccagtgtggcaggatcagcaaaactgtcagctcccaaaatccat |
| | atcctcactctgagtcttggtatccaggtattgcttcaaactggtgtctgagatttggatccc |
| | tggtattgatttctcaggactttggagggctctgacaccatgctcacagaactgggctca |
| | gagctccattttttgcagaggtgacacaggtaggaaacagtagtacatgtgttgtagac |
| | acttggttagaagctgctgcaactgccctctcccatcattataacatcttcaacacagaa |
| | cacactttgtggtcgaaaggctcagcctctctacatgaagtctg |
| 221875_x_at | [SEQ ID NO: 66] |
| | Tctaccctgcggagatcacgctgacctggcagcgggatggggaggaacagaccca |
| | ggacacagagcttgtggagaccaggcctgcaggggatggaaccttccagaagtgg |
| | gccgctgtggtggtgcctnctggagaggaacagagatacacatgccatgtgcagcac |
| | gaggggctgccccagcccctcatcctgagatgggagcagtctccccagcccaccatc |
| | cccatcgtgggcatcgttgctggccttgttgtccttggagctgtggtcactggagctgtggt |
| | cgctgctgtgatgtggaggaagaagagctcagatagaaacagagggagctactctc |
| | aggctgcagtgtgagacagcttccttgtgtgggactgagaagcaagatatcaatgtag |
| | cagaattgcacttgtgcctcacgaacata |
| 222838_at | [SEQ ID NO: 67] |
| | Aacacctgtgctaggtcagtctggcacgtaagatgaacatccctaccaacacagagc |
| | tcaccatctcttatacttaagtgaaaaacatggggaaggggaaaggggaatggctgct |
| | tttgatatgttccctgacacatatcttgaatggagacctccctaccaagtgatgaaagtgtt |
| | gaaaaacttaataacaaatgcttgttgggcaagaatgggattgaggattatcttctctca |
| | gaaaggcattgtgaaggaattgagccagatctctctccctactgcaaaaccctattgta |
| | gta |
| 222962_s_at | [SEQ ID NO: 68] |
| | Aaactttcccatctagataatgatgatcacatagtcttgatgtacggacattaaaagcca |
| | gatttcttcattcaattctgttatctctgttttactctttgaaattgatcaagccactgaatcactt |
| | tgcatttcagtttatatatatagagagaaagaaggtgtctgctcttacattattgtggagcc |
| | ctgtgatagaaatatgtaaaatctcatattattttttttttaatttttttattttttatgacagggtct |
| | cactatgtcaccctggctggagtgcagtagtgcgatcgcggcacactgc |
| 223575_at | [SEQ ID NO: 69] |
| | Aaatgactgcattcgtctcttttttaaaggtagagattaaactgtatagacagcataggg |
| | atgaaaggaaccaagcgtttctgtgggattgagactggtacgtgtacgatgaacctgct |
| | gctttgttttctgagaagaggtttgaagacattttattaacagcttaatttttctcttttactccat |
| | aggaacttattttaatagtaacattaacaacaagaatactaagactgtttgggaattttaa |
| | aaagctactagtgagaaaccaaatgataggttgtagagcctgatgactccaaacaaa |
| | gccatcacccgcattcttcctccttcttctggtgctacagctccaagggcccttcaccttca |
| | tgtctgaaatgg |
| 223593_at | [SEQ ID NO: 70] |
| | ggcagctgcagacaagtggttaactggtttggcagaatggcatgttcctgctgctggaa |
| | tgtttttatggattaaagttaaaggcattaatgatgtaaaagaactgattgaagaaaagg |
| | ccgttaagatgggggtattaatgctccctggaaatgctttctacgtcgatagctcagctcc |
| | tagcccttacttgagagcatccttctcttcagcttctccagaacagatggatgtggccttcc |
| | aggtattagcacaacttataaaagaatctttatgaagaaattaaactaggttgggcatg |
| | gtgcgtcacacctataatcccagcactttgggaggcagaggagggaggatcacttga |
| | acccaggaattcaggctgcagtaagctacgatcacaccactgcactctggcctgcatg |
| | cactctggcctgcatggcagaacaagaccctgtctctaaaaaaagagaaagaaatc |
| | aaactaatcatgctgctcat |
| 225996_at | [SEQ ID NO: 71] |
| | Acagttcaaccagtgaccgacttctctctcatgctgtttaccccacacacaatttcccact |
| | caattctgaaaataagaacctgttaataggttggaaagctgtgtactctattcatatattgtt |
| | ctttcatgctagtggagagtggtgtcattagcatcttaattttagagttgtgaaatgattttac |
| | caattaggaattgaatgtgtattttttttctgtttaataagaagagcaaatttgaataaataa |
| | gctggtgtagataaacttaataatcatgctttttcttgtttggagataggtgatgtgttgtcat |
| | atcctgtgatacaggtcactcatctggccttctgtttctgaagtttaagtctggtttgaatatg |
| | taataatactactcagcatttcttgttgcctaagtgagacgaaacttaaatgttatgatattt |
| | acttcatgtattcttgtactgttcatttcaat |
| 226084_at | [SEQ ID NO: 72] |
| | aatggcttctatgatcagaactgggaaaacagtgnatcttatggtggaagaggtnctca |
| | gcaagtgtacagtatttaccttcctttgtcttacatnggctttttaaattttccattaatttcaac |
| | ataattatgggaacaagtgtacagaagaattttttttttaagatatgtgagaacttttcatag |
| | atgaactttttaacaaatgttttcatttacaggaaattgcaaagaaaattctcaagtgata |
| | gtctttttttttaagtgtttcgtaagacaaaaattgaataatgttttttgaagttctggcaagatt |
| | gaagtctgatattgcagtaatgatatttattaaaaacccataactaccaggaataatgat |
| | acctcccaccccttgattcccataacataaaagtgctacttgagagtgggggagaatg |
| | gcatggtaggctacttttcagggccttgacaagtacatcacccagtggtatcctacatac |
| | ttctttcaagatcttcaaccatgaggtaaaagagccaagttcaaagaaccctagcaca |
| | aatttgctttgg |
| 228316_at | [SEQ ID NO: 73] |
| | Acagggtcagactcatagggtcatggagtacatacagcagttgaaggactttactacc |
| | gatgacctgttgcagctattaatgtcatgtccccaagttgaattaattcagtgtctcactaa |
| | agagttgaatgagaaacaaccatctttatcttttggtcttgctatacttcatctgttctctgca |
| | gacatgaaaaaagttggcattaagctacttcaagaaatcaataaaggtgggatagat |
| | gcagtagaaagtcttatgataaatgattccttttgctccatagaaaagtggcaagaagtg |
| | gcaaatatatgttcacagaatggctttgacaaattatctaatgacatcacgtctattcttcg |
| | atctcaggctgcagttacagaaatttctgaagaggatgacgcagtcaacctaatggaa |
| | catgtgttttggtagttctatatcttaaccagctgagggagcttgtacaacaccttatg |
| 228362_s_at | [SEQ ID NO: 74] |
| | gtactggcccttcggattgaaagtatacagtgatgaaatttgctgccactctttcatgcttg |
| | gagtgttatattcttttggatgcgagccctcaaagaaacatttaatattctcttttgccaattc |
| | agttgcatgctctgtggctttacttttaaggatctgctgctcctgttccaaatagattttccag |
| | aatttcagctgcagaaaactaactggagataggcatcgggtgacagatgtaaaaatc |
| | agaagaatgatgataacaactgctatcaagatccagcccaac |
| 228400_at | [SEQ ID NO: 75] |
| | Aataacttcatttcctacaaggtataaaaagtggtcaagtgaatgtgaaggggcttttct |
| | acacaggaatatattatcgggaacaaagtatttcctgctgccttaactctttgggatgcat |
| | aggataaaatgataaagaccattttaatatcagaaagggttgtcttattaatttttaaataa |
| | aacttcacatttcttaatggggagctcattcagaaactaaataatggtttctcaaagtgtg |
| | gtcaggatacgatctgcatcagaatccttggaatgcttgttaaaaataccaattgctatg |
| | acaaaaccaagtctgctggaaactgcatttcagcaggtttcccatgttattctgatgtatttt |
| | aacatttgagagccactaccaatcatctgtacagttcctactg |
| 228492_at | [SEQ ID NO: 76] |
| | Aaccaatacacaaaattttcctatgtcagaatgtggtggagcataatagattgtatttggt |
| | gtgcttgcgattttttttttccatagaatttattaagtgaagtttctaaaactttgcttctcctgat |
| | cccggtgaagtgtacatcataagaatccatagtactttgaagtaccattgcaccaagat |
| | gtctgactgaattcatagtcacacttttatttgaaagaaagaattgttgtagttttttttcattat |
| | tctaaaactcttgttgttagatacaagatttaattaagatctaagctcctgcttatttaatgta |
| | attctaaggtaccattttagaaaaaacatttgttttaagattccaagaaacctgtgagttaa |
| | tactatatttaaaagagaattggtaaattttgaatgtgtgtaatattttggaacctgtttaaaa |
| | accaaatatacctgcaaatagatacagcctatcctatactattta |
| 228532_at | [SEQ ID NO: 77] |
| | Tgctgctgatagcctttatcttcctcatcataaagagctacagaaaatatcactccaagc |
| | cccaggccccagatcctcactcagatcctccagccaagctttcatccatcccagggga |
| | atcacttacctatgccagcacaactttcaaactctcagaagnnnnnnnnnnnnnnnn |
| | nnnnnnnatgctcaaattaaagtaacaaactaactcagcttttccaatgaggcttgaat |
| | ccatttcctctcatctcagccctatcttcacacatcactttcacttttttacaaattttggacca |
| | ccacctgtgtgaaactgcagtcggagttgtttagatgtgatctggcaatgctatccagcat |
| | ctttggagaccaatggtcagtcttttcctggccagaggaaagattgatggccctcccact |
| | tgaactgacagcctgtganncccttgggggcatagactgccttccttggacccttccaa |
| | agtgtgtggtacngagctcagtgcacagagtattcacccagcatcatgaatcaacttg |
| 229152_at | [SEQ ID NO: 78] |
| | tgaagaaagttctcctcctgatcacagccatcttggcagtggctgttggtttcccagtctct |
| | caagaccaggaacgagaaaaaagaagtatcagtgacagcgatgaattagcttcag |
| | ggttttttgtgttcccttacccatatccatttcgcccacttccaccaattccatttccaagattt |
| | ccatggtttagacgtaattttcctattccaatacctgaatctgcccctacaactccccttcct |
| | agcgaaaagtaaacaagaaggaaaagtcacgataaacctggtcacctgaaattga |
| | aattgagccacttccttgaagaatcaaaattcctgttaataaaagaaaaacaaatgtaa |
| | ttgaaatagcacacagcattctctagtcaatatctttagtgatcttctttaata |
| 229390_at | [SEQ ID NO: 79] |
| | gctgatttagcttatggaagaggaaccagaaatttgtccttgaataatgnttcccgtgttg |
| | ggctggatcttgatagcagttgttatcatcattcttctgatttttacatctgtcacccgatgcct |
| | atctccagttagttttctgcagctgaaattctggaaaatctatttggaacaggagcagca |
| | gatccttaaaagtaaagccacagagcatgcaactgaattggcaaaagagaatattaa |
| | atgtttctttgagggctcgcatccaaaagaatataacactccaagcatgaaagagtgg |
| | cagcaaatttcatcactgtatactttcaatccgaagggccagtactacagcatgttgcac |
| | aaatatgtcaacagaaaagagaagactcacagtatcaggtctactgaaggagatac |
| | ggtgattcctgttcttggctttgtagattcatctggtataaacagcactcctgagttatgacct |
| | tttgaatgagtag |
| 229391_s_at | [SEQ ID NO: 80] |
| | Gtgttgggctggatcttgatagcagttgttatcatcattcttctgatttttacatctgtcacccg |
| | atgcctatctccagttagttttctgcagctgaaattctggaaaatctatttggaacaggag |
| | cagcagatccttaaaagtaaagccacagagcatgcaactgaattggcaaaagaga |
| | atattaaatgtttctttgagggctcgcatccaaaagaatataacactccaagcatgaaa |
| | gagtggcagcaaatttcatcactgtatactttcaatccgaagggccagtactacagcat |
| 229543_at | [SEQ ID NO: 81] |
| | tctactcattcaaaaggtcataactcaggagtgctgtttataccagatgaatctacaaag |
| | ccaagaacaggaatcaccgtatctccttcagtagacctgatactgtgagtcttctcttttct |
| | gttgacatatttgt |
| 229625_at | [SEQ ID NO: 82] |
| | ttagctcctcaagcatatctgactggcatgatcctgcattgtggttacctggaagggaaa |
| | aacaacccctgggaattttatccaggaagttggaacaatcacaaacaaaagtggga |
| | ggcagaaggaannggcacattaatcctnnnnnnnnttatctttttctcctnagaggca |
| | caagtgaaagcagaagctgaaaaggctgaagcgcaaaggttggcggcgattcaaa |
| | ggcagaacgagcaaatgatgcaggagagggagagactccatcaggaacaagtga |
| | gacaaatggagatagccaaacaaaattggctggcagagcaacagaaaatgcagg |
| | aacaacagatgcaggaacaggctgcacagctcagcacaacattccaagctcaaaa |
| | tagaagccttctcagtgagctccagcacgcccagaggactgttaataacgatgatcca |
| | tgtgttttactctaaagtgctaaatatgggagtttcctttttttactctttgtcactgatgacaca |
| | acagaaaagaaactgtagaccttgggacaatca |
| 231229_at | [SEQ ID NO: 83] |
| | Gcacgtccaaggtgatcctgagggctgtggcggacnaaggggacctgcaagtatnt |
| | gtccctgnncaccctgaagaaggctgtttccaccacgggntacgacatggcccgaaa |
| | tgcctatcacttcaagcgtgtgctcaaggggctggtggacaagggctcagcaggtgac |
| | cggcangggggcctcaggctccttcaccctgggcaagaagcaggcctccaagtcca |
| | agctcaaggtcaagaggcaacgacagcagaggtggcgctctgggcagcgccccttt |
| | ggacagcacaggtcactactgggctccaaacaggggcacaagcggcttatcaagg |
| | gggttcgaagggtggccaagtgccactgcaattaatgaggcaggccaggcaagca |
| | gtcaggggtgccaagancgccattggctcagtgcagtgggaa |
| 231577_s_at | [SEQ ID NO: 84] |
| | ggaacaggagcaactactaaaagagggatttcaaaaagaaagcagaataatgaa |
| | aaatgagatacaggatctccagacgaaaatgagacgacgaaaggcatgtaccata |
| | agctaaagaccagagccttcctgtcacccctaaccaaggcataattgaaacaatttta |
| | gaatttggaacaagcgtcactacatttgataataattagatcttgcatcataacaccaaa |
| | agtttataaaggcatgtggtacaatgatcaaaatc |
| 232234_at | [SEQ ID NO: 85] |
| | aacacctcttaagtctagcacactgcagtgaggccaggcacctcagtgctgggcagg |
| | ggcatcagaaggtgctaagccctctctccacaatgccaagacggagaccacagcct |
| | acaccaaatccagcccttgatttccctgctgcctccataaacagaaagaggtctgctgg |
| | atccgctaagggatcagggagaggaagaaagagggatggggtgggaggcacccc |
| | ctccagtgctcctactggttcccaagctacaggtggggtgggaaaggctttatcaggtat |
| | catcaacaggttctcaattaaagatttgatttattcaagtatgtgaaaaaattctacaatgg |
| | aaactcttattagatgctgcnnnnnnngtgctatggaccacgcacatacagccatgct |
| | gtttcag |
| 232311_at | [SEQ ID NO: 86] |
| | acataccttgggttgatccacttaggaacctcagataataacatctgccacgtatagag |
| | caattgctatgtcccaggcactctactagacacttcatacagtttagaaaatcagatggg |
| | tgtagatcaaggcaggagcaggaaccaaaaagaaaggcataaacataagaaaa |
| | aaaatggaaggggtggnaaacagagtacaataacatgagtaatttgatgggggctat |
| | tatgaactgagaaatgaactttgaaaagtatcttggggccaaatcatgtagactcttgag |
| | tgatgtgttaaggaatgctatgagtgctgagagggcatcagaagtccttgagagcctcc |
| 232375_at | [SEQ ID NO: 87] |
| | gaatatttgaatctacctagtgagtntntagngcatgnttttgtcnggnatcctggaaan |
| | gcnnnccncaaaaagntannntttgccccnttcaaaancatgcaccctgaagaagc |
| | tgtttgtacaggattgggtttattctgttattaagacaaaggcatcatggcctttgggtgag |
| | aggcccgtgtgtgtttgggatttggcaatcagcatnccatctctgtcatcaccattattgag |
| | aaaatagatggattggttccctctctgcagtcctgtggagcagttggactgctctctctgct |
| | ctcaggatgatactgtgagaacaatttaaatatgctaagcacatgtcaggaaacagtttt |
| | gtggtctttggacactcgctgtagccattccgttccatttcaggtgatt |
| 232481_s_at | [SEQ ID NO: 88] |
| | gaagtccatcctttggtccaaagcatctggaagaggaagaagagaggaatgagaaa |
| | gaaggaagtgatgcaaaacatctccaaagaagtcttttggaacaggaaaatcattca |
| | ccactcacagggtcaaatatgaaatacaaaaccacgaaccaatcaacagaatttttat |
| | ccttccaagatgccagctcattgtacagaaacattttagaaaaagaaagggaacttca |
| | gcaactgggaatcacagaatacctaaggaaaaacattgctcagctccagcctgatat |
| | ggaggcacattatcctggagcccacgaagagctgaagttaatggaaacattaatgta |
| | ctcacgtccaaggaaggtattagtggaacagacaaaaaatgagtattttgaacttaaa |
| | gctaatttacatgctgaacctgactatttagaagtcctggagcagcaaacatagatgga |
| | gagtttgagggctttcgcagaaatgctgtgattctgttttaagtccataccttgtaaataagt |
| | gccttacgtgagtgtgtcatcaatcagaacctaagc |
| 234907_x_at | [SEQ ID NO: 89] |
| | Agaagagattctgctgtctacatcaatacacctgaatagttggacagaaaattgaaatc |
| | ttttaactaattctaactatgaagcacagtgaaatagaaagttaggct |
| 235175_at | [SEQ ID NO: 90] |
| | Gacagtgagctggcacagagttagggaaattgactgtgtctcatattggctagtgaga |
| | gtgatctgttggaattgtatatcaaaattttaatgtacatacattttgtctagcaattctactatt |
| | gggtatttatatagtacatataaatatnaatgtatatgtttagtaaatatatacttatagttag |
| | taaatatantttatatctatttagtaaatatactaaatgtcaggnntctgagnccaagctn |
| | aagccatcatatnccctgtgacctgcatgntacatncgtccagatggnctgaagcaag |
| | tgannnntcacaaaagaagtgaaaatggcctgttcctgccttaactgatgacattacct |
| | tgtgaaattccttctcctggctcatcctggctcaaaagctcccccactaagcaacttgtga |
| | cacccacctctgcccgcagagaacaaccccctttgactgtaattttcctttaccaaccca |
| | aatcctgtaaaatggtcccaacctatctcc |
| 235276_at | [SEQ ID NO: 91] |
| | Accctgcactcccaaagattttgtgcagatgggtagttccnttttttaaaaattgtgcagat |
| | atggaaaattgtgacttacttcatgaccagaactatctagaatatgtgtgggggtataaa |
| | catcttgcttaaccaaatatctatgtaggcagaggtaaccaggagagaagcaagactt |
| | gctgcctaaaggagcccaccattttacttttcacatttaatctgccacgttgaatcaattgg |
| | aataaaacctgactcgcaggtgactggacaggaaatcccaaagttccaccatttctat |
| | gctta |
| 236328_at | [SEQ ID NO: 92] |
| | gaaacccatgctcttactatgaaagaacgttagtacccaggttttccatgagattctctac |
| | acaggcaagaagctccatagaagtggcatttgaagggtgtggcagaggcagtgctgt |
| | gtttatcacactggttccatttccttgcaaataagaagtctatttcccagtaacccttgcagt |
| | taagagtgtgcccatgtgattgagttctagccaatggagtgtgagcaaaagtgatataa |
| | gccactttcaggtctagcctttacaaacatcctcaggcttctctatccctgccaaggtgac |
| | cttggaggctgcttattccagactgggttgatagaaggtcactacttcatctgtgttgga |
| 237515_at | [SEQ ID NO: 93] |
| | Atgaatcagtgttactaggacttatncagtacttaaaatagcaacttggcattctttattttg |
| | tttcctggttgttttatttggagggataataaatgtctaagttatttccattaaaattttgaaatg |
| | tttgtatactttatgtgtgccattttaaagtatatgcaagttctaagcaataatctgcatgttat |
| | acaaggttgacatattttgtcctgaaatttttagttaacatttcaagaatgataaaatgaac |
| | accctgtaaattacccttctccccctcccctccatgaaaaccttgggattttcttgtgctag |
| | aacacntaccacaatgtggtgcaaagctttgt |
| 238524_at | [SEQ ID NO: 94] |
| | Aaatgtacccttgatttgatgctaatgctgtatttagggctgaaggaagcacacactaaa |
| | tatctgagtgcttttcagattccatctatgctgaaaaagaatctaggagaataaacncatt |
| | tcaattagcccttaanannnnnnnnnanaannnnagcccactaaagcccagtagg |
| | gcataggagagaacactgcaccaggattcagatctggattctaanttttgttctgaaaa |
| | atagcaagtgacactggcatgccatttaacctctccgggcctcaatttccactatagata |
| | gtacctgatgtgtcagtaagacaactgatgtaactttgccaaacaagtagaattatcctt |
| | cctcctttgtcctgctctgtcctagcttttaatacttggtctgccctaacattttcctgtatgtattt |
| | ctttatcccagatattcgaacaattgctagcaaggaaaagtaatgacggattttcatttcc |
| | caatatagtctggcaaagaaatgaaaggtttacttctccttgctaattcaat |
| 238581_at | [SEQ ID NO: 95] |
| | Aacaatgtgcagctttcaactgggtggaggctgctattctgtggacagtgagatgtttcct |
| | tggcactgtcaatagacaatctgcgtagagaaattccaagctgaaagccaataatgtt |
| | ataataaaatagagattcttcagaagatgaaaggaattaccagcatggaaattgtgtc |
| | ataggcttaagggctaaagaagaagccttttcttttctgttcaccctcaccaagagcaca |
| | acttaaatagggcattttataacctgaacacaatttatattggacttaattattatgtgtaat |
| | atgtttataatcctttagatcttataaatatgtggtataaggaatgccatataatgtgccaa |
| | aaatctgagtgcatttaatttaatgcttgcttatagtgcta |
| 238587_at | [SEQ ID NO: 96] |
| | gcttctacaagtgtgccacatcaatccggtaatgccccagtgttattcacagacagaac |
| | tttgtttcctgtgattttaaaataccgcgtctgttcctccatggaccagagtaattggcacatt |
| | ttaatgcataagctgggggtttcattttcccaggctctcttcaccatcactgcattggtagct |
| | aggagcttattgcttcaccccagtatggagttcagattacagtgttttccattacatttagat |
| | tcatagaatctgaatggctgattaaatggccatctgatggctgaaagaggggcgtattttt |
| | cactctgtagtgaaaggcttggaggagtttctactt |
| 239012_at | [SEQ ID NO: 97] |
| | taaaaataagtcgccagctctctcctttataaacagtctttagactggtttgtatcatgccc |
| | cttgatgtaccagagatatgtttaaccaacctagttttgttgattctgacaatctcacacac |
| | atttaagaatttaccatttttcaggcacttttcaatgttaaaaaaaattaaatccaattattga |
| | aaatcagtttgacaaacaacccccactccatnncccnggcnanaaaaaaaaaaaa |
| | anaanaacaaaagcagctaattcagtgatacaaactctgtaaggtggcaaattcccc |
| | caactcgccaaggaaatagcacatatttattntctcccatctttactccaaatttgggacc |
| | tcttcctctgataacacagtcttttaggttacttgaaatcagcccccatttaaagactctttg |
| | cggcaccaagc |
| 244061_at | [SEQ ID NO: 98] |
| | Gaaatggcacattttctggatgtgagagttggtcaaaagatcacaaaaaaagtcaaa |
| | aaataattctactctgtgaatgaaaaatggatatttnngtacttaccctcataagcattaa |
| | aagaaaataatgcatgaaattccatagaaatgtgcctatcatgttatactgactcaaac |
| | cagaagacctagagtatgatattgctaatataatacatgtggtgggtatgagtggaagt |
| | atgtgtgtgagatttatcattgccatagtgtaaaagagttgaattagcttccacttgactag |
| | atgagagctcttagttcttatt |
| 244393_x_at | [SEQ ID NO: 99] |
| | Cccagccgctataacttttaacaattcccatatgtcctttattccactaagatgagtgcagt |
| | atatatttccatctgtccaaggcttcctaaatgtagccaangccaagccaacaccagtc |
| | acatgatcnaaatcaaagggcatttggggaatccaggctgtgattcagggaagttcca |
| | agtgtctgatgaagtgtttgttttacatctttgtgtcccttgcaggtctagcactgtgctatgta |
| | ggtaacatgtgctcc |
| AFFX- | [SEQ ID NO: 100] |
| HUMISGF3A/ | Ctggatatatcaagactgagttgatttctgtgtctgaagttcacccttctagacttcagacc |
| M97935_MB_at | acagacaacctgctccccatgtctcctgaggagtttgacgaggtgtctcggatagtggg |
| | ctctgtagaattcgacagtatgatgaacacagtatagagcatgaatttttttcatcttctctg |
| | gcgacagttttccttctcatctgtgattccctcctgctactctgttccttcacatcctgtgtttct |
| | agggaaatgaaagaaaggccagcaaattcgctgcaacctgttgatagcaagtgaatt |
| | tttctctaactcagaaacatcagttactctgaagggcatcatgcatcttactgaaggtaaa |
| | attgaaaggcattctctgaagagtgggtttcacaagtgaaaaacatccagatacaccc |
| | aaagtatcaggacgagaatgagggtcctttgggaaaggagaagttaagcaacatct |
| | agcaaatgttatgcataaagtcagtgcccaactgttataggttgttggataaatcagtggt |
| | tatttagggaactgcttgacgtaggaacggtaaatttctgtgggag |

In one aspect the invention provides a gene profile generated by performing pre-processing steps to produce a normalized gene or probeset intensity matrix and subjecting this matrix to a signal to noise statistical analysis to identify the differentially expressed genes or probesets and then ranking the genes or probesets in order of most differentially expressed gene.

In one embodiment a threshold may be established by plotting a measure of the expression of the relevant gene or an “index” derived from the gene intensity vector for each patient. Generally the responders and the non-responders will be clustered about a different axis/focal point. A threshold can be established in the gap between the clusters by classical statistical methods or simply plotting a “best fit line” to establish the middle ground between the two groups. Values, for example, above the pre-defined threshold can be designated as responders and values, for example below the pre-designated threshold can be designated as non-responders.

In one embodiment the performance of any given classifier can be analysed. Exhaustive performance analysis is done by varying the level of the threshold and calculating, for each value of the threshold, the predictive ability of the model (sensitivity, specificity, positive and negative prediction value, accuracy). This analysis can assist in selecting an appropriate threshold for a given classifier.

In addition performance analysis of the classifier can be done for a given threshold value to evaluate the sensitivity, specificity, positive and negative prediction values and accuracy of the model.

In a suitable embodiment of profiles provided by one or more aspects of the invention the effect of genes that are closely correlated with gender are excluded.

In one embodiment is provided a method of classifying tumor samples according to their gene profile assessed by Q-PCR using a subset of the genes found discriminant in melanoma (Example 1).

In one embodiment is provided a method of classifying NSCLC cancer tumor samples according to their gene profile assessed by Q-PCR using all or a subset of the genes found discriminant in melanoma.

A classifier might comprise the use of a supervised principal component analysis and Cox proportional hazards model; in addition to the gene expression profile, in this approach one might use the overall survival (OS), the DFI or the DFS of the samples in the training set together with tumor stage and surgery status to calculate the model parameters and subsequently calculate a risk index for a testing set; based on the testing set gene expression.

Once the gene profile has been identified and the analysis on the samples has been performed then there are a number of ways of presenting the results, for example as a heat map showing responders in one colour and non-responders in another colour. Nevertheless more qualitative information can be represented as an index that shows the results as a spectrum with a threshold, for example above the threshold patients are considered responders and below the threshold patients are considered to be non-responders. The advantage of presenting the information as a spectrum is that it allows a physician to decide whether to provide treatment for those patients thought to be non-responders, but who are located near the threshold.

“Immunotherapy” in the context of the invention means therapy based on stimulating an immune response, generally to an antigen, wherein the response results in the treatment, amelioration and/or retardation of the progression of a disease associated therewith. Treatment in this context would not usually include prophylactic treatment.

“Cancer immunotherapy” in the context of this specification means immunotherapy for the treatment of cancer. In one aspect the immunotherapy is based on a cancer testis antigen, such as Mage (discussed in more detail below).

Advantageously the novel method of the invention allows the identification of patients likely to respond to appropriate immunotherapy treatment. This facilitates the appropriate channeling of resources to patients who will benefit from them and what is more allow patients who will not benefit from the treatment to use alternative treatments that may be more beneficial for them.

This invention may be used for identifying cancer patients that are likely to respond to appropriate immunotherapy, for example patients with melanoma, breast, bladder, lung, NSCLC, head and neck cancer, squamous cell carcinoma, colon carcinoma and oesophageal carcinoma, such as in patients with MAGE-expressing cancers. In an embodiment, the invention may be used in an adjuvant (post-operative, for example disease-free) setting in such cancers, particularly lung and melanoma. The invention also finds utility in the treatment of cancers in the metastatic setting.

Immune activation gene is intended to mean a gene that facilitates, increases or stimulates an appropriate immune response. Immune response gene and immune activation gene are used interchangeably herein.

### Microarrays

An important technique for the analysis of the genes expressed by cells, such as cancer/tumour cells, is DNA microarray (also known as gene chip technology), where hundreds or more probe sequences (such as 55,000 probe sets) are attached to a glass surface. The probe sequences are generally all 25 mers or 60 mers and are sequences from known genes. These probes are generally arranged in a set of 11 individual probes for any particular gene (a probe set) and are fixed in a predefined pattern on the glass surface. Once exposed to an appropriate biological sample these probes hybridise to the relevant RNA or DNA of a particular gene. After washing, the chip is “read” by an appropriate method and a quantity such as colour intensity recorded. The differential expression of a particular gene is proportional to the measure/intensity recorded. This technology is discussed in more detail below.

A microarray is an array of discrete regions, typically nucleic acids, which are separate from one another and are typically arrayed at a density of between, about 100/cm² to 1000/cm², but can be arrayed at greater densities such as 10000/cm². The principle of a microarray experiment, is that mRNA from a given cell line or tissue is used to generate a labeled sample typically labeled cDNA, termed the ‘target’, which is hybridized in parallel to a large number of, nucleic acid sequences, typically DNA sequences, immobilised on a solid surface in an ordered array.

Tens of thousands of transcript species can be detected and quantified simultaneously. Although many different microarray systems have been developed the most commonly used systems today can be divided into two groups, according to the arrayed material: complementary DNA (cDNA) and oligonucleotide microarrays. The arrayed material has generally been termed the probe since it is equivalent to the probe used in a northern blot analysis. Probes for cDNA arrays are usually products of the polymerase chain reaction (PCR) generated from cDNA libraries or clone collections, using either vector-specific or gene-specific primers, and are printed onto glass slides or nylon membranes as spots at defined locations. Spots are typically 10-300 μm in size and are spaced about the same distance apart. Using this technique, arrays consisting of more than 30,000 cDNAs can be fitted onto the surface of a conventional microscope slide. For oligonucleotide arrays, short 20-25mers are synthesized in situ, either by photolithography onto silicon wafers (high-density-oligonucleotide arrays from Affymetrix or by ink-jet technology (developed by Rosetta Inpharmatics, and licensed to Agilent Technologies). Alternatively, presynthesized oligonucleotides can be printed onto glass slides. Methods based on synthetic oligonucleotides offer the advantage that because sequence information alone is sufficient to generate the DNA to be arrayed, no time-consuming handling of cDNA resources is required. Also, probes can be designed to represent the most unique part of a given transcript, making the detection of closely related genes or splice variants possible. Although short oligonucleotides may result in less specific hybridization and reduced sensitivity, the arraying of presynthesized longer oligonucleotides (50-100mers) has recently been developed to counteract these disadvantages.

Thus in performing a microarray to ascertain whether a patient presents a gene signature of the present invention, the following steps are performed: obtain mRNA from the sample and prepare nucleic acids targets, contact the array under conditions, typically as suggested by the manufactures of the microarray (suitably stringent hybridisation conditions such as 3×SSC, 0.1% SDS, at 50° C.) to bind corresponding probes on the array, wash if necessary to remove unbound nucleic acid targets and analyse the results.

It will be appreciated that the mRNA may be enriched for sequences of interest such as those in Table 1 by methods known in the art, such as primer specific cDNA synthesis. The population may be further amplified, for example, by using PCR technology. The targets or probes are labeled to permit detection of the hybridisation of the target molecule to the microarray. Suitable labels include isotopic or fluorescent labels which can be incorporated into the probe.

Once a target gene/profile has been identified there are several alternative analytical methods to microarray that can be used to measure whether the gene(s) is/are differentially expressed.

In one aspect, the invention provides a microarray comprising polynucleotide probes complementary and hybridisable to a sequence of the gene product of at least one of the genes selected from the genes listed in Table 1. Suitably, polynucleotide probes or probe sets complementary and hybridisable to the genes of Table 1 constitute at least 50%, at least 60%, at least 70%, at least 80%, at least 90% or substantially all of the probes or probe sets on said microarray.

Suitably, the microarray comprises polynucleotide probes complementary and hybridisable to a sequence of the gene product of the genes listed in Table 2.

Suitably, the solid surface with detection agents or microarray according to the invention comprise detection agents or probes that are capable of detecting mRNA or cDNA expressed from, for example, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, 54, 56, 57, 58, 59, 60, 61, 62, 63, 64, 65, 66, 67, 68, 69, 70, 71, 72, 73, 74, 75, 76, 77, 78, 79 80, 81, 82 or 83 genes in Table 1.

In some instance, PCR is a more sensitive technique than microarray and therefore can detect lower levels of differentially expressed genes.

In an alternative embodiment, a patient may be diagnosed to ascertain whether his/her tumor expresses the gene signature of the invention utilising a diagnostic kit based on PCR technology, in particular Quantitative PCR (For a review see Ginzinger D Experimental haematology 30 (2002) p 503-512 and Giuliette et al Methods, 25 p 386 (2001).

Analytical techniques include real-time polymerase chain reaction, also called quantitative real time polymerase chain reaction (QRT-PCR or Q-PCR), which is used to simultaneously quantify and amplify a specific part of a given DNA molecule present in the sample.

The procedure follows the general pattern of polymerase chain reaction, but the DNA is quantified after each round of amplification (the “real-time” aspect). Three common methods of quantification are the use of (1) fluorescent dyes that intercalate with double-strand DNA, (2) modified DNA oligonucleotide probes that fluoresce when hybridized with a complementary DNA and (3) Taqman probes complementary to amplified sequence that are hydrolyzed by DNA polymerase during elongation which release a fluorescent dye.

The basic idea behind real-time polymerase chain reaction is that the more abundant a particular cDNA (and thus mRNA) is in a sample, the earlier it will be detected during repeated cycles of amplification. Various systems exist which allow the amplification of DNA to be followed and they often involve the use of a fluorescent dye which is incorporated into newly synthesised DNA molecules during real-time amplification. Real-time polymerase chain reaction machines, which control the thermocycling process, can then detect the abundance of fluorescent DNA and thus the amplification progress of a given sample. Typically, amplification of a given cDNA over time follows a curve, with an initial flat-phase, followed by an exponential phase. Finally, as the experiment reagents are used up, DNA synthesis slows and the exponential curve flattens into a plateau.

Alternatively the mRNA or protein product of the target gene(s) may be measured by Northern Blot analysis, Western Blot and/or immunohistochemistry.

In one aspect the analysis to identify the profile/signature is performed on a patient sample wherein a cancer testis antigen is expressed.

When a single gene is analysed, for example, by Q-PCR then the gene expression can be normalised by reference to a gene that remains constant, for example genes with the symbol H3F3A, EIF4G2, HNRNPC, GUSB, PGK1, GAPDH or TFRC may be suitable for employing in normalisation. The normalisation can be performed by subtracting the value obtained for the constant gene from the Ct value obtained for the gene under consideration.

One parameter used in quantifying the differential expression of genes is the fold change, which is a metric for comparing a gene's mRNA-expression level between two distinct experimental conditions. Its arithmetic definition differs between investigators. However, the higher the fold change the more likely that the differential expression of the relevant genes will be adequately separated, rendering it easier to decide which category (responder or non-responder) the patient falls into.

The fold change may, for example be at least 2, at least 10, at least 15, at least 20 or 30.

Another parameter also used to quantify differential expression is the “p” value. It is thought that the lower the p value the more differentially expressed the gene is likely to be, which renders it a good candidate for use in profiles of the invention. P values may for example include 0.1 or less, such as 0.05 or less, in particular 0.01 or less. P values as used herein include corrected “P” values and/or also uncorrected “P” values.

Another parameter to identify genes that could be used for sample classification is signal to noise, this algorithm measures the difference in expression level between the two groups being compared weighted by the sum of the intragroup standard deviation. It thus can be used to rank genes with highest expression difference between groups with low intragroup dispersion.

The invention also extends to separate embodiments according to the invention described herein, which comprise, consist essentially of, or consists of the components/elements described herein.

The invention extends to the functional equivalents of genes listed herein, for example as characterised by hierarchical classification of genes such as described by Hongwei Wu et al 2007 (Hierarchical classification of equivalent genes in prokaryotes—Nucleic Acid Research Advance Access).

Whilst not wishing to be bound by theory, it is thought that it is not necessarily the gene per se that is characteristic of the signature but rather it is the gene function which is fundamentally important. Thus a functionally equivalent gene to an immune activation gene such as those listed in Table 1 may be employed in the signature, see for example, Journal of the National Cancer Institute Vol 98, No. 7 Apr. 5, 2006.

The genes were identified by specific probes and thus a skilled person will understand that the description of the genes above is a description based on current understanding of what hybridises to the probe. However, regardless of the nomenclature used for the genes by repeating the hybridisation to the relevant probe under the prescribed conditions the requisite gene can be identified.

The invention extends to use of the profile(s) according to the invention for predicting or identifying a patient as a responder or non-responder to immunotherapy, such as cancer immunotherapy, for example cancer testis immunotherapy, in particular Mage immunotherapy, especially for melanoma.

Thus the invention includes a method of analyzing a patient derived sample, based on expression of the profile/gene(s) according to the invention for the purpose of characterising the patient from which the sample was derived as a responder or non-responder to immunotherapy according to the present invention.

In one aspect the invention provides a method for measuring expression levels of polynucleotides from genes identified herein, in a sample for the purpose of identifying if the patient, from whom the sample was derived, is likely to be a responder or non-responder to immunotherapy such a cancer immunotherapy according to the present invention comprising the steps:

isolating the RNA from the sample,

optionally amplifying the copies of the cDNA from the sample for said genes, and

quantifying the levels of cDNA in the sample.

In some embodiments, the invention provides a diagnostic kit comprising at least one component for performing an analysis on a patient derived sample to identify a profile according to the invention, the results of which may be used to designate a patient from which the sample was derived as a responder or non-responder to immunotherapy.

The kit may comprise materials/reagents for PCR (such as QPCR), microarray analysis, immunohistochemistry or other analytical technique that may be used for accessing differential expression of one or more genes.

The invention also provides a diagnostic kit comprising a set of probes capable of hybridising to the mRNA or cDNA of one or more, such as at least 5 genes described herein in relation to the invention, for example a diagnostic kit comprising a set of probes capable of hybridising to the mRNA or its cDNA of at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, 54, 56, 57, 58, 59, 60, 61, 62, 63, 64, 65, 66, 67, 68, 69, 70, 71, 72, 73, 74, 75, 76, 77, 78, 79 80, 81, 82 or 83 genes in Table 1.

In another embodiment this invention relates to diagnostic kits. For example, diagnostic kits containing such microarrays comprising a microarray substrate and probes that are capable of hybridising to mRNA or cDNA expressed from, for example, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, 54, 56, 57, 58, 59, 60, 61, 62, 63, 64, 65, 66, 67, 68, 69, 70, 71, 72, 73, 74, 75, 76, 77, 78, 79 80, 81, 82 or 83 genes in Table 1 that are capable of demonstrating the gene signature of the invention.

In one aspect the invention provides microarrays adapted for identification of a signature according to the invention.

In some embodiments, the invention also extends to substrates and probes suitable for hybridising to an mRNA or cDNA moiety expressed from one or more genes employed in the invention, for example from Table 1.

Commercially available microarrays contain many more probes than are required to characterise the differential expression of the genes under consideration at any one time, to aid the accuracy of the analysis. Thus one or more probe sets may recognise the same gene.

Thus in one embodiment multiple probes or probe sets are used to identify differential expression, such as upregulation of a gene according to any aspect of the invention herein described.

The diagnostic kit may, for example comprise probes, which are arrayed in a microarray.

Specifically, prepared microarrays, for example, containing one or more probe sets described herein can readily be prepared by companies such as Affymetrix, thereby providing a specific test and optionally reagents for identifying the profile, according to the invention.

In an embodiment the microarrays or diagnostic kits will additionally be able to test for the presence or absence of the relevant cancer testis antigen expressing gene such as the Mage gene.

Thus in one aspect the invention provides a probe and/or probe set suitable for said hybridisation, under appropriate conditions. The invention also extends to use of probes, for example as described herein or functional equivalents thereof, for the identification of a gene profile according to the present invention.

In some embodiments, the invention herein described extends to use of all permutations of the probes listed herein (or functional analogues thereof) for identification of the said signature.

In one aspect the invention provides use of a probe for the identification of differential expression of at least one gene product of an immune activation gene for establishing if a gene profile according to the present invention is present in a patient derived sample.

In embodiments of the present invention in which hybridisation is employed, hybridisation will generally be performed under stringent conditions, such as 3×SSC, 0.1% SDS, at 50° C.

Once the target gene(s)/profile has/have been identified then it is well within the skilled person's ability to design alternative probes that hybridise to the same target. Therefore the invention also extends to probes, which under appropriate conditions measure the same differential expression of the gene(s) of the present invention to provide a signature/profile as described.

The invention also extends to use of the relevant probe in analysis of whether a cancer patient will be a responder or non-responder to treatment with an appropriate immunotherapy.

The invention also extends to use (and processes employing same) of known microarrays for identification of a signature according to the invention.

A nucleic acid probe may be at least 10, 15, 20, 25, 30, 35, 40, 50, 75, 100 or more nucleotides in length and may comprise the full length gene. Probes for use in the invention are those that are able to hybridise specifically to the mRNA (or its cDNA) expressed from the genes listed in Table 1 under stringent conditions.

The present invention further relates to a method of screening the effects of a drug on a tissue or cell sample comprising the step of analysing the expression profile, employing any embodiment of the invention described herein before and after drug treatment. The invention therefore provides a method for screening for a drug, which would alter the gene profile to that of a patient having improved survival following treatment with, for example, Mage antigen specific cancer immunotherapy (ie. to alter the gene profile to that of a responder), to enable the patient to benefit from, for example, Mage antigen specific cancer immunotherapy.

The present invention further provides a method of patient diagnosis comprising, for example, the step of analysing the expression profile according to any embodiment of the invention described herein and comparing it with a standard to diagnose whether the patient would benefit from Mage specific immunotherapy.

The invention includes a method of patient diagnosis comprising the step of analysing the expression profile according to any embodiment of the invention from a tumour tissue sample given by a patient and assessing, for example whether 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, 54, 56, 57, 58, 59, 60, 61, 62, 63, 64, 65, 66, 67, 68, 69, 70, 71, 72, 73, 74, 75, 76, 77, 78, 79 80, 81, 82 or 83 of said genes in Table 1 are expressed.

Thus in clinical applications, tissue samples from a human patient may be screened for the presence and/or absence of the expression of, any embodiment of the invention described herein.

In an alternative aspect the invention provides a method further comprising the steps of analyzing a tumour derived sample to determine which antigen(s) are expressed by the tumour and hence enabling administration of an a therapeutically effective amount of an appropriate antigen specific cancer immunotherapeutic, for example where the tumour is found to be MAGE (such as Mage A3) positive, appropriate treatment may, for example, include administration of Mage A3 antigen specific immunotherapy.

A sample such as tumour tissue of a patient is deemed to present the gene signature of the invention if one or more genes, such as substantially all the genes of any embodiment of the invention are differentially expressed (such as upregulated), and can be detected by microarray analysis or other appropriate analysis for example as described herein.

Further specific embodiments are described below.

In some embodiments the method comprises the steps of:

1 analysing a patient derived sample for the expression of the gene products of one or more genes of Table 1,

2 normalisation of the expression level of the gene products;

3 comparing the normalised expression level with a standard, wherein the standard is a value for, or a function of, the expression of a gene product or products of Table 1 in a patient or patients who have a known responder or non responder status, such that comparison of the standard information with information concerning expression of the same genes in the patient derived sample allows a conclusion to be drawn about responder or non-responder status in the patient;

4 characterising the patient from which the sample was derived as a responder or non-responder; and

5 optionally including the step of selecting the patient for at least one administration of an appropriate immunotherapeutic if the patient is characterized as a responder to the immunotherapeutic.

In one aspect normalisation is carried out using an ‘internal’ reference such as the expression of a house keeping gene or genes from the same sample. In one aspect the normalisation is carried out using an external reference, such as that derived from a different individual or individuals.

In one aspect the characterisation of the sample is carried our using a microarray. In one aspect the characterisation of the sample is carried our using a nucleic acid amplification technique such as PCR.

In one aspect the characterisation of a new sample using a microarray-based technique includes the pre-processing step of sample and gene normalisation to produce gene expression values comparable to the standard or training set. The sample normalisation may be carried out using the GCRMA algorithm (Wu, 2004) exemplified in Appendix 1, for example with reference GCRMA parameters calculated from suitable training data. Examples of parameters that may be calculated on a training data are reference quantiles and probe effects. Gene normalisation may be carried out using a Z-score calculation wherein a probe set specific mean is subtracted from the probe set value and this mean-centred expression value is then weighted by a probe set specific standard deviation.

In one aspect the characterisation of a new sample using Q-PCR involves a pre-processing step of normalisation of patient raw data using certain reference or housekeeping genes. Z-score calculation may be carried out using parameters from a standard or training set.

In one aspect, the steps of comparing and characterizing a melanoma patient utilises the 100 probe sets or 83 genes listed in Table 1 for characterising a patient as a responder (R) or gene signature (GS)+ or a non responder (NR,GS−) using the following algorithm:

Algorithm 1

library(genefilter)
#### load testset to classify (normalized microarray data)
load(“testset.RData”) ### ExpressionSet containing samples to
classify
testset<-data ###(modify xx according to batch number)
### Load training set parameters ##############
load(“M8.train.parameters.RData”)
PS<-M8.train.parameters[[1]]
M8.train.means<-M8.train.parameters[[2]]
M8.train.sd<-M8.train.parameters[[3]]
M8.train.U<-M8.train.parameters[[4]]
M8.trainPC1barRs<-M8.train.parameters[[5]]
M8.trainPC1sdRs<-M8.train.parameters[[6]]
M8.trainPC1barNRs<-M8.train.parameters[[7]]
M8.trainPC1sdNRs<-M8.train.parameters[[8]]
################################## Use SPCA on test set -
#######################
testset<-testset[PS,]
test<-(exprs(testset)−M8.train.means)/M8.train.sd
PCtest<-t(test) %*% M8.train.U
PC1test<-PCtest[,1]
distanceR<-c( )
distanceNR<-c( )
probR<-c( )
probNR<-c( )
SPCAclass<-c( )
for (i in 1:ncol(test)) {
distancesR<-abs(PCtest[i,1]−M8.trainPC1barRs)/M8.trainPC1sdRs
distancesNR<-abs(PCtest[i,1]−M8.trainPC1barNRs)/M8.trainPC1sdNRs
distanceR<-c(distanceR,distancesR)
distanceNR<-c(distanceNR,distancesNR)
probRs<-exp(−distancesR/2)/(exp(−distancesR/2)+exp(−
distancesNR/2))
probNRs<-exp(−distancesNR/2)/(exp(−distancesR/2)+exp(−
distancesNR/2))
probR<-c(probR,probRs)
probNR<-c(probNR,probNRs)
}
cutoff=0.43
clust<-ifelse(as.vector(probR)>cutoff, R,NR))

### Where

testset is a matrix with 100 rows containing the normalized microarray data for the 100 PS
M8.train.parameters is an object of class list containing:
   1. a character list of the 100 PS
   2. a vector of 100 mean values for each PS in the train set
   3. a vector of 100 sd values for each PS in the train set
   4. a matrix of 100 rows and 56 columns containing the U matrix of the svd decomposition of the train matrix
   5. the PC1 mean value of the responder group in the train
   6. the PC1 sd value of the responder group in the train
   7. the PC1 mean value of the non-responder group in the train
   8. the PC1 sd value of the non-responder group in the train
      The mean and sd of each group in the training set (rounded to three significant digits) are:

| | |
|---|---|
| mean_PC_{1R} | −4.622 |
| sd_PC_{1R} | 5.727 |
| mean_PC_{1NR} | 2.991 |
| sd_PC_{1NR} | 7.051 |

### Mean, Standard Deviations (Sd) and PC₁Coefficients for the 100 PS Classifier Features

| | Mean | Sd | PC1 |
|---|---|---|---|
| 213793_s_at | 6.638 | 1.437 | 0.0827 |
| 223593_at | 4.245 | 1.721 | 0.0698 |
| 225996_at | 5.369 | 2.116 | 0.0625 |
| 204556_s_at | 3.515 | 1.49 | 0.0594 |
| 223575_at | 5.664 | 1.785 | 0.0556 |
| 205097_at | 7.907 | 1.526 | 0.0553 |
| 231229_at | 6.464 | 1.711 | 0.0504 |
| 1562051_at | 3.576 | 1.847 | 0.0503 |
| 244393_x_at | 4.702 | 1.444 | 0.0494 |
| 200615_s_at | 6.286 | 1.232 | 0.0407 |
| 228316_at | 5.362 | 1.369 | 0.0402 |
| 201474_s_at | 4.506 | 1.331 | 0.0376 |
| 222962_s_at | 5.177 | 1.139 | 0.0372 |
| 236328_at | 7.034 | 1.936 | 0.0339 |
| 232481_s_at | 3.731 | 2.053 | 0.0328 |
| 228400_at | 3.458 | 1.437 | 0.0279 |
| 211149_at | 4.061 | 2.272 | 0.0266 |
| 228492_at | 4.538 | 2.983 | 0.0254 |
| 237515_at | 5.513 | 1.86 | 0.0245 |
| 226084_at | 9.153 | 1.388 | 0.0234 |
| 205499_at | 4.675 | 1.719 | 0.0002 |
| 234907_x_at | 3.95 | 1.465 | −0.0051 |
| 1553132_a_at | 4.068 | 1.29 | −0.0504 |
| 239012_at | 6.533 | 1.694 | −0.0656 |
| 238587_at | 6.039 | 1.292 | −0.0717 |
| 219551_at | 4.637 | 1.569 | −0.0789 |
| AFFX- | 7.445 | 1.504 | −0.0819 |
| HUMISGF3A/M97935_MB_at | | | |
| 1562031_at | 6.386 | 1.521 | −0.0871 |
| 238524_at | 4.961 | 1.623 | −0.0883 |
| 217436_x_at | 8.377 | 1.127 | −0.0891 |
| 1552612_at | 7.216 | 1.841 | −0.0929 |
| 244061_at | 6.081 | 1.918 | −0.0935 |
| 209774_x_at | 6.653 | 1.952 | −0.0953 |
| 221081_s_at | 6.805 | 2.062 | −0.0956 |
| 206082_at | 6.505 | 2.038 | −0.0988 |
| 209770_at | 10.821 | 1.153 | −0.1002 |
| 232375_at | 8.732 | 1.379 | −0.1007 |
| 211911_x_at | 10.865 | 1.461 | −0.1042 |
| 1552613_s_at | 7.491 | 1.275 | −0.1043 |
| 221875_x_at | 10.907 | 1.258 | −0.1044 |
| 214470_at | 6.927 | 1.801 | −0.1049 |
| 232311_at | 7.001 | 1.484 | −0.105 |
| 208729_x_at | 10.389 | 1.419 | −0.106 |
| 207536_s_at | 4.073 | 1.75 | −0.1061 |
| 204806_x_at | 10.065 | 1.283 | −0.1062 |
| 1554240_a_at | 4.02 | 1.761 | −0.1068 |
| 207795_s_at | 3.698 | 1.803 | −0.1073 |
| 202659_at | 6.944 | 1.284 | −0.1077 |
| 210606_x_at | 3.915 | 1.892 | −0.1083 |
| 235276_at | 7.632 | 1.905 | −0.1084 |
| 208885_at | 10.544 | 1.865 | −0.1084 |
| 202643_s_at | 5.855 | 1.381 | −0.1087 |
| 204533_at | 8.875 | 3.111 | −0.1088 |
| 229152_at | 6.925 | 3.232 | −0.1092 |
| 1563473_at | 7.07 | 2.31 | −0.1112 |
| 204529_s_at | 7.139 | 2.08 | −0.1115 |
| 235175_at | 8.682 | 2.268 | −0.1118 |
| 204897_at | 9.206 | 1.692 | −0.1123 |
| 204070_at | 8.233 | 2.205 | −0.1125 |
| 210439_at | 4.539 | 1.825 | −0.1131 |
| 1555759_a_at | 4.213 | 1.638 | −0.1133 |
| 204224_s_at | 9.809 | 1.798 | −0.1137 |
| 202644_s_at | 8.64 | 1.472 | −0.114 |
| 231577_s_at | 8.659 | 1.996 | −0.114 |
| 210982_s_at | 11.946 | 1.662 | −0.1145 |
| 1555852_at | 6.989 | 1.89 | −0.1149 |
| 209813_x_at | 4.135 | 1.808 | −0.1152 |
| 205685_at | 6.927 | 1.728 | −0.1153 |
| 238581_at | 4.289 | 1.801 | −0.1158 |
| 229543_at | 8.937 | 2.328 | −0.1159 |
| 229390_at | 9.644 | 2.315 | −0.1159 |
| 208894_at | 11.493 | 1.628 | −0.1161 |
| 222838_at | 7.302 | 2.672 | −0.1164 |
| 228532_at | 8.693 | 1.684 | −0.1165 |
| 209606_at | 5.957 | 2.038 | −0.1168 |
| 217478_s_at | 9.575 | 1.559 | −0.1173 |
| 229391_s_at | 9.135 | 2.228 | −0.1175 |
| 211144_x_at | 4.32 | 1.949 | −0.1179 |
| 228362_s_at | 8.288 | 2.398 | −0.1179 |
| 212671_s_at | 8.72 | 2.387 | −0.1182 |
| 203915_at | 9.242 | 3.331 | −0.1191 |
| 229625_at | 7.32 | 2.116 | −0.1197 |
| 211902_x_at | 7.387 | 1.956 | −0.1197 |
| 209671_x_at | 5.905 | 2.044 | −0.1197 |
| 1552497_a_at | 4.827 | 2.195 | −0.1205 |
| 215806_x_at | 4.544 | 1.973 | −0.1215 |
| 216920_s_at | 5.641 | 1.862 | −0.1221 |
| 210972_x_at | 7.322 | 2.354 | −0.1224 |
| 205890_s_at | 8.864 | 2.983 | −0.1225 |
| 232234_at | 6.877 | 2.249 | −0.1228 |
| 207651_at | 7.222 | 2.531 | −0.1229 |
| 202531_at | 7.451 | 1.809 | −0.1234 |
| 206666_at | 6.816 | 2.698 | −0.1242 |
| 213193_x_at | 6.825 | 2.768 | −0.1257 |
| 204116_at | 6.106 | 2.683 | −0.126 |
| 213539_at | 7.398 | 2.851 | −0.1263 |
| 211339_s_at | 5.602 | 2.061 | −0.1266 |
| 210915_x_at | 6.533 | 2.733 | −0.1267 |
| 211796_s_at | 6.946 | 2.921 | −0.1271 |
| 205758_at | 7.338 | 3.285 | −0.1275 |

In one aspect, the steps of comparing and characterizing a melanoma patient utilises any one of the 100 probe sets or 83 genes mentioned in table 13 individually to characterise a patient using the algorithm specified above wherein single gene expression values are used instead of first principal component (PC1).

In one aspect, the steps of comparing and characterizing a melanoma patient utilises the 22 genes listed in Table 5 for characterising a patient as a responder (R) or gene signature (GS)+ or a non responder (NR, GS−) using the following algorithm:

Algorithm 2

### Script for classification of test-samples fresh metatasic
melanoma TLDA2 22 genes
### based on Mage008TLDA.SPCA.DA.Mel4patent.R
### needs M8.train.parameters.22genes.TLDA2.RData (training set
parameters)
library(genefilter)
#### load testset to classify (log-scaled normalized PCR data)
load(“testset.RData”) ### ExpressionSet containing samples to
classify
### Load training set parameters ##############
load(“M8.train.parameters.22genes.TLDA2.RData”)
PS<-M8.train.parameters[[1]]
M8.train.means<-M8.train.parameters[[2]]
M8.train.sd<-M8.train.parameters[[3]]
M8.train.U<-M8.train.parameters[[4]]
M8.trainPC1barRs<-M8.train.parameters[[5]]
M8.trainPC1sdRs<-M8.train.parameters[[6]]
M8.trainPC1barNRs<-M8.train.parameters[[7]]
M8.trainPC1sdNRs<-M8.train.parameters[[8]]
######################### Use SPCA on test set -
#######################
testset<-testset[PS,]
test<-(exprs(testset)−M8.train.means)/M8.train.sd
PCtest<-t(test) %*% M8.train.U
PC1test<-PCtest[,1]
distanceR<-c( )
distanceNR<-c( )
probR<-c( )
probNR<-c( )
SPCAclass<-c( )
for (i in 1:ncol(test)) {
distancesR<-abs(PCtest[i,1]−M8.trainPC1barRs)/M8.trainPC1sdRs
distancesNR<-abs(PCtest[i,1]−M8.trainPC1barNRs)/M8.trainPC1sdNRs
distanceR<-c(distanceR,distancesR)
distanceNR<-c(distanceNR,distancesNR)
probRs<-exp(−distancesR/2)/(exp(−distancesR/2)+exp(−
distancesNR/2))
probNRs<-exp(−distancesNR/2)/(exp(−distancesR/2)+exp(−
distancesNR/2))
probR<-c(probR,probRs)
probNR<-c(probNR,probNRs)
}
cutoff=0.47
clust<-ifelse(as.vector(probR)>cutoff,R,NR)
####################
###(modify xx next line according to batch number)
write.table(cbind(pData(testset),probR),file=
“testset_batch_xx_TLDA2_22genes_classification.txt”,sep=“\t”)

### Where

Testset.RData is a matrix with 22 rows containing the normalized log-scaled PCR data for the 22 genes
M8.train.parameters is an object of class list containing:
   1. a character list of the 22 gene names
   2. a vector of 22 mean values for each gene in the train set
   3. a vector of 22 sd values for each gene in the train set
   4. a matrix of 22 rows and 22 columns containing the U matrix of the svd decomposition of the train matrix
   5. the PC1 mean value of the responder group in the train
   6. the PC1 sd value of the responder group in the train
   7. the PC1 mean value of the non-responder group in the train
   8. the PC1 sd value of the non-responder group in the train

Mean, Standard Deviations (Sd) and PC1 Coefficients for 22 Genes Classifier Features

| | | | PC1 |
|---|---|---|---|
| Gene | Mean | Sd | coefficient |
| C4orf7 | −1.397 | 1.244 | −0.1834 |
| CCL5 | −0.545 | 0.691 | −0.2441 |
| JAK2 | −1.105 | 0.354 | −0.1636 |
| IRF1 | −0.430 | 0.500 | −0.2345 |
| CXCL9 | −0.276 | 0.923 | −0.2349 |
| IL2RG | −0.657 | 0.721 | −0.2444 |
| CXCL10 | −0.830 | 0.896 | −0.2181 |
| SLC26A2 | −0.745 | 0.307 | 0.0660 |
| CD86 | −1.504 | 0.461 | −0.2272 |
| CD8A | −1.342 | 0.879 | −0.1881 |
| UBD | −0.570 | 0.945 | −0.2385 |
| GZMK | −1.470 | 0.734 | −0.2414 |
| GPR171 | −1.683 | 0.698 | −0.2180 |
| PSCDBP | −1.335 | 0.647 | −0.2212 |
| CXCL2 | −2.163 | 0.633 | −0.1437 |
| ICOS | −1.714 | 0.697 | −0.2029 |
| TRBC1 | −2.714 | 1.313 | −0.2026 |
| TRA@; TRAJ17; TRDV2; TRAC; TRAV20 | −0.762 | 0.666 | −0.2464 |
| TARP; TRGC2 | −2.405 | 0.877 | −0.1904 |
| ITK | −1.862 | 0.896 | −0.2178 |
| CD3D | −1.478 | 0.806 | −0.2452 |
| HLA-DMA | −0.380 | 0.470 | −0.2284 |

The mean and sd of each group in the training set (rounded to three significant digits) are:

| | |
|---|---|
| mean_PC_{1R} | −2.055 |
| sd_PC_{1R} | 2.920 |
| mean_PC_{1NR} | 1.210 |
| sd_PC_{1NR} | 3.951 |

In one aspect, the steps of comparing and characterizing a melanoma patient utilises any one of the 22 genes mentioned in Table 11 individually to characterise a patient using the algorithm specified above wherein single gene expression values are used instead of first principal component (PC1).

In one aspect, the steps of comparing and characterizing a NSCLC patient utilises the 23 genes listed in Table 7 for characterising a patient as a responder (non-relapse or gene signature+(GS+),1) or a non responder (relapse, GS−,0) using the following algorithm:

Algorithm 3

### Script for classification of test-samples fresh resected
NSCLC TLDAmerge 23 genes
### based on
Mage004.SPCA.Cox.classifier.contruction.TLDAmerge.23genes.DFI.
Squamous.R
### needs M4.train.parameters.23genes.TLDAmerge.RData (training
set parameters)
library(genefilter)
#### load testset to classify (log-scaled normalized PCR data)
load(“testset.RData”) ### ExpressionSet containing samples to
classify
### Load training set parameters ##############
load(“M4.train.parameters.23genes.TLDAmerge.RData”)
PS<-M4.train.parameters[[1]]
M4.train.means<-M4.train.parameters[[2]]
M4.train.sd<-M4.train.parameters[[3]]
M4.train.U<-M4.train.parameters[[4]]
M4.train.Btreatment<-M4.train.parameters[[5]]
M4.train.Binteraction<-M4.train.parameters[[6]]
M4.train.medianHR<-M4.train.parameters[[7]]
################################## Use SPCA on test set -
#######################
testset<-testset[PS,]
test<-(exprs(testset)−M4.train.means)/M4.train.sd
PCtest<-t(test) %*% M4.train.U
PC1test<-PCtest[,1]
HR=M4.train.Btreatment+PC1test*M4.train.Binteraction
classification=ifelse(HR<M4.train.medianHR,1,0)
####################
###(modify xx next line according to batch number)
write.table(cbind(pData(testset),probR),file=
“testset_batch_xx_M4_TLDAmerge_23genes_classification.txt”,
sep=“\t”)

### Where

Testset.RData is a matrix with 23 rows containing the normalized log-scaled PCR data for the 23 genes
M4.train.parameters is an object of class list containing:
   1. a character list of the 23 gene names
   2. a vector of 23 mean values for each gene in the train set
   3. a vector of 23 sd values for each gene in the train set
   4. a matrix of 23 rows and 23 columns containing the U matrix of the svd decomposition of the train matrix
   5. the Bₜᵣₑₐₜₘₑₙₜ in risk score computation
   6. the B_{PC1interaction} in risk score computation
   7. the median risk score in train

Mean, Standard Deviations (Sd) and PC1 Coefficients for 23 Genes Classifier Features

| | | | PC1 |
|---|---|---|---|
| Gene | Mean | sd | coefficient |
| C4orf7 | −2.35768 | 1.455544 | −0.12114 |
| CCL5 | −0.9599 | 0.350039 | −0.23097 |
| JAK2 | −1.36811 | 0.260374 | −0.19931 |
| IRF1 | −0.52347 | 0.276644 | −0.2256 |
| CXCL9 | −0.87804 | 0.563437 | −0.21386 |
| IL2RG | −0.83528 | 0.358042 | −0.24997 |
| CXCL10 | −1.36857 | 0.615177 | −0.17136 |
| SLC26A2 | −1.44043 | 0.255169 | −0.05637 |
| CD86 | −1.7699 | 0.499237 | −0.13267 |
| CD8A | −1.33733 | 0.375334 | −0.25173 |
| UBD | −0.71367 | 0.546652 | −0.21295 |
| GZMK | −1.77411 | 0.529496 | −0.24628 |
| GPR171 | −1.81327 | 0.32409 | −0.19376 |
| PSCDBP | −1.17746 | 0.387117 | −0.24162 |
| CXCL2 | −1.16947 | 0.696255 | −0.09696 |
| ICOS | −2.15436 | 0.403522 | −0.23497 |
| TRBC1 | −2.62512 | 1.013281 | −0.12679 |
| TRA@; TRAJ17; TRDV2; TRAC; | −1.19671 | 0.3944 | −0.25817 |
| TRAV20 | | | |
| TARP; TRGC2 | −2.22752 | 0.481252 | −0.19299 |
| ITK | −1.85777 | 0.394118 | −0.26077 |
| CD3D | −1.64584 | 0.397626 | −0.25514 |
| HLA-DMA | −0.81144 | 0.380465 | −0.22948 |
| SLAMF7 | −1.33744 | 0.464338 | −0.21762 |

### Where Bₜᵣₑₐₜₘₑₙₜ0-0.2429033

and B_{PC1interaction}=0.1720062 were obtained from the training set.

The risk score of the new sample is compared to the median risk score of the training set=−0.323947288 and the sample is classified GS+ (Responder, Non-Relapse,1) if Risk score is lower than this value.

In one aspect, the steps of comparing and characterizing a NSCLC patient utilises any one of the 23 genes mentioned in Table 12 individually to characterise a patient using the algorithm specified above wherein single gene expression values are used instead of first principal component (PC1).

In one aspect, the steps of comparing and characterizing a NSCLC patient utilises the 22 genes listed in Table 9 for characterising a patient as a responder (non-relapse or gene signature+(GS+),1) or a non responder (relapse, GS−,0) using the following algorithm:

Algorithm 4

### Script for classification of test-samples fresh resected
NSCLC TLDAmerge 22 genes
### based on Mage004.SPCA.Cox.classifier.contruction.
DFI.Squamous.R
### needs M4.train.parameters.22genes.TLDA2.RData (training set
parameters)
library(genefilter)
#### load testset to classify (log-scaled normalized PCR data)
load(“testset.RData”) ### ExpressionSet containing samples to
classify
### Load training set parameters ##############
load(“M4.train.parameters.22genes.TLDA2.RData”)
PS<-M4.train.parameters[[1]]
M4.train.means<-M4.train.parameters[[2]]
M4.train.sd<-M4.train.parameters[[3]]
M4.train.U<-M4.train.parameters[[4]]
M4.train.Btreatment<-M4.train.parameters[[5]]
M4.train.Binteraction<-M4.train.parameters[[6]]
M4.train.medianHR<-M4.train.parameters[[7]]
################################## Use SPCA on test set -
#######################
testset<-testset[PS,]
test<-(exprs(testset)−M4.train.means)/M4.train.sd
PCtest<-t(test) %*% M4.train.U
PC1test<-PCtest[,1]
HR=M4.train.Btreatment+PC1test*M4.train.Binteraction
classification=ifelse(HR<M4.train.medianHR,1,0)
####################
###(modify xx next line according to batch number)
write.table(cbind(pData(testset),probR),file=
“testset_batch_xx_M4_TLDA2_22genes_classification.txt”,sep=“\t”)

### Where

Testset.RData is a matrix with 22 rows containing the normalized log-scaled PCR data for the 22 genes
M4.train.parameters is an object of class list containing:
   1. a character list of the 22 gene names
   2. a vector of 22 mean values for each gene in the train set
   3. a vector of 22sd values for each gene in the train set
   4. a matrix of 22 rows and 22 columns containing the U matrix of the svd decomposition of the train matrix
   5. the Bₜᵣₑₐₜₘₑₙₜ in risk score computation
   6. the B_{PC1interaction} in risk score computation
   7. the median risk score in train

### Mean, Standard Deviations (Sd) and PC1 Coefficients for 22 Genes Classifier Features

| | | | PC1 |
|---|---|---|---|
| Gene | Means | Sd | coefficients |
| C4orf7 | −2.37682 | 1.432191 | −0.12613 |
| CCL5 | −0.97196 | 0.363545 | −0.23868 |
| JAK2 | −1.38351 | 0.272662 | −0.20067 |
| IRF1 | −0.5328 | 0.284196 | −0.23035 |
| CXCL9 | −0.88518 | 0.561561 | −0.21758 |
| IL2RG | −0.84755 | 0.369696 | −0.25893 |
| CXCL10 | −1.38526 | 0.608373 | −0.17545 |
| SLC26A2 | −1.45138 | 0.259368 | −0.06122 |
| CD86 | −1.78136 | 0.493304 | −0.1445 |
| CD8A | −1.35019 | 0.38214 | −0.26018 |
| UBD | −0.72426 | 0.545598 | −0.21573 |
| GZMK | −1.7857 | 0.526042 | −0.25378 |
| GPR171 | −1.81382 | 0.353983 | −0.1875 |
| PSCDBP | −1.19407 | 0.398912 | −0.24969 |
| CXCL2 | −1.17377 | 0.679063 | −0.10145 |
| ICOS | −2.16745 | 0.40877 | −0.24479 |
| TRBC1 | −2.63145 | 0.999466 | −0.12889 |
| TRA@; TRAJ17; TRDV2; TRAC; | −1.20289 | 0.392963 | −0.26276 |
| TRAV20 | | | |
| TARP; TRGC2 | −2.27109 | 0.528402 | −0.19113 |
| ITK | −1.87391 | 0.405727 | −0.26852 |
| CD3D | −1.66653 | 0.409356 | −0.26013 |
| HLA-DMA | −0.81888 | 0.400541 | −0.23598 |

Where Bₜᵣₑₐₜₘₑₙₜ=−0.193146993 and B_{PC1interaction}=−0.163704817 were obtained from the training set.

The risk score of the new sample is compared to the median risk score of the training set=−0.25737421 and the sample is classified GS+ (Responder, Non-Relapse,1) if Risk score is lower than this value.

### Immunotherapeutics

In a further aspect the invention provides a method of treating a responder patient with an appropriate immunotherapy, for example cancer immunotherapy such as cancer testis immunotherapy, after identification of the same as a responder thereto.

Thus, in some embodiments, the invention provides a method of treating a patient comprising the step of administering a therapeutically effective amount of an appropriate immunotherapy (for example cancer immunotherapy, such as Mage cancer immunotherapy), after first characterising the patient as a responder based on differential expression of at least one immune activation gene, for example as shown by appropriate analysis of a sample derived from the patient. In particular wherein the patient is characterised as a responder based on one or more embodiments described herein.

In one aspect the immunotherapy comprises an appropriate adjuvant (immunostimulant), see description below.

In yet a further embodiment of the invention there is provided a method of treating a patient suffering from, for example, a Mage expressing tumour, the method comprising determining whether the patient expresses the gene signature of the invention and then administering, for example, a Mage specific immunotherapeutic. In a further embodiment, the patient is treated with, for example, the Mage specific immunotherapy to prevent or ameliorate recurrence of disease, after first receiving treatment such as resection by surgery of any tumour or other chemotherapeutic or radiotherapy treatment.

A further aspect of the invention is a method of treating a patient suffering from a Mage expressing tumour, the method comprising determining whether the patient's tumour expresses a profile according to any embodiment of the invention from a biological sample given by a patient and then administering a Mage specific immunotherapeutic to said patient.

Also provided is a method of treating a patient susceptible to recurrence of Mage expressing tumour having been treated to remove/treat a Mage expressing tumour, the method comprising determining whether the patient's tumour expresses one or more genes selected from any embodiment of the invention from a biological sample given by a patient and then administering a Mage specific immunotherapeutic.

The invention also provides as method of treatment or use employing:
MAGE specific immunotherapeutic comprising a MAGE antigen or peptide thereof,
MAGE antigen comprising a MAGE-A3 protein or peptide,
MAGE antigen comprising the peptide EVDPIGHLY,
MAGE antigen or peptide fused or conjugated to a carrier protein, for example in which the carrier protein is selected from protein D, NS1 or CLytA or fragments thereof, and/or
MAGE specific immunotherapeutic further comprises an adjuvant, for example in which the adjuvant comprises one or more or combinations of: 3D-MPL; aluminium salts; CpG containing oligonucleotides; saponin-containing adjuvants such as QS21 or ISCOMs; oil-in-water emulsions; and liposomes.

The invention also extends to use of an immunotherapy such as a cancer immunotherapy, in particular Mage immunotherapy in the manufacture of a medicament for the treatment of a patient such as a cancer patient designated as a responder, thereto.

It was observed that one patient initially characterised as a non-responder was subsequently characterised as responder after radiation therapy. Interestingly the inventors also believe that it may be possible to induce a responders profile in at least some non-responders, for example by subjecting the patient to radiation therapy, or administering an inflammatory stimulant such as interferon or a TLR 3 (for example as described in WO 2006/054177), 4, 7, 8 or TLR 9 agonist (for example containing a CpG motif, in particular administering a high dose thereof such as 0.1 to 75 mg per Kg adminstered, for example weekly). See for example Krieg, A. M., Efler, S. M., Wittpoth, M., Al Adhami, M. J. & Davis, H. L. Induction of systemic TH1-like innate immunity in normal volunteers following subcutaneous but not intravenous administration of CPG 7909, a synthetic B-class CpG oligodeoxynucleotide TLR9 agonist. J. Immunother. 27, 460-471 (2004).

The high dose of CpG may, for example be inhaled or given subcutaneously.

The invention further provides the use of Mage specific immunotherapy in the manufacture of a medicament for the treatment of patients suffering from Mage expressing tumour or patients who have received treatment (e.g. surgery, chemotherapy or radiotherapy) to remove/treat a Mage expressing tumour, said patient expressing the gene signature of the invention.

The immunotherapy may then be administered to for example responders or once the responders profile has been induced.

In one aspect the invention provides use of Mage specific immunotherapy in the manufacture of a medicament for the treatment of patients suffering from a Mage expressing tumour, said patient characterised by their tumour expressing one or more genes selected from any embodiment of the invention.

The invention also provides use of Mage specific immunotherapy in the manufacture of a medicament for the treatment of patients susceptible to recurrence from Mage expressing tumour said patient characterised by their tumour one or more genes selected from any embodiments of the invention.

Advantageously, the invention may allow treatment providers to target those populations of patients that will obtain a clinical benefit from receiving an appropriate immunotherapy. It is expected that after screening at least 60% of patients such as 70, 75, 80, 85% or more of patients deemed/characterised as responders will receive a clinical benefit from the immunotherapy, which is a significant increase over the current levels observed with therapy such as cancer therapy generally.

Advantageously if the cancer immunotherapy is given concomitantly or subsequent to chemotherapy it may assist in raising the patient's immune responses, which may have been depleted by the chemotherapy.

In a further embodiment the immunotherapy may be given prior to surgery, chemotherapy and/or radiotherapy.

Antigen Specific Cancer Immunotherapeutics (ASCIs) suitable for use in the invention may, for example include those capable of raising a Mage specific immune response. Such immunotherapeutics may be capable of raising an immune response to a Mage gene product, for example a Mage-A antigen such as Mage-A3. The immunotherapeutic will generally contain at least one epitope from a Mage gene product. Such an epitope may be present as a peptide antigen optionally linked covalently to a carrier and optionally in the presence of an adjuvant. Alternatively larger protein fragments may be used. For example, the immunotherapeutic for use in the invention may comprise an antigen that corresponds to or comprises amino acids 195-279 of MAGE-A1. The fragments and peptides for use must however, when suitably presented be capable of raising a Mage specific immune response. Examples of peptides that may be used in the present invention include the MAGE-3.A1 nonapeptide EVDPIGHLY [Seq. ID No] (see Marchand et al., International Journal of Cancer 80(2), 219-230), and the following MAGE-A3 peptides:

| | |
|---|---|
| FLWGPRALV; | [SEQ. ID NO: 107] |
| MEVDPIGHLY; | [SEQ. ID NO: 108] |
| VHFLLLKYRA; | [SEQ. ID NO: 109] |
| LVHFLLLKYR; | [SEQ. ID NO: 110] |
| LKYRAREPVT; | [SEQ. ID NO: 111] |
| ACYEFLWGPRALVETS; | [SEQ. ID NO: 112] |
| AND | |
| TQHFVQENYLEY; | [SEQ. ID NO: 113] |

Alternative ASCIs include cancer testis antigens such as NY-ESO1, LAGE 1, LAGE 2, for example details of which can be obtained from www.cancerimmunity.orq/CTdatabase. ASCIs also include other antigens that might not be cancer testis specific such as PRAME and WT1.

The cancer immunotherapy may be based, for example on one or more of the antigens discussed below.

In one embodiment of the present invention, the antigen to be used may consist or comprise a MAGE tumour antigen, for example, MAGE 1, MAGE 2, MAGE 3, MAGE 4, MAGE 5, MAGE 6, MAGE 7, MAGE 8, MAGE 9, MAGE 10, MAGE 11 or MAGE 12. The genes encoding these MAGE antigens are located on chromosome X and share with each other 64 to 85% homology in their coding sequence (De Plaen, 1994). These antigens are sometimes known as MAGE A1, MAGE A2, MAGE A3, MAGE A4, MAGE A5, MAGE A6, MAGE A7, MAGE A8, MAGE A9, MAGE A 10, MAGE A11 and/or MAGE A12 (The MAGE A family). In one embodiment, the antigen is MAGE A3.

In one embodiment, an antigen from one of two further MAGE families may be used: the MAGE B and MAGE C group. The MAGE B family includes MAGE B1 (also known as MAGE Xp1, and DAM 10), MAGE B2 (also known as MAGE Xp2 and DAM 6) MAGE B3 and MAGE B4—the Mage C family currently includes MAGE C1 and MAGE C2.

In general terms, a MAGE protein can be defined as containing a core sequence signature located towards the C-terminal end of the protein (for example with respect to MAGE A1 a 309 amino acid protein, the core signature corresponds to amino acid 195-279).

The consensus pattern of the core signature is thus described as follows wherein x represents any amino acid, lower case residues are conserved (conservative variants allowed) and upper case residues are perfectly conserved.

Core Sequence Signature

LixvL(2x)l(3x)g(2x)apEExiWexl(2x)m(3-4x)Gxe(3-4x)gxp(2x)llt(3x)VqexYLxYxqVPxsxP(2x)yeFLWGprA(2x)Et(3x)kv

Conservative substitutions are well known and are generally set up as the default scoring matrices in sequence alignment computer programs. These programs include PAM250 (Dayhoft M. O. et al., (1978), “A model of evolutionary changes in proteins”, In “Atlas of Protein sequence and structure” 5(3) M. O. Dayhoft (ed.), 345-352), National Biomedical Research Foundation, Washington, and Blosum 62 (Steven Henikoft and Jorja G. Henikoft (1992), “Amino acid substitution matricies from protein blocks”), Proc. Natl. Acad. Sci. USA 89 (Biochemistry): 10915-10919.

In general terms, substitution within the following groups are conservative substitutions, but substitutions between groups are considered non-conserved. The groups are:

i) Aspartate/asparagine/glutamate/glutamine

ii) Serine/threonine

iii) Lysine/arginine

iv) Phenylalanine/tyrosine/tryptophane

v) Leucine/isoleucine/valine/methionine

vi) Glycine/alanine

In general and in the context of this invention, a MAGE protein will be approximately 50% or more identical, such as 70, 80, 90, 95 96, 97, 98 or 99% identical, in this core region with amino acids 195 to 279 of MAGE A1.

MAGE protein derivatives are also known in the art, see: WO 99/40188. Such derivatives are suitable for use in therapeutic vaccine formulations (Immunotherapeutic) which are suitable for the treatment of a range of tumour types.

Several CTL epitopes have been identified on the MAGE-3 protein. One such epitope, MAGE-3.A1, is a nonapeptide sequence located between amino acids 168 and 176 of the MAGE-3 protein which constitutes an epitope specific for CTLs when presented in association with the MHC class I molecule HLA.A1. Recently two additional CTL epitopes have been identified on the peptide sequence of the MAGE-3 protein by their ability to mount a CTL response in a mixed culture of melanoma cells and autologous lymphocytes. These two epitopes have specific binding motifs for the HLA.A2 (Van der Bruggen, 1994) and HLA.B44 (Herman, 1996) alleles respectively.

In a further embodiment of the invention, the tumour antigen may comprise or consist of one of the following antigens, or an immunogenic portion thereof which is able to direct an immune response to the antigen: SSX-2; SSX-4; SSX-5; NA17; MELAN-A; Tyrosinase; LAGE-1; NY-ESO-1; PRAME; P790; P510; P835; B305D; B854; CASB618 (as described in WO00/53748); CASB7439 (as described in WO01/62778); C1491; C1584; and C1585.

In one embodiment, the antigen may comprise or consist of P501S (also known as prostein). The P501S antigen may be a recombinant protein that combines most of the P501S protein with a bacterial fusion protein comprising the C terminal part of protein LytA of Streptococcus pneumoniae in which the P2 universal T helper peptide of tetanus toxoid has been inserted, ie. a fusion comprising CLytA-P2-CLyta (the “CPC” fusion partner), as described in WO03/104272.

In one embodiment, the antigen may comprise or consist of WT-1 expressed by the Wilm's tumor gene, or its N-terminal fragment WT-1F comprising about or approximately amino acids 1-249; the antigen expressed by the Her-2/neu gene, or a fragment thereof. In one embodiment, the Her-2/neu antigen may be one of the following fusion proteins which are described in WO00/44899.

In a further embodiment, the antigen may comprise or consist of “HER-2/neu ECD-ICD fusion protein,” also referred to as “ECD-ICD” or “ECD-ICD fusion protein,” which refers to a fusion protein (or fragments thereof) comprising the extracellular domain (or fragments thereof) and the intracellular domain (or fragments thereof) of the HER-2/neu protein. In one embodiment, this ECD-ICD fusion protein does not include a substantial portion of the HER-2/neu transmembrane domain, or does not include any of the HER-2/neu transmembrane domain.

In a further embodiment, the antigen may comprise or consist of “HER-2/neu ECD-PD fusion protein,” also referred to as “ECD-PD” or “ECD-PD fusion protein,” or the “HER-2/neu ECD-ΔPD fusion protein,” also referred to as “ECD-ΔPD” or “ECD-ΔPD fusion protein,” which refers to fusion proteins (or fragments thereof) comprising the extracellular domain (or fragments thereof) and phosphorylation domain (or fragments thereof, e.g., ΔPD) of the HER-2/neu protein. In one embodiment, the ECD-PD and ECD-ΔPD fusion proteins do not include a substantial portion of the HER-2/neu transmembrane domain, or does not include any of the HER-2/neu transmembrane domain.

In one embodiment, the antigen may comprise a Mage or other appropriate protein linked to an immunological fusion or expression enhancer partner. Fusion proteins may include a hybrid protein comprising two or more antigens relevant to a given disease or may be a hybrid of an antigen and an expression enhancer partner.

In one embodiment the MAGE antigen may comprise the full length MAGE protein. In an alternative embodiment the Mage antigen may comprise amino acids 3 to 312 of the MAGE antigen.

In alternative embodiments the MAGE antigen may comprise 100, 150, 200, 250 or 300 amino acids from the MAGE protein, provided that the antigen is capable of generating an immune response against MAGE, when employed in an immunotherapeutic treatment.

The antigen and partner may be chemically conjugated, or may be expressed as a recombinant fusion protein. In an embodiment in which the antigen and partner are expressed as a recombinant fusion protein, this may allow increased levels to be produced in an expression system compared to non-fused protein. Thus the fusion partner may assist in providing T helper epitopes (immunological fusion partner), preferably T helper epitopes recognised by humans, and/or assist in expressing the protein (expression enhancer) at higher yields than the native recombinant protein. In one embodiment, the fusion partner may be both an immunological fusion partner and expression enhancing partner.

In one embodiment of the invention, the immunological fusion partner that may be used is derived from protein D, a surface protein of the gram-negative bacterium, Haemophilus influenza B (WO 91/18926) or a derivative thereof. The protein D derivative may comprise the first ⅓ of the protein, or approximately or about the first ⅓ of the protein, in particular it may comprise the first N-terminal 100-110 amino acids or approximately the first N-terminal 100-110 amino acids.

In one embodiment the fusion protein comprises the first 109 residues (or 108 residues therefrom) or amino acids 20 to 127 of protein D.

Other fusion partners that may be used include the non-structural protein from influenzae virus, NS1 (hemaglutinin). Typically the N terminal 81 amino acids of NS1 may be utilised, although different fragments may be used provided they include T-helper epitopes.

In another embodiment the immunological fusion partner is the protein known as LytA. LytA is derived from Streptococcus pneumoniae which synthesise an N-acetyl-L-alanine amidase, amidase LytA, (coded by the LytA gene (Gene, 43 (1986) page 265-272) an autolysin that specifically degrades certain bonds in the peptidoglycan backbone. The C-terminal domain of the LytA protein is responsible for the affinity to the choline or to some choline analogues such as DEAE. This property has been exploited for the development of E. coli C-LytA expressing plasmids useful for expression of fusion proteins. Purification of hybrid proteins containing the C-LytA fragment at its amino terminus has been described (Biotechnology: 10, (1992) page 795-798). In one embodiment, the C terminal portion of the molecule may be used. The embodiment may utilise the repeat portion of the LytA molecule found in the C terminal end starting at residue 178. In one embodiment, the LytA portion may incorporate residues 188-305.

In one embodiment of the present invention, the Mage protein may comprise a derivatised free thiol. Such antigens have been described in WO 99/40188. In particular carboxyamidated or carboxymethylated derivatives may be used.

In one embodiment of the present invention, the tumour associated antigen comprises a Mage-A3-protein D molecule. This antigen and those summarised below are described in more detail in WO 99/40188.

In further embodiments of the present invention, the tumour associated antigen may comprise any of the following fusion proteins: a fusion protein of Lipoprotein D fragment, MAGE1 fragment, and histidine tail; fusion protein of NS1-MAGE3, and Histidine tail; fusion protein of CLYTA-MAGE1-Histidine; fusion protein of CLYTA-MAGE3-Histidine.

A further embodiment of the present invention comprises utilising a nucleic acid immunotherapeutic, which comprises a nucleic acid molecule encoding a Mage specific tumour associated antigens as described herein. Such sequences may be inserted into a suitable expression vector and used for DNA/RNA vaccination. Microbial vectors expressing the nucleic acid may also be used as vectored delivered immunotherapeutics. Such vectors include for example, poxvirus, adenovirus, alphavirus and listeria.

Conventional recombinant techniques for obtaining nucleic acid sequences, and production of expression vectors of are described in Maniatis et al., Molecular Cloning—A Laboratory Manual; Cold Spring Harbor, 1982-1989.

For protein based immunotherapeutics the proteins of the present invention are provided either in a liquid form or in a lyophilised form.

It is generally expected that each human dose will comprise 1 to 1000 μg of protein, and for example 30-300 μg such as 25, 30, 40, 50, 60, 70, 80 or 90 μg.

The method(s) as described herein may comprise a composition further comprises a vaccine adjuvant, and/or immunostimulatory cytokine or chemokine.

Suitable vaccine adjuvants for use in the present invention are commercially available such as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminium salts such as aluminium hydroxide gel (alum) or aluminium phosphate; salts of calcium, iron or zinc; an insoluble suspension of acylated tyrosine; acylated sugars; cationically or anionically derivatised polysaccharides; polyphosphazenes; biodegradable microspheres; monophosphoryl lipid A and quil A. Cytokines, such as GM-CSF or interleukin-2, -7, or -12, and chemokines may also be used as adjuvants.

In formulations it may be desirable that the adjuvant composition induces an immune response predominantly of the Th1 type. High levels of Th1-type cytokines (e.g., IFN-γ, TNFα, IL-2 and IL-12) tend to favour the induction of cell mediated immune responses to an administered antigen. According to one embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, Ann. Rev. Immunol. 7: 145-173, 1989.

Accordingly, suitable adjuvants that may be used to elicit a predominantly Th1-type response include, for example a combination of monophosphoryl lipid A, such as 3-de-O-acylated monophosphoryl lipid A (3D-MPL) together with an aluminium salt. 3D-MPL or other toll like receptor 4 (TLR4) ligands such as aminoalkyl glucosaminide phosphates as disclosed in WO 98/50399, WO 01/34617 and WO 03/065806 may also be used alone to generate a predominantly Th1-type response.

Other known adjuvants, which may preferentially induce a TH1 type immune response, include TLR9 agonists such as unmethylated CpG containing oligonucleotides. The oligonucleotides are characterised in that the CpG dinucleotide is unmethylated. Such oligonucleotides are well known and are described in, for example WO 96/02555.

Suitable oligionucleotides include:

| | |
|---|---|
| SEQ ID NO: 102 | TCC ATG ACG TTC CTG ACG TT |
| | (CpG 1826) |
| SEQ ID NO: 103 | TCT CCC AGC GTG CGC CAT (CpG 1758) |
| SEQ ID NO: 104 | ACC GAT GAC GTC GCC GGT GAC GGC ACC |
| | ACG |
| SEQ ID N0: 105 | TCG TCG TTT TGT CGT TTT GTC GTT |
| | (CpG 2006, CpG 7909) |
| SEQ ID NO: 106 | TCC ATG ACG TTC CTG ATG CT |
| | (CpG 1668) |

CpG-containing oligonucleotides may also be used alone or in combination with other adjuvants. For example, an enhanced system involves the combination of a CpG-containing oligonucleotide and a saponin derivative particularly the combination of CpG and QS21 as disclosed in WO 00/09159 and WO 00/62800.

The formulation may additionally comprise an oil in water emulsion and/or tocopherol.

Another suitable adjuvant is a saponin, for example QS21 (Aquila Biopharmaceuticals Inc., Framingham, Mass.), that may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other suitable formulations comprise an oil-in-water emulsion and tocopherol. A particularly potent adjuvant formulation involving QS21, 3D-MPL and tocopherol in, for example, an oil-in-water emulsion is described in WO 95/17210.

In another embodiment, the adjuvants may be formulated in a liposomal composition.

The amount of 3D-MPL used is generally small, but depending on the immunotherapeutic formulation may be in the region of 1-1000 μg per dose, for example 1-500 μg per dose, and such as 1 to 100 μg per dose, particularly 25, 30, 40, 50, 60, 70, 80 or 90 μg per dose.

In an embodiment, the adjuvant system comprises three immunostimulants: a CpG oligonucleotide, 3D-MPL & QS21 either presented in a liposomal formulation or an oil in water emulsion such as described in WO 95/17210.

The amount of CpG or immunostimulatory oligonucleotides in the adjuvants or immunotherapeutics of the present invention is generally small, but depending on the immunotherapeutic formulation may be in the region of 1-1000 μg per dose, for example 1-500 μg per dose.

The amount of saponin for use in the adjuvants of the present invention may be in the region of 1-1000 μg per dose, for example 1-500 μg per dose, such as 1 to 100 μg per dose, particularly 25, 30, 40, 50, 60, 70, 80 or 90 μg per dose.

Generally, it is expected that each human dose will comprise 0.1-1000 μg of antigen, for example 0.1-500 μg, such as 0.1-100 μg, particularly 0.1 to 50 μg, especially 25 or 50 μg. An optimal amount for a particular immunotherapeutic can be ascertained by standard studies involving observation of appropriate immune responses in vaccinated subjects. Following an initial vaccination, subjects may receive one or several booster immunisation adequately spaced.

Other suitable adjuvants include Montanide ISA 720 (Seppic, France), SAF (Chiron, Calif., United States), ISCOMS (CSL), MF-59 (Chiron), Ribi Detox, RC-529 (GSK, Hamilton, Mont.) and other aminoalkyl glucosaminide 4-phosphates (AGPs).

Accordingly there is provided an immunogenic composition for use in the method of the present invention comprising an antigen as disclosed herein and an adjuvant, wherein the adjuvant comprises one or more of 3D-MPL, QS21, a CpG oligonucleotide or a combination of two or more of these adjuvants. The antigen within the immunogenic composition may be presented in an oil in water or a water in oil emulsion vehicle or in a liposomal formulation.

In one embodiment, the adjuvant may comprise one or more of 3D-MPL, QS21 and an immunostimulatory CpG oligonucleotide. In an embodiment all three immunostimulants are present. In another embodiment 3D-MPL and QS21 are presented in an oil in water emulsion, and in the absence of a CpG oligonucleotide.

A composition for use in the method of the present invention may comprise a pharmaceutical composition comprising tumour associated antigen as described herein, or a fusion protein thereof, and a pharmaceutically acceptable excipient.

Use of the word comprising in the context of this specification in intended to be non-limiting ie means including.

Embodiments are specifically envisaged where aspects of the invention comprising a certain element or elements are limited to said aspects consisting or consisting essentially of the relevant elements as separate embodiments.

The examples below are shown to illustrate the methodology, which may be employed to prepare particles of the invention.

Discussion of documents in this specification is intended to give context to the invention and aid understanding of the same. In no way is it intended to be an admission that the document or comment is known or is common general knowledge in the relevant field.

In one or more aspects the invention provides an embodiment as described in any one of paragraphs 1 to 101 below.

1) Thus the invention may employ one or more genes from Table 1.

2) In another aspect the invention employs one or more genes according to paragraph 1, wherein the gene has the symbol STAT1, optionally in combination with one or more genes labeled as 1.2 to 1.100 identified in Table 1.

3) In another aspect the invention employs one or more genes according to paragraph 1, wherein the gene has the symbol PSMB9, optionally in combination with one or more genes selected from the group consisting of genes labeled as 1.1 and 1.3 to 1.100 identified in Table 1.

4) In another aspect the invention employs one or more genes according to paragraph 1, wherein the gene has the symbol JAK2, optionally in combination with one or more genes selected from the group consisting of genes labeled as 1.1 to 1.2 and 1.4 to 1.100 identified in Table 1.

5) In another aspect the invention employs one or more genes according to paragraph 1, wherein the gene has the symbol ITGA3, optionally in combination with one or more genes selected from the group consisting of genes labeled as 1.1 to 1.3 and 1.5 to 1.100 identified in Table 1.

6) In another aspect the invention employs one or more genes according to paragraph 1, wherein the gene has the symbol PSMB10, optionally in combination with one or more genes selected from the group consisting of genes labeled as 1.1 to 1.4 and 1.6 to 1.100 identified in Table 1.

7) In another aspect the invention employs one or more genes according to paragraph 1, wherein the gene has the symbol CXCL9, optionally in combination with one or more genes selected from the group consisting of genes labeled as 1.1 to 1.5 and 1.7 to 1.100 identified in Table 1.

8) In another aspect the invention employs one or more genes according to paragraph 1, wherein the gene has the symbol RARRES3, optionally in combination with one or more genes selected from the group consisting of genes labeled as 1.1 to 1.6 and 1.8 to 1.100 identified in Table 1.

9) In another aspect the invention employs one or more genes according to paragraph 1, wherein the gene has the symbol IL2RG, optionally in combination with one or more genes selected from the group consisting of genes labeled as 1.1 to 1.7 and 1.9 to 1.100 identified in Table 1.

10) In another aspect the invention employs one or more genes according to paragraph 1, wherein the gene has the symbol CXCL10, optionally in combination with one or more genes selected from the group consisting of genes labeled as 1.1 to 1.8 and 1.10 to 1.100 identified in Table 1.

11) In another aspect the invention employs one or more genes according to paragraph 1, wherein the gene has the symbol CD8A, optionally in combination with one or more genes selected from the group consisting of genes labeled as 1.1 to 1.9 and 1.11 to 1.100 identified in Table 1.

12) In another aspect the invention employs one or more genes according to paragraph 1, wherein the gene has the symbol UBD, optionally in combination with one or more genes selected from the group consisting of genes labeled as 1.1 to 1.10 and 1.12 to 1.100 identified in Table 1.

13) In another aspect the invention employs one or more genes according to paragraph 1, wherein the gene has the symbol GPR171, optionally in combination with one or more genes selected from the group consisting of genes labeled as 1.1 to 1.11 and 1.13 to 1.100 identified in Table 1.

14) In another aspect the invention employs one or more genes according to paragraph 1, wherein the gene has the symbol KLRD1, optionally in combination with one or more genes selected from the group consisting of genes labeled as 1.1 to 1.12 and 1.14 to 1.100 identified in Table 1.

15) In another aspect the invention employs one or more genes according to paragraph 1, wherein the gene has the symbol HLA-B, optionally in combination with one or more genes selected from the group consisting of genes labeled as 1.1 to 1.13 and 1.15 to 1.100 identified in Table 1.

16) In another aspect the invention employs one or more genes according to paragraph 1, wherein the gene has the symbol LCP1, optionally in combination with one or more genes selected from the group consisting of genes labeled as 1.1 to 1.14 and 1.16 to 1.100 identified in Table 1.

17) In another aspect the invention employs one or more genes according to paragraph 1, wherein the gene has the symbol HLA-DRA, optionally in combination with one or more genes selected from the group consisting of genes labeled as 1.1 to 1.15 and 1.17 to 1.100 identified in Table 1.

18) In another aspect the invention employs one or more genes according to paragraph 1, wherein the gene has the symbol CYTIP, optionally in combination with one or more genes selected from the group consisting of genes labeled as 1.1 to 1.16 and 1.18 to 1.100 identified in Table 1.

19) In another aspect the invention employs one or more genes according to paragraph 1, wherein the gene has the symbol IL23A, optionally in combination with one or more genes selected from the group consisting of genes labeled as 1.1 to 1.17 and 1.19 to 1.100 identified in Table 1.

20) In another aspect the invention employs one or more genes according to paragraph 1, wherein the gene has the symbol TRA@, optionally in combination with one or more genes selected from the group consisting of genes labeled as 1.1 to 1.18 and 1.20 to 1.100 identified in Table 1.

21) In another aspect the invention employs one or more genes according to paragraph 1, wherein the gene has the symbol HLA-DRA, optionally in combination with one or more genes selected from the group consisting of genes labeled as 1.1 to 1.19 and 1.21 to 1.100 identified in Table 1.

22) In another aspect the invention employs one or more genes according to paragraph 1, wherein the gene has the symbol TARP, optionally in combination with one or more genes selected from the group consisting of genes labeled as 1.1 to 1.20 and 1.22 to 1.100 identified in Table 1.

23) In another aspect the invention employs one or more genes according to paragraph 1, wherein the gene has the symbol ITK, optionally in combination with one or more genes selected from the group consisting of genes labeled as 1.1 to 1.21 and 1.23 to 1.100 identified in Table 1.

24) In another aspect the invention employs one or more genes according to paragraph 1, wherein the gene has the symbol the gene is the one identified by probe set 211796_s_at, optionally in combination with one or more genes selected from the group consisting of genes labeled as 1.1 to 1.22 and 1.24 to 1.100 identified in Table 1.

25) In another aspect the invention employs one or more genes according to paragraph 1, wherein the gene has the symbol HLA-B, optionally in combination with one or more genes selected from the group consisting of genes labeled as 1.1 to 1.23 and 1.25 to 1.100 identified in Table 1.

26) In another aspect the invention employs one or more genes according to paragraph 1, wherein the gene has the symbol HLA-DQA1, optionally in combination with one or more genes selected from the group consisting of genes labeled as 1.1 to 1.24 and 1.26 to 1.100 identified in Table 1.

27) In another aspect the invention employs one or more genes according to paragraph 1, wherein the gene has the symbol HOMER1, optionally in combination with one or more genes selected from the group consisting of genes labeled as 1.1 to 1.25 and 1.27 to 1.100 identified in Table 1.

28) In another aspect the invention employs one or more genes according to paragraph 1, wherein the gene has the symbol TRGC2, optionally in combination with one or more genes selected from the group consisting of genes labeled as 1.1 to 1.26 and 1.28 to 1.100 identified in Table 1.

29) In another aspect the invention employs one or more genes according to paragraph 1, wherein the gene is the one identified by probe set 216920_s_at, optionally in combination with one or more genes selected from the group consisting of genes labeled as 1.1 to 1.27 and 1.29 to 1.100 identified in Table 1.

30) In another aspect the invention employs one or more genes according to paragraph 1, wherein the gene has the symbol HLA-A, optionally in combination with one or more genes selected from the group consisting of genes labeled as 1.1 to 1.28 and 1.30 to 1.100 identified in Table 1.

31) In another aspect the invention employs one or more genes according to paragraph 1, wherein the gene has the symbol HLA-DMA, optionally in combination with one or more genes selected from the group consisting of genes labeled as 1.1 to 1.29 and 1.31 to 1.100 identified in Table 1.

32) In another aspect the invention employs one or more genes according to paragraph 1, wherein the gene has the symbol HLA-F, optionally in combination with one or more genes selected from the group consisting of genes labeled as 1.1 to 1.30 and 1.32 to 1.100 identified in Table 1.

33) In another aspect the invention employs one or more genes according to paragraph 1, wherein the gene has the symbol SLAMF7, optionally in combination with one or more genes selected from the group consisting of genes labeled as 1.1 to 1.31 and 1.33 to 1.100 identified in Table 1.

34) In another aspect the invention employs one or more genes according to paragraph 1, wherein the gene has the symbol KIAA1549, optionally in combination with one or more genes selected from the group consisting of genes labeled as 1.1 to 1.32 and 1.34 to 1.100 identified in Table 1.

35) In another aspect the invention employs one or more genes according to paragraph 1, wherein the gene has the symbol LONRF2, optionally in combination with one or more genes selected from the group consisting of genes labeled as 1.35 to 1.100 identified in Table 1.

36) In another aspect the invention employs one or more genes according to paragraph 1, wherein the gene has the symbol FAM26F, optionally in combination with one or more genes selected from the group consisting of genes labeled as 1.1 to 1.34 and 1.36 to 1.100 identified in Table 1.

37) In another aspect the invention employs one or more genes according to paragraph 1, wherein the gene has the symbol C1orf162, optionally in combination with one or more genes selected from the group consisting of genes labeled as 1.1 to 1.35 and 1.37 to 1.100 identified in Table 1.

38) In another aspect the invention employs one or more genes according to paragraph 1, wherein the gene has the symbol FAM26F, optionally in combination with one or more genes selected from the group consisting of genes labeled as 1.1 to 1.36 and 1.38 to 1.100 identified in Table 1.

39) In another aspect the invention employs one or more genes according to paragraph 1, wherein the gene has the symbol GBP5, optionally in combination with one or more genes selected from the group consisting of genes labeled as 1.1 to 1.37 and 1.39 to 1.100 identified in Table 1.

40) In another aspect the invention employs one or more genes according to paragraph 1, wherein the gene is the one identified by probe set 232375_at, optionally in combination with one or more genes selected from the group consisting of genes labeled as 1.1 to 1.38 and 1.40 to 1.100 identified in Table 1.

41) In another aspect the invention employs one or more genes according to paragraph 1, wherein the gene has the symbol SLITRK6, optionally in combination with one or more genes selected from the group consisting of genes labeled as 1.1 to 1.39 and 1.41 to 1.100 identified in Table 1.

42) In another aspect the invention employs one or more genes according to paragraph 1, wherein the gene has the symbol GBP4, optionally in combination with one or more genes selected from the group consisting of genes labeled as 1.1 to 1.40 and 1.42 to 1.100 identified in Table 1.

43) In another aspect the invention employs one or more genes according to paragraph 1, wherein the gene has the symbol EPSTI1 optionally in combination with one or more genes selected from the group consisting of genes labeled as 1.1 to 1.41 and 1.43 to 1.100 identified in Table 1.

44) In another aspect the invention employs one or more genes according to paragraph 1, wherein the gene has the symbol AKR1C2 optionally in combination with one or more genes selected from the group consisting of genes labeled as 1.1 to 1.42 and 1.44 to 1.100 identified in Table 1.

45) In another aspect the invention employs one or more genes according to paragraph 1, wherein the gene has the symbol ITGAL optionally in combination with one or more genes selected from the group consisting of genes labeled as 1.1 to 1.43 and 1.45 to 1.100 identified in Table 1.

46) In another aspect the invention employs one or more genes according to paragraph 1, wherein the gene has the symbol CDC42SE2, optionally in combination with one or more genes selected from the group consisting of genes labeled as 1.1 to 1.44 and 1.46 to 1.100 identified in Table 1.

47) In another aspect the invention employs one or more genes according to paragraph 1, wherein the gene has the symbol DZIP1, optionally in combination with one or more genes selected from the group consisting of genes labeled as 1.1 to 1.45 and 1.47 to 1.100 identified in Table 1.

48) In another aspect the invention employs one or more genes according to paragraph 1, wherein the gene has the symbol PTGER4, optionally in combination with one or more genes selected from the group consisting of genes labeled as 1.1 to 1.46 and 1.48 to 1.100 identified in Table 1.

49) In another aspect the invention employs one or more genes according to paragraph 1, wherein the gene has the symbol HCP5, optionally in combination with one or more genes selected from the group consisting of genes labeled as 1.1 to 1.47 and 1.49 to 1.100 identified in Table 1.

50) In another aspect the invention employs one or more genes according to paragraph 1, wherein the gene has the symbol UTY, optionally in combination with one or more genes selected from the group consisting of genes labeled as 1.1 to 1.48 and 1.50 to 1.100 identified in Table 1.

51) In another aspect the invention employs one or more genes according to paragraph 1, wherein the gene has the symbol KLRB1, optionally in combination with one or more genes selected from the group consisting of genes labeled as 1.1 to 1.49 and 1.51 to 1.100 identified in Table 1.

52) In another aspect the invention employs one or more genes according to paragraph 1, wherein the gene has the symbol FAM26F, optionally in combination with one or more genes selected from the group consisting of genes labeled as 1.1 to 1.50 and 1.52 to 1.100 identified in Table 1.

53) In another aspect the invention employs one or more genes according to paragraph 1, wherein the gene has the symbol HILS1, optionally in combination with one or more genes selected from the group consisting of genes labeled as 1.1 to 1.51 and 1.53 to 1.100 identified in Table 1.

54) In another aspect the invention employs one or more genes according to paragraph 1, wherein the gene has the symbol C20orf24, optionally in combination with one or more genes selected from the group consisting of genes labeled as 1.1 to 1.52 and 1.54 to 1.100 identified in Table 1.

55) In another aspect the invention employs one or more genes according to paragraph 1, wherein the gene has the symbol B2M, optionally in combination with one or more genes selected from the group consisting of genes labeled as 1.1 to 1.53 and 1.55 to 1.100 identified in Table 1.

56) In another aspect the invention employs one or more genes according to paragraph 1, wherein the gene has the symbol ZNF285A, optionally in combination with one or more genes selected from the group consisting of genes labeled as 1.1 to 1.54 and 1.56 to 1.100 identified in Table 1.

57) In another aspect the invention employs one or more genes according to paragraph 1, wherein the gene has the symbol TMEM56, optionally in combination with one or more genes selected from the group consisting of genes labeled as 1.1 to 1.55 and 1.57 to 1.100 identified in Table 1.

58) In another aspect the invention employs one or more genes according to paragraph 1, wherein the gene has the symbol IRF1, optionally in combination with one or more genes selected from the group consisting of genes labeled as 1.1 to 1.56 and 1.58 to 1.100 identified in Table 1.

59) In another aspect the invention employs one or more genes according to paragraph 1, wherein the gene has the symbol TRGV9, optionally in combination with one or more genes selected from the group consisting of genes labeled as 1.1 to 1.57 and 1.59 to 1.100 identified in Table 1.

60) In another aspect the invention employs one or more genes according to paragraph 1, wherein the gene has the symbol NA identified by probe set 238524_at, optionally in combination with one or more genes selected from the group consisting of genes labeled as 1.1 to 1.58 and 1.60 to 1.100 identified in Table 1.

61) In another aspect the invention employs one or more genes according to paragraph 1, wherein the gene has the symbol SLC26A2, optionally in combination with one or more genes selected from the group consisting of genes labeled as 1.1 to 1.59 and 1.61 to 1.100 identified in Table 1.

62) In another aspect the invention employs one or more genes according to paragraph 1, wherein the gene has the symbol CXCL2, optionally in combination with one or more genes selected from the group consisting of genes labeled as 1.1 to 1.60 and 1.62 to 1.100 identified in Table 1.

63) In another aspect the invention employs one or more genes according to paragraph 1, wherein the gene has the symbol ICOS, optionally in combination with one or more genes selected from the group consisting of genes labeled as 1.1 to 1.61 and 1.63 to 1.100 identified in Table 1.

64) In another aspect the invention employs one or more genes according to paragraph 1, wherein the gene is the one identified by probe set 213193_x_at, optionally in combination with one or more genes selected from the group consisting of genes labeled as 1.1 to 1.62 and 1.64 to 1.100 identified in Table 1.

65) In another aspect the invention employs one or more genes according to paragraph 1, wherein the gene has the symbol CCL5, optionally in combination with one or more genes selected from the group consisting of genes labeled as 1.1 to 1.63 and 1.65 to 1.100 identified in Table 1.

66) In another aspect the invention employs one or more genes according to paragraph 1, wherein the gene has the symbol LOC284757 optionally in combination with one or more genes selected from the group consisting of genes labeled as 1.1 to 1.64 and 1.66 to 1.100 identified in Table 1.

67) In another aspect the invention employs one or more genes according to paragraph 1, wherein the gene has the symbol CD86, optionally in combination with one or more genes selected from the group consisting of genes labeled as 1.1 to 1.65 and 1.67 to 1.100 identified in Table 1.

68) In another aspect the invention employs one or more genes according to paragraph 1, wherein the gene has the symbol KLRD1, optionally in combination with one or more genes selected from the group consisting of genes labeled as 1.1 to 1.66 and 1.68 to 4.488 identified in Table 1.

69) In another aspect the invention employs one or more genes according to paragraph 1, wherein the gene is the one identified by probe set 211902_x_at, optionally in combination with one or more genes selected from the group consisting of genes labeled as 1.1 to 1.67 and 1.69 to 1.100 identified in Table 1.

70) In another aspect the invention employs one or more genes according to paragraph 1, wherein the gene has the symbol SLAMF6, optionally in combination with one or more genes selected from the group consisting of genes labeled as 1.1 to 1.68 and 1.70 to 1.100 identified in Table 1.

71) In another aspect the invention employs one or more genes according to paragraph 1, wherein the gene has the symbol TOX, optionally in combination with one or more genes selected from the group consisting of genes labeled as 1.1 to 1.69 and 1.71 to 1.100 identified in Table 1.

72) In another aspect the invention employs one or more genes according to paragraph 1, wherein the gene has the symbol GZMK, optionally in combination with one or more genes selected from the group consisting of genes labeled as 1.1 to 1.70 and 1.72 to 1.100 identified in Table 1.

73) In another aspect the invention employs one or more genes according to paragraph 1, wherein the gene has the symbol CDC42SE2, optionally in combination with one or more genes selected from the group consisting of genes labeled as 1.1 to 1.71 and 1.73 to 1.100 identified in Table 1.

74) In another aspect the invention employs one or more genes according to paragraph 1, wherein the gene has the symbol PPP1R16B, optionally in combination with one or more genes selected from the group consisting of genes labeled as 1.1 to 1.72 and 1.74 to 1.100 identified in Table 1.

75) In another aspect the invention employs one or more genes according to paragraph 1, wherein the gene has the symbol EAF2, optionally in combination with one or more genes selected from the group consisting of genes labeled as 1.1 to 1.73 and 1.75 to 1.100 identified in Table 1.

76) In another aspect the invention employs one or more genes according to paragraph 1, wherein the gene has the symbol USP9Y, optionally in combination with one or more genes selected from the group consisting of genes labeled as 1.1 to 1.74 and 1.76 to 1.100 identified in Table 1.

77) In another aspect the invention employs one or more genes according to paragraph 1, wherein the gene has the symbol FAM26F, optionally in combination with one or more genes selected from the group consisting of genes labeled as 1.1 to 1.75 and 1.77 to 1.100 identified in Table 1.

78) In another aspect the invention employs one or more genes according to paragraph 1, wherein the gene has the symbol FLJ31438, optionally in combination with one or more genes selected from the group consisting of genes labeled as 1.1 to 1.76 and 1.78 to 1.100 identified in Table 1.

79) In another aspect the invention employs one or more genes according to paragraph 1, wherein the gene has the symbol SHROOM3, optionally in combination with one or more genes selected from the group consisting of genes labeled as 1.1 to 1.77 and 1.79 to 1.100 identified in Table 1.

80) In another aspect the invention employs one or more genes according to paragraph 1, wherein the gene has the symbol TNFAIP3, optionally in combination with one or more genes selected from the group consisting of genes labeled as 1.1 to 1.78 and 1.80 to 1.100 identified in Table 1.

81) In another aspect the invention employs one or more genes according to paragraph 1, wherein the gene has the symbol HLA-F, optionally in combination with one or more genes selected from the group consisting of genes labeled as 1.1 to 1.79 and 1.81 to 1.100 identified in Table 1.

82) In another aspect the invention employs one or more genes according to paragraph 1, wherein the gene has the symbol CD3D, optionally in combination with one or more genes selected from the group consisting of genes labeled as 1.1 to 1.80 and 1.82 to 1.100 identified in Table 1.

83) In another aspect the invention employs one or more genes according to paragraph 1, wherein the gene has the symbol MAP1B, optionally in combination with one or more genes selected from the group consisting of genes labeled as 1.1 to 1.81 and 1.83 to 1.100 identified in Table 1.

84) In another aspect the invention employs one or more genes according to paragraph 1, wherein the gene has the symbol SRPX2, optionally in combination with one or more genes selected from the group consisting of genes labeled as 1.1 to 1.82 and 1.84 to 1.100 identified in Table 1.

85) In another aspect the invention employs one or more genes according to paragraph 1, wherein the gene has the symbol AADAT, optionally in combination with one or more genes selected from the group consisting of genes labeled as 1.1 to 1.83 and 1.85 to 1.100 identified in Table 1.

86) In another aspect the invention employs one or more genes according to paragraph 1, wherein the gene has the symbol ARHGAP15, optionally in combination with one or more genes selected from the group consisting of genes labeled as 1.1 to 1.84 and 1.86 to 1.100 identified in Table 1.

87) In another aspect the invention employs one or more genes according to paragraph 1, wherein the gene has the symbol MCM10, optionally in combination with one or more genes selected from the group consisting of genes labeled as 1.1 to 1.85 and 1.87 to 1.100 identified in Table 1.

88) In another aspect the invention employs one or more genes according to paragraph 1, wherein the gene has the symbol TC2N, optionally in combination with one or more genes selected from the group consisting of genes labeled as 1.1 to 1.86 and 1.88 to 1.100 identified in Table 1.

89) In another aspect the invention employs one or more genes according to paragraph 1, wherein the gene has the symbol AP2B1, optionally in combination with one or more genes selected from the group consisting of genes labeled as 1.1 to 1.87 and 1.89 to 1.100 identified in Table 1.

90) In another aspect the invention employs one or more genes according to paragraph 1, wherein the gene has the symbol GOLGA7, optionally in combination with one or more genes selected from the group consisting of genes labeled as 1.1 to 1.88 and 1.90 to 1.100 identified in Table 1.

91) In another aspect the invention employs one or more genes according to paragraph 1, wherein the gene has the symbol TNFRSF9, optionally in combination with one or more genes selected from the group consisting of genes labeled as 1.1 to 1.89 and 1.91 to 1.100 identified in Table 1.

92) In another aspect the invention employs one or more genes according to paragraph 1, wherein the gene has the symbol RNF144B, optionally in combination with one or more genes selected from the group consisting of genes labeled as 1.1 to 1.90 and 1.92 to 1.100 identified in Table 1.

93) In another aspect the invention employs one or more genes according to paragraph 1, wherein the gene is the one identified by probe set 209671_x_at, optionally in combination with one or more genes selected from the group consisting of genes labeled as 1.1 to 1.91 and 1.93 to 1.100 identified in Table 1.

94) In another aspect the invention employs one or more genes according to paragraph 1, wherein the gene has the symbol UBASH3B, optionally in combination with one or more genes selected from the group consisting of genes labeled as 1.1 to 1.92 and 1.94 to 1.100 identified in Table 1.

95) In another aspect the invention employs one or more genes according to paragraph 1, wherein the gene has the symbol BTN3A1, optionally in combination with one or more genes selected from the group consisting of genes labeled as 1.1 to 1.93 and 1.95 to 1.100 identified in Table 1.

96) In another aspect the invention employs one or more genes according to paragraph 1, wherein the gene has the symbol GCH1, optionally in combination with one or more genes selected from the group consisting of genes labeled as 1.1 to 1.94 and 1.96 to 1.100 identified in Table 1.

97) In another aspect the invention employs one or more genes according to paragraph 1, wherein the gene has the symbol DENND2D, optionally in combination with one or more genes selected from the group consisting of genes labeled as 1.1 to 1.95 and 1.97 to 1.100 identified in Table 1.

98) In another aspect the invention employs one or more genes according to paragraph 1, wherein the gene has the symbol C4orf7, optionally in combination with one or more genes selected from the group consisting of genes labeled as 1.1 to 1.96 and 1.98 to 1.100 identified in Table 1.

99) In another aspect the invention employs one or more genes according to paragraph 1, wherein the gene has the symbol TNFAIP3, optionally in combination with one or more genes selected from the group consisting of genes labeled as 1.1 to 1.97 and 1.99 to 1.100 identified in Table 1.

100) In another aspect the invention employs one or more genes according to paragraph 1, wherein the gene has the symbol GBP5, optionally in combination with one or more genes selected from the group consisting of genes labeled as 1.100 identified in Table 1.

101) In another aspect the invention employs one or more genes according to paragraph 1, wherein the gene has the symbol GBP1, optionally in combination with one or more genes selected from the group consisting of genes labeled as 1.1 to 1.99.

In one or more aspects the invention provides an embodiment as described in any one of paragraphs 1 to 101 below. The expression “the gene”, in paragraphs 3 to 101 when referring to any one of paragraphs 2 to 100, is not intended to replace the specific gene mentioned in paragraphs 2 to 100 but to add to it.

1) Thus the invention may employ one or more genes from Table 1.

2) In another aspect the invention employs one or more genes according to paragraph 1, wherein the gene has the symbol STAT1, optionally in combination with one or more genes labeled as 1.2 to 1.100 identified in Table 1.

3) In another aspect the invention employs one or more genes according to paragraph 1 or 2, wherein the gene has the symbol PSMB9, optionally in combination with one or more genes labeled as 1.3 to 1.100 identified in Table 1.

4) In another aspect the invention employs one or more genes according to any one one of paragraphs 1-3, wherein the gene has the symbol JAK2, optionally in combination with one or more genes labeled as 1.4 to 1.100 identified in Table 1.

5) In another aspect the invention employs one or more genes according to any one one of paragraphs 1-4, wherein the gene has the symbol ITGA3, optionally in combination with one or more genes labeled as 1.5 to 1.100 identified in Table 1.

6) In another aspect the invention employs one or more genes according to any one one of paragraphs 1-5, wherein the gene has the symbol PSMB10, optionally in combination with one or more genes labeled as 1.6 to 1.100 identified in Table 1.

7) In another aspect the invention employs one or more genes according to any one one of paragraphs 1-6, wherein the gene has the symbol CXCL9, optionally in combination with one or more genes labeled as 1.7 to 1.100 identified in Table 1.

8) In another aspect the invention employs one or more genes according to any one one of paragraphs 1-7, wherein the gene has the symbol RARRES3, optionally in combination with one or more genes labeled as 1.8 to 1.100 identified in Table 1.

9) In another aspect the invention employs one or more genes according to any one one of paragraphs 1-8, wherein the gene has the symbol IL2RG, optionally in combination with one or more genes labeled as 1.9 to 1.100 identified in Table 1.

10) In another aspect the invention employs one or more genes according to any one one of paragraphs 1-9, wherein the gene has the symbol CXCL10, optionally in combination with one or more genes labeled as 1.10 to 1.100 identified in Table 1.

11) In another aspect the invention employs one or more genes according to any one one of paragraphs 1-10, wherein the gene has the symbol CD8A, optionally in combination with one or more genes labeled as 1.11 to 1.100 identified in Table 1.

12) In another aspect the invention employs one or more genes according to any one one of paragraphs 1-11, wherein the gene has the symbol UBD, optionally in combination with one or more genes labeled as 1.12 to 1.100 identified in Table 1

13) In another aspect the invention employs one or more genes according to any one one of paragraphs 1-12, wherein the gene has the symbol GPR171, optionally in combination with one or more genes labeled as 1.13 to 1.100 identified in Table 1.

14) In another aspect the invention employs one or more genes according to any one of paragraphs 1-13, wherein the gene has the symbol KLRD1, optionally in combination with one or more genes labeled as 1.14 to 1.100 identified in Table 1.

15) In another aspect the invention employs one or more genes according to any one of paragraphs 1-14, wherein the gene has the symbol HLA-B, optionally in combination with one or more genes labeled as 1.15 to 1.100 identified in Table 1.

16) In another aspect the invention employs one or more genes according to any one of paragraphs 1-15, wherein the gene has the symbol LCP1, optionally in combination with one or more genes labeled as 1.16 to 1.100 identified in Table 1.

17) In another aspect the invention employs one or more genes according to any one of paragraphs 1-16, wherein the gene has the symbol HLA-DRA, optionally in combination with one or more genes labeled as 1.17 to 1.100 identified in Table 1.

18) In another aspect the invention employs one or more genes according to any one of paragraphs 1-17, wherein the gene has the symbol CYTIP, optionally in combination with one or more genes labeled as 1.18 to 1.100 identified in Table 1.

19) In another aspect the invention employs one or more genes according to any one of paragraphs 1-18, wherein the gene has the symbol IL23A, optionally in combination with one or more genes labeled as 1.19 to 1.100 identified in Table 1.

20) In another aspect the invention employs one or more genes according to any one of paragraphs 1-19, wherein the gene has the symbol TRA@, optionally in combination with one or more genes labeled as 1.20 to 1.100 identified in Table 1.

21) In another aspect the invention employs one or more genes according to any one of paragraphs 1-20, wherein the gene has the symbol HLA-DRA, optionally in combination with one or more genes labeled as 1.21 to 1.100 identified in Table 1.

22) In another aspect the invention employs one or more genes according to any one of paragraphs 1-21, wherein the gene has the symbol TARP, optionally in combination with one or more genes labeled as 1.22 to 1.100 identified in Table 1.

23) In another aspect the invention employs one or more genes according to any one of paragraphs 1-22, wherein the gene has the symbol ITK, optionally in combination with one or more genes labeled as 1.23 to 1.100 identified in Table 1.

24) In another aspect the invention employs one or more genes according to any one of paragraphs 1-23, wherein the gene is the one identified by probe set 211796_s_at, optionally in combination with one or more genes labeled as 1.24 to 1.100 identified in Table 1.

25) In another aspect the invention employs one or more genes according to any one of paragraphs 1-24, wherein the gene has the symbol HLA-B, optionally in combination with one or more genes labeled as 1.25 to 1.100 identified in Table 1.

26) In another aspect the invention employs one or more genes according to any one of paragraphs 1-25, wherein the gene has the symbol HLA-DQA1, optionally in combination with one or more genes labeled as 1.26 to 1.100 identified in Table 1.

27) In another aspect the invention employs one or more genes according to any one of paragraphs 1-26, wherein the gene has the symbol HOMER1, optionally in combination with one or more genes labeled as 1.27 to 1.100 identified in Table 1.

28) In another aspect the invention employs one or more genes according to any one of paragraphs 1-27, wherein the gene has the symbol TRGC2, optionally in combination with one or more genes labeled as 1.28 to 1.100 identified in Table 1.

29) In another aspect the invention employs one or more genes according to any one of paragraphs 1-28, wherein the gene is the one identified by probe set 216920_s_at, optionally in combination with one or more genes labeled as 1.29 to 1.100 identified in Table 1.

30) In another aspect the invention employs one or more genes according to any one of paragraphs 1-29, wherein the gene has the symbol HLA-A, optionally in combination with one or more genes labeled as 1.30 to 1.100 identified in Table 1.

31) In another aspect the invention employs one or more genes according to any one of paragraphs 1-30, wherein the gene has the symbol HLA-DMA, optionally in combination with one or more genes labeled as 1.31 to 1.100 identified in Table 1.

32) In another aspect the invention employs one or more genes according to any one of paragraphs 1-31, wherein the gene has the symbol HLA-F, optionally in combination with one or more genes labeled as 1.32 to 1.100 identified in Table 1.

33) In another aspect the invention employs one or more genes according to any one of paragraphs 1-32, wherein the gene has the symbol SLAMF7, optionally in combination with one or more genes labeled as 1.33 to 1.100 identified in Table 1.

34) In another aspect the invention employs one or more genes according to any one of paragraphs 1-33, wherein the gene has the symbol KIAA1549, optionally in combination with one or more genes labeled as 1.34 to 1.100 identified in Table 1.

35) In another aspect the invention employs one or more genes according to any one of paragraphs 1-34, wherein the gene has the symbol LONRF2, optionally in combination with one or more genes labeled as 1.35 to 1.100 identified in Table 1.

36) In another aspect the invention employs one or more genes according to any one of paragraphs 1-35, wherein the gene has the symbol FAM26F, optionally in combination with one or more genes labeled as 1.36 to 1.100 identified in Table 1.

37) In another aspect the invention employs one or more genes according to any one of paragraphs 1-36, wherein the gene has the symbol C1orf162, optionally in combination with one or more genes labeled as 1.37 to 1.100 identified in Table 1.

38) In another aspect the invention employs one or more genes according to any one of paragraphs 1-37, wherein the gene has the symbol FAM26F, optionally in combination with one or more genes labeled as 1.38 to 1.100 identified in Table 1.

39) In another aspect the invention employs one or more genes according to any one of paragraphs 1-38, wherein the gene has the symbol GBP5, optionally in combination with one or more genes labeled as 1.39 to 1.100 identified in Table 1.

40) In another aspect the invention employs one or more genes according to any one of paragraphs 1-39, wherein the gene is the one identified by probe set 232375_at, optionally in combination with one or more genes labeled as 1.40 to 1.100 identified in Table 1.

41) In another aspect the invention employs one or more genes according to any one of paragraphs 1-40, wherein the gene has the symbol SLITRK6, optionally in combination with one or more genes labeled as 1.41 to 1.100 identified in Table 1.

42) In another aspect the invention employs one or more genes according to any one of paragraphs 1-41, wherein the gene has the symbol GBP4, optionally in combination with one or more genes labeled as 1.42 to 1.100 identified in Table 1.

43) In another aspect the invention employs one or more genes according to any one of paragraphs 1-42, wherein the gene has the symbol EPSTI1 optionally in combination with one or more genes labeled as 1.43 to 1.100 identified in Table 1.

44) In another aspect the invention employs one or more genes according to any one of paragraphs 1-43, wherein the gene has the symbol AKR1C2 optionally in combination with one or more genes labeled as 1.44 to 1.100 identified in Table 1.

45) In another aspect the invention employs one or more genes according to any one of paragraphs 1-44, wherein the gene has the symbol ITGAL optionally in combination with one or more genes labeled as 1.45 to 1.100 identified in Table 1.

46) In another aspect the invention employs one or more genes according to any one of paragraphs 1-45, wherein the gene has the symbol CDC42SE2, optionally in combination with one or more genes labeled as 1.46 to 1.100 identified in Table 1.

47) In another aspect the invention employs one or more genes according to any one of paragraphs 1-46, wherein the gene has the symbol DZIP1, optionally in combination with one or more genes labeled as 1.47 to 1.100 identified in Table 1.

48) In another aspect the invention employs one or more genes according to any one of paragraphs 1-47, wherein the gene has the symbol PTGER4, optionally in combination with one or more genes labeled as 1.48 to 1.100 identified in Table 1.

49) In another aspect the invention employs one or more genes according to any one of paragraphs 1-48, wherein the gene has the symbol HOPS, optionally in combination with one or more genes labeled as 1.49 to 1.100 identified in Table 1.

50) In another aspect the invention employs one or more genes according to any one of paragraphs 1-49, wherein the gene has the symbol UTY, optionally in combination with one or more genes labeled as 1.50 to 1.100 identified in Table 1.

51) In another aspect the invention employs one or more genes according to any one of paragraphs 1-50, wherein the gene has the symbol KLRB1, optionally in combination with one or more genes labeled as 1.51 to 1.100 identified in Table 1.

52) In another aspect the invention employs one or more genes according to any one of paragraphs 1-51, wherein the gene has the symbol FAM26F, optionally in combination with one or more genes labeled as 1.52 to 1.100 identified in Table 1.

53) In another aspect the invention employs one or more genes according to any one of paragraphs 1-52, wherein the gene has the symbol HILS1, optionally in combination with one or more genes labeled as 1.53 to 1.100 identified in Table 1.

54) In another aspect the invention employs one or more genes according to any one of paragraphs 1-53, wherein the gene has the symbol C20orf24, optionally in combination with one or more genes labeled as 1.54 to 1.100 identified in Table 1.

55) In another aspect the invention employs one or more genes according to any one of paragraphs 1-54, wherein the gene has the symbol B2M, optionally in combination with one or more genes labeled as 1.55 to 1.100 identified in Table 1.

56) In another aspect the invention employs one or more genes according to any one of paragraphs 1-55, wherein the gene has the symbol ZNF285A, optionally in combination with one or more genes labeled as 1.56 to 1.100 identified in Table 1.

57) In another aspect the invention employs one or more genes according to any one of paragraphs 1-56, wherein the gene has the symbol TMEM56, optionally in combination with one or more genes labeled as 1.57 to 1.100 identified in Table 1.

58) In another aspect the invention employs one or more genes according to any one of paragraphs 1-57, wherein the gene has the symbol IRF1, optionally in combination with one or more genes labeled as 1.58 to 1.100 identified in Table 1.

59) In another aspect the invention employs one or more genes according to any one of paragraphs 1-58, wherein the gene has the symbol TRGV9, optionally in combination with one or more genes labeled as 1.59 to 1.100 identified in Table 1.

60) In another aspect the invention employs one or more genes according to any one of paragraphs 1-59, wherein the gene has the symbol NA identified by probe set 238524_at, optionally in combination with one or more genes labeled as 1.60 to 1.100 identified in Table 1.

61) In another aspect the invention employs one or more genes according to any one of paragraphs 1-60, wherein the gene has the symbol SLC26A2, optionally in combination with one or more genes labeled as 1.61 to 1.100 identified in Table 1.

62) In another aspect the invention employs one or more genes according to any one of paragraphs 1-61, wherein the gene has the symbol CXCL2, optionally in combination with one or more genes labeled as 1.62 to 1.100 identified in Table 1.

63) In another aspect the invention employs one or more genes according to any one of paragraphs 1-62, wherein the gene has the symbol ICOS, optionally in combination with one or more genes labeled as 1.63 to 1.100 identified in Table 1.

64) In another aspect the invention employs one or more genes according to any one of paragraphs 1-63, wherein the gene is the one identified by probe set 213193_x_at, optionally in combination with one or more genes labeled as 1.64 to 1.100 identified in Table 1.

65) In another aspect the invention employs one or more genes according to any one of paragraphs 1-64, wherein the gene has the symbol CCL5, optionally in combination with one or more genes labeled as 1.65 to 1.100 identified in Table 1.

66) In another aspect the invention employs one or more genes according to any one of paragraphs 1-65, wherein the gene has the symbol LOC284757 optionally in combination with one or more genes labeled as 1.66 to 1.100 identified in Table 1.

67) In another aspect the invention employs one or more genes according to any one of paragraphs 1-66, wherein the gene has the symbol CD86, optionally in combination with one or more genes labeled as 1.67 to 1.100 identified in Table 1.

68) In another aspect the invention employs one or more genes according to any one of paragraphs 1-67, wherein the gene has the symbol KLRD1, optionally in combination with one or more genes labeled as 1.68 to 4.488 identified in Table 1.

69) In another aspect the invention employs one or more genes according to any one of paragraphs 1-68, wherein the gene is the one identified by probe set 211902_x_at, optionally in combination with one or more genes labeled as 1.69 to 1.100 identified in Table 1.

70) In another aspect the invention employs one or more genes according to any one of paragraphs 1-69, wherein the gene has the symbol SLAMF6, optionally in combination with one or more genes labeled as 1.70 to 1.100 identified in Table 1.

71) In another aspect the invention employs one or more genes according to any one of paragraphs 1-70, wherein the gene has the symbol TOX, optionally in combination with one or more genes labeled as 1.71 to 1.100 identified in Table 1.

72) In another aspect the invention employs one or more genes according to any one of paragraphs 1-71, wherein the gene has the symbol GZMK, optionally in combination with one or more genes labeled as 1.72 to 1.100 identified in Table 1.

73) In another aspect the invention employs one or more genes according to any one of paragraphs 1-72, wherein the gene has the symbol CDC42SE2, optionally in combination with one or more genes labeled as 1.73 to 1.100 identified in Table 1.

74) In another aspect the invention employs one or more genes according to any one of paragraphs 1-73, wherein the gene has the symbol PPP1R16B, optionally in combination with one or more genes labeled as 1.74 to 1.100 identified in Table 1.

75) In another aspect the invention employs one or more genes according to any one of paragraphs 1-74, wherein the gene has the symbol EAF2, optionally in combination with one or more genes labeled as 1.75 to 1.100 identified in Table 1.

76) In another aspect the invention employs one or more genes according to any one of paragraphs 1-75, wherein the gene has the symbol USP9Y, optionally in combination with one or more genes labeled as 1.76 to 1.100 identified in Table 1.

77) In another aspect the invention employs one or more genes according to any one of paragraphs 1-76, wherein the gene has the symbol FAM26F, optionally in combination with one or more genes labeled as 1.77 to 1.100 identified in Table 1.

78) In another aspect the invention employs one or more genes according to any one of paragraphs 1-77, wherein the gene has the symbol FLJ31438, optionally in combination with one or more genes labeled as 1.78 to 1.100 identified in Table 1.

79) In another aspect the invention employs one or more genes according to any one of paragraphs 1-78, wherein the gene has the symbol SHROOM3, optionally in combination with one or more genes labeled as 1.79 to 1.100 identified in Table 1.

80) In another aspect the invention employs one or more genes according to any one of paragraphs 1-79, wherein the gene has the symbol TNFAIP3, optionally in combination with one or more genes labeled as 1.80 to 1.100 identified in Table 1.

81) In another aspect the invention employs one or more genes according to any one of paragraphs 1-80, wherein the gene has the symbol HLA-F, optionally in combination with one or more genes labeled as 1.81 to 1.100 identified in Table 1.

82) In another aspect the invention employs one or more genes according to any one of paragraphs 1-81, wherein the gene has the symbol CD3D, optionally in combination with one or more genes labeled as 1.82 to 1.100 identified in Table 1.

83) In another aspect the invention employs one or more genes according to any one of paragraphs 1-82, wherein the gene has the symbol MAP1B, optionally in combination with one or more genes labeled as 1.83 to 1.100 identified in Table 1.

84) In another aspect the invention employs one or more genes according to any one of paragraphs 1-83, wherein the gene has the symbol SRPX2, optionally in combination with one or more genes labeled as 1.84 to 1.100 identified in Table 1.

85) In another aspect the invention employs one or more genes according to any one of paragraphs 1-84, wherein the gene has the symbol AADAT, optionally in combination with one or more genes labeled as 1.85 to 1.100 identified in Table 1.

86) In another aspect the invention employs one or more genes according to any one of paragraphs 1-85, wherein the gene has the symbol ARHGAP15, optionally in combination with one or more genes labeled as 1.86 to 1.100 identified in Table 1.

87) In another aspect the invention employs one or more genes according to any one of paragraphs 1-86, wherein the gene has the symbol MCM10, optionally in combination with one or more genes labeled as 1.87 to 1.100 identified in Table 1.

88) In another aspect the invention employs one or more genes according to any one of paragraphs 1-87, wherein the gene has the symbol TC2N, optionally in combination with one or more genes labeled as 1.88 to 1.100 identified in Table 1.

89) In another aspect the invention employs one or more genes according to any one of paragraphs 1-88, wherein the gene has the symbol AP2B1, optionally in combination with one or more genes labeled as 1.89 to 1.100 identified in Table 1.

90) In another aspect the invention employs one or more genes according to any one of paragraphs 1-89, wherein the gene has the symbol GOLGA7, optionally in combination with one or more genes labeled as 1.90 to 1.100 identified in Table 1.

91) In another aspect the invention employs one or more genes according to any one of paragraphs 1-90, wherein the gene has the symbol TNFRSF9, optionally in combination with one or more genes labeled as 1.91 to 1.100 identified in Table 1.

92) In another aspect the invention employs one or more genes according to any one of paragraphs 1-91, wherein the gene has the symbol RNF144B, optionally in combination with one or more genes labeled as 1.92 to 1.100 identified in Table 1.

93) In another aspect the invention employs one or more genes according to any one of paragraphs 1-92, wherein the gene is the one identified by probe set 209671_x_at, optionally in combination with one or more genes labeled as 1.93 to 1.100 identified in Table 1.

94) In another aspect the invention employs one or more genes according to any one of paragraphs 1-93, wherein the gene has the symbol UBASH3B, optionally in combination with one or more genes labeled as 1.94 to 1.100 identified in Table 1.

95) In another aspect the invention employs one or more genes according to any one of paragraphs 1-94, wherein the gene has the symbol BTN3A1, optionally in combination with one or more genes labeled as 1.95 to 1.100 identified in Table 1.

96) In another aspect the invention employs one or more genes according to any one of paragraphs 1-95, wherein the gene has the symbol GCH1, optionally in combination with one or more genes labeled as 1.96 to 1.100 identified in Table 1.

97) In another aspect the invention employs one or more genes according to any one of paragraphs 1-96, wherein the gene has the symbol DENND2D, optionally in combination with one or more genes labeled as 1.97 to 1.100 identified in Table 1.

98) In another aspect the invention employs one or more genes according to any one of paragraphs 1-97, wherein the gene has the symbol C4orf7, optionally in combination with one or more genes labeled as 1.98 to 1.100 identified in Table 1.

99) In another aspect the invention employs one or more genes according to any one of paragraphs 1-98, wherein the gene has the symbol TNFAIP3, optionally in combination with one or more genes labeled as 1.99 to 1.100 identified in Table 1.

100) In another aspect the invention employs one or more genes according to any one of paragraphs 1-99, wherein the gene has the symbol GBP5, optionally in combination with one or more genes labeled as 1.100 identified in Table 1.

101) In another aspect the invention employs one or more genes according to any one of paragraph 1 to 100, wherein the gene has the symbol GBP1.

### EXPERIMENTAL EXAMPLES

### Example 1

### MAGE008 Mage Melanoma Clinical Trial

In this on-going trial, the recMAGE-A3 protein (recombinant mage fusion protein) is combined with two different immunological adjuvants: either AS02B (QS21, MPL) or AS15 (QS21, MPL and CpG7909). The objectives were to discriminate between the adjuvants in terms of safety profile, clinical response and immunological response.

In this experiment two adjuvant compositions are made up of mixtures of two immunostimulants:
1. QS21 (Purified, naturally occurring saponin molecule from the South-American tree Quillaja Saponaria Molina), and
2. MPL (3 de-O-acetylated monophosphoryl lipid A—detoxified derivative of lipid A, derived from S. minnesota LPS).
   AS02B is an oil-in-water emulsion of QS21 and MPL.

In animal models these adjuvants have been successfully shown to induce both humoral and TH 1 types of cellular-mediated immune responses, including CD4 and CD8 T-cells producing IFNα (Moore et al., 1999; Gérard et al., 2001). Moreover, the injection of recombinant protein formulated in this type of adjuvant leads to the induction of a systemic anti-tumor response: indeed, vaccinated animals were shown to be protected against challenges with murine tumor cells genetically engineered to express the tumor antigen, and regressing tumors were shown to be highly infiltrated by CD8, CD4 and NK cells and by macrophages.

The second adjuvant system is AS15: it contains a third immunostimulant, namely CpG7909 (otherwise known as CpG 2006 supra), in addition to MPL and QS21, in a liposome formulation. In animal models (mainly mice), it has been shown that the addition of CpG7909 further improves the induced immune and anti-tumor responses (Krieg and Davis, 2001; Ren et al., 2004). CpG oligodeoxynucleotides (ODNs) directly stimulate dendritic-cell activation through TLR9 triggering. In addition, in mice, the systemic application of CpG7909 greatly increases the infiltration of transferred T-cells into tumors (Meidenbauer et al., 2004).

### Study Overview

1. Design

The MAGE008 trial is:

open

randomized

two-arm (AS02B vs. AS15)

with 68 patients in total.

As described above, the recMAGE-A3 protein is combined with either AS02B or AS15 adjuvant system.

2. Patient Population

The recMAGE-A3 protein is administered to patients with progressive metastatic melanoma with regional or distant skin and/or lymph-node lesions (unresectable stage III and stage IV M1a). The expression of the MAGE-A3 gene by the tumor was assessed by quantitative PCR. The selected patients did not receive previous treatment for melanoma (recMAGE-A3 is given as first-line treatment) and had no visceral disease.

3. Schedule of Immunization

### Method of Treatment Schedules

The immunization schedule followed in the MAGE008 clinical trial was:
Cycle 1: 6 vaccinations at intervals of 2 weeks (Weeks 1, 3, 5, 7, 9, 11)
Cycle 2: 6 vaccinations at intervals of 3 weeks (Weeks 15, 18, 21, 24, 27, 30)
Cycle 3: 4 vaccinations at intervals of 6 weeks (Weeks 34, 40, 46, 52)
Long Term Treatment: 4 vaccinations at intervals of 3 months, for example followed by
   4 vaccinations at intervals of 6 months
      For both of the above treatment regimes additional vaccinations may be given after treatment, as required.

In order to screen potential participants in the above clinical trial we received biopsies of the tumor prior to any immunization. RNA was extracted from the biopsy for the MAGE-A3 quantitative PCR and this RNA was also use for gene expression profiling by microarrays. The goal was to identify in pre-vaccination biopsies a set of genes associated with the clinical response and to develop a mathematical model that would predict patient clinical outcome, so that patients likely to benefit from this antigen-specific cancer immunotherapeutic are properly identified and selected. Gene profiling analysis has been performed only on biopsies from patients who signed the informed consent for microarray analysis.

### 1. Materials and Methods

1.1. Tumor Specimens and RNA Purification

65 tumor biopsies taken previous to vaccination from 65 patients were used from the Mage008 Mage-3 melanoma clinical trial. These were fresh frozen preserved in the RNA stabilizing solution RNAlater.

Total RNA was purified using the Tripure method (Roche Cat. No. 1 667 165). The provided protocol was followed subsequently by the use of an RNeasy Mini kit—clean-up protocol with DNAse treatment (Qiagen Cat. No. 74106). RNA from the samples whose melanin content was high (determined by visual inspection) was further treated using CsCl centrifugation.

Quantification of RNA was initially completed using optical density at 260 nm and Quant-IT RiboGreen RNA assay kit (Invitrogen—Molecular probes R11490).

1.2. RNA Labeling and Amplification for Microarray Analysis

Due to the small biopsy size received during the clinical study, an amplification method was used in conjunction with the labeling of the RNA for microarray analysis: the Nugen 3′ ovation biotin kit (Labelling of 50 ng of RNA—Ovation biotin system Cat; 2300-12, 2300-60). A starting input of 50 ng of total RNA was used.

1.3. Microarray Chips, Hybridizations and Scanning

The Affymetrix HG-U133.Plus 2.0 gene chips were hybridized, washed and scanned according to the standard Affymetrix protocols.

1.1.1 Definition of Patients Used for Gene Signature Analysis

A binary classification approach was employed to assign patients to gene signature (GS) positive (GS+) or to GS negative (GS−) groups. The training set consisted of 56 evaluable patients who gave informed consent for gene signature analysis with good quality microarray data and with at least 6 vaccinations.

For this gene signature analysis, Responders (R) were defined as patients presenting objective signs of clinical activity and these included; objective response (Complete Response (CR), Partial Response (PR), stable disease (SD), Mixed Response (MR). Non-Responders (NR) were defined as Progressive Disease (PD). Only evaluable patients with at least 6 vaccinations were used for gene profile analysis since this is approximately when immune response was detected.

Responders (R) for gene profile analysis are the patients presenting signs of biological activity and these include: complete and partial responders (CR, PR), stable disease (SD), progressive disease (PD) with Mixed Response 1 (MxR1) and PD MxR2 with disappearance of at least one target lesion.

Non-Responders (NR): PD No MxR, PD MxR2 that did not show disappearance of at least one target lesion and Progressive Disease No MxR

The training set distribution in the two arms of this clinical study (comparing two immunological adjuvants) consisted of 22 R (14 in AS15 arm and 8 in AS02B arm) and 34 NR (13 AS15, 21 AS02B).

### Sample Normalization

After amplification and labelling of the RNA, hybridization to the HG-U133 plus2 Affymetrix GeneChip was performed. The CEL files obtained after scanning were normalized using a modified version of the GCRMA algorithm (Wu, 2004) in gcrma package from Bioconductor using all patients with good quality microarray data (based on scaling factor and gcrma normalization). This algorithm was adapted to store the pre-processing parameters obtained with this set of arrays. The parameters are of two types: the average empirical distribution necessary for quantile normalization, and the probe-specific effects to perform probeset (PS) summarization. These parameters were obtained from 65 samples and applied to the 56 samples in the training set to obtain summarized values for each probeset.

1.4. Absent/Present and Non-Specific Filtering

Affymetrix probe sets (PS) called Absent in all 65 samples used for normalization were removed using an R implementation of the PANP program (1.8.0 software version). This reduces the dataset from 54,613 to about 28,100 PS.

The interquartile range (IQR) filtered probe sets (PS) of normalized hybridization samples are filtered independently of the outcome associated to each sample. The objective of this non-specific filtering is to get rid of genes showing roughly constant expression across samples as they tend to provide little discrimination power (Heidebreck et al., 2004).

An interquantile filter which only retains PS with interquartile range equal or higher than 1.7 in the expression matrix of the training set (56 samples) was implemented. This step reduced the PS size from 28,100 down to about 5045.

### Feature Normalization

The summarized and filtered PS were subsequently normalized with a Z-score calculation. The Z-score for each individual patient expression PS value is calculated as follows: a PS-specific mean is subtracted from the PS value, and this mean-centered expression value is then weighted by a PS-specific standard deviation. The PS-specific means and standard deviations involved in the Z-score calculation are those calculated from the training set.

### Feature Selection

The selection of relevant PS to be used as features in the classification of the clinical outcome patient data consists in a signal to noise score is obtained using the normalized and z-scored expression matrix for the 56 samples in training set:

$$s 2 n = \frac{{\overline{x}}_{R} - {\overline{x}}_{NR}}{{sd}_{R} + {sd}_{NR}}$$

x̅_{R}=Mean of Responders
x̅_{NR}=Mean or Non-Responders
sd_{R}=Standard deviation Responders

sd_{NR}=Standard deviation Non-Responders

The 100 PS with highest absolute signal to noise score were selected as classifier features (Table 1). This number was estimated as appropriate since it is a feasible number of genes to measure with another technology (i.e. Q-RT-PCR).

The above methodology of gene selection was tested by crossvalidation as described in the next section.

### Leave One Out Crossvalidation (LOOCV) of Classification Method

In order to obtain an estimation of the performance of the methodology and choose an appropriate cutoff for the classifier; a classification scheme was developed and tested using crossvalidation by leave-one-out with re-calculation of reporter list at each cross-validation loop

First, a non-specific filter was applied that discarded probesets (PS) whose interquantile range (IQR) was less than 1.7 (˜5000 PS remaining in each crossvalidation). Subsequently, the Z-score normalization was performed within each training set and applied to the test sample. Genes were ranked using signal-to-noise (s2n) as described by Golub et al. (Golub, 1999), and the best 100 PS (absolute s2n score) were selected as classifier features.

A classification algorithm based on supervised principal component—discriminant analysis (SPCA) was built using the selected PS (Bair and Tibshirani, PLOS Biol 2004 and Tibshirani et al., PNAS 2002). The classifier is based on singular value decomposition of the expression matrix of the training set with only the PS selected as classifier features. The mean and standard deviation of each group (R and NR) of the training set in the first principal component (PC₁) are calculated. For classifying a test sample, its z-scored expression values are projected in the PC₁ defined by the train set and the distances in PC₁ to the mean of each group are used to calculate a probability that a sample belong to the Responder or Non-Responder group. The classifier outcome is thus an index which is the probability of a sample being Responder (GS+), ranging from 0 to 1.

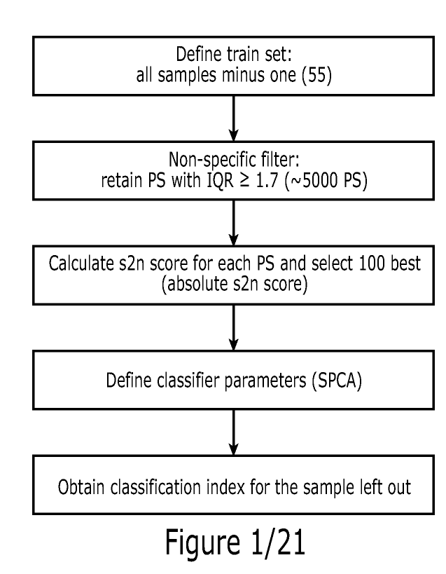

FIG. 1/21 shows the scheme for the LOOCV.

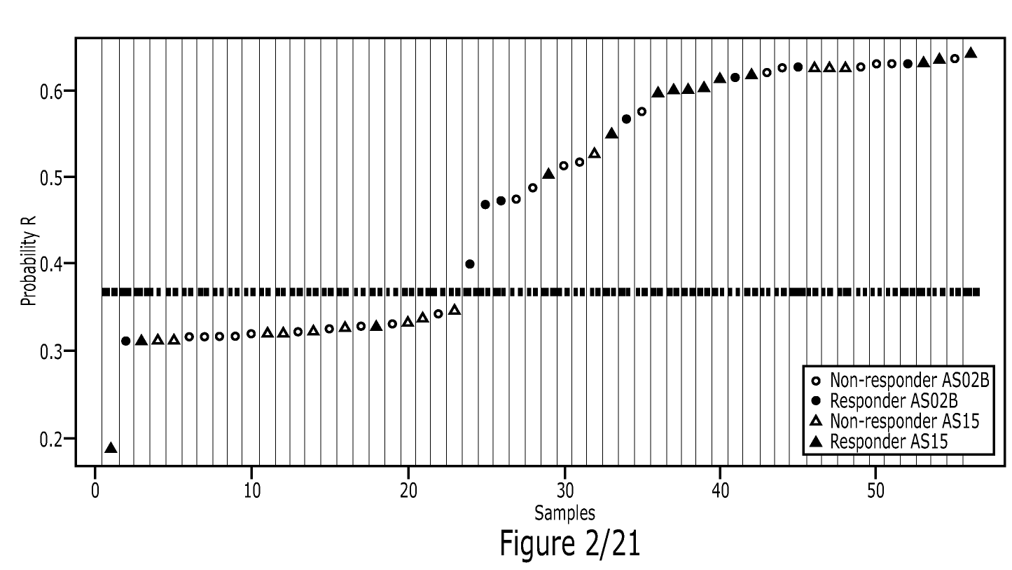

FIG. 2/21 shows the results of the LOOCV selecting the best 100 PS for classification in each loop.

Sensitivity (Se) and specificity (Sp) were used as performance indicators. Se is defined as the proportion of true positives (TP) among samples predicted as Responders, and Sp is defined as the proportion of true negatives (TN) among patients predicted as Non-Responders.

It can be seen from the graph of FIG. 2/21 that any value between 0.41 and 0.47 would have the same sensitivity and specificity. It was decided to take a cut off of 0.43. This cutoff would classify 32/56 samples as Responder (R) and sensitivity would be 17/22 (0.77) with specificity of 19/34 (0.56). Notably, the sensitivity and specificity only in the AS15 arm are higher; 0.79 and 0.69 respectively. Importantly, all objective responders (CR and PR) are correctly classified.

The stability of selected features in each of the 56 classifiers built by LOOCV was compared with features that were selected using all samples.

TABLE 1A
100 PS SELECTED USING ALL SAMPLES AND THE TIMES SELECTED
IN LOOCV

| | | Gene symbol | | times |
|---|---|---|---|---|
| | | according to | Gene symbol | selected |
| | | R2.9 | according to | in |
| | Affy ID | annotation | Affymetrix annotation | LOOCV |
| 1.1 | 1554240_a_at | ITGAL | ITGAL | 56 |
| 1.2 | 1555852_at | PSMB9 | NA | 56 |
| 1.3 | 1562031_at | JAK2 | JAK2 | 56 |
| 1.4 | 201474_s_at | ITGA3 | ITGA3 | 56 |
| 1.5 | 202659_at | PSMB10 | PSMB10 | 56 |
| 1.6 | 203915_at | CXCL9 | CXCL9 | 56 |
| 1.7 | 204070_at | RARRES3 | RARRES3 | 56 |
| 1.8 | 204116_at | IL2RG | IL2RG | 56 |
| 1.9 | 204533_at | CXCL10 | CXCL10 | 56 |
| 1.1 | 205758_at | CD8A | CD8A | 56 |
| 1.11 | 205890_s_at | UBD | GABBR1 /// UBD | 56 |
| 1.12 | 207651_at | GPR171 | GPR171 | 56 |
| 1.13 | 207795_s_at | KLRD1 | KLRD1 | 56 |
| 1.14 | 208729_x_at | HLA-B | HLA-B | 56 |
| 1.15 | 208885_at | LCP1 | LCP1 | 56 |
| 1.16 | 208894_at | HLA-DRA | HLA-DRA | 56 |
| 1.17 | 209606_at | CYTIP | CYTIP | 56 |
| 1.18 | 210915_x_at | IL23A | TRBC1 | 56 |
| 1.19 | 210972_x_at | TRA@ | TRA@ /// TRAC /// | 56 |
| | | | TRAJ17 /// TRAV20 | |
| 1.20 | 210982_s_at | HLA-DRA | HLA-DRA | 56 |
| 1.21 | 211144_x_at | TARP | TARP /// TRGC2 | 56 |
| 1.22 | 211339_s_at | ITK | ITK | 56 |
| 1.23 | 211796_s_at | IL23A | TRBC1 /// TRBC2 | 56 |
| 1.24 | 211911_x_at | HLA-B | HLA-B | 56 |
| 1.25 | 212671_s_at | HLA-DQA1 | HLA-DQA1 /// HLA-DQA2 | 56 |
| 1.26 | 213793_s_at | HOMER1 | HOMER1 | 56 |
| 1.27 | 215806_x_at | TRGC2 | TARP /// TRGC2 | 56 |
| 1.28 | 216920_s_at | TARP | TARP /// TRGC2 | 56 |
| 1.29 | 217436_x_at | HLA-A | HLA-A /// HLA-A29.1 /// | 56 |
| | | | HLA-B /// HLA-G /// HLA- | |
| | | | H /// HLA-J | |
| 1.30 | 217478_s_at | HLA-DMA | HLA-DMA | 56 |
| 1.31 | 221875_x_at | HLA-F | HLA-F | 56 |
| 1.32 | 222838_at | SLAMF7 | SLAMF7 | 56 |
| 1.33 | 223575_at | KIAA1549 | KIAA1549 | 56 |
| 1.34 | 225996_at | LONRF2 | LONRF2 | 56 |
| 1.35 | 228362_s_at | FAM26F | FAM26F | 56 |
| 1.36 | 228532_at | C1orf162 | C1orf162 | 56 |
| 1.37 | 229391_s_at | FAM26F | FAM26F | 56 |
| 1.38 | 229625_at | GBP5 | GBP5 | 56 |
| 1.39 | 232375_at | STAT1* | NA | 56 |
| 1.40 | 232481_s_at | SLITRK6 | SLITRK6 | 56 |
| 1.41 | 235175_at | GBP4 | GBP4 | 56 |
| 1.42 | 235276_at | EPSTI1 | EPSTI1 | 56 |
| 1.43 | 244393_x_at | AKR1C2* | NA | 56 |
| 1.44 | AFFX- | STAT1 | STAT1 | 56 |
| | HUMISGF3A/M97935_MB_at | | | |
| 1.45 | 1552613_s_at | CDC42SE2 | CDC42SE2 | 55 |
| 1.46 | 204556_s_at | DZIP1 | DZIP1 | 55 |
| 1.47 | 204897_at | PTGER4 | PTGER4 | 55 |
| 1.48 | 206082_at | HCP5 | HCP5 | 55 |
| 1.49 | 211149_at | UTY | LOC100130224 /// UTY | 55 |
| 1.50 | 214470_at | KLRB1 | KLRB1 | 55 |
| 1.51 | 229543_at | FAM26F | FAM26F | 55 |
| 1.52 | 231229_at | HILS1 | HILS1 | 55 |
| 1.53 | 232234_at | C20orf24 | SLA2 | 55 |
| 1.54 | 232311_at | B2M | B2M | 55 |
| 1.55 | 236328_at | ZNF285A | ZNF285A | 55 |
| 1.56 | 237515_at | TMEM56 | TMEM56 | 55 |
| 1.57 | 202531_at | IRF1 | IRF1 | 54 |
| 1.58 | 209813_x_at | TRGV9 | TARP | 54 |
| 1.59 | 238524_at | NA | NA | 54 |
| 1.60 | 205097_at | SLC26A2 | SLC26A2 | 53 |
| 1.61 | 209774_x_at | CXCL2 | CXCL2 | 53 |
| 1.62 | 210439_at | ICOS | ICOS | 53 |
| 1.63 | 213193_x_at | IL23A | TRBC1 | 53 |
| 1.64 | 1555759_a_at | CCL5 | CCL5 | 52 |
| 1.65 | 1562051_at | LOC284757 | LOC284757 | 52 |
| 1.66 | 205685_at | CD86 | CD86 | 50 |
| 1.67 | 210606_x_at | KLRD1 | KLRD1 | 50 |
| 1.68 | 211902_x_at | TRA@ | TRA@ | 50 |
| 1.69 | 1552497_a_at | SLAMF6 | SLAMF6 | 48 |
| 1.70 | 204529_s_at | TOX | TOX | 48 |
| 1.71 | 206666_at | GZMK | GZMK | 48 |
| 1.72 | 1552612_at | CDC42SE2 | CDC42SE2 | 47 |
| 1.73 | 1563473_at | PPP1R16B* | NA | 45 |
| 1.74 | 219551_at | EAF2 | EAF2 | 45 |
| 1.75 | 228492_at | USP9Y | LOC100130216 /// | 44 |
| | | | USP9Y | |
| 1.76 | 229390_at | FAM26F | FAM26F | 43 |
| 1.77 | 228316_at | FLJ31438* | C2orf63 | 42 |
| 1.78 | 228400_at | SHROOM3 | SHROOM3 | 42 |
| 1.79 | 202643_s_at | TNFAIP3 | TNFAIP3 | 41 |
| 1.80 | 204806_x_at | HLA-F | HLA-F | 41 |
| 1.81 | 213539_at | CD3D | CD3D | 41 |
| 1.82 | 226084_at | MAP1B | MAP1B | 41 |
| 1.83 | 205499_at | SRPX2 | SRPX2 | 40 |
| 1.84 | 223593_at | AADAT | AADAT | 40 |
| 1.85 | 244061_at | ARHGAP15* | NA | 40 |
| 1.86 | 222962_s_at | MCM10 | MCM10 | 39 |
| 1.87 | 1553132_a_at | TC2N | TC2N | 38 |
| 1.88 | 200615_s_at | AP2B1 | AP2B1 | 38 |
| 1.89 | 234907_x_at | GOLGA7* | NA | 38 |
| 1.90 | 207536_s_at | TNFRSF9 | TNFRSF9 | 36 |
| 1.91 | 239012_at | RNF144B | RNF144B | 34 |
| 1.92 | 209671_x_at | TRA@ | TRA@ /// TRAC | 32 |
| 1.93 | 238587_at | UBASH3B | UBASH3B | 31 |
| 1.94 | 209770_at | BTN3A1 | BTN3A1 | 27 |
| 1.95 | 204224_s_at | GCH1 | GCH1 | 25 |
| 1.96 | 221081_s_at | DENND2D | DENND2D | 25 |
| 1.97 | 229152_at | C4orf7 | C4orf7 | 24 |
| 1.98 | 202644_s_at | TNFAIP3 | TNFAIP3 | 19 |
| 1.99 | 238581_at | GBP5 | GBP5 | 17 |
| 1.100 | 231577_s_at | GBP1 | GBP1 | 15 |

*Annotation from R2.6 that became NA in R2.9

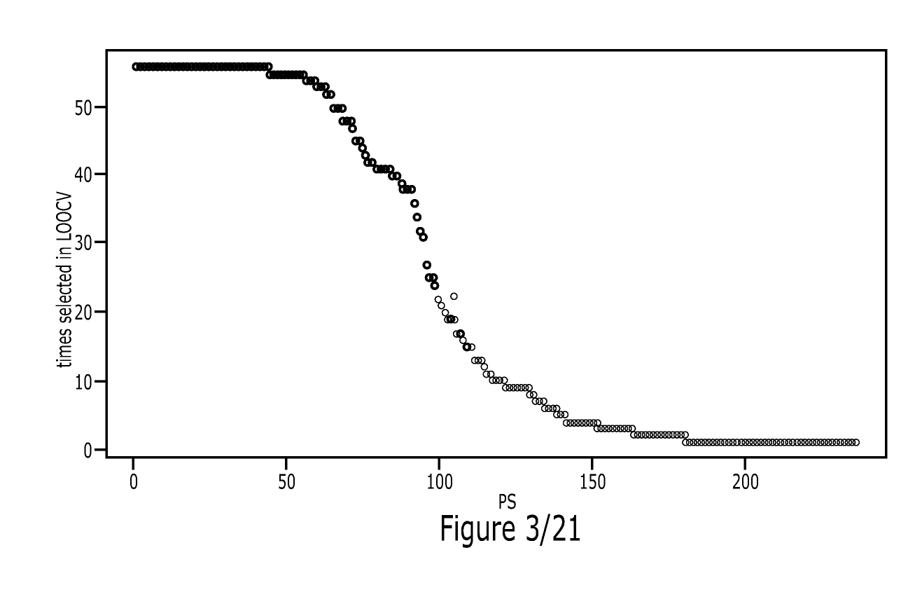

FIG. 3/21 shows the number of times that a PS was within the 100 top s2n in each LOOCV. The PS selected also using all samples are indicated in black. 68 of the 100 PS selected using all samples were also selected in at least 50 of the LOOCVs, the list of 100 PS selected using all samples would be the classifier features to be used in predicting the response of independent patients (Table 1).

### Impact of Gene Signature on Overall Survival (OS)

In Cox regression, hazard represent the probability that the event (death, disease progression) occurs during a period of time. A baseline hazard is assumed to be shared by all samples and covariates that are explanatory variables that have an effect on the hazard are added to the model. Hazard ratio quantifies the effect a covariate has on hazard. It reflects the relative risk of a variable.

For example, a treatment with a hazard ratio of 0.4 as in Table 2 below means that a gene signature positive patient has a 60% reduced risk of death per period of time compared to gene signature negative patients. Note that 0.4 is the mean of the expected HR and the 95% confidence intervals are also estimated in the model.

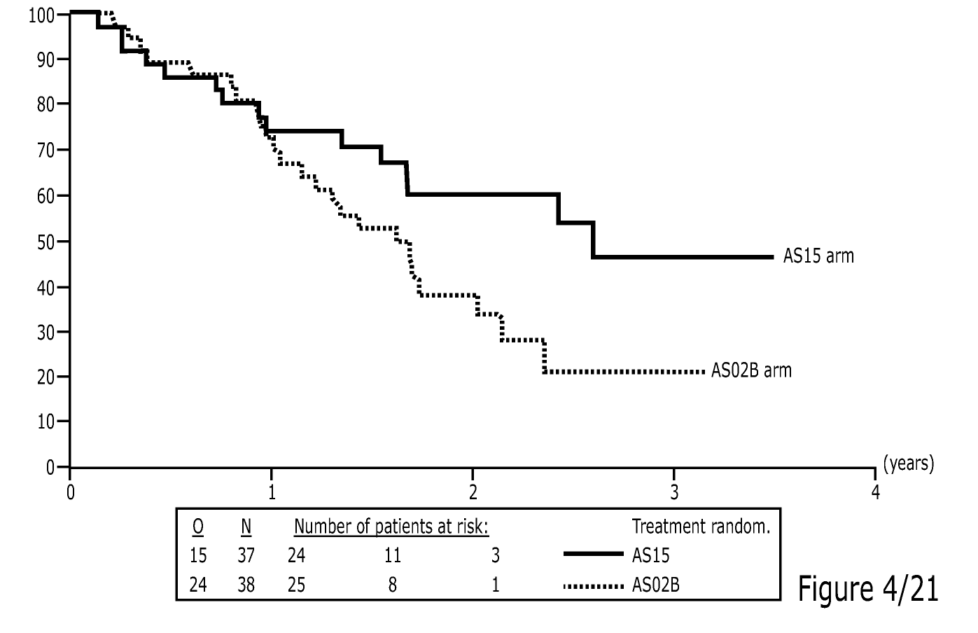

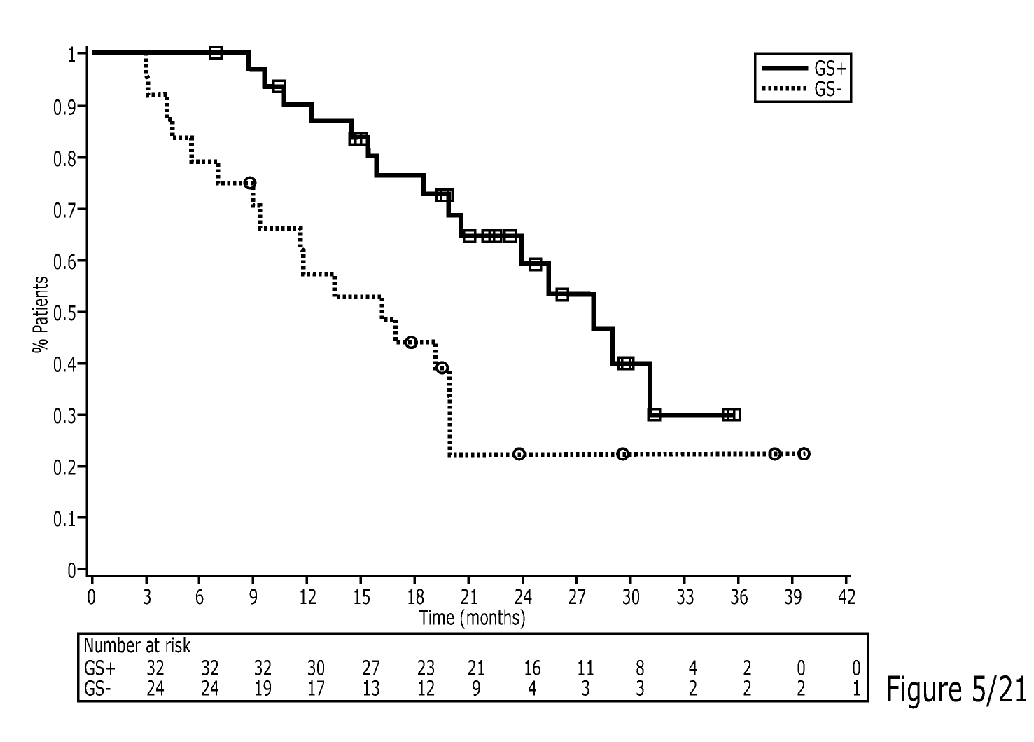

FIG. 4/21 shows the Kaplan-Meier curves (KM) for OS by adjuvant with all patients in the Phase II melanoma trial; Hazard Ratio (HR): 0.55 (95% Cl [0.28; 1.06]). The estimated hazard ratio when using only the 56 patients in training set is 0.41 (95% Cl [0.191; 0.88]). To estimate the impact of the GS on the overall survival (OS), the classification obtained by LOOCV with a cutoff of 0.43 was used (section 1.4); the graph in FIG. 5/21 shows the KM for OS by GS.

Fitting a multivariate Cox-model with adjuvant and GS as covariates yields the following HR for GS:

| | | lower | upper |
|---|---|---|---|
| | HR | 0.95 | 0.95 |
| GS+ vs GS− | 0.4 | 0.197 | 0.813 |

The estimated median survival times by GS are:

| | median | | |
|---|---|---|---|
| | survival | lower | upper |
| | (months) | 0.95 | 0.95 |
| GS− | 16.2 | 9.4 | Inf |
| GS+ | 28 | 20.5 | Inf |

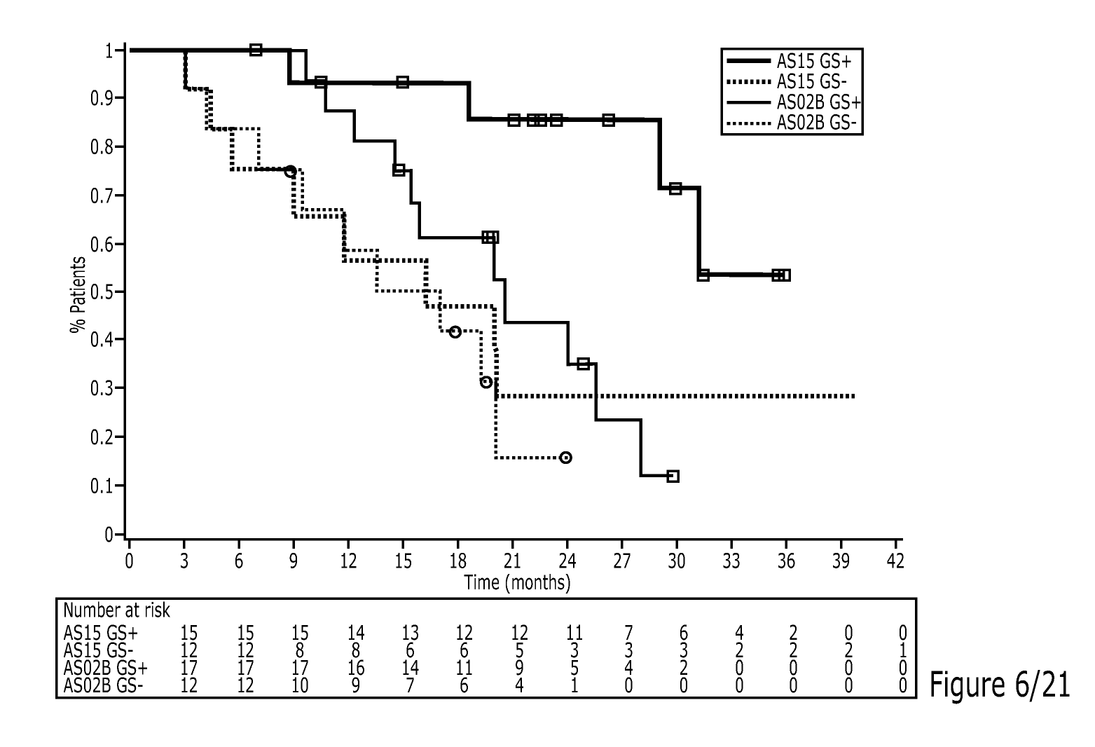

The Overall Survival Kaplan-Meier curves by adjuvant and gene signature based on LOOCV classification are shown in FIG. 6/21 and the HR is as follows.

| | | lower | upper |
|---|---|---|---|
| | HR | 0.95 | 0.95 |
| AS15 GS+ vs | 0.268 | 0.080 | 0.896 |
| GS− | | | |
| AS02B GS+ vs | 0.433 | 0.165 | 1.140 |
| GS− | | | |

As discussed above, a classifier based on a given gene expression profile to predict clinical response to MAGE-A3 ASCI has been developed and crossvalidated in the Phase II melanoma trial (GSK 249553/008). The classifier performance was estimated using LOOCV obtaining a sensitivity of 0.77 and specificity of 0.56. The specificity in the AS15 arm only is 0.79 and sensitivity 0.69. This classification resulted in a significant reduction in the hazard ratio for overall survival in the GS+ population, with a more important effect in the AS15 arm.

The stability of classifier feature selection was also evaluated and it was found to be robust to removing one sample in the training set. The biology of the signature linked to clinical efficacy of the MAGE-A3-ASCI (top 100 PS by s2n using all 56 patients in the training set; Table 1) is relevant to the ASCI mode of action since it contains genes that suggest the presence of a specific tumor microenvironment (chemokines) that favor presence of immune effector cells in the tumor of responder patients which show upregulation of T-cell markers. A recent gene expression profiling study in metastatic melanoma revealed that tumors could be segregated based on presence or absence of T-cell associated transcripts (Harlin, 2009). The presence of lymphocytes in tumors correlated with the expression of a subset of six chemokines (CCL2, CCL3, CCL4, CCL5, CXCL9, CXCL10), three out of these six genes (CCL5, CXCL9, CXCL10) are present in the 100 PS. Interestingly, HLA molecules were also found to be upregulated in the responder patients. It has been postulated that downregulation of HLA molecules in the tumor cells might be a mechanism to evade immune surveillance (Aptsiauri, 2008).

The top biological functions from Ingenuity Pathway Analysis confirmed the enrichment of immune related genes in the 100 PS signature (p-value is the range obtained for sub-functions):

| | | number of |
|---|---|---|
| Biological Function | p-value | genes |
| Antigen Presentation | 5.53E−14-5.06E−03 | 27 |
| Cell-To-Cell Signaling and Interaction | 5.40E−13-7.60E−03 | 28 |
| Cellular Development | 1.58E−11-6.75E−03 | 27 |
| Cell Death | 1.18E−09-5.80E−03 | 28 |
| Cellular Movement | 3.56E−08-7.60E−03 | 19 |
| Cell-mediated Immune Response | 5.53E−14-7.60E−03 | 32 |
| Humoral Immune Response | 5.53E−14-7.60E−03 | 29 |
| Hematological System Development | 4.44E−13-7.60E−03 | 32 |
| and Function | | |
| Tissue Morphology | 4.44E−13-7.60E−03 | 23 |
| Immune Cell Trafficking | 6.77E−13-7.60E−03 | 23 |

### 4. Clinical Outcome Prediction of a New Sample

The steps described here to perform the clinical outcome prediction have been written as R scripts. Before performing the clinical outcome prediction for a given patient, two successive normalizations of the patient Affymetrix genechip data are undertaken; the sample and gene normalizations. The goal of these normalizations is to produce gene expression values for the patient that will be comparable, by being correctly scaled to the training set data from which the prediction scheme was developed. The training set consists of 56 samples from the phase II melanoma trial. Details regarding the training set and sample normalization have been described in the preceding sections and in further detail in the following paragraph.

### 4.1 Sample Normalization

The sample normalization, also known as pre-processing is carried out starting with the CEL file for each sample and will take care of the following aspects:
1. Correct for background raw Affymetrix oligonucleotide probe intensities;
2. Normalize the background corrected probe intensities using a quantile normalization procedure.
3. Convert the probe intensities into a single probe set intensity following a probes-to-PS mapping defined in a Chip Definition File (CDF). The CDF file is specific for the genechip array (hgu133plus2) used and provided by Affymetrix. This last step is called summarization

The goal of this step is to fit the distribution of the probe set (PS) intensities of the unknown patient data towards the PS intensity distributions of the training set. This is done using the GCRMA algorithm (Wu, 2004). This algorithm was adapted to account for pre-processing parameters that are defined on a reference microarray data set. The parameters are of two types: the average empirical distribution necessary for quantile normalization, and the probe-specific effects to perform PS summarization.

The reference GCRMA parameters were built with 65 samples from the phase II melanoma trial study and these are applied to a new patient sample using a code based on the refplus R package.

The Appendix 1 code chunk is a modification of the code contained in the RefPlus R package (Harbron et al., 2007), available in Bioconductor. The RefPlus code is modified to perform a GCRMA normalization of a given sample hybridization, taking into account normalization parameters calculated from a reference data set. The reference dataset is the data set described in the previous sections (65 patients). RefPlus is initially designed for reference data set normalization, but uses the RMA algorithm rather than the GCRMA. The only difference between RMA and GCRMA lies in the background correction step. RefPlus was enabled to perform GCRMA background correction by replacing the bg.correct.rma R function embedded in the rmaplus R function by the bg.adjust.gcrma R function. The RefPlus code modification was done in October 2007 and is available from GlaxoSmithKline. To normalize a sample with GCRMA-enabled, modified RefPlus code of Appendix 1, one would have to call the GCRMA background correction enabled-rmaplus function, with, as parameters, besides the data to normalize (of class AffyBatch), the reference quantiles (r.q option) and probe effect (p.e option) that are calculated on the reference data set. The reference quantiles and probe effects are contained in the rq.txt and pe.txt files, available from GSK and submitted to the USPTO on Compact Disc as referenced above.

To normalize a sample with GCRMA-enabled, modified RefPlus code of Appendix 1 (FIG. 5), one would have to call the GCRMA background correction enabled-rmaplus function, with, as parameters, besides the data to normalize (of class AffyBatch), the reference quantiles (r.q option) and probe effect (p.e option) that are calculated on the reference data set. The reference quantiles and probe effects are contained in the rq.txt and pe.txt files, available from the Head of Corporate Intellectual Property at GSK, named VR63933P_rq.txt and VR63933P_pe.txt, respectively. These files have also been submitted to the USPTO on a Compact Disc in respect of the U.S. priority application Ser. No. 61/278,387 filed 6 Oct. 2009 and may be obtained by ordering the file history of U.S. Ser. No. 61/278,387 from the USPTO at such time as it is available.

In the meantime, these files are also available as zip files at https://sites.google.com/site/vr63933/vr63933r files (note that there is a “_” between the letter “r” and the word “files” in the https address). The files on the website are named VR63933P_rq.zip and VR63933P_pe.zip, respectively. To obtain copies of these two files, navigate to the address provided in this paragraph and select the hypertext “Download” for each file. Choose the “Save” option at the prompt and save to a desired location. Open the files as one would normally open a zip file and save them as ASCII (.txt) files at a desired location. Then follow the instructions in the first two paragraphs of the present application.

The summarized probe sets (PS) are subsequently normalized with a Z-score calculation; this is applied to the PS selected as classifier features. The goal of this second normalization step is to make identical the genes which share a similar expression pattern throughout the data but have different absolute expression value ranges.

The Z-score for each individual patient expression PS value is calculated as follows: a PS-specific mean is subtracted from the PS value, and this mean-centered expression value is then weighted by a PS-specific standard deviation. The PS-specific means and standard deviations involved in the Z-score calculation are those calculated from the training set (Table 4).

Once the patient raw data has been normalized with the training set parameters, they can be subjected to a decision rule (classifier or classification scheme) for prediction of the clinical outcome for the patient.

### 4.2 Algorithm for Classification of a New Samples

For prediction of the patient clinical outcome based on the normalized patient PS, a supervised principal component (SPCA)—discriminant analysis (DA) decision rule is applied (adapted from Bair, 2004; Tibshirani, 2002). The prediction process invoking the SPCA-DA works as follows:
The probe sets used for classification are only the classifier features (100 PS) and were identified during model development based on the training set (Table 1)
The normalized expression profile (classifier features) of the patient to classify is projected in the first principal component (PC₁) space defined by the training set using a linear combination of the classifier features (the coefficients for each feature in the linear combination was obtained by singular value decomposition of the training set and they are provided in Table 4)
The standardized distance of the test sample in PC1 to the mean of the Responder and non responder group is obtained using the following equation:

$${d}_{iK} = \frac{\vert {PC}_{1 i} - {mean\_PC}_{1 K} \vert}{{sd\_PC}_{1 K}}$$

i=test sample
K=Responder (R) or Non-Responder (NR)
mean_PC_{1K}=PC₁ mean of R or NR group in training set
sd_PC_{1K}=PC₁ standard deviation of R or NR group in training set
The mean and sd of each group in the training set (rounded to three significant digits) are:

| | |
|---|---|
| mean_PC_{1R} | −4.622 |
| sd_PC_{1R} | 5.727 |
| mean_PC_{1NR} | 2.991 |
| sd_PC_{1NR} | 7.051 |

The index (probability of sample being Responder) for each sample is obtained with:

$${P}_{R} = \frac{{e}^{- \frac{{d}_{iR}}{2}}}{{e}^{- \frac{{d}_{iR}}{2}} + {e}^{- \frac{{d}_{iNR}}{2}}}$$

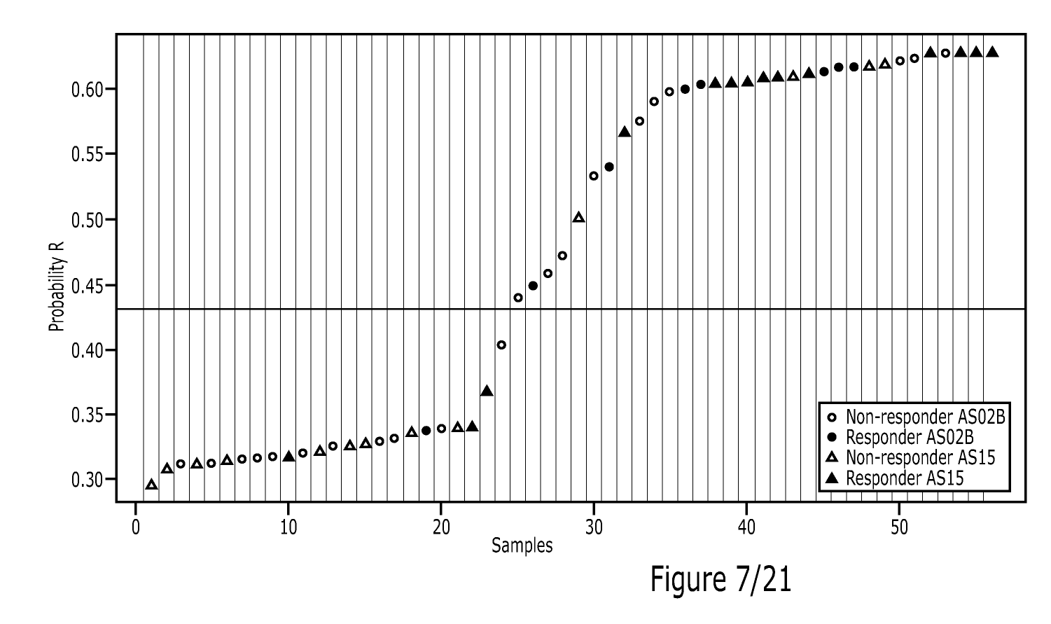

A sample is classified as gene signature positive (Responder,R) if its P_{R} is greater than 0.43
   Applying this classifier to the training set for the purpose of exemplifying the method, produces FIG. 7/21.

Algorithm for predicting a new sample

library(genefilter)
#### load testset to classify (normalized microarray data)
load(“testset.RData”) ### ExpressionSet containing samples to
classify
testset<-data ###(modify xx according to batch number)
### Load training set parameters ##############
load(“M8.train.parameters.RData”)
PS<-M8.train.parameters[[1]]
M8.train.means<-M8.train.parameters[[2]]
M8.train.sd<-M8.train.parameters[[3]]
M8.train.U<-M8.train.parameters[[4]]
M8.trainPC1barRs<-M8.train.parameters[[5]]
M8.trainPC1sdRs<-M8.train.parameters[[6]]
M8.trainPC1barNRs<-M8.train.parameters[[7]]
M8.trainPC1sdNRs<-M8.train.parameters[[8]]
################################## Use SPCA on test set -
#######################
testset<-testset[PS,]
test<-(exprs(testset)−M8.train.means)/M8.train.sd
PCtest<-t(test) %*% M8.train.U
PC1test<-PCtest[,1]
distanceR<-c( )
distanceNR<-c( )
probR<-c( )
probNR<-c( )
SPCAclass<-c( )
for (i in 1:ncol(test)) {
distancesR<-abs(PCtest[i,1]−M8.trainPC1barRs)/M8.trainPC1sdRs
distancesNR<-abs(PCtest[i,1]−M8.trainPC1barNRs)/M8.trainPC1sdNRs
distanceR<-c(distanceR,distancesR)
distanceNR<-c(distanceNR,distancesNR)
probRs<-exp(−distancesR/2)/(exp(−distancesR/2)+exp(−
distancesNR/2))
probNRs<-exp(−distancesNR/2)/(exp(−distancesR/2)+exp(−
distancesNR/2))
probR<-c(probR,probRs)
probNR<-c(probNR,probNRs)
}
cutoff=0.43
clust<-ifelse(as.vector(probR)>cutoff, R,NR))

### Where

testset is a matrix with 100 rows containing the normalized microarray data for the 100 PS
M8.train.parameters is an object of class list containing:
   1. a character list of the 100 PS
   2. a vector of 100 mean values for each PS in the train set
   3. a vector of 100 sd values for each PS in the train set
   4. a matrix of 100 rows and 56 columns containing the U matrix of the svd decomposition of the train matrix
   5. the PC1 mean value of the responder group in the train
   6. the PC1 sd value of the responder group in the train
   7. the PC1 mean value of the non-responder group in the train
   8. the PC1 sd value of the non-responder group in the train

TABLE 4
Mean, Standard Deviations (Sd) and PC₁ Coefficients for the 100 PS
classifier features

| | Mean | Sd | PC1 |
|---|---|---|---|
| 213793_s_at | 6.638 | 1.437 | 0.0827 |
| 223593_at | 4.245 | 1.721 | 0.0698 |
| 225996_at | 5.369 | 2.116 | 0.0625 |
| 204556_s_at | 3.515 | 1.49 | 0.0594 |
| 223575_at | 5.664 | 1.785 | 0.0556 |
| 205097_at | 7.907 | 1.526 | 0.0553 |
| 231229_at | 6.464 | 1.711 | 0.0504 |
| 1562051_at | 3.576 | 1.847 | 0.0503 |
| 244393_x_at | 4.702 | 1.444 | 0.0494 |
| 200615_s_at | 6.286 | 1.232 | 0.0407 |
| 228316_at | 5.362 | 1.369 | 0.0402 |
| 201474_s_at | 4.506 | 1.331 | 0.0376 |
| 222962_s_at | 5.177 | 1.139 | 0.0372 |
| 236328_at | 7.034 | 1.936 | 0.0339 |
| 232481_s_at | 3.731 | 2.053 | 0.0328 |
| 228400_at | 3.458 | 1.437 | 0.0279 |
| 211149_at | 4.061 | 2.272 | 0.0266 |
| 228492_at | 4.538 | 2.983 | 0.0254 |
| 237515_at | 5.513 | 1.86 | 0.0245 |
| 226084_at | 9.153 | 1.388 | 0.0234 |
| 205499_at | 4.675 | 1.719 | 0.0002 |
| 234907_x_at | 3.95 | 1.465 | −0.0051 |
| 1553132_a_at | 4.068 | 1.29 | −0.0504 |
| 239012_at | 6.533 | 1.694 | −0.0656 |
| 238587_at | 6.039 | 1.292 | −0.0717 |
| 219551_at | 4.637 | 1.569 | −0.0789 |
| AFFX-HUMISGF3A/ | 7.445 | 1.504 | −0.0819 |
| M97935_MB_at | | | |
| 1562031_at | 6.386 | 1.521 | −0.0871 |
| 238524_at | 4.961 | 1.623 | −0.0883 |
| 217436_x_at | 8.377 | 1.127 | −0.0891 |
| 1552612_at | 7.216 | 1.841 | −0.0929 |
| 244061_at | 6.081 | 1.918 | −0.0935 |
| 209774_x_at | 6.653 | 1.952 | −0.0953 |
| 221081_s_at | 6.805 | 2.062 | −0.0956 |
| 206082_at | 6.505 | 2.038 | −0.0988 |
| 209770_at | 10.821 | 1.153 | −0.1002 |
| 232375_at | 8.732 | 1.379 | −0.1007 |
| 211911_x_at | 10.865 | 1.461 | −0.1042 |
| 1552613_s_at | 7.491 | 1.275 | −0.1043 |
| 221875_x_at | 10.907 | 1.258 | −0.1044 |
| 214470_at | 6.927 | 1.801 | −0.1049 |
| 232311_at | 7.001 | 1.484 | −0.105 |
| 208729_x_at | 10.389 | 1.419 | −0.106 |
| 207536_s_at | 4.073 | 1.75 | −0.1061 |
| 204806_x_at | 10.065 | 1.283 | −0.1062 |
| 1554240_a_at | 4.02 | 1.761 | −0.1068 |
| 207795_s_at | 3.698 | 1.803 | −0.1073 |
| 202659_at | 6.944 | 1.284 | −0.1077 |
| 210606_x_at | 3.915 | 1.892 | −0.1083 |
| 235276_at | 7.632 | 1.905 | −0.1084 |
| 208885_at | 10.544 | 1.865 | −0.1084 |
| 202643_s_at | 5.855 | 1.381 | −0.1087 |
| 204533_at | 8.875 | 3.111 | −0.1088 |
| 229152_at | 6.925 | 3.232 | −0.1092 |
| 1563473_at | 7.07 | 2.31 | −0.1112 |
| 204529_s_at | 7.139 | 2.08 | −0.1115 |
| 235175_at | 8.682 | 2.268 | −0.1118 |
| 204897_at | 9.206 | 1.692 | −0.1123 |
| 204070_at | 8.233 | 2.205 | −0.1125 |
| 210439_at | 4.539 | 1.825 | −0.1131 |
| 1555759_a_at | 4.213 | 1.638 | −0.1133 |
| 204224_s_at | 9.809 | 1.798 | −0.1137 |
| 202644_s_at | 8.64 | 1.472 | −0.114 |
| 231577_s_at | 8.659 | 1.996 | −0.114 |
| 210982_s_at | 11.946 | 1.662 | −0.1145 |
| 1555852_at | 6.989 | 1.89 | −0.1149 |
| 209813_x_at | 4.135 | 1.808 | −0.1152 |
| 205685_at | 6.927 | 1.728 | −0.1153 |
| 238581_at | 4.289 | 1.801 | −0.1158 |
| 229543_at | 8.937 | 2.328 | −0.1159 |
| 229390_at | 9.644 | 2.315 | −0.1159 |
| 208894_at | 11.493 | 1.628 | −0.1161 |
| 222838_at | 7.302 | 2.672 | −0.1164 |
| 228532_at | 8.693 | 1.684 | −0.1165 |
| 209606_at | 5.957 | 2.038 | −0.1168 |
| 217478_s_at | 9.575 | 1.559 | −0.1173 |
| 229391_s_at | 9.135 | 2.228 | −0.1175 |
| 211144_x_at | 4.32 | 1.949 | −0.1179 |
| 228362_s_at | 8.288 | 2.398 | −0.1179 |
| 212671_s_at | 8.72 | 2.387 | −0.1182 |
| 203915_at | 9.242 | 3.331 | −0.1191 |
| 229625_at | 7.32 | 2.116 | −0.1197 |
| 211902_x_at | 7.387 | 1.956 | −0.1197 |
| 209671_x_at | 5.905 | 2.044 | −0.1197 |
| 1552497_a_at | 4.827 | 2.195 | −0.1205 |
| 215806_x_at | 4.544 | 1.973 | −0.1215 |
| 216920_s_at | 5.641 | 1.862 | −0.1221 |
| 210972_x_at | 7.322 | 2.354 | −0.1224 |
| 205890_s_at | 8.864 | 2.983 | −0.1225 |
| 232234_at | 6.877 | 2.249 | −0.1228 |
| 207651_at | 7.222 | 2.531 | −0.1229 |
| 202531_at | 7.451 | 1.809 | −0.1234 |
| 206666_at | 6.816 | 2.698 | −0.1242 |
| 213193_x_at | 6.825 | 2.768 | −0.1257 |
| 204116_at | 6.106 | 2.683 | −0.126 |
| 213539_at | 7.398 | 2.851 | −0.1263 |
| 211339_s_at | 5.602 | 2.061 | −0.1266 |
| 210915_x_at | 6.533 | 2.733 | −0.1267 |
| 211796_s_at | 6.946 | 2.921 | −0.1271 |
| 205758_at | 7.338 | 3.285 | −0.1275 |

### Example 2

### Melanoma Classifier Using Q-RT-PCR Data

The RNA used for gene expression profiling by microarray was tested in a custom Taqman Low Density Array (ABI, PN 4342259) containing 22 genes from the 100PS (83 genes) and 5 reference genes for normalization (GUSB, PGK1, H3F3A, EIF4G2, HNRNPC) (Table 3).

For this analysis; a total of 54 melanoma samples were included (52 also used for microarray analysis and 2 additional ones for which the microarray hybridization was not of good quality).

TABLE 5
ABI Taqman Assay numbers for 22 genes plus reference genes used to
build PCR based classifier in melanoma samples
22 genes in 100PS measured by PCR

| Gene symbol | Gene Name | Taqman Assay |
|---|---|---|
| CCL5 | chemokine (C-C motif) | Hs00174575_m1 |
| | ligand 5 | |
| JAK2 | Janus kinase 2 (a protein | Hs01078136_m1 |
| | tyrosine kinase) | |
| IRF1 | interferon regulatory | Hs00971960_m1 |
| | factor 1 | |
| CXCL9 | chemokine (C—X—C motif) | Hs00171065_m1 |
| | ligand 9 | |
| IL2RG | interleukin 2 receptor, | Hs00173950_m1 |
| | gamma (severe | |
| | combined | |
| | immunodeficiency) | |
| CXCL10 | chemokine (C—X—C motif) | Hs00171042_m1 |
| | ligand 10 | |
| SLC26A2 | solute carrier family 26 | Hs00164423_m1 |
| | (sulfate transporter), | |
| | member 2 | |
| CD86 | CD86 molecule | Hs01567025_m1 |
| CD8A | CD8a molecule | Hs00233520_m1 |
| UBD | ubiquitin D | Hs00197374_m1 |
| GZMK | granzyme K (granzyme | Hs00157878_m1 |
| | 3; tryptase II) | |
| GPR171 | G protein-coupled | Hs00664328_s1 |
| | receptor 171 | |
| PSCDBP | pleckstrin homology, | Hs00188734_m1 |
| (synonym: CYTIP) | Sec7 and coiled-coil | |
| | domains, | |
| | binding protein | |
| CXCL2 | chemokine (C—X—C motif) | Hs00236966_m1 |
| | ligand 2 | |
| ICOS | inducible T-cell co- | Hs99999163_m1 |
| | stimulator | |
| TRBC1 | T cell receptor beta | Hs00411919_m1 |
| | constant 2 | |
| TRA@; TRAJ17; | T cell receptor alpha | Hs00948942_m1 |
| TRDV2; TRAC; | locus | |
| TRAV20 | | |
| TARP; TRGC2 | TCR gamma alternate | Hs00827007_m1 |
| | reading frame protein; T | |
| | cell | |
| | receptor gamma | |
| | constant 2 | |
| ITK | IL2-inducible T-cell | Hs00950634_m1 |
| | kinase | |
| C4orf7 | chromosome 4 open | Hs00395131_m1 |
| | reading frame 7 | |
| CD3D | CD3d molecule, delta | Hs00174158_m1 |
| | (CD3-TCR complex) | |
| HLA-DMA | major histocompatibility | Hs00185435_m1 |
| | complex, class II, DM | |
| | alpha | |
| PGK1 | Housekeeping gene | Hs99999906_m1 |
| GUSB | Housekeeping gene | Hs99999908_m1 |
| HNRNPC | Housekeeping gene | Hs01028910_g1 |
| EIF4G2 | Housekeeping gene | Hs01034743_g1 |
| H3F3A | Housekeeping gene | Hs02598545_g1 |

cDNA synthesis from 500 ng (OD₂₆₀ measurement) of total RNA was performed in a 20 μl mixture containing 1× first strand buffer, 0.5 mM of each dNTP, 10 mM of dithiothreitol, 20 U of rRNase inhibitor (Promega cat.N2511), 250 ng of Random hexamers and 200 U of M-MLV reverse transcriptase (Life Technologies cat. 28025-013) for 1 h30 at 42° C. cDNA corresponding to 200 ng of total RNA was mixed in a total volume of 200 μl containing TaqMan buffer, 5 mM MgCl2, 0.4 mM dUTP, 0.625 U of Ampli Taq Gold DNA polymerase, 0.05 U of UNG and loaded in the TaqMan Low Density Array according to manufacturer recommendations. Taqman Low Density Array was run on an Applied Biosystem 7900HT. The amplification profile was 1 cycle of 2 min at 50° C., 1 cycle of 10 min at 94.5° C. and 40 cycles of 30 s at 97° C. and 1 min at 59.7° C. Raw data were analyzed using SDS 2.2 software (ABI). Ct values were obtained with automatic baseline and 0.15 as threshold value.

### Leave One Out Crossvalidation of SPCA-DA Classification Using the 22 Genes Q-PCR Data:

A classification scheme was developed and tested using crossvalidation by leave-one-out using all 22 genes measured by Q-PCR (i.e. without classifier feature recalculation).

First, the Z-score normalization was performed within each training set and applied to the test sample. Next, the same classification algorithm applied to microarray data based on supervised principal component—discriminant analysis (SPCA-DA) was built and applied to each of the samples left out in that loop (Bair and Tibshirai, PLOS Biol 2004 and Tibshirani et al., PNAS 2002).

Using the 0.43 cut-off from microarray, 33/54 samples are classified as GS+, sensitivity is 85% ( 17/20) with specificity 53% ( 18/34). Like in microarray, AS15 arm has better performance, 92% sensitivity and 57% specificity.

Using a cut-off of 0.47 calculated on PCR data, 31/54 samples are classified as GS+, sensitivity is 85% ( 17/20) and specificity is 59% ( 20/34).

52 samples tested on PCR were in the microarray model. We compared the classification of corresponding samples on LOO SPCA-DA microarray with 100PS (with feature selection) and LOO SPCA-DA PCR with 22 genes (without feature selection), both with cut-off of probability at 0.43. The concordance of sample classification between the leave one out model is 49 out of 52 samples having the same label in both classification (misclassified being borderline samples).

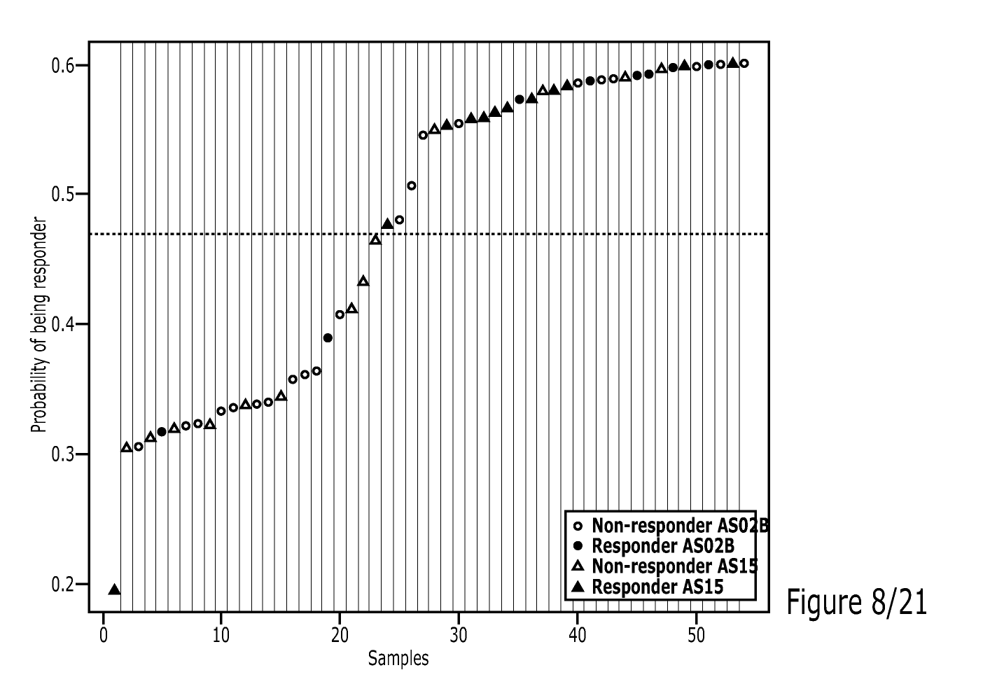

FIG. 8/21 shows the classifier indexes obtained by LOO SPCA-DA PCR with 22 genes (without feature selection).
Classification of a New Sample Using the Parameters Derived from the Training Set

For prediction of a new patient clinical outcome based on the Q-PCR expression levels for the 22 genes in the classifier, a supervised principal component (SPCA)—discriminant analysis (DA) decision rule is applied (adapted from Bair, 2004; Tibshirani, 2002) as shown previously for the microarray based classifier of example 1.

Once the patient raw data has been normalized using the reference genes and log transformed (this will be called expression matrix), they can be subjected to a decision rule (classifier or classification scheme) for prediction of the clinical outcome for the patient.
The expression matrix is z-scored using mean and standard deviation (Sd) from the training set (Table 6)
The z-scored normalized expression profile (classifier features) of the patient to classify is projected in the first principal component (PC₁) space defined by the training set using a linear combination of the classifier features (the coefficients for each of the 22 features in the linear combination was obtained by singular value decomposition of the training set and they are provided in Table 6).

TABLE 6
Mean, Standard deviations (Sd) and PC1 coefficients for 22 genes
classifier features

| | | | PC1 |
|---|---|---|---|
| Gene | Mean | Sd | coefficient |
| C4orf7 | −1.397 | 1.244 | −0.1834 |
| CCL5 | −0.545 | 0.691 | −0.2441 |
| JAK2 | −1.105 | 0.354 | −0.1636 |
| IRF1 | −0.430 | 0.500 | −0.2345 |
| CXCL9 | −0.276 | 0.923 | −0.2349 |
| IL2RG | −0.657 | 0.721 | −0.2444 |
| CXCL10 | −0.830 | 0.896 | −0.2181 |
| SLC26A2 | −0.745 | 0.307 | 0.0660 |
| CD86 | −1.504 | 0.461 | −0.2272 |
| CD8A | −1.342 | 0.879 | −0.1881 |
| UBD | −0.570 | 0.945 | −0.2385 |
| GZMK | −1.470 | 0.734 | −0.2414 |
| GPR171 | −1.683 | 0.698 | −0.2180 |
| PSCDBP | −1.335 | 0.647 | −0.2212 |
| CXCL2 | −2.163 | 0.633 | −0.1437 |
| ICOS | −1.714 | 0.697 | −0.2029 |
| TRBC1 | −2.714 | 1.313 | −0.2026 |
| TRA@; TRAJ17; TRDV2; TRAC; TRAV20 | −0.762 | 0.666 | −0.2464 |
| TARP; TRGC2 | −2.405 | 0.877 | −0.1904 |
| ITK | −1.862 | 0.896 | −0.2178 |
| CD3D | −1.478 | 0.806 | −0.2452 |
| HLA-DMA | −0.380 | 0.470 | −0.2284 |

The standardized distance of the test sample in PC1 to the mean of the Responder and non responder group is obtained using the following equation:

$${d}_{iK} = \frac{\vert {PC}_{1 i} - {mean\_PC}_{1 K} \vert}{{sd\_PC}_{1 K}}$$

i=test sample
K=Responder (R) or Non-Responder (NR)
mean_PC_{1K}=PC₁ mean of R or NR group in training set
sd_PC_{1K}=PC₁ standard deviation of R or NR group in training set
The mean and sd of each group in the training set (rounded to three significant digits) are:

| | |
|---|---|
| mean_PC_{1R} | −2.055 |
| sd_PC_{1R} | 2.920 |
| mean_PC_{1NR} | 1.210 |
| sd_PC_{1NR} | 3.951 |

The index (probability of sample being Responder) for each sample is obtained with:

$${P}_{R} = \frac{{e}^{- \frac{{d}_{iR}}{2}}}{{e}^{- \frac{{d}_{iR}}{2}} + {e}^{- \frac{{d}_{iNR}}{2}}}$$

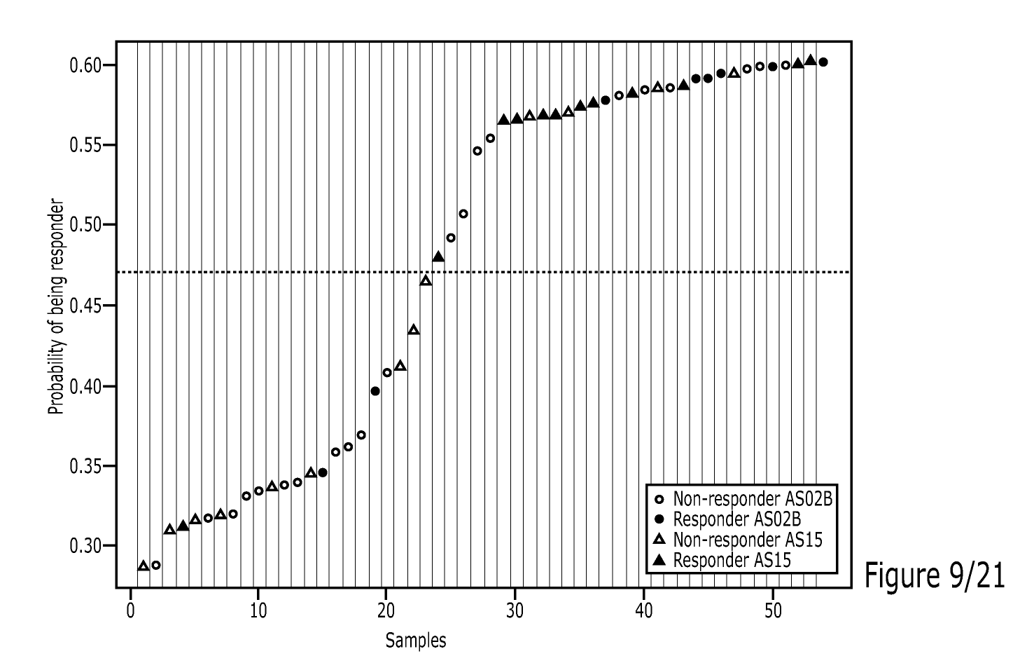

A sample is classified as gene signature positive (Responder,R) if its P_{R} is greater than 0.47
   Applying this classifier to the training set, produces FIG. 9/21 which shows that the 22 genes can classify the train set with sensitivity of 0.85 ( 17/20) and specificity of 0.59 ( 20/34), for a 69% concordance.

### Outcome Prediction Code

### Script for classification of test-samples fresh metatasic
melanoma TLDA2 22 genes
### based on Mage008TLDA.SPCA.DA.Mel4patent.R
### needs M8.train.parameters.22genes.TLDA2.RData (training set
parameters)
library(genefilter)
#### load testset to classify (log-scaled normalized PCR data)
load(“testset.RData”) ### ExpressionSet containing samples to
classify
### Load training set parameters ##############
load(“M8.train.parameters.22genes.TLDA2.RData”)
PS<-M8.train.parameters[[1]]
M8.train.means<-M8.train.parameters[[2]]
M8.train.sd<-M8.train.parameters[[3]]
M8.train.U<-M8.train.parameters[[4]]
M8.trainPC1barRs<-M8.train.parameters[[5]]
M8.trainPC1sdRs<-M8.train.parameters[[6]]
M8.trainPC1barNRs<-M8.train.parameters[[7]]
M8.trainPC1sdNRs<-M8.train.parameters[[8]]
######################### Use SPCA on test set -
#######################
testset<-testset[PS,]
test<-(exprs(testset)−M8.train.means)/M8.train.sd
PCtest<-t(test) %*% M8.train.U
PC1test<-PCtest[,1]
distanceR<-c( )
distanceNR<-c( )
probR<-c( )
probNR<-c( )
SPCAclass<-c( )
for (i in 1:ncol(test)) {
distancesR<-abs(PCtest[i,1]−M8.trainPC1barRs)/M8.trainPC1sdRs
distancesNR<-abs(PCtest[i,1]−M8.trainPC1barNRs)/M8.trainPC1sdNRs
distanceR<-c(distanceR,distancesR)
distanceNR<-c(distanceNR,distancesNR)
probRs<-exp(−distancesR/2)/(exp(−distancesR/2)+exp(−
distancesNR/2))
probNRs<-exp(−distancesNR/2)/(exp(−distancesR/2)+exp(−
distancesNR/2))
probR<-c(probR,probRs)
probNR<-c(probNR,probNRs)
}
cutoff=0.47
clust<-ifelse(as.vector(probR)>cutoff,R,NR)
####################
###(modify xx next line according to batch number)
write.table(cbind(pData(testset),probR),file=
“testset_batch_xx_TLDA2_22genes_classification.txt”,sep=“\t”)

### Where

Testset.RData is a matrix with 22 rows containing the normalized log-scaled PCR data for the 22 genes
M8.train.parameters is an object of class list containing:
   1. a character list of the 22 gene names
   2. a vector of 22 mean values for each gene in the train set
   3. a vector of 22 sd values for each gene in the train set
   4. a matrix of 22 rows and 22 columns containing the U matrix of the svd decomposition of the train matrix
   5. the PC1 mean value of the responder group in the train
   6. the PC1 sd value of the responder group in the train
   7. the PC1 mean value of the non-responder group in the train
   8. the PC1 sd value of the non-responder group in the train

### Example 3

Classification of NSCLC Samples with a Subset of 23 Genes Assessed by PCR Background

### NSCLC Phase II Clinical Trial

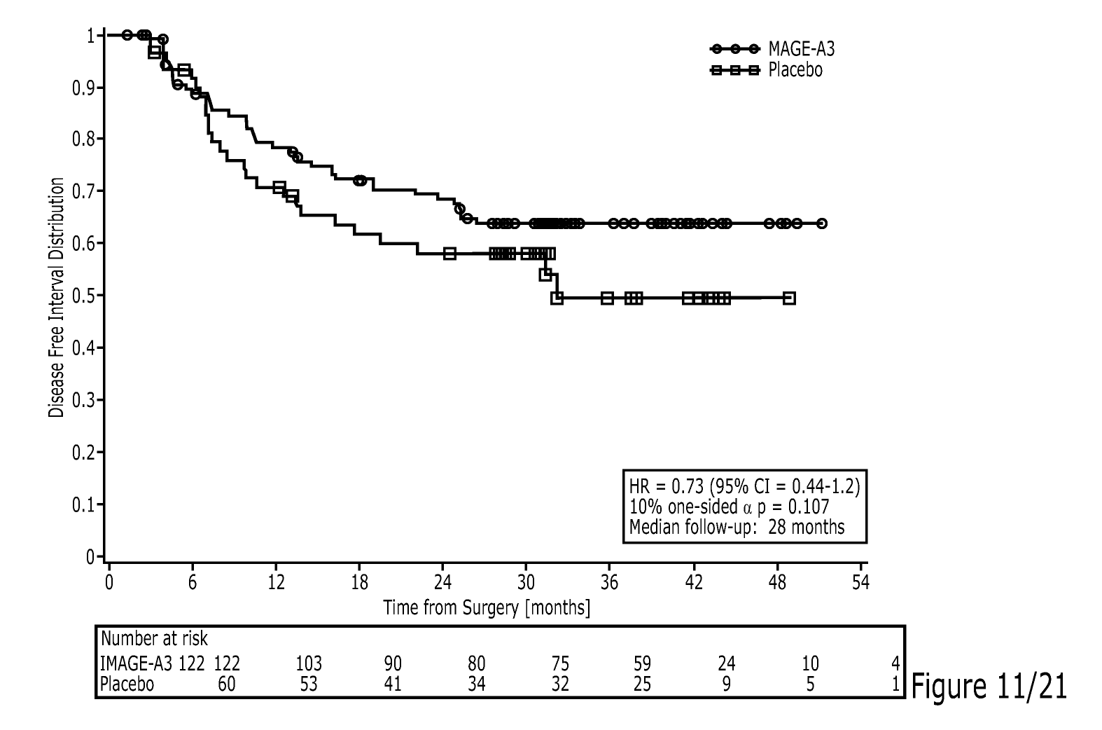

This is a double blind placebo controlled proof-of-concept trial in MAGE-A3 positive, stage IB and II NSCLC patients after complete surgical resection of the tumor (CPMS 249553/004). The ASCI (Antigen-Specific Cancer Immonotherapeutics) agent is the recombinant MAGE-A3 fusion protein in fusion with Protein-D and a Hist-tail. It is combined with AS02B immunological adjuvant. AS02B is an oil-in-water emulsion of QS21 and MPL. QS21 is a purified, naturally occurring saponin molecule from the South-American tree Quillaja Saponaria Molina, and MPL 3 de-O-acetylated monophosphoryl lipid A—detoxified derivative of lipid A, derived from S. minnesota LPS. This double-blind, randomized, placebo-controlled trial was designed to evaluate the time to recurrence (FIG. 11/21).

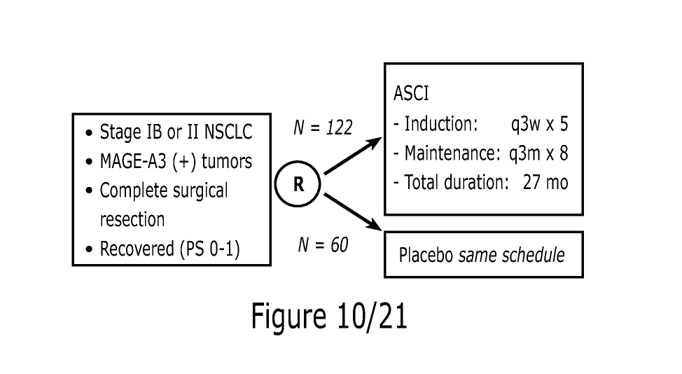

FIG. 10/21 shows the NSCLC Phase II trial design. A total of 182 patients with MAGE-A3-positive, completely resected, stage IB or II NSCLC were enrolled over 2 years and randomly assigned to receive either the ASCI targeting MAGE-A3 or placebo (2:1 ratio). A maximum of 13 doses were administered over a period of 27 months. The main analysis was performed after a median follow-up period of 28 months from resection date and was released in November 2006.

This trial provided the first evidence of activity for a cancer immunotherapy in this patient population. At the time of the main analysis, 67 patients had shown disease recurrence: 41 in the recMAGE-A3+AS02B ASCI arm (33.6%) and 26 in the placebo arm (43.3%). A Cox regression analysis was used to calculate the relative improvement in Disease-Free Interval (DFI) while taking into account the individual time-to-event of each patient. The results show a 27% relative reduction in risk of cancer recurrence after a 28-month median follow-up in the group receiving the ASCI when compared to placebo (Hazard ratio=0.73; CI=0.44-1.2; p=0.108, one-sided logrank test) (FIG. 11/21).

Hazard ratios for Disease-Free Survival (DFS) and Overall Survival (OS) were 0.73 (CI: 0.45-1.16), and 0.66 (CI=0.36-1.20), respectively.

These results were further confirmed at the time of final analysis (December 2007-median follow-up of 44 months): HR 0.75 for DFI (CI=0.46-1.23), 0.76 for DFS (CI=0.48-1.21) and 0.81 for OS (CI=0.47-1.40).

FIG. 11/21 shows the Kaplan-Meier curve for Disease-Free Interval for the NSCLC trial. Samples from this study were used to determine use of the melanoma signature as potential biomarkers predictive of the ASCI-treatment clinical response in this patient population.

Classification of NSCLC Samples with PCR Data:

A subset of 23 genes from 100PS (Table-1) was used to build a LOO classifier with the samples from the MAGE-A3 NSCLC clinical trial (MAGE004; GlaxoSmithKline)

TABLE 7
ABI Taqman Assay numbers for 23 genes used to build PCR based
classifier in NSCLC samples (reference genes same as melanoma
classifier in example 2)
23 genes in 100PS measured by PCR

| Gene | | |
|---|---|---|
| symbol | Gene Name | Taqman Assay |
| CCL5 | chemokine (C-C motif) ligand 5 | Hs00174575_m1 |
| JAK2 | Janus kinase 2 (a protein tyrosine kinase) | Hs01078136_m1 |
| IRF1 | interferon regulatory factor 1 | Hs00971960_m1 |
| CXCL9 | chemokine (C—X—C motif) ligand 9 | Hs00171065_m1 |
| IL2RG | interleukin 2 receptor, gamma (severe | Hs00173950_m1 |
| | combined | |
| | immunodeficiency) | |
| CXCL10 | chemokine (C—X—C motif) ligand 10 | Hs00171042_m1 |
| SLC26A2 | solute carrier family 26 (sulfate | Hs00164423_m1 |
| | transporter), member 2 | |
| CD86 | CD86 molecule | Hs01567025_m1 |
| CD8A | CD8a molecule | Hs00233520_m1 |
| UBD | ubiquitin D | Hs00197374_m1 |
| GZMK | granzyme K (granzyme 3; tryptase II) | Hs00157878_m1 |
| GPR171 | G protein-coupled receptor 171 | Hs00664328_s1 |
| PSCDBP | pleckstrin homology, Sec7 and coiled-coil | Hs00188734_m1 |
| | domains, | |
| | binding protein | |
| CXCL2 | chemokine (C—X—C motif) ligand 2 | Hs00236966_m1 |
| ICOS | inducible T-cell co-stimulator | Hs99999163_m1 |
| TRBC1 | T cell receptor beta constant 2 | Hs00411919_m1 |
| TRA@; | T cell receptor alpha locus | Hs00948942_m1 |
| TRAJ17; | | |
| TRDV2; | | |
| TRAC; | | |
| TRAV20 | | |
| TARP; | TCR gamma alternate reading frame | Hs00827007_m1 |
| TRGC2 | protein; T cell | |
| | receptor gamma constant 2 | |
| ITK | IL2-inducible T-cell kinase | Hs00950634_m1 |
| C4orf7 | chromosome 4 open reading frame 7 | Hs00395131_m1 |
| CD3D | CD3d molecule, delta (CD3-TCR | Hs00174158_m1 |
| | complex) | |
| HLA- | major histocompatibility complex, class II, | Hs00185435_m1 |
| DMA | DM alpha | |
| SLAMF7 | SLAM family member 7 | Hs00900280_m1 |

### Methods

129 tumor specimens (pre-vaccination) were used from MAGE-A3 NSCLC clinical trial (MAGE004; GlaxoSmithKline). These were fresh frozen samples preserved in the RNAlater, a RNA stabilizing solution. Total RNA was purified using the Tripure method (Roche Cat. No. 1 667 165). The recommended protocol was followed subsequently by the use of an RNeasy Mini kit—clean-up protocol with DNAse treatment (Qiagen Cat. No. 74106). Quantification of RNA was initially completed using optical density at 260 nm.

cDNA synthesis from 500 ng of total RNA was performed in a 20 μl mixture containing 1× first strand buffer, 0.5 mM of each dNTP, 10 mM of dithiothreitol, 20 U of rRNase inhibitor (Promega cat.N2511), 250 ng of Random hexamers and 200 U of M-MLV reverse transcriptase (Life Technologies cat. 28025-013) for 1 h30 at 42° C. cDNA corresponding to 200 ng of total RNA was mixed in a total volume of 200 μl containing TaqMan buffer, 5 mM MgCl2, 0.4 mM dUTP, 0.625 U of Ampli Taq Gold DNA polymerase, 0.05 U of UNG and loaded in the TaqMan Low Density Array according to manufacturer recommendations.

Taqman Low Density Array was run on an Applied Biosystem 7900HT. The amplification profile was 1 cycle of 2 min at 50° C., 1 cycle of 10 min at 94.5° C. and 40 cycles of 30 s at 97° C. and 1 min at 59.7° C. Raw data were analyzed using SDS 2.2 software (ABI). Ct values were obtained with automatic baseline and 0.15 as threshold value.

### Leave One Out Crossvalidation of SPCA-Cox Classification Using the 23 Genes Q-PCR Data:

This clinical trial contained a placebo and treated arm, a classifier was developed that uses disease free interval (DFI) to estimate a risk score based on a Cox proportional hazards model with an interaction between treatment and gene profile (summarized as principal component 1) in addition to treatment, gene profile, stage, surgery and histologic type as covariates.

Ct values for each gene were normalized with the geometric mean of the 5 reference genes and log-transformed. Subsequently, the genes were normalized by Z-score in each training set and these parameters applied to test set.

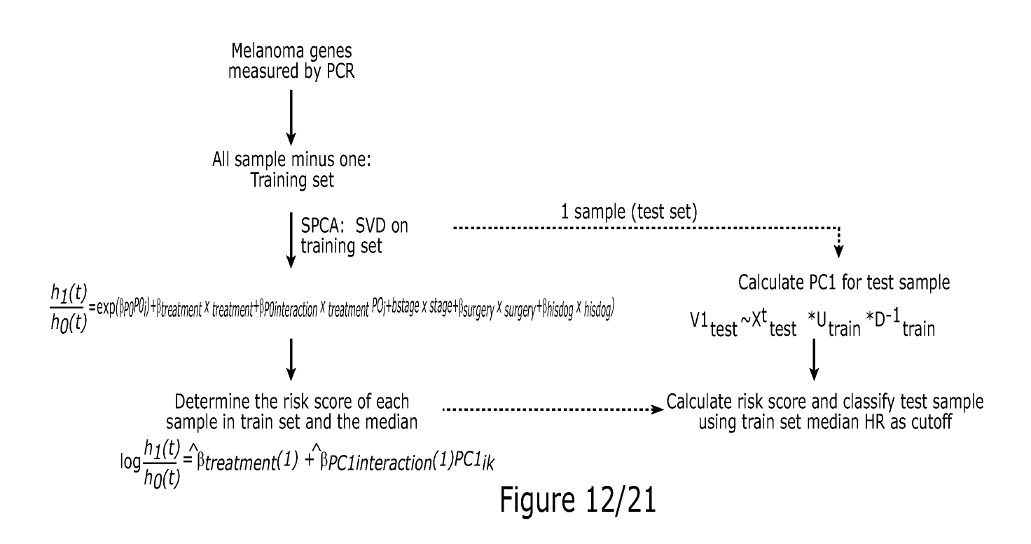

After z-score normalization, a singular value decomposition (SVD) is performed in the training set to obtain the first Principal Component (PC1). This first component is used in a Cox regression with interaction with treatment to estimate the covariates coefficient in the train set; the Cox regression is adjusted for histology, stage and type of surgery effects. The coefficients from this regression are used to calculate Risk Score in the training set and the test sample (left out sample). The median Risk Score of the train set is used as cut-off value to call a patient gene signature (GS)+ or gene signature (GS)−. This methodology is called Cox-SPCA and is illustrated in FIG. 12/21.

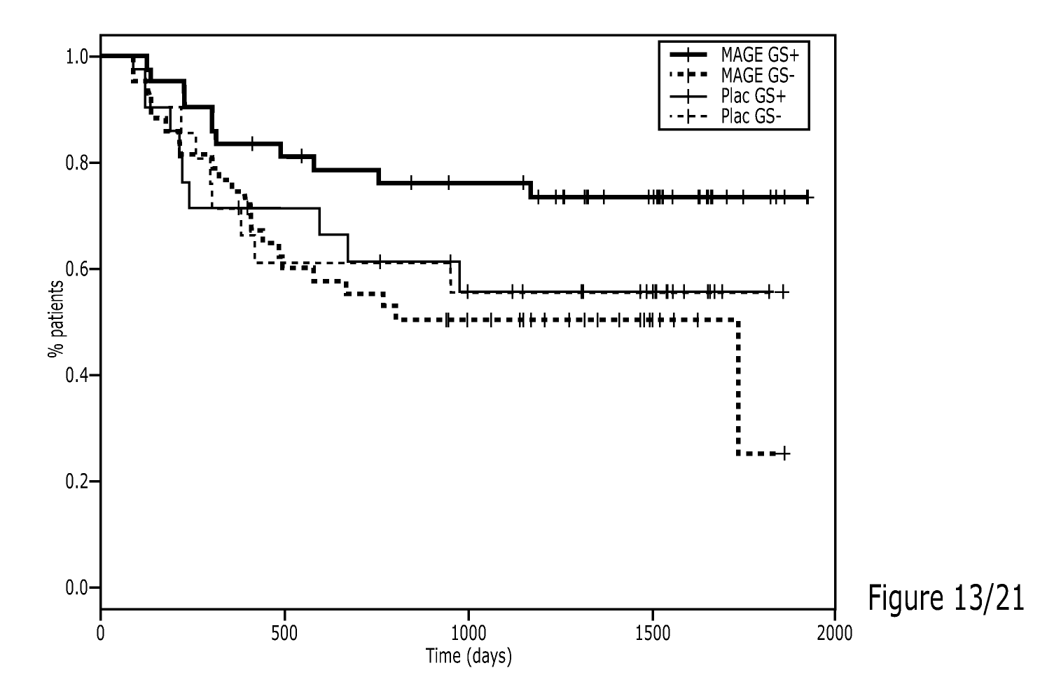

FIGS. 13/21 and 14/21 show survival curves by gene profile based on the LOOCV classification with median as cut-off and distribution of risk score among placebo and vaccine arm, respectively. The Risk score distribution is as follows:

Impact of GS on HR

| | HR treatment | CI |
|---|---|---|
| GS+ | 0.466 | [0.187; 1.162] |
| GS− | 1.216 | [0.555; 2.67] |

### Classification of a New Sample Using the Cox-SPCA Algorithm

For prediction of a new patient clinical outcome based on the Q-PCR expression levels for the 23 genes in the classifier, a supervised principal component (SPCA)—Cox decision rule is applied:

Once the patient raw data has been normalized using the reference genes and log transformed, they can be subjected to a decision rule (classifier or classification scheme) for prediction of the clinical outcome for the patient.
The expression matrix is z-scored using the parameters of the training set (Table 8)

TABLE 8
Mean, Standard deviations (Sd) and PC1 coefficients for 23 genes
classifier features

| | | | PC1 |
|---|---|---|---|
| Gene | Mean | sd | coefficient |
| C4orf7 | −2.35768 | 1.455544 | −0.12114 |
| CCL5 | −0.9599 | 0.350039 | −0.23097 |
| JAK2 | −1.36811 | 0.260374 | −0.19931 |
| IRF1 | −0.52347 | 0.276644 | −0.2256 |
| CXCL9 | −0.87804 | 0.563437 | −0.21386 |
| IL2RG | −0.83528 | 0.358042 | −0.24997 |
| CXCL10 | −1.36857 | 0.615177 | −0.17136 |
| SLC26A2 | −1.44043 | 0.255169 | −0.05637 |
| CD86 | −1.7699 | 0.499237 | −0.13267 |
| CD8A | −1.33733 | 0.375334 | −0.25173 |
| UBD | −0.71367 | 0.546652 | −0.21295 |
| GZMK | −1.77411 | 0.529496 | −0.24628 |
| GPR171 | −1.81327 | 0.32409 | −0.19376 |
| PSCDBP | −1.17746 | 0.387117 | −0.24162 |
| CXCL2 | −1.16947 | 0.696255 | −0.09696 |
| ICOS | −2.15436 | 0.403522 | −0.23497 |
| TRBC1 | −2.62512 | 1.013281 | −0.12679 |
| TRA@; TRAJ17; TRDV2; TRAC; | −1.19671 | 0.3944 | −0.25817 |
| TRAV20 | | | |
| TARP; TRGC2 | −2.22752 | 0.481252 | −0.19299 |
| ITK | −1.85777 | 0.394118 | −0.26077 |
| CD3D | −1.64584 | 0.397626 | −0.25514 |
| HLA-DMA | −0.81144 | 0.380465 | −0.22948 |
| SLAMF7 | −1.33744 | 0.464338 | −0.21762 |

The z-scored normalized expression profile (classifier features) of the patient to classify is projected in the first principal component (PC₁) space defined by the training set using a linear combination of the classifier features (the coefficients for each of the 23 features in the linear combination was obtained by singular value decomposition of the training set and they are provided in Table 8)
A risk score for the new sample is calculated using the equation:

$$log \frac{{h}_{i} ( t )}{{h}_{0} ( t )} {\hat{β}}_{treatment} ( 1 ) + {\hat{β}}_{PC 1 interaction} ( 1 ) PC {1}_{ik}$$

### Where Bₜᵣₑₐₜₘₑₙₜ=−0.232051457

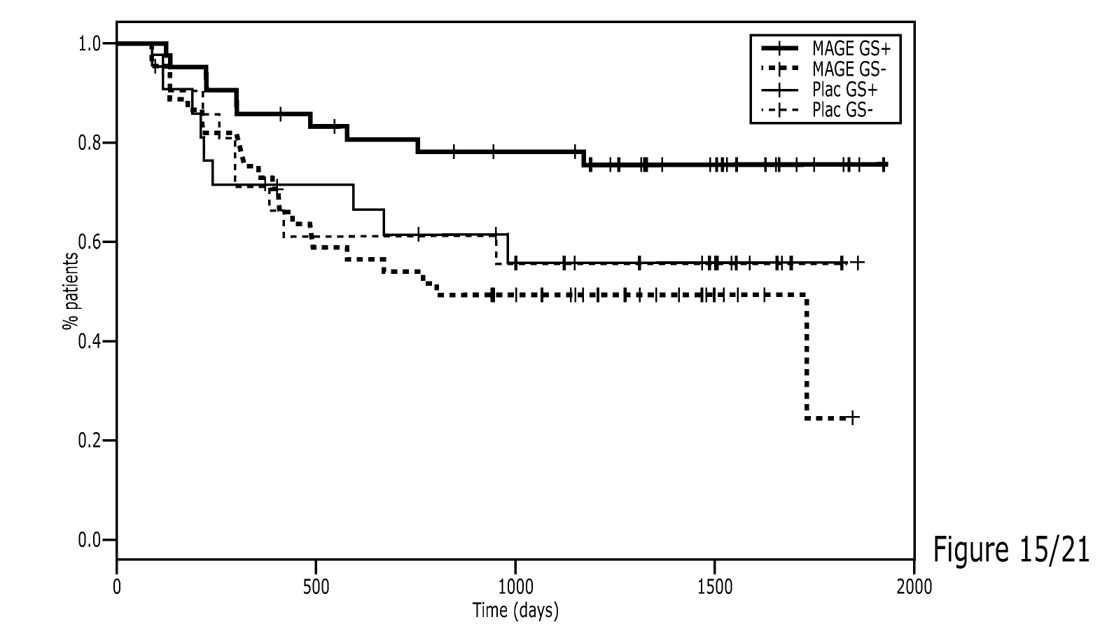

and B_{PC1interaction}=0.176736586 were obtained from the training set
The risk score of the new sample is compared to the median risk score of the training set=−0.315324195
and the sample is classified GS+ (Responder, Non-Relapse,1) if Risk score is lower than this value.
FIGS. 15/21 and 16/21 show the clinical outcome based on the Q-PCR expression levels for the 23 genes in the classifier. The impact of GS on HR is as follows:

Impact of GS on HR

| | HR treatment | CI |
|---|---|---|
| GS+ | 0.426 | [0.167; 1.090] |
| GS− | 1.248 | [0.572; 2.720] |

### Outcome Prediction Code

### Script for classification of test-samples fresh resected
NSCLC TLDAmerge 23 genes
### based on
Mage004.SPCA.Cox.classifier.contruction.TLDAmerge.23genes.DFI.
Squamous.R
### needs M4.train.parameters.23genes.TLDAmerge.RData (training
set parameters)
library(genefilter)
#### load testset to classify (log-scaled normalized PCR data)
load(“testset.RData”) ### ExpressionSet containing samples to
classify
### Load training set parameters ##############
load(“M4.train.parameters.23genes.TLDAmerge.RData”)
PS<-M4.train.parameters[[1]]
M4.train.means<-M4.train.parameters[[2]]
M4.train.sd<-M4.train.parameters[[3]]
M4.train.U<-M4.train.parameters[[4]]
M4.train.Btreatment<-M4.train.parameters[[5]]
M4.train.Binteraction<-M4.train.parameters[[6]]
M4.train.medianHR<-M4.train.parameters[[7]]
################################## Use SPCA on test set -
#######################
testset<-testset[PS,]
test<-(exprs(testset)-M4.train.means)/M4.train.sd
PCtest<-t(test) %*% M4.train.U
PC1test<-PCtest[,1]
HR=M4.train.Btreatment+PC1test*M4.train.Binteraction
classification=ifelse(HR<M4.train.medianHR,1,0)
####################
###(modify xx next line according to batch number)
write.table(cbind(pData(testset),probR),file=
“testset_batch_xx_M4_TLDAmerge_23genes_classification.txt”,
sep=“\t”)

### Where

Testset.RData is a matrix with 23 rows containing the normalized log-scaled PCR data for the 23 genes
M4.train.parameters is an object of class list containing:
   1. a character list of the 23 gene names
   2. a vector of 23 mean values for each gene in the train set
   3. a vector of 23 sd values for each gene in the train set
   4. a matrix of 23 rows and 23 columns containing the U matrix of the svd decomposition of the train matrix
   5. the Bₜᵣₑₐₜₘₑₙₜ in risk score computation
   6. the B_{PC1interaction} in risk score computation
   7. the median risk score in train

### Example 4

### Classification of NSCLC Samples with a Subset of 22 Genes Assessed by PCR

A subset of 22 genes from 100PS (Table-1) was used to build a LOO classifier with the samples from the MAGE-A3 NSCLC clinical trial (MAGE004; GlaxoSmithKline)

TABLE 9
ABI Taqman Assay numbers for 22 genes used to build PCR based
classifier in NSCLC samples (reference genes same as melanoma
classifier in example 2)
22 genes in 100PS measured by PCR

| Gene | | |
|---|---|---|
| symbol | Gene Name | Taqman Assay |
| CCL5 | chemokine (C-C motif) ligand 5 | Hs00174575_m1 |
| JAK2 | Janus kinase 2 (a protein tyrosine kinase) | Hs01078136_m1 |
| IRF1 | interferon regulatory factor 1 | Hs00971960_m1 |
| CXCL9 | chemokine (C—X—C motif) ligand 9 | Hs00171065_m1 |
| IL2RG | interleukin 2 receptor, gamma (severe | Hs00173950_m1 |
| | combined | |
| | immunodeficiency) | |
| CXCL10 | chemokine (C—X—C motif) ligand 10 | Hs00171042_m1 |
| SLC26A2 | solute carrier family 26 (sulfate | Hs00164423_m1 |
| | transporter), member 2 | |
| CD86 | CD86 molecule | Hs01567025_m1 |
| CD8A | CD8a molecule | Hs00233520_m1 |
| UBD | ubiquitin D | Hs00197374_m1 |
| GZMK | granzyme K (granzyme 3; tryptase II) | Hs00157878_m1 |
| GPR171 | G protein-coupled receptor 171 | Hs00664328_s1 |
| PSCDBP | pleckstrin homology, Sec7 and coiled-coil | Hs00188734_m1 |
| (CYTIP) | domains, | |
| | binding protein | |
| CXCL2 | chemokine (C—X—C motif) ligand 2 | Hs00236966_m1 |
| ICOS | inducible T-cell co-stimulator | Hs99999163_m1 |
| TRBC1 | T cell receptor beta constant 2 | Hs00411919_m1 |
| TRA@; | T cell receptor alpha locus | Hs00948942_m1 |
| TRAJ17; | | |
| TRDV2; | | |
| TRAC; | | |
| TRAV20 | | |
| TARP; | TCR gamma alternate reading frame | Hs00827007_m1 |
| TRGC2 | protein; T cell | |
| | receptor gamma constant 2 | |
| ITK | IL2-inducible T-cell kinase | Hs00950634_m1 |
| C4orf7 | chromosome 4 open reading frame 7 | Hs00395131_m1 |
| CD3D | CD3d molecule, delta (CD3-TCR | Hs00174158_m1 |
| | complex) | |
| HLA- | major histocompatibility complex, class II, | Hs00185435_m1 |
| DMA | DM alpha | |

### Methods

137 tumor specimens (pre-vaccination) were used from MAGE-A3 NSCLC clinical trial (MAGE004; GlaxoSmithKline). These were fresh frozen samples preserved in the RNAlater, a RNA stabilizing solution.

Total RNA was purified using the Tripure method (Roche Cat. No. 1 667 165). The recommended protocol was followed subsequently by the use of an RNeasy Mini kit—clean-up protocol with DNAse treatment (Qiagen Cat. No. 74106). Quantification of RNA was initially completed using optical density at 260 nm.

cDNA synthesis from 500 ng of total RNA was performed in a 20 μl mixture containing 1× first strand buffer, 0.5 mM of each dNTP, 10 mM of dithiothreitol, 20 U of rRNase inhibitor (Promega cat.N2511), 250 ng of Random hexamers and 200 U of M-MLV reverse transcriptase (Life Technologies cat. 28025-013) for 1 h30 at 42° C. cDNA corresponding to 200 ng of total RNA was mixed in a total volume of 200 μl containing TaqMan buffer, 5 mM MgCl2, 0.4 mM dUTP, 0.625 U of Ampli Taq Gold DNA polymerase, 0.05 U of UNG and loaded in the TaqMan Low Density Array according to manufacturer recommendations.

Taqman Low Density Array was run on an Applied Biosystem 7900HT. The amplification profile was 1 cycle of 2 min at 50° C., 1 cycle of 10 min at 94.5° C. and 40 cycles of 30 s at 97° C. and 1 min at 59.7° C. Raw data were analyzed using SDS 2.2 software (ABI). Ct values were obtained with automatic baseline and 0.15 as threshold value.

### Leave One Out Crossvalidation of SPCA-Cox Classification Using the 22 Genes Q-PCR Data:

This clinical trial contained a placebo and treated arm, a classifier was developed that uses disease free interval (DFI) to estimate a risk score based on a Cox proportional hazards model with an interaction between treatment and gene profile (summarized as principal component 1) in addition to treatment, gene profile, stage, surgery and histologic type as covariates

Ct values for each gene were normalized with the geometric mean of the 5 reference genes and log-transformed. Subsequently, the genes were normalized by Z-score in each training set and these parameters applied to test set.

After z-score normalization, a singular value decomposition (SVD) is performed in the training set to obtain the first Principal Component (PC1). This first component is used in a Cox regression with interaction with treatment to estimate the covariates coefficient in the train set; the Cox regression is adjusted for histology, stage and type of surgery effects. The coefficients from this regression are used to calculate Risk Score in the training set and the test sample (left out sample). The median Risk Score of the train set is used as cut-off value to call a patient GS+ or GS−. This methodology is called Cox-SPCA in further document. The methodology is illustrated in FIG. 12/21.

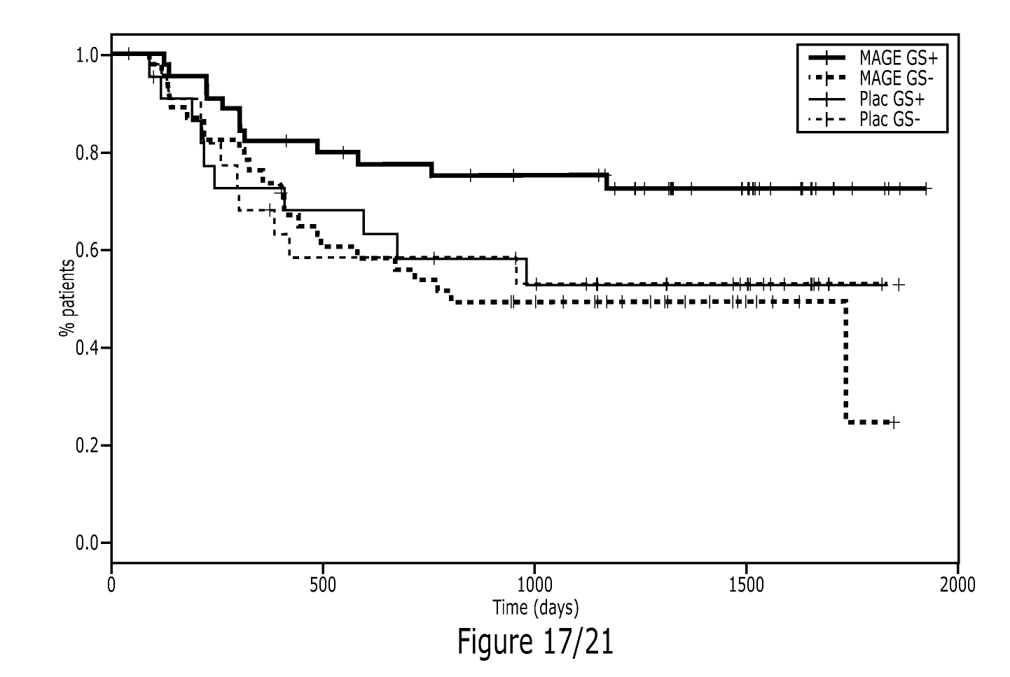

FIGS. 17/21 and 18/21 show survival curves by gene profile based on the LOOCV classification with median as cut-off and distribution of risk score among placebo and vaccine arm, respectively.

### Risk Score Distribution

Impact of GS on HR

| | HR treatment | CI |
|---|---|---|
| GS+ | 0.460 | [0.193; 1.097] |
| GS− | 1.197 | [0.564; 2.541] |

### Classification of a New Sample Using the Cox-SPCA Algorithm

For prediction of a new patient clinical outcome based on the Q-PCR expression levels for the 22 genes in the classifier, a supervised principal component (SPCA)-Cox decision rule is applied:

Once the patient raw data has been normalized using the reference genes and log transformed, they can be subjected to a decision rule (classifier or classification scheme) for prediction of the clinical outcome for the patient.
The expression matrix is z-scored using the parameters of the training set (Table 10)

TABLE 10
Mean, Standard deviations (Sd) and PC1 coefficients for 22 genes
classifier features

| | | | PC1 |
|---|---|---|---|
| Gene | Means | Sd | coefficients |
| C4orf7 | −2.37682 | 1.432191 | −0.12613 |
| CCL5 | −0.97196 | 0.363545 | −0.23868 |
| JAK2 | −1.38351 | 0.272662 | −0.20067 |
| IRF1 | −0.5328 | 0.284196 | −0.23035 |
| CXCL9 | −0.88518 | 0.561561 | −0.21758 |
| IL2RG | −0.84755 | 0.369696 | −0.25893 |
| CXCL10 | −1.38526 | 0.608373 | −0.17545 |
| SLC26A2 | −1.45138 | 0.259368 | −0.06122 |
| CD86 | −1.78136 | 0.493304 | −0.1445 |
| CD8A | −1.35019 | 0.38214 | −0.26018 |
| UBD | −0.72426 | 0.545598 | −0.21573 |
| GZMK | −1.7857 | 0.526042 | −0.25378 |
| GPR171 | −1.81382 | 0.353983 | −0.1875 |
| PSCDBP | −1.19407 | 0.398912 | −0.24969 |
| CXCL2 | −1.17377 | 0.679063 | −0.10145 |
| ICOS | −2.16745 | 0.40877 | −0.24479 |
| TRBC1 | −2.63145 | 0.999466 | −0.12889 |
| TRA@; TRAJ17; TRDV2; TRAC; | −1.20289 | 0.392963 | −0.26276 |
| TRAV20 | | | |
| TARP; TRGC2 | −2.27109 | 0.528402 | −0.19113 |
| ITK | −1.87391 | 0.405727 | −0.26852 |
| CD3D | −1.66653 | 0.409356 | −0.26013 |
| HLA-DMA | −0.81888 | 0.400541 | −0.23598 |

The z-scored normalized expression profile (classifier features) of the patient to classify is projected in the first principal component (PC₁) space defined by the training set using a linear combination of the classifier features (the coefficients for each of the 22 features in the linear combination was obtained by singular value decomposition of the training set and they are provided in Table 10)
A risk score for the new sample is calculated using the equation:

$$log \frac{{h}_{i} ( t )}{{h}_{0} ( t )} {\hat{β}}_{treatment} ( 1 ) + {\hat{β}}_{PC 1 interaction} ( 1 ) PC {1}_{ik}$$

Where Bₜᵣₑₐₜₘₑₙₜ=−0.193146993 and B_{PC1interaction}=0.163704817 were obtained from the training set

The risk score of the new sample is compared to the median risk score of the training set=−0.25737421 and the sample is classified GS+ (Responder, Non-Relapse,1) if Risk score is lower than this value.

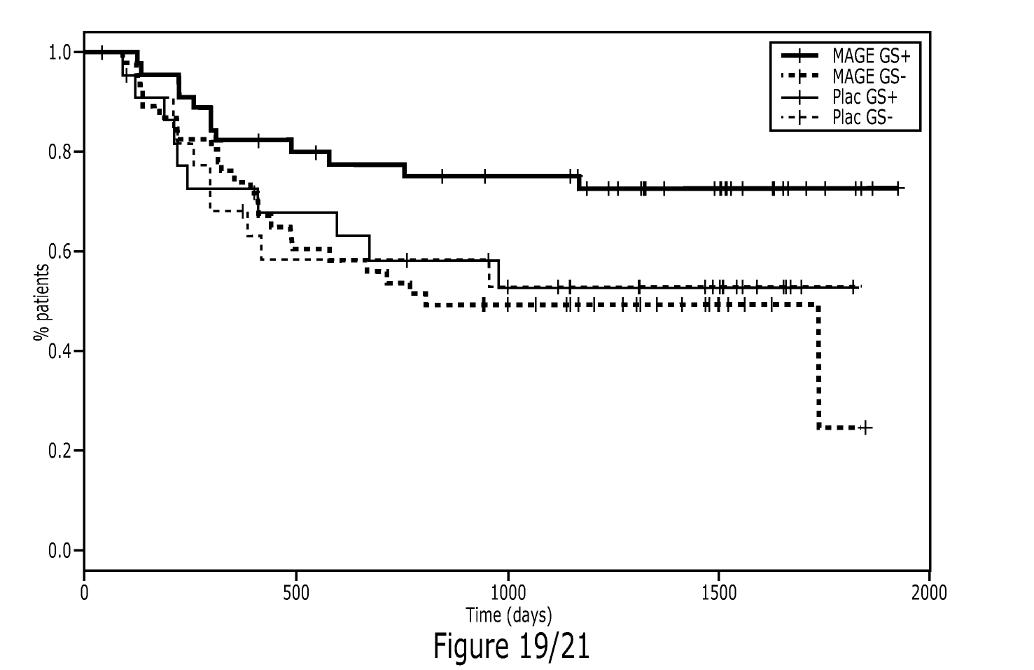

FIGS. 19/21 and 20/21 show the clinical outcome based on the Q-PCR expression levels for the 22 genes in the classifier.

Impact of GS on HR

| | HR treatment | CI |
|---|---|---|
| GS+ | 0.474 | [0.1990; 1.130] |
| GS− | 1.143 | [0.542; 2.438] |

### Outcome Prediction Code

### Script for classification of test-samples fresh resected
NSCLC TLDAmerge 22 genes
### based on Mage004.SPCA.Cox.classifier.contruction.
DFI.Squamous.R
### needs M4.train.parameters.22genes.TLDA2.RData (training set
parameters)
library(genefilter)
#### load testset to classify (log-scaled normalized PCR data)
load(“testset.RData”) ### ExpressionSet containing samples to
classify
### Load training set parameters ##############
load(“M4.train.parameters.22genes.TLDA2.RData”)
PS<-M4.train.parameters[[1]]
M4.train.means<-M4.train.parameters[[2]]
M4.train.sd<-M4.train.parameters[[3]]
M4.train.U<-M4.train.parameters[[4]]
M4.train.Btreatment<-M4.train.parameters[[5]]
M4.train.Binteraction<-M4.train.parameters[[6]]
M4.train.medianHR<-M4.train.parameters[[7]]
################################## Use SPCA on test set -
#######################
testset<-testset[PS,]
test<-(exprs(testset)-M4.train.means)/M4.train.sd
PCtest<-t(test) %*% M4.train.U
PC1test<-PCtest[,1]
HR=M4.train.Btreatment+PC1test*M4.train.Binteraction
classification=ifelse(HR<M4.train.medianHR,1,0)
####################
###(modify xx next line according to batch number)
write.table(cbind(pData(testset),probR),file=
“testset_batch_xx_M4_TLDA2_22genes_classification.txt”,sep=“\t”)

### Where

Testset.RData is a matrix with 22 rows containing the normalized log-scaled PCR data for the 22 genes
M4.train.parameters is an object of class list containing:
   1. a character list of the 22 gene names
   2. a vector of 22 mean values for each gene in the train set
   3. a vector of 22 sd values for each gene in the train set
   4. a matrix of 22 rows and 22 columns containing the U matrix of the svd decomposition of the train matrix
   5. the Bₜᵣₑₐₜₘₑₙₜ in risk score computation
   6. the B_{PC1interaction} in risk score computation
   7. the median risk score in train

### Example 5

### Classification Performance of Individual Genes Measured by Q-PCR in Melanoma Samples

Each of the 22 genes from example 2 were evaluated for univariate classification performance by using the algorithm applied to multivariate classification in melanoma samples using single gene expression values instead of the first principal component. After normalizing the expression values using the reference genes and performing a z-score, the expression levels for each individual gene were used to build the classifier using all samples in training set. The t-test p-value for differential expression of each gene in the training set and the fold change of Responders vs Non-Responders was calculated. The probability of each sample in the training set being responder was obtained and the best cutoff was determined for each gene by maximizing the concordance with clinical label and the results are shown in the next table:

TABLE 11

| | Concordance | t-test p- | |
|---|---|---|---|
| Gene | (%) | value | Fold Change |
| CCL5 | 72 | 0.003 | 3.7 |
| JAK2 | 67 | 0.010 | 1.8 |
| IRF1 | 72 | 0.004 | 2.5 |
| CXCL9 | 76 | 0.010 | 4.6 |
| IL2RG | 69 | 0.006 | 3.5 |
| CXCL10 | 69 | 0.004 | 5.2 |
| SLC26A2 | 63 | 0.030 | 0.7 |
| CD86 | 67 | 0.049 | 1.8 |
| CD8A | 74 | 0.095 | 2.6 |
| UBD | 70 | 0.001 | 7.0 |
| GZMK | 67 | 0.023 | 2.9 |
| GPR171 | 65 | 0.084 | 2.2 |
| PSCDBP | 65 | 0.005 | 3.1 |
| CXCL2 | 83 | 0.003 | 3.3 |
| ICOS | 67 | 0.004 | 3.5 |
| C4orf7 | 74 | 0.008 | 8.2 |
| TRA@; TRAJ17; TRDV2; TRAC; | 72 | 0.001 | 4.1 |
| TRAV20 | | | |
| TARP; TRGC2 | 70 | 0.003 | 5.1 |
| ITK | 76 | 0.062 | 3.0 |
| TRBC1 | 74 | 0.076 | 4.5 |
| CD3D | 69 | 0.011 | 3.7 |
| HLA-DMA | 70 | 0.012 | 2.1 |

The results obtained for the individual genes are comparable to the % concordance of 69% obtained in multivariate classification with all the genes in example 2.

### Example 6

### Classification Performance of Individual Genes Measured by Q-PCR in NSCLC Samples

Each of the 23 genes from example 3 were evaluated for classification performance by using the algorithm applied to multivariate classification in NSCLC samples (Cox-SPCA) using single gene expression values instead of the first principal component.

After normalizing the expression values using the reference genes and performing a z-score, the expression levels for each individual gene were used to build a classifier as described in example 3. The risk score for each sample in the training set was obtained and the samples were assigned to GS+ or GS− based on different cutoffs. Performance of each cutoff was assessed by calculating the treatment HR associated with this cutoff in each GS+ and GS− group. The best cutoff per gene was determined individually by maximizing the interaction coefficient of the classification, that is maximizing the difference between treatment HR in GS+ and GS−. Table below shows treatment HR in GS+ and GS− obtained using this optimization process and the p-values associated with those HR.

TABLE 12

| | | GS+ p- | | GS− p- |
|---|---|---|---|---|
| Gene | GS+ HR | value | GS− HR | value |
| C4orf7 | 0.182 | 0.03 | 1.133 | 0.71 |
| CCL5 | 0.169 | 0.04 | 1.061 | 0.86 |
| JAK2 | 0.427 | 0.091 | 0.992 | 0.98 |
| IRF1 | 0.521 | 0.088 | 1.567 | 0.46 |
| CXCL9 | 0.166 | 0.027 | 1.040 | 0.91 |
| IL2RG | 0.244 | 0.056 | 1.162 | 0.66 |
| CXCL10 | 0.648 | 0.2 | 1.607 | 0.57 |
| SLC26A2 | 0.680 | 0.25 | 1.910 | 0.35 |
| CD86 | 0.479 | 0.13 | 1.159 | 0.7 |
| CD8A | 0.209 | 0.024 | 1.204 | 0.6 |
| UBD | 0.230 | 0.016 | 1.413 | 0.37 |
| GZMK | 0.086 | 0.0082 | 1.364 | 0.37 |
| GPR171 | 0.402 | 0.045 | 1.715 | 0.23 |
| PSCDBP | 0.340 | 0.025 | 1.514 | 0.28 |
| CXCL2 | 0.635 | 0.16 | 2.476 | 0.26 |
| ICOS | 0.585 | 0.13 | 2.122 | 0.2 |
| TRBC1 | 0.387 | 0.12 | 1.101 | 0.78 |
| TRA@; TRAJ17; TRDV2; | 0.288 | 0.026 | 1.413 | 0.36 |
| TRAC; TRAV20 | | | | |
| TARP; TRGC2 | 0.747 | 0.51 | 1.003 | 1 |
| ITK | 0.152 | 0.039 | 1.167 | 0.65 |
| CD3D | 0.217 | 0.033 | 1.202 | 0.59 |
| HLA-DMA | 0.394 | 0.17 | 1.094 | 0.79 |
| SLAMF7 | 0.354 | 0.029 | 1.222 | 0.63 |

### Example 7

### Classification Performance of Individual Genes Measured by Microarray in Melanoma Samples

Each of the 100 PS from example 1 were evaluated for univariate classification performance by using the algorithm applied to multivariate classification in melanoma samples using single gene expression values instead of the first principal component.

After normalizing the expression values (gcrma) and performing a z-score, the expression levels for each individual PS were used to build the classifier using all samples in training set. The t-test p-value for differential expression of each PS in the training set and the fold change of Responders vs Non-Responders was calculated. The probability of each sample in the training set being responder was obtained and the best cutoff was determined for each gene by maximizing the concordance with clinical label and the results are shown in the next table:

TABLE 13

| | Concordance | p-value t- | |
|---|---|---|---|
| Probeset | (%) | test | FC |
| 225996_at | 71 | 0.0002 | 0.2 |
| 205890_s_at | 75 | 0.0002 | 7.4 |
| 223575_at | 75 | 0.0002 | 0.3 |
| 232481_s_at | 73 | 0.0011 | 0.3 |
| 213793_s_at | 77 | 0.0004 | 0.4 |
| 217436_x_at | 77 | 0.0004 | 2.1 |
| 228400_at | 70 | 0.0025 | 0.4 |
| 204116_at | 73 | 0.0005 | 5.4 |
| 232375_at | 75 | 0.0005 | 2.4 |
| 244393_x_at | 70 | 0.0007 | 0.4 |
| 215806_x_at | 75 | 0.0004 | 3.6 |
| 221875_x_at | 75 | 0.0005 | 2.2 |
| 1555852_at | 79 | 0.0010 | 3.1 |
| 208729_x_at | 75 | 0.0007 | 2.4 |
| 204806_x_at | 75 | 0.0006 | 2.2 |
| 211144_x_at | 75 | 0.0006 | 3.4 |
| 222838_at | 73 | 0.0018 | 4.6 |
| 211911_x_at | 79 | 0.0008 | 2.4 |
| 208894_at | 71 | 0.0018 | 2.6 |
| 203915_at | 71 | 0.0023 | 6.5 |
| 226084_at | 79 | 0.0007 | 0.4 |
| 216920_s_at | 75 | 0.0010 | 3.1 |
| 236328_at | 75 | 0.0008 | 0.3 |
| 1562031_at | 77 | 0.0012 | 2.5 |
| 212671_s_at | 71 | 0.0018 | 3.9 |
| 204533_at | 68 | 0.0018 | 6.0 |
| 207795_s_at | 75 | 0.0009 | 3.0 |
| 217478_s_at | 73 | 0.0020 | 2.4 |
| 209606_at | 73 | 0.0014 | 3.3 |
| 201474_s_at | 71 | 0.0037 | 0.5 |
| 211796_s_at | 73 | 0.0019 | 5.3 |
| 204070_at | 71 | 0.0017 | 3.6 |
| 204556_s_at | 68 | 0.0031 | 0.4 |
| 1554240_a_at | 75 | 0.0012 | 2.9 |
| 235276_at | 71 | 0.0022 | 2.9 |
| 202659_at | 73 | 0.0018 | 2.1 |
| 210982_s_at | 71 | 0.0028 | 2.5 |
| 205758_at | 70 | 0.0020 | 6.5 |
| 211149_at | 66 | 0.0042 | 0.3 |
| 237515_at | 68 | 0.0024 | 0.4 |
| 210972_x_at | 68 | 0.0019 | 3.8 |
| 231229_at | 71 | 0.0018 | 0.4 |
| 208885_at | 68 | 0.0031 | 2.8 |
| 211339_s_at | 71 | 0.0022 | 3.2 |
| 235175_at | 73 | 0.0026 | 3.5 |
| 229391_s_at | 73 | 0.0037 | 3.3 |
| 214470_at | 64 | 0.0030 | 2.7 |
| 210915_x_at | 73 | 0.0031 | 4.5 |
| AFFX- | 71 | 0.0033 | 2.3 |
| HUMISGF3A/ | | | |
| M97935_MB_at | | | |
| 206082_at | 75 | 0.0027 | 3.1 |
| 228362_s_at | 73 | 0.0040 | 3.6 |
| 1562051_at | 63 | 0.0076 | 0.4 |
| 205097_at | 68 | 0.0028 | 0.4 |
| 229625_at | 70 | 0.0032 | 3.2 |
| 228532_at | 70 | 0.0044 | 2.4 |
| 222962_s_at | 71 | 0.0036 | 0.5 |
| 209774_x_at | 73 | 0.0032 | 2.9 |
| 238524_at | 73 | 0.0030 | 2.4 |
| 202643_s_at | 66 | 0.0034 | 2.1 |
| 232234_at | 73 | 0.0030 | 3.4 |
| 204897_at | 68 | 0.0044 | 2.4 |
| 232311_at | 70 | 0.0037 | 2.2 |
| 229543_at | 73 | 0.0051 | 3.3 |
| 202531_at | 71 | 0.0031 | 2.7 |
| 210606_x_at | 71 | 0.0028 | 2.8 |
| 207651_at | 75 | 0.0036 | 3.9 |
| 209813_x_at | 73 | 0.0028 | 2.7 |
| 228492_at | 64 | 0.0059 | 0.2 |
| 219551_at | 71 | 0.0031 | 2.4 |
| 1555759_a_at | 75 | 0.0031 | 2.4 |
| 205499_at | 66 | 0.0063 | 0.4 |
| 1552613_s_at | 66 | 0.0048 | 1.9 |
| 228316_at | 70 | 0.0041 | 0.5 |
| 210439_at | 70 | 0.0042 | 2.6 |
| 234907_x_at | 77 | 0.0029 | 2.2 |
| 211902_x_at | 70 | 0.0035 | 2.9 |
| 205685_at | 71 | 0.0049 | 2.5 |
| 213193_x_at | 73 | 0.0044 | 4.3 |
| 1552612_at | 70 | 0.0054 | 2.6 |
| 1552497_a_at | 70 | 0.0034 | 3.3 |
| 223593_at | 75 | 0.0068 | 0.4 |
| 200615_s_at | 71 | 0.0041 | 0.5 |
| 206666_at | 66 | 0.0050 | 4.1 |
| 204529_s_at | 70 | 0.0037 | 3.1 |
| 1563473_at | 66 | 0.0050 | 3.3 |
| 1553132_a_at | 73 | 0.0033 | 2.0 |
| 229390_at | 71 | 0.0064 | 3.2 |
| 213539_at | 68 | 0.0058 | 4.3 |
| 244061_at | 66 | 0.0043 | 2.8 |
| 209770_at | 68 | 0.0047 | 1.8 |
| 238587_at | 66 | 0.0088 | 1.9 |
| 207536_s_at | 71 | 0.0037 | 2.6 |
| 221081_s_at | 64 | 0.0070 | 2.8 |
| 209671_x_at | 71 | 0.0041 | 3.0 |
| 239012_at | 68 | 0.0069 | 2.3 |
| 229152_at | 68 | 0.0052 | 5.3 |
| 202644_s_at | 66 | 0.0065 | 2.1 |
| 238581_at | 71 | 0.0048 | 2.6 |
| 231577_s_at | 75 | 0.0065 | 2.7 |
| 204224_s_at | 64 | 0.0091 | 2.4 |

The results obtained for the individual PS are comparable to the % concordance of 68% obtained in multivariate classification with all the genes in example 1.

### REFERENCES

Dave S S, Wright G, Tan B et al. Prediction of survival in follicular lymphoma based on molecular features of tumor-infiltrating immune cells. N. Engl. J. Med. 2004; 351:2159-2169
Hu Z, Fan C, Oh D S et al. The molecular portraits of breast tumors are conserved across microarray platforms. BMC. Genomics 2006; 7:96.
Weigelt B, Hu Z, He X et al. Molecular portraits and 70-gene prognosis signature are preserved throughout the metastatic process of breast cancer. Cancer Res. 2005; 65:9155-9158.
Golub T, Slonim D, Tamayo P et al. Molecular classification of cancer: class discovery and class prediction by gene expression monitoring. Science 1999; 286: 531-536
Bair E, Tibshirani R. Semi-supervised methods to predict patient survival from gene expression data. PLoS Biology 2004; 2(4):511-522.
Tibshirani R, Hastie T, Narasimhan B et al. Diagnosis of multiple cancer types by shrunken centroids of gene expression. PNAS 2002; 99(10): 6567-6572
Harlin H, Meng Y, Peterson A C et al. Chemokine expression in melanoma metastases associated with CD8+ T-cell recruitment. Cancer Res. 2009; 69(7):3077-85. Epub 2009 Mar. 17
Wu H, Mao F, Olman V, Xu Y Hierarchical classification of functionally equivalent genes in prokaryotes. Nucleic Acids Res. 2007; 35(7):2125-40. Epub 2007 Mar. 11.
Van 't Veer L J, Dai H, van de Vijver M J, He Y D, Hart A A, Mao M, Peterse H L, van der Kooy K, Marton M J, Witteveen A T, et al. (2002)
Van 't Veer L J, Dai H, van de Vijver M J, He Y D, Hart A A, Mao M, Peterse H L, van der Kooy K, Marton M J, Witteveen A T, et al. (2002) Gene expression profiling predicts clinical outcome of breast cancer. Nature, 415(6871), 530-556.
Ginzinger D G., Gene quantification using real-time quantitative PCR: an emerging technology hits the mainstream Exp Hematol. 2002 June; 30(6):503-12. Review.
Balch C M. Cutaneous melanoma: prognosis and treatment results worldwide. Semin Surg Oncol. 1992 November-December; 8(6):400-14.
Weynants P, Lethé B, Brasseur F, Marchand M, Boon T. Expression of mage genes by non-small-cell lung carcinomas. Int J Cancer. 1994 Mar. 15; 56(6):826-9.
Gaugler B, Van den Eynde B, van der Bruggen P, Romero P, Gaforio J J, De Plaen E, Lethé B, Brasseur F, Boon T. Human gene MAGE-3 codes for an antigen recognized on a melanoma by autologous cytolytic T lymphocytes. J Exp Med. 1994 Mar. 1; 179(3):921-30.
Patard J J, Brasseur F, Gil-Diez S, Radvanyi F, Marchand M, François P, Abi-Aad A, Van Cangh P, Abbou C C, Chopin D, et al. Expression of MAGE genes in transitional-cell carcinomas of the urinary bladder. Int J Cancer. 1995 Feb. 20; 64(1):60-4.
Moore A, McCarthy L, Mills K H. The adjuvant combination monophosphoryl lipid A and QS21 switches T cell responses induced with a soluble recombinant HIV protein from Th2 to Th1. Vaccine. 1999 Jun. 4; 17(20-21):2517-27.
Gérard C M, Baudson N, Kraemer K, Bruck C, Garçon N, Paterson Y, Pan Z K, Pardoll D. Therapeutic potential of protein and adjuvant vaccinations on tumour growth. Vaccine. 2001 Mar. 21; 19(17-19):2583-9.
Maniatis et al., Molecular Cloning—A Laboratory Manual; Cold Spring Harbor, 1982-1989
Krieg A M, Davis H L. Enhancing vaccines with immune stimulatory CpG DNA. Curr Opin Mol Ther. 2001 February; 3(1):15-24. Review.
Ren J, Zheng L, Chen Q, Li H, Zhang L, Zhu H. Co-administration of a DNA vaccine encoding the prostate specific membrane antigen and CpG oligodeoxynucleotides suppresses tumor growth. J Transl Med. 2004 Sep. 9; 2(1):29.
Wu Z, Irizarry R A, Gentleman R, Martinez-Murillo F, Spencer F. A model-based background adjustment for oligonucleotide expression arrays. J Am Stat Ass. 2004; 99: 909-917

Appendix 1 - GCRMA-enabled, modified RefPlus R code

```
require(affyPLM)
pe <- read.table(“VR63933P_pe.txt”)
pe <- unstack(pe)
rq <- scan(“VR63933P_rq.txt ”)
gcrmaplus <- function (Future, gcrmapara, r.q, p.e, bg = TRUE)
{
  if (missing(r.q) & (missing(gcrmapara))) {
    stop(“Missing Reference Quantiles”)
  }
  if (missing(p.e) & (missing(gcrmapara))) {
    stop(“missing Probe Effects”)
  }
  if (!missing(gcrmapara)) {
    r.q = gcrmapara[[1]]
    p.e = gcrmapara[[2]]
    cat(“Use gcrmapara.\n”)
  }
  else {
    cat(“Use Reference.Quantiles and Probe.Effects.\n”)
  }
  if (bg == TRUE)
    Future <- bg.adjust.gcrma(Future)
  PM = pm(Future)
  pm(Future) <- normalize.quantiles2(PM, r.q)
  rm(PM)
  future <- gcrmaref.predict(Future, p.e)
  return(future)
}
gcrmaref.predict <- function (Future, p.e)
{
  PMindex <- pmindex(Future)
  PM <- log2(pm(Future))
  PM <- sweep(PM, 1, unlist(p.e))
  pm(Future) <- PM
  PMlist <- lapply(PMindex, function(x, y) intensity(y)[x,],
    Future)
  future <- t(sapply(PMlist, colMedians))
  colnames(future) <- sampleNames(Future)
  return(future)
}
normalize.quantiles2 <- function (X, Reference.Quantiles)
{
  apply(X, 2, function(x, y) y[rank(x)], Reference.Quantiles)
}
colMedians <- function (mat) rowMedians(t(mat))
```

We claim:

1. A method of characterising a patient as a responder or non-responder to a therapy comprising the steps of:
(a) analysing a patient derived sample for differential expression of the gene products of one or more genes of Table 1, and
(b) characterising the patient from which the sample was derived as a responder or non-responder, based on the results of step (a),
wherein the characterisation step is performed by reference or comparison to a standard or a training set or using an algorithm whose parameters were obtained from a standard or training set.

2. A method of treating a patient comprising the steps of:
(a) obtaining an analysis of a patient derived sample for differential expression of the gene products of one or more genes of Table 1, wherein the results characterise a patient as a responder or non-responder to an immunotherapeutic and wherein the characterisation step is performed by reference or comparison to a standard or a training set or using an algorithm whose parameters were obtained from a standard or training set; and
(b) selecting the patient for at least one administration of an appropriate immunotherapeutic if the patient is characterized as a responder to the immunotherapeutic.

3. A method of determining whether a patient is a responder or a non-responder to an immunotherapeutic comprising the steps of:
(a) obtaining a patient derived sample; and
(b) analysing the patient derived sample for differential expression of the gene products of one or more genes of Table 1, wherein the results determine whether the patient is characterised as a responder or non-responder to an immunotherapeutic and wherein the characterisation step is performed by reference or comparison to a standard or a training set or using an algorithm whose parameters were obtained from a standard or training set.

4. A method as claimed in any of claims 1 to 3 wherein the one or more genes of Table 1 are at least 63 genes listed in Table 1 or substantially all the genes specified in Tables 2, 5 or 7.

5. A method for characterising a patient as a responder or non-responder to therapy comprising analysing, in a patient-derived sample, a gene product recognised by one or more of the probe sets listed in Table 1, the target sequences of which are shown in Table 3,
wherein the characterisation step is performed by reference or comparison to a standard or a training set or using an algorithm whose parameters were obtained from a standard or training set.

6. A method as claimed in claim 5 wherein the one or more probe sets of Table 1 are at least 74 of the probe sets listed in Table 1 or all the probe sets for genes in Tables 2, 5 or 7.

7. A method as defined in any of claims 1, or 3 to 6 comprising the further step of identifying a patient as a responder, and selecting the patient for therapy.

8. A method according to any of claims 1 to 7, in which the standard is a patient-derived sample or samples from a patient or patients, respectively, having a known clinical outcome.

9. A method according to any of claims 1 to 8, wherein the therapy or treatment is cancer immunotherapy, preferably cancer immunotherapy for melanoma and/or lung cancer.

10. A method according to claim 9, wherein the cancer immunotherapy is MAGE.

11. A method according to claim 10, wherein the MAGE immunotherapy is MAGE A3 immunotherapy.

12. A method according to any of claims 1 to 11, wherein the one or more genes of Table 1 are at least 63, at least 68, at least 70, at least 75, at least 80 or substantially all the genes listed in Table 1 and/or any combination thereof.

13. A method according to any of claims 5 to 11, wherein the one or more probe sets of Table 1 are at least 74, at least 75, at least 80, at least 85, at least 90 or all the probe sets listed in Table 1 and/or any combination thereof.

14. A method according to any of claims 1 to 13, in which the one or more genes are upregulated in comparison to their normal expression.

15. A method according to any of claims 1 to 14, in which at least 80% of the genes are upregulated in comparison to their normal expression.

16. A method according to any of claims 1 to 15, further comprising the step of determining whether the gene products are upregulated and/or down-regulated.

17. A method according to claim 16, wherein a determination that the gene products are upregulated and/or downregulated indicates a responder.

18. A method according to any of claims 1 to 17 in which genes are immune related genes.

19. A method according to any preceding claim comprising use of a probe for the identification of the one or more gene products.

20. A method according to any preceding claim comprising use of a microarray kit or PCR for analysing gene expression.

21. Use of a gene list of at least 63 of the genes in Table 1 or data generated therefrom or at least 74 of the probe sets in Table 1 or data generated therefrom to perform an analysis of whether a patient will be a likely responder or non-responder to a therapy, such as cancer immunotherapy.

22. Use as claimed in claim 20 wherein the gene list comprises or consists of substantially all the genes or probe sets in Table 1.

23. A microarray comprising polynucleotide probes complementary and hybridisable to a sequence of the gene product of at least one gene selected from the genes listed in Table 1, in which polynucleotide probes or probe sets complementary and hybridisable to the genes of Table 1 constitute at least 50% of the probes or probe sets on said microarray.

24. A microarray comprising polynucleotide probes complementary and hybridisable to a sequence of the gene product of at least one gene selected from the genes listed in Table 1.

25. A microarray as claimed in claim 23 or claim 24 comprising polynucleotide probes complementary and hybridisable to a sequence of the gene product of the genes listed in Table 2.

26. A diagnostic kit comprising means for measuring the expression, for example probes hybridising to mRNA or cDNA gene products, of the one or more of the genes listed in Table 1 or of the gene products of the genes listed in Table 1 for performing the method of any one of claims 1 to 20.

27. A method of treating a patient characterised as a responder according to the method of claims 1 to 20 or use of the microarray of claims 23 to 25 or the diagnostic kit of claim 26, comprising administering a composition comprising a tumour associated antigen to the patient.

28. A composition comprising a tumour associated antigen for the treatment of patients determined to have, or characterised as, a responder according to the method of claims 1 to 20 or use of the microarray of claims 23 to 25 or the diagnostic kit of claim 26.

29. Use of a composition comprising a tumour associated antigen in the preparation of a medicament for the treatment of patients determined to have or characterised as a responder according to the method of claims 1 to 20 or use of the microarray of claims 23 to 25 or the diagnostic kit of claim 26.

30. A method, composition or use according to any one of claims 27 to 29, in which the tumour associated antigen is a MAGE antigen.

31. A method, composition or use according to any one of claims 27 to 30, in which the composition further comprises an adjuvant.

32. A solid surface to which are linked to a plurality of detection agents of at least 63 of the genes listed in Table 1, which detection agents are capable of detecting the expression of the genes or polypeptides encoded by the genes.
